(19) United States
(12) Patent Application Publication
(10) Pub. No.: US 2007/0026393 A1
(43) Pub. Date: Feb. 1, 2007

(54) Detection of variations in the dna methylation profile

(76) Inventors: Kurt Berlin, Stahnsdorf (DE); Christian Piepenbrock, Berlin (DE); Alexander Olek, Berlin (DE)

Correspondence Address: Kriegsman & Kriegsman, 665 Franklin Street, Framingham, MA, 01702, US

(21) Appl. No.: 10/240,970

(22) PCT Filed: Apr. 6, 2001
(86) PCT No.: PCT/DE01/01486

§ 371 Date: Jul. 11, 2003

(30) Foreign Application Priority Data
Apr. 6, 2000 (DE) 100190588

(51) Int. Cl. C12Q 1/68; C07H 21/04

(52) U.S. Cl. 435/6; 536/24.3

(57) ABSTRACT

The invention describes a set of oligonucleotides as probes for the detection of relevant variations of DNA methylation in a target group of genes, the use thereof for the detection of gene variants with respect to DNA methylation, a medical device which uses a set of oligonucleotides, a method for investigating the methylation state of an individual as well as a method for creating a model for evaluating the probability of occurrence of a health problem of an individual. Such disorders can be: undesired drug interactions cancer diseases CNS malfunctions, damage or disease symptoms of aggression or behavioral disturbances clinical, psychological and social consequences of brain lesions psychotic disturbances and personality disorders dementia and/or associated syndromes cardiovascular disorder, malfunction and damage malfunction, damage or disorder of the gastrointestinal tract malfunction, damage or disorder of the respiratory system lesion, inflammation, infection, immunity and/or convalescence malfunction, damage or disease of the body as an abnormality in the development process malfunction, damage or disorder of the skin, the muscles, the connective tissue or the bones endocrine and metabolic malfunction, damage or disorder headaches or sexual malfunction.

The invention concerns a set of oligonucleotides as probes for the detection of relevant variations of DNA methylation in a target group of genes, the use thereof for the detection of gene variants with respect to DNA methylation, a medical device which uses a set of oligonucleotides, a method for the investigation of the methylation state of an individual as well as a method for the construction of a model for evaluating the probability of occurrence of a health problem in an individual.

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also correlated with a modified methylation pattern of individual genes or of the genome. The present invention describes sets of oligomers and a method for the detection of relevant variations of the DNA methylation in a target group of genes, which are associated with adverse events for patients/individuals or are associated with specific disorders.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used me-method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

With just a few exceptions (e.g. Zechnigk, M. et al., Eur. J. Hum. Gen. 1997, 5, 94-98), the bisulfite technique has only been applied in research up to now. However, short, specific segments of a known gene are always amplified after a bisulfite treatment and either completely sequenced (Olek, A. und Walter, J., Nat. Genet. 1997, 17, 275-276) or individual cytosine positions are detected by a “primer extension reaktion” (Gonzalgo, M. L. and Jones, P. A., Nucl. Acids Res. 1997, 25, 2529-2531, WO Patent 95-00669) or an enzyme step (Xiong, Z. and Laird, P. W., Nucl. Acids. Res. 1997, 25, 2532-2534). Detection by hybridization has also been described (Olek et al., WO 99 28498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97 46705, WO 95 15373 and WO 45560.

An overview of the state of the art in oligomer array production can be derived also from a special issue of Nature Genetics which appeared in January 1999 (Nature Genetics Supplement, Volume 21, January 1999), the literature cited therein and U.S. Pat. No. 5,994,065 on methods for the production of solid supports for target molecules such as oligonucleotides in the case of reduced nonspecific background signal.

Probes with multiple fluorescent labels are used for scanning an immobilized DNA array. The fluorescence of the hybridized probes is detected, for example, by means of confocal optics.

More recent methods for the detection of mutations, which also can be used, in principle but with several modifications, for methylation analyses after the bisulfite treatment of DNA samples, are listed below:
As a special case of sequencing, single-base primer extension (Genetic Bit Analysis) is worthy of mention (Head, S R., Rogers, Y H., Parikh K., Lan, G., Anderson, S., Goelet, P., Boycejacino M T., Nucleic Acids Research. 25(24): 5065-5071, 1997; Picoult-Newberg, L., Genome Res. 9(2): 167-174, 1999). A combined amplification and sequencing is described in U.S. Pat. NO. 5,928,906, in which a base-specific termination on matrix molecules is utilized. Another method utilizes a ligase/polymerase reaction for the identification of nucleotides (U.S. Pat. No. 5,952,174).

Genomic DNA is obtained from DNA of cells, tissue or other test samples by standard methods. This standard methodology is found in references such as Fritsch and Maniatis, eds., Molecular Cloning: A Laboratory Manual, 1989.

At present, it is not state of the art to investigate large quantities of samples relative to a multiple number of methylation positions that are important for diseases.

The object of the present invention is to make up sets of oligomer probes that bind to genes which are of importance relative to adverse events for patients or relative to specific groups of diseases, so that comprehensive prognostic information will be possible for the patient in question by analysis of the respective methylation state. The data detected in this way are combined to make up methylation patterns. In addition, a method will be created which makes possible to a great extent the analysis of methylation positions with the use of the above-mentioned oligomer probes.

The object is [solved] according to the invention by a set of nucleotide probes and a method for the investigation of the methylation profile of a patient or individual. The presence or absence of the relevant methylation variants from the target group of genes is detected by means of the set of oligonucleotide probes and/or by means of the method. A diagnosis is made from the methylation pattern by comparing it to methylation patterns in a database, which have already been assigned to specific phenotypes, prognoses or effects on the patient.

This object is solved according to the invention by a set of nucleotide probes according to one or more of the claims. In addition, to solve the object, a method or a device can be utilized, which contains a set of the above-mentioned nucleotide probes. Advantageous embodiments of the invention are characterized in the respective subclaims.

The set of oligonucleotide probes according to the invention or a device which contains this set preferably serves for the prognosis and planned treatment of patients who suffer from the consequences of subsequent adverse events or in whom there is a risk of the occurrence of the following adverse events:
undesired drug interactions
cancer diseases
CNS malfunctions, damage or disease
symptoms of aggression or behavioral disturbances
clinical, psychological and social consequences of brain lesions
psychotic disturbances and personality disorders
dementia and/or associated syndromes
cardiovascular disorder, malfunction and damage
malfunction, damage or disorder of the gastrointestinal tract
malfunction, damage or disorder of the respiratory system
lesion, inflammation, infection, immunity and/or convalescence
malfunction, damage or disorder of the body as an abnormality in the development process
malfunction, damage or disorder of the skin, the muscles, the connective tissue or the bones
endocrine and metabolic malfunction, damage or disorder
headaches or
sexual malfunction.

This list is not conclusive and it is clear to the person of average skill in the art that the concept of the invention is applicable to any disease or malfunction of an organism or individual.

The oligomer probes comprise sequences which bind to genes that are associated with these adverse events. The oligomer probes bind to sequences as they are present after a treatment of the DNA sample which converts unmethylated cytosine to uracil. The genes are listed in detail in the claims. According to the invention, these genes belong to at least one of the following protein functions: enzyme, transport, storage, structure, immunity, neuronal transmission, growth and differentiation.

The method will be described in the following for an evaluation of whether adverse events will occur or will probably occur in a patient, an individual or a population, by using the set of oligomer probes. In the first step, a DNA sample is taken from the patient or individual, in whom an adverse event was diagnosed not and from a control group in whom the event was diagnosed. In the second step of the method, the DNA samples, which have been pretreated by means of the solution of a bisulfite, hydrogen sulfite or disulfite, are hybridized with a set of oligonucleotides as probes, which bind to the sequences in which a cytosine methylation is potentially present after bisulfite treatment within the respective target group of genes, in order to detect relevant gene variants with respect to DNA methylation. A hybridization pattern results, which is translated into a methylation pattern of the genes of the respective DNA sample in the third step of the method.

The procedure would be the same if one wanted to construct a model for evaluating risks for patients or patient groups.

According to the invention, these genes belong to at least one of the following protein functions: enzyme, transport, storage, structure, immunity, neuronal transmission, growth and differentiation. Said genes are associated with a multiple number of adverse events, including:
undesired drug interactions
cancer diseases
CNS malfunctions, damage or disease
symptoms of aggression or behavioral disturbances
clinical, psychological and social consequences of brain lesions
psychotic disturbances and personality disorders
dementia and/or associated syndromes
cardiovascular disorder, malfunction and damage
malfunction, damage or disorder of the gastrointestinal tract
malfunction, damage or disorder of the respiratory system
lesion, inflammation, infection, immunity and/or convalescence
malfunction, damage or disorder of the body as an abnormality in the development process
malfunction, damage or disorder of the skin, the muscles, the connective tissue or the bones
endocrine and metabolic malfunction, damage or disorder
headaches or
sexual malfunction.

The oligonucleotide probes are characterized by the fact that they are complementary to the DNA sequences of the target group of genes or correspond to them as they are present after a chemical treatment which converts unmethylated cytosines to uracil, preferably a treatment with sodium bisulfite.

In the last step of the method, the frequency of alleles with different methylation patterns is calculated and compared with the frequencies of alleles in patients and individuals with adverse events and the corresponding control group.

Preferably, at least one of the steps of the method will be conducted with a computer.

In a preferred variant of the method, the methylation pattern of an individual is compared with the entries already present in the database or with a model derived therefrom, in order to evaluate the risk of the occurrence of adverse events.

The set of oligonucleotides is preferably comprised of oligonucleotides, which are arranged on a support at known loci in a rectangular or hexagonal grid. This support is comprised of silicon, glass, polystyrene, aluminum, steel, iron, copper, nickel, silver, or gold.

A technical medical device which contains said set of oligonucleotides is preferably used for the detection of gene variants relative to methylation and different gene expression.

In a preferred variant of the method, said set of oligonucleotides or a device that contains them is used for predicting probable therapeutic consequences or adverse events as a consequence of a therapeutic intervention or as a consequence of taking specific medications.

In a preferred variant of the method, said set of oligonucleotides or a device is used for predicting probable symptoms when the above-listed adverse events occur and for predicting the probability of the occurrence of consequential disorders or other symptoms.

In another preferred variant of the method, said set of oligonucleotides or a device is used for the following objectives:
Development of new strategies for therapeutic intervention and in clinical studies
Modeling and evaluating the effects of disorders and providing measures for predicting health for individuals, ethnic groups, patient groups, and populations
Optimizing therapeutic intervention

Another subject of the present invention is a kit for conducting an assay, with which the risk of a patient or individual of being subject to adverse events can be estimated. This kit comprises possibilities for testing for the presence or absence of relevant variations with respect to DNA methylation of the above-listed genes in a sample of genomic DNA. Reagents for use in the detection method are also included, such as well script, which describes the probability of a patient or individual of experiencing to undesired events.

The object of the invention is thus solved by a set of oligonucleotides as probes for the detection of relevant variations of DNA methylation in a target group of genes, characterized in that the oligonucleotides are complementary to the DNA sequence of the target group of genes or correspond to them as they are present after a chemical treatment which converts unmethylated cytosines to uracil, wherein the target group essentially comprises genes that are associated with a health problem of an individual.

It is preferred according to the invention that the set of oligonucleotides is characterized in that the health problems are: undesired drug interactions; cancer; CNS malfunctions, damage or disease; symptoms of aggression or behavioral disturbances; clinical, psychological and social consequences of a brain lesion; dementia and/or associated syndromes; psychotic disturbances and personality disorders; cardiovascular disease, malfunction or damage; malfunction, damage or disease of the gastrointestinal tract; damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction; headaches; sexual malfunctions.

It is additionally preferred that the set of oligonucleotides is characterized in that the genes that are associated with undesired drug interactions are selected from Table 1, the genes that are associated with cancer are selected from Table 2, the genes associated with symptoms and consequences of CNS malfunction are selected from Table 3, the genes associated with symptoms of aggression or behavioral disturbances are selected from Table 4, the genes associated with the consequences of clinical, psychological and social consequences of a brain lesion are selected from Table 5, the syndromes associated with dementia and/or associated syndromes are selected from Table 6, the genes that are associated with psychotic disturbances and personality disorders are selected from Table 7, the genes associated with cardiovascular disease, malfunction or damage are selected from Table 8, the genes associated with malfunction, damage or disease of the gastrointestinal tract are selected from Table 9, the genes associated with malfunction, damage or disease of the respiratory system are selected from Table 10, the genes associated with lesion, inflammation, infection, immunity and/or convalescence are selected from Table 11, the genes associated with malfunction, damage or disease of the body as a consequence of an abnormality in the development process are selected from Table 12, the genes associated with a malfunction, damage or disease of the skin, the muscles, the connective tissue or the bones are selected from Table 13, the genes associated with endocrine and metabolic malfunction, damage or disease are selected from Table 14, the genes that are associated with headaches are selected from Table 15, and the genes that are associated with sexual malfunctions are selected from Table 16.

According to the invention, a set of oligonucleotides is preferred, in which oligonucleotides with up to 5% of the listed genes are not included.

In particular, a set of oligonucleotides is preferred, in which oligonucleotides with at least 95% of the listed genes are included, together with a limited number of additional, unlisted oligonucleotides.

A set of oligonucleotides is advantageous, in which up to 5% of the corresponding oligonucleotides of the listed genes are replaced by a complete set of 25% of other unlisted oligonucleotides.

In addition, a set of oligonucleotides for a target group of genes is advantageous, in which the chemically pretreated DNA sequence of the genes to be detected coincides at least up to 95% with the correspondingly pretreated DNA sequence of the genes from the above list.

A set of oligonucleotides is particularly advantageous, wherein the chemical pre-treatment is conducted by means of the solution of a bisulfite, hydrogen sulfite or disulfite.

A set of oligonucleotides that consists of a subgroup of the target group of genes is particularly advantageous.

According to the invention, a set of oligonucleotides is preferred in which the oligonucleotides are arranged on a support at known loci in a rectangular or hexagonal grid.

In addition, a set of oligonucleotides is preferred, wherein the oligonucleotides are arranged on a support, which is comprised of silicon, glass, polystyrene, aluminum, steel, iron, copper, nickel, silver or gold.

A set is particularly preferred, wherein the oligonucleotide probes are labeled via their mass, electrostatics, charge or fluorescence, or are labeled with radionuclides.

In particular, a set of oligonucleotides is preferably used in a biological investigation for the detection of said gene variants relative to DNA methylation.

A technical medical device is advantageous, which contains a set of oligonucleotides according to the invention, for use in an investigation for the detection of said gene variants, particularly as an indication of a higher risk of a patient or individual of developing symptoms and consequential signs of cancer or as an indication for a higher risk of the development of CNS malfunction, damage or disorder or for the patient or individual to experience symptoms and consequences of CNS malfunction, damage or disorder.

A technical medical device is particularly advantageous, which contains a set of oligonucleotides according to the invention, for use in an investigation for the detection of varying gene expression and/or for the prognosis and for the management of patients who suffer from the risk of developing symptoms and consequential signs of cancer and/or for use in an investigation of whether a patient or individual may have developed CNS malfunction, damage or disorder or whether it is probable that the patient or individual will experience the symptoms and consequences of CNS malfunction, damage or disorder.

According to the invention, a method for the investigation of the DNA methylation profile of a patient or an individual, which detects the presence or lack of the relevant methylation variants from the target group of genes is preferred in which a chemically pretreated nucleic acid sample of said patient or individual is hybridized to a set of oligonucleotides according to the invention and relating the hybridization pattern with the variations.

Particularly preferred is the use of a set of oligonucleotides according to the invention or a device according to the invention for the prognosis and/or planned treatment in patients, who suffer from a health problem or in whom the risk of onset of a health problem exists.

The use according to the invention is particularly advantageous for the prognosis and/or planned treatment for patients who suffer from the consequences of undesired drug interactions or in whom the risk of the occurrence of undesired drug interactions exists, who suffer from the risk of developing symptoms and consequential signs of cancer, who are affected by an increased risk of CNS malfunction, damage or disorder, who suffer from the consequences of symptoms of aggression or behavioral disturbances or in whom exists the risk of the occurrence of symptoms of aggression or behavioral disturbances, who suffer from the consequences of clinical, psychological and social consequences of a brain lesion or in whom exists the risk of the occurrence of consequences of clinical, psychological and social consequences of a brain lesion, who suffer from dementia and/or associated syndromes or in whom exists the risk of the occurrence of dementia and/or associated syndromes, who suffer from symptoms and consequences of psychotic disorders and personality disturbances or in whom the risk exists of the occurrence of psychotic disorders and personality disturbances, who suffer from symptoms or consequences of cardiovascular disease, malfunction or damage or in whom the risk exists of the occurrence of symptoms or consequences of cardiovascular disease, malfunction or damage, who suffer from symptoms and consequences of the malfunction, damage or disease of the gastrointestinal tract or in whom the risk exists of the occurrence of symptoms and consequences of the malfunction, damage or disease of the gastrointestinal tract, who experience clinical or social consequences that result from the malfunction, damage or disease of the respiratory system or in whom the risk exists of clinical or social consequences that result from malfunction, damage or disease of the respiratory system, who suffer from the symptoms and consequences of lesion, inflammation, infection, immunity and/or convalescence or in whom the risk exists of the occurrence of symptoms and consequences of lesion, inflammation, infection, immunity and/or convalescence, who suffer from the consequences of malfunction, damage or disorder of the body as a consequence of an abnormality in the development process or in whom the risk exists of the occurrence of malfunction, damage or disorder of the body as a consequence of an abnormality in the development process, who suffer from the consequences of a malfunction, damage or disorder of the skin, the muscles, the connective tissue or the bones or in whom the risk exists of the occurrence of a malfunction, damage or disorder of the skin, the muscles, the connective tissue or the bones, who suffer from the consequences of endocrine and metabolic malfunction, damage or disorder or in whom the risk exists of the occurrence of endocrine and metabolic malfunction, damage or disorder, who suffer from the consequences of headaches or in whom the risk exists of the occurrence of headaches, and/or who suffer from the consequences of sexual malfunctions or in whom the risk exists of the occurrence of sexual malfunctions.

The use of a set of oligonucleotides according to the invention or of a device according to the invention is preferred for predicting the probable therapeutic consequences of undesired drug interactions as a consequence of a therapeutic intervention and/or for predicting the probable therapeutic consequences and adverse results as a consequence of a therapeutic intervention.

In addition, the use of a set of oligonucleotides according to the invention or a device according to the invention is preferred for predicting probable therapeutic risks or of undesired drug interactions as a consequence of taking specific medications.

The use of a set of oligonucleotides according to the invention or of a device according to the invention is particularly preferred for predicting the probable symptoms if undesired drug interactions occur and the probability of the occurrence of consequential disorders or other symptoms and/or for predicting probable patterns of disease symptoms and the probability of the occurrence of consequential disorders or other symptoms.

The use of a set of oligonucleotides according to the invention or a device according to the invention is advantageous for the development of new strategies in therapeutic intervention and in clinical studies and/or for the prognosis or management of patients, who suffer from developing symptoms of aggression or behavioral disturbances or who belong to risk groups for said disturbances, and/or for modeling and evaluating the effects of disorders and providing precautionary measures health measures for individuals, ethnic groups, patient groups, and populations, and/or for generating a model in order to estimate the risk for individuals, ethnic groups, patient groups, and populations, for developing symptoms and consequential signs of cancer, and/or for generating a model for evaluating the risk of the development of symptoms and consequential signs of CNS malfunction, damage or disease, and/or for optimizing the therapeutic intervention.

A method is particularly advantageous for creating a model for evaluating whether a health problem will occur or will probably occur in a patient, an individual, or ethnic groups, which comprises the following steps:
a) a DNA sample is taken from the patient or individual, in whom a health problem has been diagnosed;
b) DNA samples are taken from a control group of individuals in whom the presence of this health problem has been concluded diagnostically;
c) the samples obtained in a) and b) are analyzed to determine the DNA methylation variation with the target group of genes according to the invention;
d) the frequency of alleles with different methylation patterns in samples from a) and b) is calculated;
e) the frequencies of the alleles in the samples from a) and b) are compared,
f) a statistical analysis is conducted of the results obtained under e) in order to obtain a model for evaluating the risk of the occurrence of health problems.

A method according to the invention is preferred, wherein the health problem is selected from: undesired drug interactions; cancer; symptoms and consequential signs of CNS malfunction, damage or disorder; developing symptoms of aggression or behavioral disturbances; consequences of clinical, psychological and social consequences of a brain lesion; dementia and/or associated syndromes; psychotic disturbances and personality disorders; symptoms or consequences of cardiovascular disorder, malfunction or damage; symptoms and consequences of malfunction, damage or disease of the gastrointestinal tract; malfunction, damage or disease of the respiratory system; symptoms and consequences of lesion, inflammation, infection, immunity and/or convalescence, malfunction, damage or disease of the body as a consequence of an abnormality in the development process; malfunction, damage or disorder of the skin, the muscles, the connective tissue or the bones; endocrine and metabolic malfunction, damage or disorder; headaches; sexual malfunctions.

A method is of advantage for evaluating whether a specific individual bears the risk of the occurrence of a health problem, wherein the methylation profile is compared with the model constructed according to the invention.

A method is particularly of advantage according to which at least one of the steps is conducted by a computer.

In addition, a configured assay [hit] is advantageous for utilizing the estimation of the risk of a patient or individual of experiencing adverse events and/or health problems, said kit containing:
a) Possibilities for testing for the presence or absence of key relevant polymorphic DNA variations of the core group of genes according to the invention in a sample of genomic DNA.
b) Reagents for use in the detection method.
c) A script, which describes the probability of a patient or individual of experiencing adverse events and/or health problems.

A configured assay [kit] is particularly advantageous for utilizing the estimation of the risk of a patient or an individual of experiencing adverse events and/or health problems, characterized in that the adverse event or the health problem is selected from the following:
undesired drug interactions; cancer; symptoms and consequential signs of CNS malfunction, damage or disorder; developing symptoms of aggression or behavioral disturbances;
consequences of clinical, psychological and social consequences of a brain lesion; dementia and/or associated syndromes; psychotic disturbances and personality disorders; symptoms or consequences of cardiovascular disorder, malfunction or damage; symptoms and consequences of malfunction, damage or disease of the gastrointestinal tract; malfunction, damage or disease of the respiratory system;

symptoms and consequences of lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disorder; headaches; sexual malfunctions.

LENGTHY TABLE REFERENCED HERE
US20070026393A1-20070201-T00001
Please refer to the end of the specification for access instructions.

LENGTHY TABLE REFERENCED HERE
US20070026393A1-20070201-T00002
Please refer to the end of the specification for access instructions.

LENGTHY TABLE REFERENCED HERE
US20070026393A1-20070201-T00003
Please refer to the end of the specification for access instructions.

LENGTHY TABLE REFERENCED HERE
US20070026393A1-20070201-T00004
Please refer to the end of the specification for access instructions.

LENGTHY TABLE REFERENCED HERE
US20070026393A1-20070201-T00005
Please refer to the end of the specification for access instructions.

LENGTHY TABLE REFERENCED HERE
US20070026393A1-20070201-T00006
Please refer to the end of the specification for access instructions.

LENGTHY TABLE REFERENCED HERE
US20070026393A1-20070201-T00007
Please refer to the end of the specification for access instructions.

LENGTHY TABLE REFERENCED HERE
US20070026393A1-20070201-T00008
Please refer to the end of the specification for access instructions.

LENGTHY TABLE REFERENCED HERE
US20070026393A1-20070201-T00009
Please refer to the end of the specification for access instructions.

LENGTHY TABLE REFERENCED HERE
US20070026393A1-20070201-T00010
Please refer to the end of the specification for access instructions.

LENGTHY TABLE REFERENCED HERE
US20070026393A1-20070201-T00011
Please refer to the end of the specification for access instructions.

LENGTHY TABLE REFERENCED HERE
US20070026393A1-20070201-T00012
Please refer to the end of the specification for access instructions.

LENGTHY TABLE REFERENCED HERE
US20070026393A1-20070201-T00013
Please refer to the end of the specification for access instructions.

LENGTHY TABLE REFERENCED HERE
US20070026393A1-20070201-T00014
Please refer to the end of the specification for access instructions.

LENGTHY TABLE REFERENCED HERE
US20070026393A1-20070201-T00015
Please refer to the end of the specification for access instructions.

LENGTHY TABLE REFERENCED HERE
US20070026393A1-20070201-T00016
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070026393A1) An electronic copy of the table will also be available from the USPTO upon request and payment of the fee set forth in 37 CFR 1.19(b)(3).

1. A set of oligonucleotides as probes for the detection of relevant variations of 5 DNA methylation in a target group of genes, hereby characterized in that the oligonucleotides are complementary to the DNA sequence of the target group of genes or correspond to them, as they are present after a chemical treatment, which converts unmethylated cytosines to uracil, wherein the target group essentially comprises genes that are associated with a health problem of an individual.

2. The set of oligonucleotides according to claim 1, further characterized in that the health problems are undesired drug interactions; cancer; CNS malfunctions, damage or disease, symptoms of aggression or behavioral disturbances; clinical, psychological and social consequences of a brain lesion; dementia and/or associated syndromes; psychotic disturbances and personality disorders; cardiovascular disorder, malfunction or damage malfunction, damage or disease of the gastrointestinal tract; damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction; headaches or sexual malfunctions.

3. The set of oligonucleotides according to one of claims 1 or 2, further characterized in that the genes associated with the undesired drug interactions are selected from:

| | HUGO | Protein- |
|---|---|---|
| List of genes | gene symbol | function |
| 5-Adenosyl homocysteine hydrolase | | 1 |
| Acetoacetyl 1-CoA-thiolase | ACAT1 | 1 |
| Acetoacetyl 2-CoA-thiolase | ACAT2 | 1 |
| Acetyl CoA acyltransferase | ACAA | 1 |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Actin, alpha, cardiac | ACTC | 3 |
| Actin, alpha, skeletal | ACTA1 | 3 |
| Actin, alpha, smooth, aortic | ACTA2 | 3 |
| Actin, beta | ACTB | 3 |
| Actin, gamma 2 | ACTG2 | 3 |
| Acyl CoA dehydrogenase, short chain | ACADS | 1 |
| Adenine phosphoribosyltransferase | APRT | 2 |
| Adenosine deaminase | ADA | 1 |
| Adenosine monophosphate deaminase | AMPD | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylate kinase | AK1 | 1 |
| Adenylate transferase | | 1 |
| Adenylosuccinate lyase | ADSL | 1 |
| ADP-ribosyltransferase | ADPRT | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Adrenoleukodystrophy gene | ALD | 1 |
| Albumin, ALB | ALB | 2 |
| Alkaptonuria gene | AKU | 6 |
| Alpha I acid glycoprotein | AAG, AGP | 2 |
| Alpha-1-antitrypsin | PI | 1 |
| Alpha-2-antiplasmin | PLI | 1 |
| Alpha-amylase | | 1 |
| Alpha-fetoprotein | AFP | 6 |
| Alpha-glucosidase, neutral AB | GANAB | 1 |
| Alpha-glucosidase, neutral C | GANC | 1 |
| Aminomethyltransferase | AMT | 1 |
| Aminopeptidase P | XPNPEP2 | 1 |
| Amyloid beta (A4) precursor protein-binding, | APBB1 | 5 |
| APBB1 | | |
| Amyloid beta (A4) precursor protein | APP | 5 |
| Androgen binding protein | ABP | 2 |
| Androgen receptor | AR | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Annexin I | ANX | 1 |
| Apurinic endonuclease | APE | 1 |
| Arginine vasopressin | AVP | 5 |
| Arginine vasopressin receptor 1A | AVPR1A | 5 |
| Arginine vasopressin receptor 1B | AVPR1B | 5 |
| Arginine vasopressin receptor 2 | AVPR2 | 5 |
| Aryl hydrocarbon receptor | AHR | 2 |
| Arylsulfatase E | ARSE | 1 |
| Aspartate transcarbamoylase | | 1 |
| Ataxia teiangiectasia gene, AT | ATM | 6 |
| ATP cobalamin adenoxyltransferase | | 1 |
| ATP sulphurylase | atpsk2 | 1 |
| ATP/ADP translocase | | 1 |
| Atriai natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| BCL2-associated X protein | BAX | 6 |
| Benzodiazepine receptor | | 5 |
| Beta-endorphin receptor | | 5 |
| Bile acid coenzyme A: amino acid N- | BAAT | 1 |
| acyltransferase | | |
| Bile salt export pump | BSEP, PFIC2 | 2 |
| Bile salt-stimulated lipase | CEL | 1 |
| Billrubin UDP-glucuronosyltransferase | | 1 |
| Biliverdin reductase | | 2 |
| Bleomycin hydrolase | BLMH | 1 |
| Bradykinin receptor B1 | | 4 |
| Bradykinin receptor B2 | | 4 |
| Breakpoint cluster region | BCR | 6 |
| Breast cancer 1 | BRCAI | 6 |
| Breast cancer 2 | BRCA2 | 6 |
| Brush border guanylyl cyclase | | 1 |
| Butyrylcholinesterase | BCHE | 1 |
| Ca(2+) transporting ATPase, fast twitch | ATP2A1 | 2 |
| Ca(2+) transporting ATPase, slow twitch | ATP2A2 | 2 |
| Calcineurin A1 | CALNA1 | 4 |
| Calcineurin A2 | CALNA2 | 4 |
| Calcineurin A3 | CALNA3 | 4 |
| Calcineurin B | | |
| Calcitonin receptor/Calcitonin gene-related | CALCR | 5 |
| peptide receptor | | |
| Calcium channel, voltage-dependent, alpha 1F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha- | CACNA1C | 5 |
| 1C | | |
| Calcium channel, voltage-dependent, Alpha- | CACNA1D | 5 |
| 1D | | |
| Calcium channel, voltage-dependent, Alpha- | CACNA1E | 5 |
| 1E (CACNLIA6) | | |
| Calcium channel, voltage-dependent, Alpha- | CACNA2 | 5 |
| 2/delta | | |
| Calcium channel, voltage-dependent, Beta 1 | CACNBI | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Canalicular multispecific organic anion | CMOAT | 2 |
| transporter | | |
| Cannabinoid receptor | CNR1 | 5 |
| Carbamoylphosphate synthetase 1 | CPS1 | 1 |
| Carbamoylphosphate synthetase 2 | CPS2 | 1 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carnitine transporter protein | CDSP, SCD | 2 |
| Carnosinase | | 5 |
| Cartilage-hair hypoplasia gene | CHH | 5 |
| Catalase | CAT | 4 |
| Catechol-o-methyltransferase | COMT | 1 |
| Catenin, beta | CTNNBL | 6 |
| Cell adhesion molecule, vascular, VCAM | VCAM1 | 6 |
| Cholecystokinin | CCK | 5 |
| Cholecystokinin B receptor | CCKBR | 5 |
| Cholesterol ester transfer protein | CETP | 2 |
| Choline acetyltransferase | CHAT | 1 |
| CoA transferase | | 1 |
| Colony-stimulating factor 1 | CSF1 | 6 |
| Colony-stimulating factor 2 | CSF2 | 6 |
| Colony-stimulating factor 3 | CSF3 | 6 |
| Colony-stimulating factor 3 receptor | CSF3R | 6 |
| Complex V | MTATP6 | 1 |
| Coproporphyrinogen oxidase | CPO | 1 |
| Cortico-steroid binding protein | | 2 |
| Corticosteroid nuclear receptor | | 4 |
| Corticotrophin-releasing hormone receptor | CRHR1 | 2 |
| Creb binding protein | CREBBP | 6 |
| Crystallin, alpha A | CRYAA | 3 |
| Crystallin, alpha B | CRYAB | 3 |
| Crystallin, beta B2 | CRYBB2 | 3 |
| Crystallin, gamma A | CRYGA | 3 |
| Cu2+ transporting ATPase alpha polypeptide | ATP7A | 1 |
| Cu2+ transporting ATPase beta polypeptide | ATP7B | 1 |
| Cyclic AMP response element binding protein | CREB | 6 |
| Cyclic AMP response element modulator | CREM | 6 |
| Cyclic AMP-dependent protein kinase | PKA | 1 |
| Cyclic nucleotide phosphodiesterase 1B | PDE1B | 1 |
| Cyclic nucleotide phosphodiesterase I B I | PDEIB1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin F | CCNF | 6 |
| Cyclin-dependent kinase inhibitor 1 A (P21, | CDKNIA | 6 |
| cipi) | | |
| Cyclooxygenase 1 | COX1 | 1 |
| Cyclooxygenase 2 | COX2 | 1 |
| Cyclophilin | | 4 |
| CYPL1A1 | CYPl1A1 | 1 |
| CYPlIB1 | CYPI1B1 | 1 |
| CYPL1B2 | CYPl1B2 | 1 |
| CYPI7 | CYP17 | 1 |
| CYP19 | CYPI9 | 1 |
| CYPlA1 | CYPlA1 | 1 |
| CYPIA2 | CYPlA2 | 1 |
| CYPIBL | cyplb1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2CI8 | CYP2C18 | 1 |
| CYP2CI9 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2EI | 1 |
| CYP2F1 | CYP2FI | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4AI1 | GYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystic fibrosis transmembrane conductance | CFTR | 5 |
| regulator, CFTR | | |
| Cytidine deaminase | CDA | 1 |
| Cytidine-5-prime-triphosphate synthetase | CTPS | 1 |
| Cytokine-suppressive antiinflammatory drug- | CSBPL | 4 |
| binding protein I | | |
| Cytokine-suppressive antiinflammatory drug- | CSBP2 | 4 |
| binding protein 2 | | |
| Deoxycytidine kinase DCK | | 1 |
| Deoxyuridine triphosphatase; dUTPase | | 1 |
| DHEA sulfotransferase | STD | 1 |
| Dihydrodiol dehydrogenase 1 | DDHL | 1 |
| Dihydrofolate reductase | DHFR | 1 |
| Dihydrolipoamide branched chain transacylase | DBT | 5 |
| Dihydroiipoamide dehydrogenase | DLD | 5 |
| Dihydrolipoyl dehydrogenase 2 | PDHA | 1 |
| Dihydrolipoyl transacetylase | PDHA | 1 |
| Dihydroorotase | | 1 |
| Dihyropyrimidine dehydrogenase | DPYD | 1 |
| Disrupted meiotic CDNA 1, homolog | DMC1 | 6 |
| DNA damage binding protein, DDBI | DDBI | 3 |
| DNA damage binding protein, DDB2 | DDB2 | 3 |
| DNA directed polymerase, alpha | POLA | 1 |
| DNA glycosylases | | 1 |
| DNA helicases | | 1 |
| DNA Ligase 1 | LIGI | 1 |
| DNA methyltransferase | DNMT | 1 |
| DNA polymerase 1 | | 1 |
| DNA polymerase 2 | | 1 |
| DNA polymerase 3 | | 1 |
| DNA primase | | 1 |
| DNA-damage-inducible transcript 3 | DDIT3 | 3 |
| DNA-dependant RNA polymerase | | 1 |
| Dopamine receptors Di | DRDI | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Erythropoietin | EPO | 4 |
| Erythropoietin receptor | EPOR | 4 |
| Estrogen receptor | ESR | 6 |
| Excision repair complementation group 1 | ERCCI | 1 |
| protein | | |
| Excision repair complementation group 2 | ERCC2 | 1 |
| protein | | |
| Excision repair complementation group 3 | ERCC3 | 1 |
| protein | | |
| Excision repair complementation group 4 | ERCC4 | 1 |
| protein | | |
| Excision repair complementation group 6 | ERCC6 | 1 |
| protein | | |
| Factor H | HFI | 4 |
| Factor IX | F9 | 4 |
| Factor VII | F7 | 4 |
| Factor VIII | F8 | 4 |
| Factor X | F10 | 4 |
| Fafty acid binding proteins FABPL | | 2 |
| Fafty acid binding proteins FABP2 | | 2 |
| Fafty acid binding proteins FABP3 | | 2 |
| Fafty acid binding proteins FABP4 | | 2 |
| Fafty acid binding proteins FABP5 | | 2 |
| Fafty acid binding proteins FABP6 | | 2 |
| Fibroblast growth factor | FGF1 | 6 |
| Flavin-containing monooxygenase 1 | FMO1 | 1 |
| Flavin-containing monooxygenase 2 | FMO2 | 1 |
| Flavin-containing monooxygenase 3 | FMO3 | 1 |
| Flavin-containing monooxygenase 4 | FMO4 | 1 |
| Folic acid receptor | FOLR | 6 |
| Follicle stimulating hormone receptor | FSHR, ODG1 | 6 |
| Follicle stimulating hormone, FSH | FSHB | 6 |
| Forkhead transcription factor 10 | FKHL10 | 6 |
| Forkhead transcription factor 14 | FKHL14 | 6 |
| Forkhead transcription factor 7 | FKHL7 | 6 |
| G/T mismatch binding protein | GTBP, MSH6 | 6 |
| GABA receptor, alpha 1 | GABRAI | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRBI | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Gadd45 (growth arrest & DNA-damage-inducible protein) | | 1 |
| Galactose 1-phosphate uridyl-transferase | GALT | 1 |
| Gamma-glutamyl carboxylase | GGCX | 2 |
| Gamma-glutamyltransferase I | GGTL | 2 |
| Gamma-glutamyltransferase 2 | GGT2 | 2 |
| Gastric inhibitory polypeptide receptor, GIPR | GIPR | 2 |
| Gastric lipase, LIPF | | 2 |
| Glucagon receptor | GCGR | 6 |
| Glucocorticoid receptor | GRL | 6 |
| Glucosaminyl (N-acetyl) transferase 2, I- | GCNT2 | 1 |
| branching enzyme | | |
| Glucosidase, acid beta | GBA | 1 |
| Glutamate decarboxylase, GAD | GAD1 | 1 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutamine phosphoribosylpyrophosphate | | 1 |
| amidotransferase/PRPP amidotransferase | | |
| Glutathione | GSH | 2 |
| Glutathione peroxidase, GPXL | GPXI | 1 |
| Glutathione peroxidase, GPX2 | GPX2 | 1 |
| Glutathione reductase, GSR | GSR | 1 |
| Glutathione S-transferase mu 1, GSTMI | GSTM1 | 1 |
| Glutathione S-transferase mu 4, GSTM4 | | 1 |
| Glutathione S-transferase theta 1, GSTT1 | GSTT1 | 1 |
| Glutathione S-transferase theta 2, GSTT2 | | 1 |
| Glutathione S-transferase, GSTP1 | GSTP1 | 1 |
| Glutathione S-transferase, GSTZ1 | GSTZ1 | 1 |
| Glutathione synthetase | GSS | 1 |
| Glyceraidehyde-3-phosphate dehydrogenase, | GAPDH | 1 |
| GAPDH | | |
| Glycinamide ribanucleotide (GAR) | GART | 1 |
| transformylase | | |
| Glycine receptor, alpha | GLRA2 | 5 |
| Glycine receptor, beta | | 5 |
| Glycine transporter | GLYT | 5 |
| Gonadotropin releasing hormone | GNRH | 6 |
| Gonadotropin releasing hormone receptor | GNRHR | 6 |
| Growth arrest-specific homeobox | GAX | 6 |
| Growth hormone I | GHI | 6 |
| Growth hormone 2 (placental) | GH2 | 6 |
| Growth hormone receptor | GHR | 6 |
| Growth hormone releasing hormone (GHRH) | GHRH | 6 |
| Growth hormone releasing hormone receptor | GHRHR | 6 |
| GTP cyclohydrolase I | GCH1 | 6 |
| GTPase-activating protein, GAP | RASAI | 6 |
| Guanidinoacetate N-methyltransferase | GAMT | 1 |
| Guanine nucleotide-binding protein, alpha | GNA01 | 5 |
| activating activity polypeptide, GNAO | | |
| Guanine nucleotide-binding protein, alpha | GNAI1 | 5 |
| inhibiting activity polypeptide 1, GNAI1 | | |
| Guanine nucleotide-binding protein, alpha | GNAI2 | 5 |
| inhibiting activity polypeptide 2, GNAI2 | | |
| Guanine nucleotide-binding protein, alpha | GNAI3 | 5 |
| inhibiting activity polypeptide 3, GNAI3 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNATI | 5 |
| transducing activity polypeptide, GNAT1 | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, | GNAQ | 5 |
| polypeptide | | |
| Guanylate cyclase 2D, membrane (retina- | GUCY2D | 1 |
| specific) | | |
| Guanylate cyclase activator 1A (retina) | GUCALA | 1 |
| Guanylate kinase | | 1 |
| Guanylin | GUCA2 | 2 |
| Guanylyl cyclase | | 1 |
| H(+), K(+) - ATPase | ATP4B | 5 |
| Heat shock protein, HSP60 | | 4 |
| Heat shock protein, HSP70 | | 4 |
| Heat shock protein, HSP90 | | 4 |
| Hemopexin | HPX | 4 |
| Hepatic lipase | LIPC | 1 |
| Histamine receptors, H1 | | 5 |
| Histamine receptors, H2 | | 5 |
| Histamine receptors, H3 | | 5 |
| HLH transcription factor HAND1 | HAND1 | 6 |
| HLH transcription factor HAND2 | HAND2 | 6 |
| HMG-COA lyase | HMGCL | 1 |
| HMG-COA reductase | HMGCR | 1 |
| HMG-COA synthase | HMGCS2 | 1 |
| Hormone-sensitive lipase | HSL | 1 |
| HSSB, replication protein | HPRT | 1 |
| Hypoxanthine-guanine | HGPRT | 1 |
| phosphoribosyltransferase, | | |
| Ibonucleoside diphosphate reductase | | 1 |
| Ikaros gene | IKAROS | 6 |
| Inosine monophosphate dehydrogenase | IMPDH | 1 |
| Inosine triphosphatase | ITPA | 1 |
| Inositol monophosphatase | IMPAL | 5 |
| Insulin | INS | 6 |
| Insulin receptor | INSR | 6 |
| Insulin-like growth factor 1 receptor | IGFI R | 6 |
| Insulin-like growth factor 2 receptor | IGF2R | 6 |
| Interferon alpha | IFNAL | 4 |
| Interferon beta | IFNB | 4 |
| Interferon gamma | IFNG | 4 |
| Interferon gamma receptor 1 | IFNGRL | 4 |
| Interferon gamma receptor 2 | IFNGR2 | 4 |
| Interferon regulatory factor 1 | IRF1 | 4 |
| Interferon regulatory factor 4 | IRF4 | 4 |
| Interleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) II | IL11 | 4 |
| Interleukin(IL) 11 receptor | IL11R | 4 |
| Interleukin(IL) 12 | IL12 | 4 |
| Interleukin(IL) 12 receptor, beta I | IL12RB1 | 4 |
| Interleukin(IL) 13 | IL13 | 4 |
| Interleukin(IL) 13 receptor | IL13R | 4 |
| Interleukin(IL) 2 | IL2 | 4 |
| Interleukin(IL) 2 receptor, alpha | IL2RA | 4 |
| Interleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| Interleukin(IL) 9 | IL9 | 4 |
| Interleukin(IL) 9 receptor | IL9R | 4 |
| Interleukin(IL) receptor antagonist 1 | IL1RN, IL1RA | 4 |
| Kallikrein 3 | KAK3 | 4 |
| Kinectin | KTNL | 6 |
| Kinesin, heavy chain | KNSLI | 6 |
| Kinesin, light chain | KNS2 | 6 |
| Kininbgen, High molecular weight | KNG | 4 |
| Leptin | LEP | 6 |
| Leptin receptor | LEPR | 6 |
| Leukotriene A4 hydrolase | | 4 |
| Leukotriene B4 receptor | | 4 |
| Leukotriene C4 receptor | | 4 |
| Leukotriene D4/E4 receptor | | 4 |
| LH/choriogonadotropin (CG) receptor | LHCGR | 6 |
| LIM homeobox transcription factor 1, beta | LMXL B | 6 |
| Lipoprotein lipase | LPL | 4 |
| Lipoprotein receptor, Low Density | LDLR | 2 |
| Lipoxygenase 12 (platelets) | LOG12 | 4 |
| Lipoxygenase 5 (leukocytes) | | 4 |
| Low density lipoprotein receptor-related protein | LRP | 2 |
| precursor | | |
| Lysosomal acid lipase | LIPA | 1 |
| Malonyl CoA decarboxylase | | 1 |
| Malonyl CoA transferase | | 1 |
| Maltase-glucoamylase | | 1 |
| Mannose binding protein | MBP | 4 |
| Mannosyl-(alpha-1,6-)-glycoprotein beta-1,2- | MGAT2 | 2 |
| N-acetylglucosaminyltransferase | | |
| MAPK kinase 1 | MAPKK1; MEK1 | 6 |
| MAPK kinase 4 | MAPKK4; MEK4; | 6 |
| | SERKI | |
| MAPK kinase 6 | MAPKK6; MEK6 | 6 |
| MAPKK kinase | MAPKKK | 6 |
| Matrix Gia protein | MGP | 6 |
| MEK kinase, MEKK | | 1 |
| Melanocortin 2 receptor | MC2R | 2 |
| Melanocortin 4 receptor | MC4R | 2 |
| Methionine adenosyltransferase | MAT1A, MAT2A | 1 |
| Methionine synthase | MTR | 1 |
| Methionine synthase reductase | MTRR | 1 |
| Methylguanine-DNA methyltransferase | MGMT | 1 |
| Mevalonate kinase | MVK | 1 |
| MHC Class I: Tap1 | ABCR, TAP1 | 4 |
| MHC Class II: Tap2 | TAP2, PSF2 | 4 |
| Microphthalmia-associated transcription factor | MITF | 6 |
| Mismatch repair gene, PMSLI | PMS1 | 6 |
| Mismatch repair gene, PMSL2 | PMS2 | 6 |
| Mitochondrial trifunctional protein, alpha. | HADHA | 1 |
| subunit | | |
| Mitochondrial trifunctional protein, beta subunit | HADHB | 1 |
| Mitogen-activated protein (MAP) kinase | MAPK | 6 |
| Monoamine oxidase A | MAOA | 1 |
| Monoamine oxidase B | MAOB | 1 |
| Multidrug resistance associated protein | MRP | 6 |
| Muscarinic receptor, MI | CHRMI | 5 |
| Muscarinic receptor, M2 | CHRM2 | 5 |
| Muscadnic receptor, M3 | CHRM3 | 5 |
| Muscarinic receptor, M4 | CHRM4 | 5 |
| Muscarinic receptor, M5 | CHRM5 | 5 |
| Na+, K+ ATPase, alpha | ATPIAI | 6 |
| Na+, K+ ATPase, beta 1 | ATPIBI | 6 |
| Na+, K+ ATPase, beta 2 | ATPIB2 | 6 |
| Na+, K+ ATPase, beta 3 | ATP1B3 | 6 |
| Na+/H+ exchanger 1 | NHFI | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| N-acetylgalactosamine-6-sulfate sulfatase | GALNS | 1 |
| N-acetylglucosamine-6-sulfatase | GNS | 1 |
| N-acetylglucosaminidase, alpha | NAGLU | 1 |
| N-acetyltransferase 1 | NATI | 1 |
| N-acetyltransferase 2 | NAT2 | 1 |
| N-acyl hydrolase | | 4 |
| NADH dehydrogenase (ubiquinone) | NDUFVL | 1 |
| Flavoprotein I | | |
| NADH-cytochrome b5 reductase | DIA1 | 1 |
| Nephrolithiasis 2 | NPHL2 | 2 |
| Nephronophthisis 2 | NPHP2 | 2 |
| Nephrosis 1 | NPHSI | 2 |
| Neuroendocrine convertase 1 | NEC1, PCSK1 | 1 |
| Neurokinin A | NKNA | 5 |
| Neurokinin B | NKNB | 5 |
| Neuropeptide Y | NPY | 5 |
| Neuropeptide Y receptor Y1 | NPYLR | 5 |
| Neuropeptide Y receptor Y2 | NPY2R | 5 |
| Niacin receptor | | 6 |
| Niemann-Pick disease protein | NPCL | 2 |
| Nuclear factor kappa beta | NFKB | 4 |
| Nuclear factor of activated T cells (NFAT) | NFATC | 6 |
| complex, cytosolic | | |
| Nuclear factor of activated T cells (NFAT) | NFATP | 6 |
| complex, preexisting component | | |
| Nucleoside diphosphate kinase-A | NDPKA | 1 |
| Oncogene spil | | 6 |
| Opioid receptor, delta | OPRDI | 5 |
| Opioid receptor, kappa | OPRK1 | 5 |
| Opiold receptor, mu | OPRM1 | 5 |
| Ornithine transcarbamoylase | OTC, NME1 | 1 |
| Osteoprotegerin | OPG | 6 |
| Otoferlin | OTOF | 5 |
| Oxytocin | OXT | 5 |
| Oxytocin receptor | OXTR | 5 |
| Paired-like homeodomain transcription factor 2 | PITX2 | 6 |
| Paired-like homeodomain transcription factor 3 | PITX3 | 6 |
| Paraoxonase PON1 | PON1 | 1 |
| Paraoxonase PON2 | PON2 | 1 |
| Paraoxonase PON3 | PON3 | 1 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Parvalbumin | PVALB | 6 |
| PCNA (proliferating cell nuclear antigen) | | 1 |
| Peanut-like | PNUTLI | 4 |
| Peroxisomal membrane protein I | Pxmp1 | 3 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome proliferative activated receptor, | PPARA | 2 |
| alpha | | |
| Peroxisome proliferative activated receptor, | PPARG | 2 |
| gamma | | |
| P-glycoprotein 1 | PGY1 | 2 |
| P-glycoprotein 3 | PGY3 | 2 |
| Phenylethanolamine N-methyltransferase, | PNMT | 1 |
| PNMT | | |
| Phosphodiesterase 1/nucleotide | PDNPI | 6 |
| pyrophosphatase I | | |
| Phosphodiesterase I/nucleotide | PDNP2 | 6 |
| pyrophosphatase 2 | | |
| Phosphodiesterase 1/nucleotide | PDNP3 | 6 |
| pyrophosphatase 3 | | |
| Phospholipase A2, group 10 | PLA2G 10 | 4 |
| Phospholipase A2, group 1B | PLA2G 1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase-2 | PMM2 | 2 |
| Phosphomannose isomerase-1, PMI I | MPI | 2 |
| Phosphoribosyl pyrophosphate synthetase | PRPS1 | 1 |
| Pituitary adenylate cyclase activating peptide | PACAP | 5 |
| Pituitary adenylate cyclase activating peptide | PACAPLR | 5 |
| receptor | | |
| Plasminogen activator, Tissue | PLAT, TPA | 1 |
| Platelet-activating factor receptor | PAFR | 4 |
| Plectin I | PLEC1 | 2 |
| Polycystin 1 | PKD1 | 2 |
| Polycystin 2 | PKD2 | 2 |
| Porphobilinogen deaminase | HMBS | 1 |
| Potassium channel, calcium-activated, | KCNN4 | 5 |
| Potassium channel, subfamily K, member 1 | KCNK1 | 5 |
| Potassium channel, subfamily K, member 2 | KCNK2 | 5 |
| Potassium channel, subfamily K, member 3 | KCNK3 | 5 |
| Potassium inwardly-rectifying channel JI | KCNJ1 | 5 |
| Potassium inwardly-rectifying channel JII | KCNJ11 | 5 |
| Potassium voltage-gated channel AI | KCNA1 | 5 |
| Potassium voltage-gated channel EI | KCNE1 | 5 |
| Potassium voltage-gated channel QI | KCNQ1 | 5 |
| Potassium voltage-gated channel Q2 | KCNQ2 | 5 |
| Potassium voltage-gated channel Q3 | KCNQ3 | 5 |
| POU domain, class 1, transcription factor I | POUI F1 | 6 |
| (Piti) | | |
| POU domain, class 3, transcription factor 4 | POU3F4 | 6 |
| POU domain, class 4, transcription factor 3 | POU4F3 | 6 |
| Pre-B-cell leukemia transcription factor 1 | PBx1 | 6 |
| Preproglucagon | GCG, GLP1, GLP2 | 6 |
| receptor) | | |
| Prolactin | PRL | 6 |
| Prolactin receptor | PRLR | 6 |
| Proopiomelanocortin | POMC | 5 |
| Prostacyclin synthase | | 4 |
| Prostaglandin 15-OH dehydrogenase | HGPD, PGDH | 4 |
| Prostaglandin D-DP receptor | | 4 |
| Prostaglandin E1 receptor | | 4 |
| Prostaglandin E2 receptor | | 4 |
| Prostaglandin E3 receptor | | 4 |
| Prostaglandin F-FP receptor | | 4 |
| Prostaglandin F2 alpha receptor | | 4 |
| Prostaglandin IP receptor | | 4 |
| Prostaglandin-endoperoxidase synthase 2 | PTGS2 | 6 |
| Protease nexin 2 | PN2 | 1 |
| Protein C | PROC | 4 |
| Protein kinase DNA-activated | PRKDC | 1 |
| Protein S | PROS1 | 4 |
| Pterin-4-alpha-carbinolamine | PCBD | |
| Purine nucleoside phosphorylase | NP | 1 |
| Purinergic receptor P1A1 | | 5 |
| Purinergic receptor P1A2 | | 5 |
| Purinergic receptor P1A3 | | 5 |
| Purinergic receptor P2X, 1 | P2RX1 | 5 |
| Purinergic receptor P2X, 2 | P2RX2 | 5 |
| Purinergic receptor P2X, 3 | P2RX3 | 5 |
| Purinergic receptor P2X, 4 | P2RX4 | 5 |
| Purinergic receptor P2X, 5 | P2RX5 | 5 |
| Purinergic receptor P2X, 6 | P2RX6 | 5 |
| Purinergic receptor P2X, 7 | P2RX7 | 5 |
| Purinergic receptor P2Y, 1 | P2RY1 | 5 |
| Purinergic receptor P2Y, 11 | P2RY11 | 5 |
| Purinergic receptor P2Y, 2 | P2RY2 | 5 |
| RAD51, DNA repair protein | RAD51 | 6 |
| RAD52, DNA repair protein | RAD52 | 6 |
| RAD54, DNA repair protein | RAD54 | 6 |
| RAD55, DNA repair protein | RAD55 | 6 |
| RAD57, DNA repair protein | RAD57 | 6 |
| Recombination activating gene 1 | RAG1 | 6 |
| Recombination activating gene 2 | RAG2 | 6 |
| Red cone pigment | RCP | 3 |
| Replication factor A | | 1 |
| Replication factor C | RFC2 | 1 |
| Retinaldehyde binding protein 1 | RLBPL | 2 |
| Retinoic acid receptor, alpha | RARA | 6 |
| Retinoic acid receptor, beta | RARB | 6 |
| Retinoic acid receptor, gamma | RARG | 6 |
| Retinoid X receptor, alpha | RXRA | 6 |
| Retinoid X receptor, beta | RXRB | 6 |
| Retinoid X receptor, gamma | RG | 6 |
| Retinol binding protein 1 | | 2 |
| Retinol binding protein 2 | | 2 |
| Retinol binding protein 4 | RBP4 | 2 |
| Ribonucleotide reductase, RRM | | 1 |
| Ribosephosphate pyrophosphokinase | | 1 |
| Ribosomal protein L13A | RPL13A | 6 |
| Ribosomal protein S19 | RPS19 | 1 |
| Ribosomal protein S4, X-linked | RPS4X | 1 |
| Ribosomal protein S6 kinase | RPS6KA3 | 1 |
| Ribosomal protein S9 | RPS9 | 6 |
| S-Adenosylmethionine decarboxylase, AMD | | 1 |
| Secretin | SCT | 2 |
| Secretin receptor, SCTR | SCTR | 2 |
| Serine hydroxymethyltransferase | SHMT | 1 |
| Serotonin-N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Slug protein | | 6 |
| Small nuclear ribonucleoprotein polypeptide N | SNRPN | 3 |
| Sodium channel, non-voltage gated 1, alpha | SCNNA | 5 |
| Sodium channel, non-voltage gated 1, beta | SCNN1B | 5 |
| Sodium channel, non-voltage gated 1, gamma | SCNNIG | 5 |
| Sodium channel, voltage gated, type IV, alpha | SCN4A | 5 |
| polypeptide | | |
| Sodium channel, voltage gated, type V, alpha | SCN5A | 5 |
| polypeptide | | |
| Sodium channel, voltage gated, type 1, beta | SCN1B | 5 |
| polypeptide | | |
| Solute carrier family 1 (amino acid transporter), | SLC1A6 | 2 |
| member 6 | | |
| Solute carrier family 1 (glial high affinity | SLC1A3 | 2 |
| glutamate transporter), member 3 | | |
| Solute carrier family 1 (glutamate transporter), | SLC1A1 | 2 |
| member 1 | | |
| Solute carrier family I (glutamate transporter), | SLCIA2 | 2 |
| member 2 | | |
| Solute carrier family 1(neutral amino acid | SLCIA4 | 2 |
| transporter), member 4 | | |
| Solute carrier family IO (sodium/bile acid | SLCIOA1 | 2 |
| cotransporter family), member 1 | | |
| Solute carrier family IO (sodium/bile acid | SLCIOA2 | 2 |
| cotransporter family), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 14, member 2 | SLC14A2 | 2 |
| Solute carrier family 15 (H+/peptide | SLC15A1 | 2 |
| transporter, intestinal), member 1 | | |
| Solute carrier family 15 (H+/peptide | SLC15A2 | 2 |
| transporter, kidney), member 2 | | |
| Solute carrier family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 17, member 1 | SLC17A1 | 2 |
| Solute carrier family 17, member 2 | SLC17A2 | 2 |
| Solute carrier family 18, member 3 | SLC18A3 | 2 |
| Solute carrier family 19 (folate transporter), | SLC19A1 | 2 |
| member 1 | | |
| Solute carrier family 2 (facilitated glucose | SLC2A1 | 2 |
| transporter), member 1 | | |
| Solute carrier family 2 (facilitated glucose | SLC2A2 | 2 |
| transporter), member 2 | | |
| Solute carrier family 2 (facilitated glucose | SLC2A3 | 2 |
| transporter), member 3 | | |
| Solute carrier family 2 (facilitated glucose | SLC2A4 | 2 |
| transporter), member 4 | | |
| Solute carrier family 2 (facilitated glucose | SLC2A5 | 2 |
| transporter), member 5 | | |
| Solute carrier family 20, member 1 | SLC2OA1 | 2 |
| Solute carrier family 20, member 2 | SLC2OA2 | 2 |
| Solute carrier family 20, member 3 | SLC2OA3 | 2 |
| Solute carrier family 21, member 2 | SLC21A2 | 2 |
| Solute carrier family 21, member 3 | SLC21A3 | 2 |
| Solute carrier family 22, member 1 | SLC22A1 | 2 |
| Solute carrier family 22, member 2 | SLC22A2 | 2 |
| Solute carrier family 22, member 5 | SLC22A5 | 2 |
| Solute carrier family 25, member 12 | SLC25A12 | 2 |
| Solute carrier family 3 (facilitated glucose | SLC3A1 | 2 |
| transporter), member 1 | | |
| Solute carrier family 4 (anion exchanger), | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Solute carrier family 6, member 10 | SLC6A10 | 2 |
| Solute carrier family 6, member 6 | SLC6A6 | 2 |
| Solute carrier family 6, member 8 | SLC6A8 | 2 |
| Solute carrier family 7(amino acid transporter), | SLC7A1 | 2 |
| member 1 | | |
| Solute carrier family 7(amino acid transporter), | SLC7A2 | 2 |
| member 2 | | |
| Solute carrier family 7(amino acid transporter), | SLC7A7 | 2 |
| member 7 | | |
| Solute carrier family 8 (sodium/calcium | SLC8A1 | 2 |
| exchanger), member 1 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Sorcin | SRI | 2 |
| SOSI guanine nucleotide exchange factor | sosi | 6 |
| Steroid 5 alpha reductase 1 | SRD5AI | 1 |
| Steroid 5 alpha reductase 2 | SRD5A2 | 1 |
| Steroid hormone receptor responsive DNA | | 6 |
| elements | | |
| Sterol carrier protein 2 | SCP2 | 2 |
| Succinic semi-aldehyde dehydrogenase | SSADH | 1 |
| Sucrase | | 1 |
| Sulfonylurea receptor | SUR | 6 |
| Synaptic vesicle amine transporter | SVAT | 5 |
| Tachykinin receptor, NK1R | TACR1 | 5 |
| Tachykinin receptor, NK2R | TACR2 | 5 |
| Tachykinin receptor, NK3R | TACR3 | 5 |
| Terminal deoxynucleotidyltransferase | TDT | 4 |
| Thiopurine-S-methyltransferase | TPMT | 1 |
| Thrombopoietin | THPO | 6 |
| Thromboxane A synthase I | TBXASI | 4 |
| Thromboxane A2 | TXA2 | 4 |
| Thromboxane A2 receptor | TBXA2R | 4 |
| Thymidylate synthase | TYMS | 1 |
| Thymopoietin | TMPO | 6 |
| Thyroid hormone receptor, beta | THRB | 6 |
| Thyroid-stimulating hormone receptor | TSHR | 6 |
| Thyroid-stimulating hormone, alpha | TSHA | 6 |
| Thyroid-stimulating hormone, beta | TSHB | 6 |
| Topoisomerase I | | 1 |
| Topoisomerase II | | 1 |
| Transcription factor 1, hepatic | TCF1 | 6 |
| Transcription factor 2, hepatic | TCF2 | 6 |
| Transcription factor 3 | TCF3 | 6 |
| Transcription factor binding to IGHM enhancer 3 | TFE3 | 6 |
| Transcription factor, TUPLE1 | TUPLE1 | 5 |
| Transcription termination factor, RNA | TTF1 | 6 |
| polymerase 1 | | |
| Transcription termination factor, RNA | TTF2 | 6 |
| polymerase 2 | | |
| Transcription termination factor, RNA | TTF3 | 6 |
| polymerase 3 | | |
| Transferrin | TF | 6 |
| Transferrin receptor | TFRC | 6 |
| Transthyretin | TTR | 2 |
| Tubulin | | 3 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour Suppressor gene DRA | DRA | 4 |
| Ubiquitin | | 6 |
| Ubiquitin activating enzyme, E1 | | 1 |
| Ubiquitin B | UBB | 6 |
| Ubiquitin C | UBC | 6 |
| Ubiquitin carboxyl-terminal esterase L1 | UCHL1 | 6 |
| Ubiquitin protein ligase E3A | UBE3A | 1 |
| UDP-glucose pyrophosphorylase | | 1 |
| UDP-glucuronosyltransferase 1 | ugtld, UGTL | 1 |
| UDP-glucuronosyltransferase 2 | UGT2 | 1 |
| Uncoupling protein 1 | | 2 |
| Uncoupling protein 3 | UCP3 | 2 |
| Uridine monophosphate kinase | UMPK | 4 |
| Uridine manophosphate synthetase | UMPS | 4 |
| Uridinediphosphate(UDP)-galactose-4- | GALE | 1 |
| epimerase | | |
| Vimentin | VIM | 4 |
| Vitamin B12-binding (R) protein | | 6 |
| Vitamin D receptor | VDR | 6 |
| Xanthine dehydrogenase | XDH | 1 |
| Xeroderma pigmentosum, complementation | XPA | 1 |
| group A | | |
| Xeroderma pigmentosum, complementation | XPB | 1 |
| group B | | |
| Xeroderma pigmentosum, complementation | XPC | 1 |
| group C | | |
| Xeroderma pigmentosum, complementation | | 1 |
| group D | | |
| Xeroderma pigmentosum, complementation | | 1 |
| group E | | |
| Xeroderma pigmentosum, complementation | XPF | 1 |
| group F | | |
| Xeroderma pigmentosum, complementation | ERCC5 | 1 |
| group G | | |
| X-ray repair gene | XRCC9 | 6 |
| Xylitol dehydrogenase | | 1 |
| YY1 transcription factor | YY1 | 6 |

that the gene with cancer are selected from:

| | HUGO | Protein- |
|---|---|---|
| List of genes | gene symbol | function |
| Absent in melanoma 1 gene | AIM1 | 6 |
| Actin, alpha, cardiac | ACTC | 3 |
| Actin, alpha, skeletal | ACTA1 | 3 |
| Actin, alpha, smooth, aortic | ACTA2 | 3 |
| Activin | | 6 |
| Activin A receptor, type 2B | ACVR2B | 6 |
| Activin A receptor, type 2-like kinase 1 | ACVRL1 | 6 |
| Adenomatous polyposis coli tumour supressor | APC | 6 |
| gene | | |
| Adenosine deaminase | ADA | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenyl cyclase | | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alphal | ADRA1 | 5 |
| Adrenergic receptor, alpha2 | ADRA2 | 5 |
| Adrenergic receptor, betal | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| Alcohol dehydrogenase 3 | ADH3 | 1 |
| Aidehyde dehydrogenase 1 | ALDH1 | 1 |
| Aidehyde dehydrogenase 10 | ALDH10 | 1 |
| Aidehyde dehydrogenase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldosterone receptor | MLR | 6 |
| alpha tectorin | TECTA | 6 |
| alpha1-antitrypsin | P1 | 1 |
| alpha-actinin 2 | ACTN2 | 6 |
| alpha-actinin 3 | ACTN3 | 6 |
| Alpha-fetoprotein | AFP | 6 |
| alpha-synuclein | SNCA | 5 |
| Amphiregulin | AREG | 6 |
| Amyloid beta A4 precursor protein | APP | 5 |
| Amyloid beta A4 precursor-like protein | APLP | 5 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Annexin 1 | ANX 1 | 4 |
| Antidiuretic hormone receptor | ADHR | 2 |
| Antithrombin III | AT3 | 1 |
| AP-2, alpha | TFAP2A | 6 |
| AP-2, beta | TFAP2B | 6 |
| AP-2, gamma | TFAP2C | 6 |
| Apaf-1 | | 3 |
| Apoptosis antigen 1 | APT1 | 4 |
| Apoptosis antigen ligand 1 | APT1LG 1 | 4 |
| Apoptosis-inducing factor | AIF | 4 |
| Apurinic endonuclease | APE | 1 |
| Arginine vasopressin | AVP | 5 |
| Arginosuccinate synthetase | ASS | 1 |
| Aryl hydrocarbon receptor | AHR | 2 |
| Aryil hydrocarbon receptor nuclear translocator | ARNT | 2 |
| Asparagine synthetase | AS | 1 |
| Aspartate receptor | | 5 |
| Ataxia telangiectasia complementation group D | ATD, ATDC | 6 |
| Ataxia telangiectasia gene, AT | ATM | 6 |
| ATP cobalamin adenoxyltransferase | | 1 |
| ATP sulphurylase | atpsk2 | 1 |
| ATP-binding cassette transporter 7 | ABC7 | 4 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Atrophin 1 | DRPLA | 6 |
| Bagpipe homeobox, drosophila homolog of, 1 | BAPX1 | 6 |
| B-cell CLL/lymphoma 1 | BCL1 | 4 |
| B-cell CLL/lymphoma 10 | BCL10 | 4 |
| B-cell CLL/lymphoma 3 | BCL3 | 4 |
| B-cell CLUlymphoma 4 | BCL4 | 4 |
| B-cell CLL/lymphoma 5 | BCL5 | 4 |
| B-cell CLU[ymphoma 6 | BCL6 | 4 |
| B-cell CLL/lymphoma 7 | BCL7 | 4 |
| B-cell CLUlymphoma 8 | BCL8 | 4 |
| B-cell CLL/lymphoma 9 | BCL9 | 4 |
| BCL2-associated X protein | BAX | 6 |
| BCL2-related protein A1 | BCL2A1 | 6 |
| Beckwith-Wiedemann region 1A | BWR1A | 6 |
| Benzodiazepine receptor | | 5 |
| beta 2 microglobulin | B2M | 4 |
| beta-endorphin receptor | | 5 |
| beta-synuclein | SNCB | 5 |
| Bleomycin hydrolase | BLMH | 1 |
| Bone morphogenetic protein, BMP1 | BMP1 | 6 |
| Bone morphogenetic protein, BMP2 | BMP2 | 6 |
| Bone morphogenetic protein, BMP3 | BMP3 | 6 |
| Bone morphogenetic protein, BMP4 | BMP4 | 6 |
| Bone morphogenetic protein, BMP5 | BMP5 | 6 |
| Bone morphogenetic protein, BMP6 | BMP6 | 6 |
| Bone morphogenetic protein, BMP7 | BMP7 | 6 |
| Bone morphogenetic protein, BMP8 | BMP8 | 6 |
| Bradykinin receptor B1 | | 4 |
| Bradykinin receptor B2 | | 4 |
| Brain derived neurotrophic factor | BDNF | 6 |
| Brain derived neurotrophic factor (BDNF) | BDNFR | 6 |
| receptor | | |
| Branched chain aminotransferase 1, cytosolic | BCAT1 | 1 |
| Branched chain aminotransferase 2, | BCAT2 | 1 |
| mitochondrial | | |
| BRCA1-associated RING domain gene 1 | BARD1 | 6 |
| Breakpoint cluster region | BCR | 6 |
| Breast cancer 1 | BRCA1 | 6 |
| Breast cancer 2 | BRCA2 | 6 |
| Breast cancer, ductal, 1 | BRCD1 | 6 |
| Breast cancer, ductal, 2 | BRCD2 | 6 |
| Bruton agammaglobulinaemia tyrosine kinase | BTK | 6 |
| C1 inhibitor | | 1 |
| Cadherin E | CDH1 | 6 |
| Cadherin EP | | 6 |
| Cadherin N | CDH2 | 6 |
| Cadherin P | CDH3 | 6 |
| Calbindin I | CALBI | 6 |
| Calbindin D9K- | CALB3 | 6 |
| Calcitonin receptor/Calcitonin gene-related | CALCR | 5 |
| peptide receptor | | |
| Calcitonin/Calcitonin gene-related peptide | CALCA | 5 |
| alpha | | |
| Calcium channel, voltage-dependent, alpha 1 F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha- | CACNA1C | 5 |
| 1C | | |
| Calcium channel, voltage-dependent, Alpha- | CACNA1D | 5 |
| 1 D | | |
| Calcium channel, voltage-dependent, Alpha- | CACNA1E | 5 |
| 1 E (CACNL1A6) | | |
| Calcium channel, voltage-dependent, Alpha- | CACNA2 | 5 |
| 2/detta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase 11 | CAMK2A | 6 |
| Calnexin | CANX | 6 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Cartilage-hair hypoplasia gene | CHH | 5 |
| Caspase1 | CASP1 | 6 |
| Caspase 10 | CASP10 | 6 |
| Caspase 2 | CASP2 | 6 |
| Caspase 3 | CASP3 | 6 |
| Caspase 4 | CASP4 | 6 |
| Caspase 5 | CASP5 | 6 |
| Caspase 6 | CASP6 | 6 |
| Caspase 7 | CASP7 | 6 |
| Caspase 8 | CASP8 | 6 |
| Caspase 9 | CASP9 | 6 |
| Catenin, beta | CTNNB1 | 6 |
| CD1 | CD1 | 4 |
| CD10 | CD10 | 4 |
| CD4 | CD4 | 4 |
| CEA | | 6 |
| Cell adhesion molecule, intercellular, ICAM | ICAM1 | 6 |
| Cell adhesion molecule, leukocyte-endothelial, | LECAM1 | 6 |
| LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM1 | 6 |
| PECAM | | |
| Cell adhesion molecule, vascular, VCAM | VCAM1 | 6 |
| c-erbB1 | ERBB1 | 6 |
| c-erbB2 | ERBB2 | 6 |
| c-erbB3 | ERBB3 | 6 |
| c-erbB4 | ERB64 | 6 |
| Ceruloplasmin precursor | CP | 1 |
| Chemokine receptor CXCR1 | CXCR1 | 4 |
| Chemokine receptor CXCR2 | CXCR2 | 4 |
| Cholecystokinin | CCK | 5 |
| Cholecystokinin B receptor | CCKBR | 5 |
| Ciliary neurotrophic factor (CNTF) | CNTF | 6 |
| Ciliary neurotrophic factor (CNTF) receptor | CNTFR | 6 |
| c-kit receptor tyrosine kinase | | 6 |
| Clathrin | | 2 |
| Clusterin | CLU | 6 |
| Collagen IV alpha 4 | COL4A4 | 3 |
| Collagen IV alpha 5 | COL4A5 | 3 |
| Collagen IV alpha 6 | COL4A6 | 3 |
| Colony-stimulating factor 1 | CSF1 | 6 |
| Colony-stimulating factor 1 receptor | CSF1R | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Complement component C1 inhibitor | C1NH | 4 |
| Complement component C1qa | CIQA | 4 |
| Complement component C1qb | CLQB | 4 |
| Complement component C1qg | CLQG | 4 |
| Complement component C1r | C1R | 4 |
| Complement component C1s | C1S | 4 |
| Complement component C2 | C2 | 4 |
| Complement component C3 | C3 | 4 |
| Complement component C4A | C4A | 4 |
| Complement component C4B | C4B | 4 |
| Complement component C5 | C5 | 4 |
| Complement component C6 | C6 | 4 |
| Complement component C7 | C7 | 4 |
| Complement component C8 | C8B | 4 |
| Complement component C9 | C9 | 1 |
| Complex III | | 1 |
| Core-binding factor, alpha I | CBFA1 | 6 |
| Core-binding factor, alpha 2 | CBFA2 | 6 |
| Core-binding factor, beta | CBFB | 6 |
| Corticotrophin-releasing hormone | CRH | 2 |
| Corticotrophin-releasing hormone receptor | CRHR1 | 2 |
| c-src tyrosine kinase | CSK | 6 |
| Cyclic AMP-dependent protein kinase | PKA | 1 |
| Cyclin A | CCNA | 6 |
| Cyclin B | CCNB | 6 |
| Cyclin C | CCNC | 6 |
| Cyclin D | CCND1 | 6 |
| Cyclin E | CCNE | 6 |
| Cyclin F | CCNF | 6 |
| Cyclin-dependent kinase 1 | CDK1 | 6 |
| Cyclin-dependent kinase 10 | CDKI 0 | 6 |
| Cyclin-dependent kinase 2 | CDK2 | 6 |
| Cyclin-dependent kinase 3 | CDK3 | 6 |
| Cyclin-dependent kinase 4 | CDK4 | 6 |
| Cyclin-dependent kinase 5 | CDK5 | 6 |
| Cyclin-dependent kinase 6 | CDK6 | 6 |
| Cyclin-dependent kinase 7 | CDK7 | 6 |
| Cyclin-dependent kinase 8 | CDK8 | 6 |
| Cyclin-dependent kinase 9 | CDK9 | 6 |
| Cyclin-dependent kinase inhibitor 1A (P21, | CDKN1A | 6 |
| CIP1) | | |
| Cyclin-dependent kinase inhibitor I B (P27, | CDKN1B | 6 |
| KIP1) | | |
| Cyclin-dependent kinase inhibitor IC (P57, | CDKN1C | 6 |
| KIP2) | | |
| Cyclin-dependent kinase inhibitor 2A (p16) | CDKN2A | 6 |
| Cyclin-dependent kinase inhibitor 3 | CDKN3 | 6 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| GYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| GYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| Defender against cell death 1 | DAD1 | 6 |
| Deleted in colorectal carcinoma | DCC | 6 |
| Deleted in malignant brain tumours 1 | DMBT1 | 6 |
| Deoxycytidine kinase DCK | | 1 |
| Deoxyuridine triphosphatase; dUTPase | | 1 |
| Desert hedgehog, dhh | | 6 |
| Dihydrofolate reductase | DHFR | 1 |
| Dihydrolipoyl dehydrogenase | | 1 |
| Dihyropyrimidine dehydrogenase | DPYD | 1 |
| DM-Kinase | DMPK | 1 |
| DNA damage binding protein, DDB1 | DDB1 | 3 |
| DNA damage binding protein, DDB2 | DDB2 | 3 |
| DNA directed polymerase, alpha | POLA | 1 |
| DNA glycosylases | | 1 |
| DNA helicases | | 1 |
| DNA Ligase 1 | LIG1 | 1 |
| DNA methyltransferase | DNMT | 1 |
| DNA polymerase 1 | | 1 |
| DNA polymerase 2 | | 1 |
| DNA polymerase 3 | | 1 |
| DNA primase | | 1 |
| DNA-damage-inducible transcript 3 | DDIT3 | 3 |
| DNA-dependant RNA polymerase | | 1 |
| DOPA decarboxylase | DDC | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dynamin | DNM1 | 6 |
| Dynorphin receptor | | 5 |
| Dysfedin | DYS, DYSF | 1 |
| Dyskerin | DKC1 | 3 |
| EB1 | | 6 |
| Endoglin | ENG | 3 |
| Endothelin 1 | EDN1 | 5 |
| Endothelin 2 | EDN2 | 5 |
| Endothelin 3 | EDN3 | 5 |
| Endothelin converting enzyme | ECE | 5 |
| Endothelin receptor type A | EDNRA | 5 |
| Endothelin receptor type B | EDNRB | 5 |
| Enolase | EN01 | 1 |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Estrogen receptor | ESR | 6 |
| Eukaryotic initiation translation factor | EIF4E | 6 |
| EWS RNA-binding protein | EWSR1 | 6 |
| Excision repair complementation group 1 | ERCCI | 1 |
| protein | | |
| Excision repair complementation group 2 | ERCC2 | 1 |
| protein | | |
| Excision repair complementation group 2 | ERCC3 | 1 |
| protein | | |
| Excision repair complementation group 4 | ERCC4 | 1 |
| protein | | |
| Excision repair complementation group 6 | ERCC6 | 1 |
| protein | | |
| Exostosin 1 | EXT1 | 3 |
| Exostosin 2 | EXT2 | 3 |
| FADH dehydrogenase | | 1 |
| Fanconi anemia, complementation group C | FANCC | 2 |
| Fanconi anemia, complementation group D | FANCD | 2 |
| Fc fragment of IgG, high affinity IA, receptor for | FCGRL A | 6 |
| Fc fragment of IgG, low affinity IIa, receptor for | FCGR2A | 6 |
| (CD32) | | |
| Fc fragment of IgG, low affinity llla, receptor for | FCGR3A | 6 |
| (CD16) | | |
| Ferrochelatase | FECH | 1 |
| Fibrillin 1 | FBN1 | 6 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Folic acid receptor | FOLR | 6 |
| Follicle stimulating hormone receptor | FSHR, ODG1 | 6 |
| Follicle stimulating hormone, FSH | FSHB | 6 |
| Follicular lymphoma variant translocation 1 | FVT1 | 4 |
| Forkhead rhabdomyosarcoma gene | FKHR | 6 |
| Forkhead transcription factor 14 | FKHL14 | 6 |
| Forkhead transcription factor 7 | FKHL7 | 6 |
| Fucosyltransferase 2 | FUT2 | 2 |
| Fucosyltransferase 3 | FUT3 | 2 |
| GIT mismatch binding protein | GTBP, MSH6 | 6 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| Gadd45 (growth arrest & DNA-damage-inducible protein) | | 1 |
| Galactosyltransferase 1 | GT1 | 6 |
| Galactosyltransferase, alpha 1, 3 | GGTA1 | 6 |
| Galactosyltransferase, beta 3 | B3GALT | 6 |
| Gastrin | GAS | 6 |
| Gastrin releasing peptide | GRP | 2 |
| Glioma chloride ion channel, GCC | | 6 |
| Glucagon receptor | GCGR | 6 |
| Glucagon synthase | | 2 |
| Glucocorticoid receptor | GRL | 6 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionctropic, NMDA 2D | NMDAR2D | 5 |
| Glutathione | GSH | 2 |
| Glutathione S-transferase mu 1, GSTM1 | GSTM1 | 1 |
| Glutathione S-transferase theta 1, GSTT1 | GSTT1 | 1 |
| Glutathione S-transferase, GSTZI | GSTZ1 | 1 |
| Glyceraldehyde-3-phosphate dehydrogenase, GAPDH | | 1 |
| GAPDH | | |
| Glycerol kinase | GK | 1 |
| Glycinamide ribonucleotide (GAR) | GART | 1 |
| transformylase | | |
| Glycine receptor, alpha | GLRA2 | 5 |
| Glycine receptor, beta | | 5 |
| Glycine transporter | GLYT | 5 |
| Glypican 3 | GPC3, SDYS | 6 |
| Gonadotropin releasing hormone | GNRH | 6 |
| Gonadotropin releasing hormone receptor | GNRHR | 6 |
| Growth factor receptor-bound protein 2 | GRB2 | 6 |
| Growth hormone 1 | GH1 | 6 |
| Growth hormone 2 (placental) | GH2 | 6 |
| Growth hormone receptor | GHR | 6 |
| Growth hormone releasing hormone (GHRH) | GHRH | 6 |
| Growth hormone releasing hormone receptor | GHRHR | 6 |
| Growth/differentiation factor 5 | GDF5 | 6 |
| Growth-regulated protein precursor, GRO | GRO | 4 |
| GTPase-activating protein, GAP | RASA1 | 6 |
| Guanine nucleotide-binding protein, alpha | GNAI1 | 5 |
| inhibiting activity polypeptide 1, GNA11 | | |
| Guanine nucleotide-binding protein, alpha | GNAI2 | 5 |
| inhibiting activity polypeptide 2, GNA12 | | |
| Guanine nucleotide-binding protein, alpha | GNAI3 | 5 |
| inhibiting activity polypeptide 3, GNA13 | | |
| Guanine nuclootide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, q | GNAQ | 5 |
| polypeptide | | |
| Guanylate kinase | | 1 |
| H(+), K(+) - ATPase | ATP4B | 5 |
| Hairless | HR | 6 |
| Hela tumor suppression gene | HTS1 | 6 |
| Heparin binding epidermal growth factor | HBEGF | 6 |
| Hepatitis 8 virus integration site 1 | HVBS1 | 4 |
| Hepatitis B virus integration site 2 | HVBS6 | 4 |
| High mobility group protein C | HMGIC | 6 |
| High mobility group protein Y | HMGIY | 6 |
| Histamine receptors, H1 | | 5 |
| Histamine receptors, H2 | | 5 |
| Histamine receptors, H3 | | 5 |
| HLH transcription factor HAND1 | HAND1 | 6 |
| HLH transcription factor HAND2 | HAND2 | 6 |
| HMG-CoA reductase | HMGCR | 1 |
| HMG-CoA synthase | HMGCS2 | 1 |
| Homeobox (HOX) gene A13 | HOXA13 | 6 |
| Homeobox 11 | HOX11 | 6 |
| Homeobox HB24 | HLX1 | 6 |
| Homogentisate 1,2 dioxygenase | HGD | 1 |
| Hormone-sensitive lipase | HSL | 1 |
| HSSB, replication protein | | 1 |
| Human placental lactogen | CSH1 | 6 |
| bonucleoside diphosphate redudtase | | 1 |
| Ikaros gene | IKAROS | 6 |
| lnhibin, alpha | INHA | 6 |
| lnhibin, beta A | INHBA | 6 |
| lnhibin, beta B | INHBB | 6 |
| lnhibin, beta C | INHBC | 6 |
| Inositol 1,4,5-triphosphate receptor 3 | ITPR3 | 6 |
| Insulin | INS | 6 |
| Insulin receptor | INSR | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| Integrin beta 1 | ITGB1 | 6 |
| Integrin beta 2 | ITGB2 | 6 |
| lntegrin beta 3 | ITGB3 | 6 |
| lntegrin beta 4 | ITGB4 | 6 |
| lntegrin beta 5 | ITGB5 | 6 |
| lntegrin beta 6 | ITGB6 | 6 |
| Integrin beta 7 | ITGB7 | 6 |
| lntegrin, alpha 1 | ITGA1 | 6 |
| Integrin, alpha 2 | ITGA2 | 6 |
| Integrin, alpha 4 | ITGA4 | 6 |
| lntegrin, alpha 5 | ITGA5 | 6 |
| lntegrin, alpha 6 | ITGA6 | 6 |
| lntegrin, alpha M | ITGAM | 6 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| Interleukin(IL) I receptor | ILIR | 4 |
| Interleukin(IL) 1, alpha | ILIA | 4 |
| lnterleukin(IL) 1, beta | ILIB | 4 |
| Interleukin(IL) 10 | IL10 | 4 |
| Interleukin(IL) 10 receptor | IL10R | 4 |
| Interleukin(IL) II | IL11 | 4 |
| lnterleukin(IL) 11 receptor | IL11R | 4 |
| Interleukin(IL) 12 | IL12 | 4 |
| Interleukin(IL) 12 receptor, beta I | IL12RB1 | 4 |
| Interleukin(IL) 13 | IL13 | 4 |
| Interleukin(IL) 13 receptor | IL13R | 4 |
| Interleukin(IL) 2 | IL2 | 4 |
| Interleukin(IL) 2 receptor, alpha | IL2RA | 4 |
| lnterleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| lnterleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| Interleukin(IL) 7 receptor | IL7R | 4 |
| lnterleukin(IL) 8 | IL8 | 4 |
| Interleukin(IL) 8 receptor | IL8R | 4 |
| lnterleukin(IL) 9 | IL9 | 4 |
| lnterleukin(IL) 9 receptor | IL9R | 4 |
| lnterleukin(IL) receptor antagonist I | IL1RN, IL1RA | 4 |
| Janus kinase 1 | JAK1 | 6 |
| Janus kinase 2 | JAK2 | 6 |
| Janus kinase 3 | JAK3 | 6 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta binding | LTBP2 | 6 |
| protein 2 | | |
| Leptin | LEP | 6 |
| Leptin receptor | LEPR | 6 |
| Leukaemia inhibitory factor | LIF | 6 |
| Leukaemia inhibitory factor receptor | LIFR | 6 |
| Leukotriene A4 hydrolase | | 4 |
| Leukotriene B4 receptor | | 4 |
| Leukotriene C4 receptor | | 4 |
| Leukotriene D4/E4 receptor | | 4 |
| LH/choriogonadotropin (CG) receptor | LHCGR | 6 |
| LIM homeobox protein 1 | LHX1 | 6 |
| LIM. homeobox protein 2 | LHX2 | 6 |
| LIM homeobox protein 3 | LHX3 | 6 |
| LIM homeobox protein 4 | LHX4 | 6 |
| Limbic associated membrane protein | LAMP | 6 |
| LIM-domain only protein 1 | LMO1 | 6 |
| LIM-domain only protein 2 | LMO2 | 6 |
| LIM-domain only protein 3 | LMO3 | 6 |
| LIM-domain only protein 4 | LMO4 | 6 |
| Lipoma-preferred partner gene | LPP | 6 |
| Lipoxygenase 12 (platelets) | LOG12 | 4 |
| Lipoxygenase 5 (leukocytes) | | 4 |
| Long QT-type 2 potassium channels | LQT2, KCNH2 | 2 |
| Lowe oculocerbrorenal syndrome gene | OCRL | 1 |
| Luteinizing hormone-releasing hormone | | 5 |
| Luteinizing hormone-releasing hormone | | 5 |
| receptor | | |
| Lymphoblastic leukemia derived sequence 1 | LYL1 | 4 |
| Lymphocyte-specific protein tyrosine kinase | LCK | 4 |
| Lymphoid enhancer-binding factor | LEF-1 | 6 |
| Macrophage activating factor | MAF | 4 |
| MAD (mothers against decapentaplegic, | MADH3 | 6 |
| Drosophila) homologue 3 | | |
| MAD (mothers against decapentaplegic, | MADH4 | 6 |
| Drosophila) homologue 4 | | |
| MADS box transcription-enhancer factor 2A | MEF2A | 6 |
| MADS box transcription-enhancer factor 2B | MEF2B | 6 |
| MADS box transcription-enhancer factor 2C | MEF2C | 6 |
| MADS box transcnption-enhancer factor 2D | MEF2D | 6 |
| Malignant proliferation, eosinophil gene | MPE | 4 |
| MAPK kinase 1 | MAPKK1; MEK1 | 6 |
| MAPK kinase 4 | MAPKK4; MEK4; | 6 |
| SERK1 | | |
| MAPK kinase 6 | MAPKK6; MEK6 | 6 |
| MAPKK kinase | MAPKKK | 6 |
| MAX-interacting protein 1 | MXI 1 | 6 |
| MEK kinase, MEKK | | 1 |
| Melanocortin 1 receptor | MCI R | 2 |
| Menin | MEN1 | 6 |
| Methionine adenosyltransferase | MAT1 A, MAT2A | 1 |
| Methionine synthase | MTR | 1 |
| Methionine synthase reductase | MTRR | 1 |
| Methylguanine-DNA methyltransferase | MGMT | 1 |
| MHC Class 1: A | | 4 |
| MHC Class 1: B | | 4 |
| MHC Class 1: C | | 4 |
| MHC Class 1: LMP-2, LMP-7 | | 4 |
| MHC Class 1: Tap1 | ABCR, TAP1 | 4 |
| MHC Class 11: DP | HLA-DPBL | 4 |
| MHC Class II: DQ | | 4 |
| MHC Class II: DR | | 4 |
| MHC Class II: Tap2 | TAP2, PSF2 | 4 |
| MHC Class II: Complementation group A | MHC2TA | 4 |
| MHC Class II: Complementation group B | rfxank | 4 |
| MHC Class II: Complementation group C | RFX5 | 4 |
| MHC Class II: Complementation group D | RFXAP | 4 |
| Midline 1 | MIDI | 6 |
| Mismatch, repair gene, PMSL1 | PMS1 | 6 |
| Mismatch repair gene, PMSL2 | PMS2 | 6 |
| Mitogen-activated protein (MAP) kinase | MAPK | 6 |
| Motilin | MLN | 6 |
| Msh homeobox homolog 1 | MSX1 | 6 |
| Msh homeobox homolog 2 | MSX2 | 6 |
| Mucin 18 | MUC18 | 2 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Mutated in colorectal cancers, MCC | MCC | 6 |
| MutL homolog 1 | MLHI | 6 |
| MutS homolog 2 | MSH2 | 6 |
| MutS homolog 3 | MSH3 | 6 |
| Myelin protein peripheral 22 | PMP22 | 3 |
| Myelodysplasia syndrome 1 gene | MDS1 | 6 |
| Myeloid leukemia factor-1 | MLF1 | 4 |
| N-acetyltransferase 1 | NAT1 | 1 |
| N-acetyltransferase 2 | NAT2 | 1 |
| reductase | | |
| Nerve growth factor | NGF | 6 |
| Nerve growth factor receptor | NGFR | 6 |
| Neuregulin | HGL | 6 |
| Neurexin | | 5 |
| Neurofibromin 1 | NF1 | 6 |
| Neurofibromin 2 | NF2 | 6 |
| Neurokinin A | NKNA | 5 |
| Neurokinin B | NKNB | 5 |
| Neuronal apoptosis inhibitory protein | NAIP | 4 |
| Neuropeptide Y | NPY | 5 |
| Neuropeptide Y receptor Y1 | NPY1R | 5 |
| Neuropeptide Y receptor Y2 | NPY2R | 5 |
| Neurotensin | NTS | 5 |
| Neurotensin receptor | NTSR1 | 5 |
| Neurotrophic tyrosine kinase receptor 1 | NTRK1 | 6 |
| Neutral endopeptidase | | 1 |
| Niacin receptor | | 6 |
| Nodal | NODAL | 6 |
| Norrie disease protein | NDP | 6 |
| Notch 3 | NOTCH3 | 6 |
| Notch ligand - jagged 1 | JAG 1, AGS | 6 |
| Nuclear factor kappa beta | NFKB | 4 |
| Nuclear factor of activated T cells (NFAT) | NFATC | 6 |
| complex, cytosolic | | |
| Nuclear factor of activated T cells (NFAT) | NFATP | 6 |
| complex, preexisting component | | |
| Nuclear mitotic apparatus protein 1 | NUMA1 | 6 |
| Nucleophosmin | NPM1 | 2 |
| Oligophrenin-1 | OPHN1 | 6 |
| Oncogene abl1 | ABLI | 6 |
| Oncogene abl2 | | 6 |
| Oncogene akt1 | | 6 |
| Oncogene akt2 | AKT2 | 6 |
| Oncogene axl | AXL | 6 |
| Oncogene bcl2 | | 6 |
| Oncogene bcr/abi | | 6 |
| Oncogene B-lym | | 6 |
| Oncogene B-raf | | 6 |
| Oncogene clkl | | 6 |
| Oncogene c-myc | | 6 |
| Oncogene cot | | 6 |
| Oncogene crk | | 6 |
| Oncogene crkI | | 6 |
| Oncogene ect2 | | 6 |
| Oncogene ELK1 | ELK1 | 6 |
| Oncogene ELK2 | ELK2 | 6 |
| Oncogene ems1 | | 6 |
| Oncogene ERB | | 6 |
| Oncogene ERB2 | | 6 |
| Oncogene ERBA | | 6 |
| Oncogene ERBAL2 | | 6 |
| Oncogene ERG (early reponse gene) | | 6 |
| Oncogene ETSI | | 6 |
| Oncogene ETS2 | | 6 |
| Oncogene EVI1 | EVI1 | 6 |
| Oncogene fes | | 6 |
| Oncogene fgr | | 6 |
| Oncogene fos | FOS | 6 |
| Oncogene fps | | 6 |
| Oncogene GLI1 | GLI | 6 |
| Oncogene GLI2 | GLI2 | 6 |
| Oncogene GLI3 | GLI3 | 6 |
| Oncogene gro1 | | 6 |
| Oncogene gro2 | | 6 |
| Oncogene Ha-ras | HRAS | 6 |
| Oncogene hs1 | | 6 |
| Oncogene hst | FGF4 | 6 |
| Oncogene int1 | WNT1 | 6 |
| Oncogenein | FGF3 | 6 |
| Oncogene int3 | Notch4 | 6 |
| Oncogene int4 | WNT3 | 6 |
| Oncogenejun | JUN | 6 |
| Oncogene KIT | KIT, PBT | 6 |
| Oncogene LCO | LCO | 6 |
| Oncogene I-myc | | 6 |
| Oncogene Ipsa | | 6 |
| Oncogene lyn | | 6 |
| Oncogene maf | | 6 |
| Oncogene mas1 | | 6 |
| Oncogene mcf2 | | 6 |
| Oncogene mdm2 | MDM2 | 6 |
| Oncogene mel | | 6 |
| Oncogene met | MET | 6 |
| Oncogene mos | | 6 |
| Oncogene mpl | | 6 |
| Oncogene MUM1 | MUM1 | 6 |
| Oncogene myb | MYB | 6 |
| Oncogene myc | MYC | 6 |
| Oncogene n-myc | | 6 |
| Oncogene N-ras (neuroblastoma v-ras) | NRAS | 6 |
| Oncogene ovc | | 6 |
| Oncogene pim1 | | 6 |
| Oncogene pti-1 sea | | 6 |
| Oncogene pvt1 | | 6 |
| Oncogene raf | RAF | 6 |
| Oncogene ralb | | 6 |
| Oncogene re1 | | 6 |
| Oncogene ret | RET | 6 |
| Oncogene r-myc | | 6 |
| Oncogene ros | | 6 |
| Oncogene R-ras | | 6 |
| Oncogene sis | PDGFB | 6 |
| Oncogene ski | | 6 |
| Oncogene sno | | 6 |
| Oncogene spi1 | | 6 |
| Oncogene src | | 6 |
| Oncogene tc21 | | 6 |
| Oncogene TEL | ETV6 | 6 |
| Oncogene tim | | 6 |
| Oncogene vavtrk | | 6 |
| Oncogene v-Ki-ras2 | KRAS2 | 6 |
| Oncogene yes | | 6 |
| Oncogene yuasa | | 6 |
| Oncostatin M | OSM | 6 |
| Oncostatin M receptor | OSMR | 6 |
| Opioid receptor, delta | OPRD1 | 5 |
| Opioid receptor, kappa | OPRK1 | 5 |
| Opioid receptor, mu | OPRM1 | 5 |
| Orexin | OX | 6 |
| Osteopontin | OPN | 6 |
| Oxytocin | OXT | 5 |
| Oxytocin receptor | OXTR | 5 |
| Paired box homeotic gene 3 | PAX3 | 6 |
| Paired box homeotic gene 6 | PAX6 | 6 |
| Paired box homeotic gene 7 | PAX7 | 6 |
| Paired-like homeodomain transcription factor 2 | PITX2 | 6 |
| Paired-like homeodomain transcription factor 3 | PITX3 | 6 |
| Parathyroid hormone | PTH | 6 |
| Parathyroid hormone receptor | PTHR1 | 6 |
| Parathyroid hormone related-peptide | PTHRP | 6 |
| Parvalbumin | PVALB | 6 |
| Patched (Drosophila) homolog, PTCH | PTCH | 6 |
| PCNA (proliferating cell nuclear antigen) | | 1 |
| Peanut-like 1 | PNUTL1 | 4 |
| Peroxisome proliferative activated receptor, | PPARA | 2 |
| alpha | | |
| Peroxisome proliferative activated receptor, | PPARG | 2 |
| gamma | | |
| P-glycoprotein 1 | PGY1 | 2 |
| P-glycoprotein 3 | PGY3 | 2 |
| Phenylalanine hydroxylase | PAH | 1 |
| Phosphatase & tensin homolog | PTEN | 6 |
| Phosphatidylinositol glycan, class A | PIGA | 6 |
| (paroxysmal nocturnal hemoglobinuria) | | |
| Phospholipase A2, group 10 | PLA2G 10 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C epsilon | | 4 |
| Phosphomannomutase 1 | PMM1 | 6 |
| Phosphomannomutase 2 | PMM2 | 6 |
| Plasminogen | PLG | 1 |
| Plasminogen activator inhibitor 1 | PAI1 | 1 |
| Plasminogen activator inhibitor 2 | PAI2 | 1 |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Platelet glycoprotein 1b, beta | GP1BB | 4 |
| Platelet glycoprotein I b, gamma | GP1BG | 4 |
| Platelet glycoprotein IX | GP9 | 4 |
| Platelet glycoprotein V | GP5 | 4 |
| Potassium inwardly-rectifying channel J1 | KCNJ1 | 5 |
| Potassium inwardly-rectifying channel J11 | KCNJ11 | 5 |
| Potassium voltage-gated channel A1 | KCNA1 | 5 |
| Potassium voltage-gated channel E1 | KCNE1 | 5 |
| Potassium voltage-gated channel Q1 | KCNQ1 | 5 |
| Potassium voltage-gated channel Q2 | KCNQ2 | 5 |
| Potassium voltage-gated channel Q3 | KCNQ3 | 5 |
| POU domain, class 1, transcription factor 1 | POU1F1 | 6 |
| (Pit1) | | |
| POU domain, class 3, transcription factor 4 | POU3F4 | 6 |
| POU domain, class 4, transcription factor 3 | POU4F3 | 6 |
| Pre-B-cell leukemia transcription factor 1 | PBXL | 6 |
| Preproglucagon | GCG; GLPL; GLP2 | 6 |
| Preproglucagon | | 2 |
| Prion protein | PRNP | 5 |
| Prodynorphin | | 5 |
| Prohibitin | PHB | 6 |
| Prolactin | PRL | 6 |
| Prolactin receptor | PRLR | 6 |
| Prolactin releasing hormone | PRH | 6 |
| Proliferin | PLF | 6 |
| Promyelocytic-leukemia gene | PML | 6 |
| Proopiomelanocortin | POMC | 5 |
| Prophet of Pit1 | PROP1 | 6 |
| Prostacyclin synthase | | 4 |
| Prostagiandin 15-OH dehydrogenase | HGPD; PGDH | 4 |
| Prostaglandin D - DP receptor | | 4 |
| Prostagiandin E1 receptor | | 4 |
| Prostaglandin E2 receptor | | 4 |
| Prostagiandin E3 receptor | | 4 |
| Prostaglandin F - FP receptor | | 4 |
| Prostagiandin IP receptor | | 4 |
| Prostate cancer anti-metastasis gene KAI I | KAI 1 | 6 |
| Protein kinase B | PRKB | |
| Protein kinase C, alpha | PRKCA | 1 |
| Protein phosphatase 2, regulatory subunit A, | PPP2R1B | 1 |
| beta isoform | | |
| Protein tyrosine phosphatase, non-receptor | PTPN12 | 6 |
| type 12 | | |
| Purine nucleoside phosphorylase | NP | 1 |
| Purinergic receptor P1A1 | | 5 |
| Purinergic receptor P1A2 | | 5 |
| Purinergic receptor P1A3 | | 5 |
| Purinergic receptor P2X, 1 | P2RX1 | 1 |
| Purinergic receptor P2X, 2 | P2RX2 | 5 |
| Purinergic receptor P2X, 3 | P2RX3 | 5 |
| Purinergic receptor P2X, 4 | P2RX4 | 5 |
| Pudnergic receptor P2X, 5 | P2RX5 | 5 |
| Purinergic receptor P2X, 6 | P2RX6 | 5 |
| Purinergic receptor P2X, 7 | P2RX7 | 5 |
| Purinergic receptor P2Y, 1 | P2RY1 | 5 |
| Purinergic receptor P2Y, 11 | P2RY11 | 5 |
| Purinergic receptor P2Y, 2 | P2RY2 | 5 |
| Rabphilin | | 5 |
| RAD51, DNA repair protein | RAD51 | 6 |
| RAD52, DNA repair protein | RAD52 | 6 |
| RAD54, DNA repair protein | RAD54 | 6 |
| RAD55, DNA repair protein | RAD55 | 6 |
| RAD57, DNA repair protein | RAD57 | 6 |
| RAS-associated protein, RAB3A | RAB3A | 5 |
| Ras-G-protein | RAS | 6 |
| Receptor tyrosine kinase (RTK), Nsk2 | NSK2 | 6 |
| Relaxin H1 | RLN1 | 6 |
| Relaxin H2 | RLN2 | 6 |
| Replication factor A | | 1 |
| Replication factor C | RFC2 | 1 |
| Retinoblastoma 1 | RB1 | 6 |
| Retinoic acid receptor, alpha | RARA | 6 |
| Retinoic acid receptor, beta | RARB | 6 |
| Retinoic acid receptor, gamma | RARG | 6 |
| Retinoschisis, X-linked, juvenile | RS | 6 |
| Rhabdoid tumors | SMARCSI | 6 |
| Ribonucleotide reductase, RRM | | 1 |
| Ribosomal protein L13A | RPL13A | 6 |
| Ribosomal protein L17 | RPL17 | 6 |
| Ribosomal protein S6 kinase | RPS6KA3 | 1 |
| RIGUI | RIGUI | 6 |
| Rim | | 5 |
| Ryanodine receptor 1, skeletal | RYR1 | 6 |
| 5-adenosylmethionine decarboxylase, AMD | | 1 |
| SAP (SLAM-associated protein) | SH2D1A | 4 |
| Secretin | SCT | 2 |
| Secretin receptor, SCTR | SCTR | 2 |
| Serine hydroxymethyltransferase | SHMT | 1 |
| Serine/threonine kinase 11 | STK11 | 6 |
| Serine/threonine kinase 2 | STK2 | 6 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Signal transducer and activator of transcription I | STATI | 6 |
| Signal transducer and activator of transcription 2 | STAT2 | 6 |
| Signal transducer and activator of transcription 3 | STAT3 | 6 |
| Signal transducer and activator of transcription 4 | STAT4 | 6 |
| Signal transducer and activator of transcription 5 | STAT5 | 6 |
| Signaling lymphocyte activation molecule | SLAM | 4 |
| Sine oculis homeobox, drosophila, homolog 1 | SIX1 | 6 |
| Sine oculis homeobox, drosophila, homolog 2 | SIX2 | 6 |
| Sine oculis homeobox, drosophila, homolog 5 | SIX5 | 6 |
| Small nuclear ribonucleoprotein polypeptide N | SNRPN | 3 |
| Smoothened (Drosophila) homolog | SMOH | 6 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage gated, type V, alpha | SCN5A | 5 |
| polypeptide | | |
| Sodium channel, voltage-gated, type 1, beta | SCN1B | 5 |
| polypeptide | | |
| Solute carrier family 1 (glutamate transporter), | SLC1A1 | 2 |
| member 1 | | |
| Solute carrier family 1 (glutamate transporter), | SLC1A2 | 2 |
| member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 19 (folate transporter), | SLC19A1 | 2 |
| member 1 | | |
| Solute carrier family 25, member 12 | SLC25A12 | 2 |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Sorcin | SRI | 2 |
| SOSI guanine nucleotide exchange factor | SOS1 | 6 |
| SRY-box 11 | SOX11 | 6 |
| Stem cell factor | SCF | 6 |
| Steroid hormone receptor responsive DNA | | 6 |
| elements | | |
| Steroidogenic acute regulatory protein | STAR | 2 |
| Substance P | | 5 |
| Sulfonylurea receptor | SUR | 6 |
| Suppression of tumorigenicity 3 gene | ST3 | 6 |
| Suppression of tumorigenicity 8 gene | ST8 | 6 |
| Surfeit 1 | SURF1 | 6 |
| Synapsin I a & 1b | SYN1 | 5 |
| Synapsin 2a & 2b | SYN2 | 5 |
| Synaptic vesicle protein 2 | SV2 | 5 |
| Synaptobrevin 1 | SyB1 | 5 |
| Synaptobrevin 2 | SYB2 | 5 |
| Synaptogyrin | | 5 |
| Synaptophysin | SYP | 5 |
| Synaptosomal-associated protein, 25 KD | SNAP25 | 5 |
| Synaptotagmin 1 | SYT1 | 5 |
| Synaptotagmin 2 | SYT2 | 5 |
| Syndecan 1 | SYND1 | 6 |
| Syndecan 2 | SYND2 | 6 |
| Syndecan 3 | SYND3 | 6 |
| Syndecan 4 | SYND4 | 6 |
| Synovial sarcoma gene 1 | SSX1 | 6 |
| Synovial sarcoma gene 2 | SSX2 | 6 |
| Syntaxin 1 | STX1 | 5 |
| Tachykinin receptor, NK1R | TACR1 | 5 |
| Tachykinin receptor, NK2R | TACR2 | 5 |
| Tachykinin receptor, NK3R | TACR3 | 5 |
| Talin | TLN | 6 |
| Talin, TLN | | 3 |
| T-cell acute lymphocytic leukemia 1 | TAL1 | 4 |
| T-cell acute lymphocytic leukemia 2 | TAL2 | 4 |
| T-cell receptor, alpha | TCRA | 4 |
| T-cell receptor, delta | TCRD | 4 |
| Telomerase protein component | | 1 |
| Tenascin (cytotactin) | | 3 |
| Tenascin XA | TNXA | 3 |
| Terminal deoxynucleotidyltransferase, TDT | | 1 |
| Testis-specific protein Y | TSPY | 6 |
| Thrombopoietin | THPO | 6 |
| Thromboxane A synthase 1 | TBXAS1 | 4 |
| Thromboxane A2 | TXA2 | 4 |
| Thromboxane A2 receptor | TBXA2R | 4 |
| Thy-1 T-cell antigen | THY1 | 4 |
| Thymidylate synthase | TYMS | 1 |
| Thymopoietin | TMPO | 6 |
| Thymosin | | 4 |
| Thyroid-stimulating hormone receptor | TSHR | 6 |
| Thyroid-stimulating hormone, alpha | TSHA | 6 |
| Thyroid-stimulating hormone, beta | TSHB | 6 |
| Thyrotropin releasing hormone | TRH | 5 |
| Thyrotropin releasing hormone | TRH | 6 |
| Thyrotropin releasing hormone receptor | TRHR | 5 |
| Tip-associated protein | TAP | 4 |
| Tissue inhibitor of metalloproteinase 1, TIMP1 | TIMP1 | 1 |
| Tissue inhibitor of metalloproteinase 2, TIMP2 | TIMP2 | 1 |
| Tissue inhibitor of metalloproteinase 3, TIMP3 | TIMP3 | 1 |
| Tissueinhibitorofmetalloproteinase4, TIMP4 | TIMP4 | 1 |
| Topoisomerase II | | 1 |
| Transacylase | | 1 |
| Transcobalamin 1, TCN1 | | 2 |
| Transcobalamin 2, TCN2 | TCN2 | 2 |
| Transcription factor 1, hepatic | TCF1 | 6 |
| Transcription factor 2, hepatic | TCF2 | 6 |
| Transcription factor 3 | TCF3 | 6 |
| Transcription factor binding to IGHM enhancer 3 | TFE3 | 6 |
| Transcription termination factor, RNA | TTF1 | 6 |
| polymerase 1 | | |
| Transcription termination factor, RNA | TTF2 | 6 |
| polymerase 2 | | |
| Transcription termination factor, RNA | TTF3 | 6 |
| polymerase 3 | | |
| Transferrin | TF | 6 |
| Transferrin receptor | TFRC | 6 |
| Transforming growth factor, alpha | TGFA | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Translocation in renal carcinoma on | TRC8 | 6 |
| chromosome 8 gene | | |
| Tuberous sclerosis 1 | TSC1 | 6 |
| Tuberous sclerosis 2 | TSC2 | 6 |
| Tubulin | | 3 |
| Tumor susceptibility gene 101 | TSG101 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour protein p73 | TP73 | 6 |
| Tumour protein, translationally-controlled 1 | TPT1 | 6 |
| Tumour suppresssor gene DRA | DRA | 4 |
| Twist (Drosophila) homolog | TWIST | 6 |
| Ubiquitin | | 6 |
| Ubiquitin activating enzyme, E1 | | 1 |
| Ubiquitin B | UBB | 6 |
| Ubiquitin C | UBC | 6 |
| Ubiquitin fusion degeneration 1-like | UFD1L | 6 |
| Ubiquitin protein ligase E3A | UBE3A | 1 |
| Vacuolar proton pump, subunit 1 | VPP1 | 5 |
| Vacuolar proton pump, subunit 3 | VPP3 | 5 |
| Vasoactive intestinal polypeptide | VIP | 5 |
| Vasoactive intestinal polypeptide receptor | VIPR | 5 |
| Vitamin B12-binding (R) protein | | 6 |
| Vitamin D receptor | VDR | 6 |
| v-myc avian myelocytomatosis viral oncogene | MYC | 6 |
| homolog | | |
| Von Hippel-Lindau gene | VHL | 6 |
| Werner syndrome helicase | WRN | 6 |
| Wilms tumour gene 1 | VVT I | 6 |
| Wi ms tumour gene 2 | WT2 | 6 |
| Wilms tumour gene 4 | WT4 | 6 |
| Winged helix nude | WHN | 6 |
| Wiskott-Aldrich syndrome protein | WASP, THC | 4 |
| Xeroderma pigmentosum, complementation | XPB | 1 |
| group B | | |
| Xeroderma pigmentosum, complementation | XPC | 1 |
| group C | | |
| Xeroderma pigmentosum, complementation | | 1 |
| group D | | |
| Xeroderma pigmentosum, complementation | | 1 |
| group E | | |
| Xeroderma pigmentosum, complementation | XPF | 1 |
| group F | | |
| Xeroderma pigmentosum, complementation | ERCC5 | 1 |
| group G | | |
| X-ray repair gene | XRCC9 | 6 |
| YY1 transcription factor | YY1 | 6 |
| Zinc finger protein HRX | ALL1 | 4, |

that the genes associated with symptoms and consequences of CNS malfunctions are selected from:

| | HUGO | Protein- |
|---|---|---|
| List of genes | gene symbol | function |
| 11-beta hydroxysteroid dehydrogenase 2 | HSD11B2 | 1 |
| 2,3-bisphosphoglycerate mutase | BPGM | 1 |
| 2,4-dienoyl CoA reductase | DECR | 1 |
| 3 beta hydroxysteroid dehydrogenase 2 | HSD3B2 | 1 |
| 3-oxoacid CoA transferase | OXCT | 1 |
| 4-hydroxyphenylpyruvate dioxygenase | HPD | 1 |
| 5,10-methylenetetrahydrofolate reductase | MTHFR | 1 |
| (NADPH) | | |
| 6-pyruvoyltetrahydropterin synthase | PTS | 1 |
| Acetoacetyl 2-CoA-thiolase | ACAT2 | 1 |
| Acetyl CoA acyltransferase | ACAA | 1 |
| Acetyl CoA carboxylase alpha | ACACA | 1 |
| Acetylcholine receptor, nicotinic, alpha A1 | CHRNAI | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Acyl CoA dehydrogenase, long chain | ACADL | 1 |
| Acyl CoA dehydrogenase, medium chain | ACADM | 1 |
| Acyl CoA dehydrogenase, short chain | ACADS | 1 |
| Acyl-CoA thioesterase | | 1 |
| Adaptin, beta 3A | ADTB3A | 2 |
| Adducin, alpha | ADD1 | 3 |
| Adducin, beta | ADD2 | 3 |
| Adenosine monophosphate deaminase | AMPD | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenyl cyclase | | 5 |
| Adenylate cyclase I | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylosuccinate lyase | ADSL | 1 |
| Adrenergic receptor, alpha1 | ADRAL | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Adrenoleukodystrophy gene | ALD | 1 |
| Albumin, ALB | ALB | 2 |
| Aldehyde dehydrogenase 10 | ALDH10 | 1 |
| Aldolase A | ALDOA | 1 |
| Aldolase B | ALDOB | 1 |
| Aldolase C | ALDOC | 1 |
| Aldosterone receptor | MLR | 6 |
| Alpha 2 macroglobulin | A2M | 4 |
| alpha tectorin | TECTA | 6 |
| alpha thalassemia gene | ATRX | 5 |
| alpha1-antitrypsin | PI | 1 |
| alpha2-antiplasmin | PLI | 1 |
| alpha-Galactosidase A | GLA | 1 |
| alpha-ketoglutarate dehydrogenase | | 1 |
| alpha-L-Iduronidase | IDUA | 1 |
| alpha-synuclein | SNCA | 5 |
| Aminomethyltransferase | AMT | 1 |
| Aminopeptidase P | XPNPEP2 | 1 |
| Amylo-1,6-glucosidase | AGL | 1 |
| Amyloid beta (A4) precursor protein-binding, | APBB1 | 5 |
| APBB1 | | |
| Amyloid beta A4 precursor protein | APP | 5 |
| Amyloid beta A4 precursor-like protein | APLP | 5 |
| Angiopoietin 1 | ANGPT1 | 6 |
| Angiopoietin 2 | ANGPT2 | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Antidiuretic hormone receptor | ADHR | 2 |
| Antithrombin III | AT3 | 1 |
| Apolipoprotein A I | APOA1 | 2 |
| Apolipoprotein A 11 | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Archaete-scute homolog 2 | ASH2 | 6 |
| Arginase | ARG1 | 1 |
| Arginine vasopressin | AVP | 5 |
| Arginosuccinate lyase | ASL | 1 |
| Arginosuccinate synthetase | ASS | 1 |
| Arylsulfatase A | ARSA | 1 |
| Arylsulfatase B | ARSB | 1 |
| Arylsulfatase D | ARSD | 1 |
| Arylsulfatase E | ARSE | 1 |
| Arylsulfatase F | ARSF | 1 |
| Aspaftoacylase | ASPA | 1 |
| Aspartylglucosaminidase | AGA | 1 |
| Astrotactin | ASTN | 6 |
| Ataxia telangiectasia complementation group D | ATD, ATDC | 6 |
| Ataxia telangiectasia gene, AT | ATM | 6 |
| ATP-binding cassette transporter 7 | ABC7 | 4 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Bagpipe homeobox, drosophila homolog of, 1 | BAPX1 | 6 |
| beta-Glucuronidase | GUSB | 1 |
| beta-synuclein | SNCB | 5 |
| Bilirubin UDP-glucuronosyltransferase | | 1 |
| Bloom syndrome protein | BLM | 6 |
| Bradykinin receptor B1 | | |
| Bradykinin receptor B2 | | |
| Brain derived neurotrophic factor | BDNF | 6 |
| Brain derived neurotrophic factor (BDNF) | BDNFR | 6 |
| receptor | | |
| Butyrylcholinesterase | BCHE | 1 |
| Ca(2+) transporting ATPase, slow twitch | ATP2A2 | 2 |
| Cadherin E | CDH1 | 6 |
| Cadherin EP | | 6 |
| Cadherin N | CDH2 | 6 |
| Cadherin P | CDH3 | 6 |
| Calbindin 1 | CALB1 | 6 |
| Calbindin D9K | CALB3 | 6 |
| Calcineurin A1 | CALNA1 | |
| Calcineurin A2 | CALNA2 | |
| Calcineurin A3 | CALNA3 | |
| Calcineurin B | | |
| Calcitonin/Calcitonin gene-related peptide | CALCA | 5 |
| alpha | | |
| Calcium channel, voltage-dependent, alpha | CACNA1F | 5 |
| 1 F subunit | | |
| Calcium channel, voltage-dependent, Alpha- | CACNA1B | 5 |
| 1 B (CACNLI A5) | | |
| Calcium channel, voltage-dependent, Alpha- | CACNA1C | 5 |
| I C | | |
| Calcium channel, voltage-dependent, Alpha- | CACNA1D | 5 |
| 1 D | | |
| Calcium channel, voltage-dependent, Alpha- | CACNA1E | 5 |
| I E (CACNLL A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1 A subunit | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase 11 | CAMK2A | 6 |
| Calnexin | CANX | 6 |
| Calpain | CAPN, CAPN3 | 1 |
| Calretinin | CALB2 | 5 |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Carnitine acetyltransferase | CRAT | 1 |
| Camitine acyicamitine translocase | CACT | 1 |
| Camitine transporter protein | CDSP, SCD | 2 |
| Carnosinase | | 5 |
| Caspasel | CASP1 | 6 |
| Catechol-o-methyltransferase | COMT | 1 |
| CD1 | CD1 | |
| CD4 | CD4 | |
| Cell adhesion molecule, neural, NCAM1 | NCAM1 | 6 |
| Cell adhesion molecule, neural, NCAM120 | NCAM120 | 6 |
| Cell adhesion molecule, neural, NCAM2 | NCAM2 | 6 |
| Ceroid lipofuscinosis neuronal 2 | CLN2 | 5 |
| Ceroid lipofuscinosis neuronal 3 | CLN3 | 5 |
| Ceroid lipofuscinosis neuronal 4 | CLN4 | 5 |
| Ceroid lipofuscinosis neuronal 5 | CLN5 | 5 |
| Ceroid lipofuscinosis neuronal 6 | CLN6 | 5 |
| Chemokine receptor CCR2 | CCR2 | |
| Chemokine receptor CCR3 | CCR3 | |
| Chemokine receptor CCR5 | CCR5 | 4 |
| Chemokine receptor CXCR4 | CXCR4 | 4 |
| Chloride channel 1, skeletal muscle | CLCN1 | 3 |
| Cholecystokinin | CCK | 5 |
| Cholecystokinin B receptor | CCKBR | 5 |
| Choline acetyltransferase | CHAT | 1 |
| Choroideremia gene | CHM | 3 |
| Chromogranin A | CHGA | 6 |
| Chymotrypsinogen | | 1 |
| Ciliary neurotrophic factor (CNTF) | CNTF | 6 |
| Ciliary neurotrophic factor (CNTF) receptor | CNTFR | 6 |
| Clathrin | | 2 |
| CoA transferase | | 1 |
| Cochlin | COCH | 4 |
| Cockayne syndrome gene, CKN1 | CKN1 | 6 |
| Cofilin | | 3 |
| Collagen I alpha 1 | COL1A1 | 3 |
| Collagen I alpha 2 | COL1A2 | 3 |
| Collagen II alpha 1 | COL2AI | 3 |
| Collagen III alpha 1 | COL3AI | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11AI | 3 |
| Collagen XI alpha 2 | COLL 1A2 | 3 |
| Collagen XVII alpha 1 | COL17A1 | 3 |
| Coliagenic-like tall subunit of asymmetric | COLQ | 1 |
| acetylcholinesterase | | |
| Colony-stimulating factor 1 | CSF1 | 6 |
| Colony-stimulating factor 1receptor | CSFIR | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Complex V | MTATP6 | 1 |
| Cone-rod homeobox-containing gene | CRX | 6 |
| Contactin | CNTN1 | 6 |
| Corticotrophin-releasing hormone | CRH | 2 |
| Corticotrophin-releasing hormone receptor | CRHR1 | 2 |
| Creb binding protein | CREBBP | 6 |
| Cu2+ transporting ATPase beta polypeptide | ATP7B | 1 |
| Cyclic AMP response element binding protein | CREB | 6 |
| Cyclic AMP-dependent protein kinase | PKA | 1 |
| Cyclic nucleotide gated channel alpha 1, | CNGAL | 5 |
| CNGA1 | | |
| Cyclic nucleotide gated channel alpha 3, | CNGA3 | 5 |
| CNGA3 | | |
| Cyclic nucleotide phosphodiesterase 1B | PDEIB | 1 |
| Cyclic nucleotide phosphodiesterase 1B1 | PDELBI | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin-dependent kinase 2 | CDK2 | 6 |
| Cyclooxygenase 1 | COX1 | 1 |
| Cyclooxygenase 2 | COX2 | 1 |
| CYP11A1 | CYPL1A1 | 1 |
| CYP11BI | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A33 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystatin B | CSTB | 2 |
| Cystatin C | CST3 | 2 |
| Cystinosin | CTNS | 2 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| DAX1 nuclear receptor | DAX1 | 4 |
| Deafness autosomal dominant 5 | DFNA5 | 5 |
| Deafness dystonia peptide | DDP | 5 |
| Deleted in malignant brain tumours 1 | DMBT1 | 6 |
| Delta aminolevulinate dehydratase | ALAD | 1 |
| Delta-7-dehydrocholesterol reductase | DHCR7 | 1 |
| DHEA sulfotransferase | STD | 1 |
| Diaphanous 1 | DIAPH1 | 5 |
| Diaphanous 2 | DIAPH2 | 5 |
| Dihydrolipoamide branched chain | DBT | 5 |
| transacylase | | |
| Dihydrolipoamide dehydrogenase | DLD | 5 |
| Dihydrolipoyl dehydrogenase 2 | PDHA | 1 |
| Dihydrolipoyl transacetylase | PDHA | 1 |
| Dihydroxyacetonephosphate acyltransferase | DHAPAT | 1 |
| DNA glycosylases | | 1 |
| DNA helicases | | 1 |
| DNA Ligase I | LIG1 | 1 |
| DNA methyltransferase | DNMT | 1 |
| DOPA decarboxylase | DDC | 1 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Doublecortin, DCX | DCX | 3 |
| Dynamin | DNM1 | 6 |
| Dystonia 1 | DYT1 | 3 |
| Dystonia 3 | DYT3 | 3 |
| Dystonia 6 | DYT6 | 3 |
| Dystonia 7 | DYT7 | 3 |
| Dystonia 9 | CSE | 3 |
| Dystrophia myotonica | DM, DMPK | 1 |
| Dystrophia myotonica, atypical | DM2 | 1 |
| Dystrophin | DMD | 3 |
| Ectodermal Dysplasia 1 gene | ED1 | 3 |
| Electron-transfering-flavoprotein alpha | ETFA | 2 |
| Electron-transfering-flavoprotein beta | ETFB | 2 |
| Electron-transferring flavoprotein | ETFDH | 1 |
| dehydrogenase | | |
| Emerin | EMD | 2 |
| Empty spiracles (drosophila) homologue 1 | EMX1 | 6 |
| Empty spiracies (drosophila) homologue 2 | EMX2 | 6 |
| Endobrevin | VAMP8 | 5 |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | ENO1 | 1 |
| Enoyl CoA isomerase | | 1 |
| Enoyl CoA reductase | | 1 |
| Enterokinase | PRSS7, ENTK | 1 |
| Ephrin-A | EFNA | 6 |
| Ephrin-B | EFNB | 6 |
| Epidermal growth factor | EGF | 6 |
| Epidermal growth factor receptor | EGFR | 6 |
| Epilepsy, progressive myoclonic 2 gene | EPM2A | 1 |
| EWS RNA-binding protein | EWSR1 | 6 |
| Excision repair complementation group 4 | ERCC4 | 1 |
| protein | | |
| Exostosin 1 | EXTI | 3 |
| Exostosin 2 | EXT2 | 3 |
| Factor I (No. one) | F1 | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor Xii | F12 | 4 |
| Factor XIII A & B | F13A & F13B | 4 |
| Fanconi anemia, complementation group A | FANCA | 2 |
| Fanconi anemia, complementation group C | FANCC | 2 |
| Fanconi anemia, complementation group D | FANCD | 2 |
| Fibrillin 2 | FBN2 | 6 |
| Fibrinogen alpha | FGA | 3 |
| Fibrinogen beta | FGB | 3 |
| Fibrinogen gamma | FGG | 3 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Flightless-11, Drosophila homolog of | FLII | 6 |
| Follicle stimulating hormone receptor | FSHR, ODG1 | 6 |
| Fol icle stimulating hormone, FSH | FSHB | 6 |
| Forkhead transcription factor IO | FKHL10 | 6 |
| Formiminotransferase | | 1 |
| Fragile site, folic acid type, rare, fra(X) A | FRAXA | 5 |
| Fragile site, folic acid type, rare, fra(X) E | FRAXE | 5 |
| Fragile site, folic acid type, rare, fra(X) F | FRAXF | 5 |
| Frataxin | FRDA | 6 |
| Fructose-1,6-diphosphatase | FBP1 | 1 |
| Fukuyama type congenital muscular | FCMD | 6 |
| dystrophy | | |
| GABA receptor, alpha 1 | GABRAL | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Galactocerebrosidase | GALC | 1 |
| Galactose 1-phosphate uridyi-transferase | GALT | 1 |
| Galactosyltransferase 1 | GT1 | 6 |
| Galactosyltransferase, alpha 1,3 | GGTAL | 6 |
| Galactosyltransferase, beta 3 | B3GALT | 6 |
| Galanin | GAL | 5 |
| Galanin receptor | GALNRL | 5 |
| Gamma-glutamyltransferase 1 | GGTI | 2 |
| Gap junction protein beta 2 | GJB2 | 2 |
| Gap junction protein beta 3 | GJB3 | 2 |
| Gastric Intrinsic factor, GIF | GIF | 1 |
| Gastrulation brain homeobox 2 | GBX2 | 6 |
| Geniospasm I | GSM1 | 6 |
| Gephyrin | | 5 |
| Glial-cell derived neurotrophic factor (GDNF) | | 5 |
| receptor | | |
| Glial-cell derived neurotrophic factor, GDNF | GDNF | 5 |
| Glucosidase, acid alpha | GAA | 1 |
| Glutamate decarboxylase, GAD | GAD1 | 1 |
| Glutamate dehydrogenase | GLUD1 | 1 |
| Glutamate receptor 1 | GLURI | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutamate-cysteine ligase | GLCLC | 1 |
| Glutaryl-CoA dehydrogenase | GCDH | 1 |
| Glutathione | GSH | 2 |
| Glutathione S-transferase, GSTZ1 | GSTZI | 1 |
| Glutathione synthetase | GSS | 1 |
| Glyceraldehyde-3-phosphate | GAPDH | 1 |
| dehydrogenase, GAPDH | | |
| Glycerol kinase | GK | 1 |
| Glycinamide ribonucleotide (GAR) | GART | 1 |
| transformylase | | |
| Glycine dehydrogenase | GLDC | 1 |
| Glycine receptor, alpha | GLRA2 | 5 |
| Glycine receptor, beta | | 5 |
| Glycine transporter | GLYT | 5 |
| Glycogen phosphorylase | PYGL | 1 |
| GM2 ganglioside activator protein, GM2A | GM2A | 1 |
| Gonadotropin releasing hormone receptor | GNRHR | 6 |
| GTP cylcohydrolase 1 | GCH1 | 6 |
| Guanidinoacetate N-methyltransferase | GAMT | 1 |
| Guanine nucleotide-binding protein, alpha | GNA01 | 5 |
| activating activity polypeptide, GNAO | | |
| Guanine nucleotide-binding protein, alpha | GNAI1 | 5 |
| inhibiting activity polypeptide 1, GNAI1 | | |
| Guanine nucleotide-binding protein, alpha | GNAI2 | 5 |
| inhibiting activity polypeptide 2, GNAI2 | | |
| Guanine nucleotide-binding protein, alpha | GNAI3 | 5 |
| inhibiting activity polypeptide 3, GNAI3 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNAT1 | 5 |
| transducing activity polypeptide, GNAT1 | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, q | GNAQ | 5 |
| polypeptide | | |
| Guanylate cyclase 2D, membrane (retina- | GUCY2D | 1 |
| specific) | | |
| Guanylate cyclase activator 1A (retina) | GUCA1A | 1 |
| Guanylate kinase | | 1 |
| Guanylyl cyclase | | 1 |
| Gustducin, alpha (taste-specific G protein) | GDCA | 5 |
| Haeme regulated inhibitor kinase | | |
| Haemoglobin alpha 1 | HBA1 | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Heat shock protein, HSP60 | | 4 |
| Heat shock protein, HSP70 | | 4 |
| Heat shock protein, HSP90 | | 4 |
| Heat shock protein, HSPAL | | 4 |
| Heat shock protein, HSPA2 | | 4 |
| Heparan sulfamidase | | 1 |
| Heparin binding epidermal growth factor | HBEGF | 6 |
| Heparin Cofactor II | HCF2 | 4 |
| Hepatic lipase | LIPC | 1 |
| Hexosaminidase A | HEXA, TSD | 1 |
| Hexosaminidase B | HEXB | 1 |
| Hippocampal cholinergic neurostimulating peptide, HCNP | | 5 |
| Histamine receptors, H1 | | 5 |
| Histamine receptors, H2 | | 5 |
| Histamine receptors, H3 | | 5 |
| Histidase | | 1 |
| HLA-B associated transcript 1 | BAT1 | 4 |
| HLH transcription factor HAND1 | HAND1 | 6 |
| HLH transcription factor HAND2 | HAND2 | 6 |
| HMG-CoA lyase | HMGCL | 1 |
| HMG-CoA reductase | HMGCR | 1 |
| Holocarboxylase synthetase | HLCS | 1 |
| Homeobox HB9 | HLXB9 | 6 |
| Human atonal gene | ATOH1 | 6 |
| Hypoxanthine-guanine | HPRT | 1 |
| phosphoribosyltransferase, HGPRT | | |
| Hypoxia inducible factor 1 | HIFIA | 1 |
| Hypoxia inducible factor 2 | | 1 |
| IC7 A and B | | |
| Inositol 1,4,5-triphosphate receptor I | ITPR1 | 6 |
| Inositol monophosphatase | IMPA1 | 5 |
| Inositol polyphosphate 1-phosphatase | INPP1 | 5 |
| Insulin | INS | 6 |
| Insulin receptor | INSR | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| Integrin beta 1 | ITGB1 | 6 |
| Integrin beta 2 | ITGB2 | 6 |
| Integrin beta 3 | ITGB3 | 6 |
| Integrin, alpha 1 | ITGA1 | 6 |
| Integrin, alpha M | ITGAM | 6 |
| Inter-alpha-trypsin inhibitor, IATI | | 1 |
| Interleukin(IL) 1 receptor | IL1 R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) 11 | IL11 | 4 |
| Interleukin(IL) 11 receptor | IL11R | 4 |
| Interleukin(IL) 12 | IL12 | 4 |
| Interleukin(IL) 12 receptor, beta 1 | IL12RB1 | 4 |
| Interleukin(IL) 13 | IL13 | 4 |
| Interleukin(IL) 13 receptor | IL13R | 4 |
| Interleukin(IL) 2 | IL2 | 4 |
| Intern(IL) 2 receptor, alpha | IL2RA | 4 |
| Interleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| Interleukin(IL) 9 | IL9 | 4 |
| Interleukin(IL) 9 receptor | IL9R | 4 |
| Interleukin(IL) receptor antagonist 1 | IL1RN, IL1RA | 4 |
| IP3 kinase | | 1 |
| Isovaleric acid CoA dehydrogenase | IVD | 1 |
| Kallikrein 3 | KAK3 | 4 |
| Kallman syndrome gene 1 | KAL1 | 6 |
| Ketohexokinase | KHK | 1 |
| Kininogen, High molecular weight | KNG | 4 |
| Kynureninease | | 1 |
| L1 cell adhesion molecule | L1CAM | 5 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor I | LAMR1 | 6 |
| Latent transforming growth factor-beta | LTBP2 | 6 |
| binding protein 2 | | |
| Leptin | LEP | 6 |
| Leptin receptor | LEPR | 6 |
| Leukaemia inhibitory factor | LIF | 6 |
| Leukaemia inhibitory factor receptor | LIFR | 6 |
| Leukin | | 4 |
| Leukocyte-specific transcript 1 | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| LIM homeobox protein 2 | LHX2 | 6 |
| LIM homeobox protein 3 | LHX3 | 6 |
| LIM homeobox protein 4 | LHX4 | 6 |
| Limbic associated membrane protein | LAMP | 6 |
| LIM-domain only protein 1 | LM01 | 6 |
| LIM-domain only protein 2 | LM02 | 6 |
| LIM-domain only protein 3 | LM03 | 6 |
| LIM-domain only protein 4 | LM04 | 6 |
| LIM-Kinase I (LINK-I) | | 4 |
| Lipoprotein receptor, Low Density | LDLR | 2 |
| Lipoprotein, High Density | HDLDT1 | 2 |
| Lipoprotein, Intermediate Density | | 2 |
| Lipoprotein, Low Density 1 | | 2 |
| Lipoprotein, Low Density 2 | | 2 |
| Lipoprotein, Very Low Density | VLDLR | 2 |
| Low density lipoprotein receptor-related | LRP | 2 |
| protein precursor | | |
| Lymphoid enhancer-binding factor | LEF-1 | 6 |
| MAD (mothers against decapentaplegic, | MADH4 | 6 |
| Drosophila) homologue 4 | | |
| Malonyl CoA decarboxylase | | 1 |
| Mannosidase, alpha B lysosomal | MANB | 1 |
| Mannosidase, beta A lysosomal | MANBA | 1 |
| Marenostrin | MEFV | 2 |
| Melatonin receptor 1A | MTNRLA | 5 |
| Melatonin receptor 1 B | MTNRLB | 5 |
| Methylguanine-DNA methyltransferase | MGMT | 1 |
| Methylmalonyl-CoA mutase | MUT | 1 |
| Mevalonate kinase | MVK | 1 |
| Microsomal triglyceride transfer protein | MTP | 2 |
| Microtuble associated protein | MAP | 3 |
| Mismatch repair gene, PMSL2 | PMS2 | 6 |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Msh homeobox homolog 2 | MSX2 | 6 |
| Mucolipidoses | GNPTA | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Myelin protein peripheral 22 | PMP22 | 3 |
| Myelin protein zero | MPZ | 3 |
| Myogenic factor 3 | MYF3 | 6 |
| Myogenic factor 4 | MYF4 | 6 |
| Myogenic factor 5 | MYF5 | 6 |
| Myosin 6 | MY06 | 3 |
| Myosin 7A | MY07A | 3 |
| Myotubularin | MTMI | 3 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+, K+ ATPase, beta 3 | ATP1B3 | 6 |
| N-acetylglucosamine-6-sulfatase | GNS | 1 |
| N-acetylglucosaminidase, alpha | NAGLU | 1 |
| NADH dehydrogenase | | 1 |
| reductase | | |
| NB6 | | 4 |
| Nebulin | NEB | 3 |
| Necdin | NDN | 6 |
| Nerve growth factor | NGF | 6 |
| Nerve growth factor receptor | NGFR | 6 |
| Neural retina-specific gene | NRL | 6 |
| Neuraminidase sialidase | NEU | 2 |
| Neuregulin | HGL | 6 |
| Neurite growth-promoting factor 2 | MDK | 5 |
| Neurite inhibitory protein | | 5 |
| Neuroendocrine convertase 1 | NEC1, PCSKI | 1 |
| Neurofibromin I | NF1 | 6 |
| Neurofibromin 2 | NF2 | 6 |
| Neurofilament protein, heavy | NFH | 3 |
| Neurofilament protein, NF68 | NF68 | 3 |
| Neurokinin A | NKNA | 5 |
| Neurokinin B | NKNB | 5 |
| Neuronal apoptosis inhibitory protein | NAIP | 4 |
| Neuronal molecule-1 | | 4 |
| Neuronal molecule-1 receptor | | 4 |
| Neuropeptide Y | NPY | 5 |
| Neuropeptide Y receptor Y1 | NPY1R | 5 |
| Neuropeptide Y receptor Y2 | NPY2R | 5 |
| Neurotensin | NTS | 5 |
| Neurotensin receptor | NTSR1 | 5 |
| Neutral endopeptidase | | 1 |
| Niemann-Pick disease protein | NPC1 | 2 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Notch 1 | NOTCH1 | 6 |
| Notch 2 | NOTCH2 | 6 |
| Notch 3 | NOTCH3 | 6 |
| Notch ligand-jagged 1 | JAG 1, AGS | 6 |
| Nuclear factor I-kappa-B-like gene | IKBL | 4 |
| Nucleoside diphosphate kinase-A | NDPKA | 1 |
| Oncogene bcl2 | | 6 |
| Oncogene GLI I | GLI | 6 |
| Oncogene GLI2 | GLI2 | 6 |
| Oncogene GLI3 | GLI3 | 6 |
| Oncogene sis | PDGFB | 6 |
| Opioid receptor, delta | OPRD1 | 5 |
| Opioid receptor, kappa | OPRK1 | 5 |
| Opioid receptor, mu | OPRM1 | 5 |
| Ornithine delta-aminotransferase | OAT | 1 |
| Ornithine transcarbamoylase | OTC, NME1 | 1 |
| Orthodenticle (Drosophila) homolog I | OTX1 | 6 |
| Orthodenticle (Drosophila) homolog 2 | OTX2 | 6 |
| Otoferlin | OTOF | 5 |
| Paired box homeotic gene 2 | PAX2 | 6 |
| Paired box homeotic gene 3 | PAX3 | 6 |
| Palmitoyi-protein thioesterase | PPT | 2 |
| Parkin | PARK2 | 5 |
| Patched (Drosophila) homolog, PTCH | PTCH | 6 |
| Peanut-like 1 | PNUTLL | 4 |
| PeptidylglyCihe alpha-amidating | PAM | 1 |
| monooxygenase | | |
| Peripherin, PRPH | | 3 |
| Peroxisomal membrane protein 1 | PXMP1 | 3 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome receptor 1 | PXR1 | 2 |
| Persyn | | 3 |
| Phosphate regulating gene with homologies | PHEX | 6 |
| to endopeptidases on the X chromosome | | |
| Phosphatidylinositol transfer protein | PITPN | 6 |
| Phosphoglucose isomerase | GPI | 1 |
| Phosphoglycerate kinase I | PGKL | 1 |
| Phospholipase A2, group 10 | PLA2G I 0 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase 2 | PMM2 | 6 |
| Phosphoribogyl pyrophosphate synthetase | PRPS1 | 1 |
| Phytanoyi-CoA hydroxylase | PHYH | 6 |
| Plakophilin 1 | PKP1 | 2 |
| Plasminogen | PLG | 1 |
| Plasminogen activator inhibitor 1 | PAI 1 | 1 |
| Plasminogen activator inhibitor 2 | PAI2 | 1 |
| Plasminogen activator receptor, Urokinase | UPAR, PLAUR | 3 |
| Plasminogen activator, Tissue | PLAT, TPA | 1 |
| Plasminogen activator, Urokinase | UPA, PLAU | 1 |
| Platelet derived growth factor | PDGF | 6 |
| Platelet derived growth factor receptor | PDGFR | 6 |
| Platelet-activating factor receptor | PAFR | 4 |
| Plectin 1 | PLEC1 | 2 |
| Postsynaptic density-95 protein | PSD95 | 5 |
| Potassium channel, calcium-activated | KCNN4 | 5 |
| Potassium channel, subfamily K, member 1 | KCNK1 | 5 |
| Potassium channel, subfamily K, member 2 | KCNK2 | 5 |
| Potassium channel, subfamily K, member 3 | KCNK3 | 5 |
| Potassium inwardly-rectifying channel J 1 | KCNJ1 | 5 |
| Potassium voltage-gated channel A1 | KCNA1 | 5 |
| Potassium voltage-gated channel E1 | KCNE1 | 5 |
| Potassium voltage-gated channel Q1 | KCNQ1 | 5 |
| Potassium voltage-gated channel Q2 | KCNQ2 | 5 |
| Potassium voltage-gated channel Q3 | KCNQ3 | 5 |
| Potassium voltage-gated channel Q4 | KCNQ4 | 5 |
| POU domain, class 1, transcription factor 1 | POU1F1 | 6 |
| (Pit1) | | |
| POU domain, class 3, transcription factor 4 | POU3F4 | 6 |
| POU domain, class 4, transcription factor 3 | POU4F3 | 6 |
| Prekallikrein | | 4 |
| Preproenkephalin | PENK | 5 |
| Presenitin | PSEN1 | 2 |
| Presenilin 2 | PSEN2 | 2 |
| Prion protein | PRNP | 5 |
| Procollagen N-protease | | 1 |
| Proline dehydrogenase | PRODH | 1 |
| Pro-melanin-concentrating hormone | PMCH | 6 |
| Proopiomelanocortin | POMC | 5 |
| Prosaposin | PSAP | 5 |
| Prostacyclin synthase | | 4 |
| Prostaglandin 15-OH dehydrogenase | HGPD, PGDH | 4 |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin I2 receptor | | 2 |
| Prostaglandin IP receptor | | 4 |
| Protease nexin 2 | PN2 | 1 |
| Protective protein for beta-galactosidase | PPGB | 1 |
| Protein C | PROC | 4 |
| Protein C inhibitor | PCI | 4 |
| Protein kinase C, alpha | PRKCA | 1 |
| Protein kinase C, gamma | PRKCG | 1 |
| Protein kinase G | | 1 |
| Protein phosphatase 1, regulatory (inhibitor) | PPP1R3 | 1 |
| subunit 3 | | |
| Protein S | PROS1 | |
| Prothrombin precursor | F2 | |
| Purine nucleoside phosphorylase | NP | 1 |
| Pyrroline-5-carboxylate synthetase | PYCS | 1 |
| Pyruvate carboxylase | PC | 1 |
| Pyruvate decarboxylase | PDHA | 1 |
| Ras-G-protein | RAS | 6 |
| Rathke pouch homeobox, RPX | RPX | 6 |
| Renin | REN | 1 |
| Replication factor C | RFC2 | 1 |
| Retinal pigment epithelium specific protein | RPP65 | 3 |
| (65 kD) | | |
| Retinaldehyde binding protein 1 | RLBP1 | 2 |
| Retinoblastoma 1 | RB1 | 6 |
| Rhodopsin kinase | RHOK | 1 |
| RIGUI | RIGUI | 6 |
| S100 calcium-binding protein A3 | S I00A3 | 5 |
| S100 calcium-binding protein A4 | S100A4 | 5 |
| S100 calcium-binding protein A5 | S100A5 | 5 |
| S100 calcium-binding protein A7 | S100A7 | 5 |
| S100 calcium-binding protein A8 | S100A8 | 5 |
| S100 calcium-binding protein A9 | S100A9 | 5 |
| S100 calcium-binding protein P | S100P | 5 |
| Secretase, alpha | | 5 |
| Secretase, beta | | 5 |
| Secretase, gamma | | 5 |
| Selectin E | SELE | 5 |
| Selectin L | SELL | 5 |
| Selectin P | SELP | 5 |
| Semaphorin A4 | SEMA4 | 3 |
| Semaphorin A5 | SEMA5 | 3 |
| Semaphorin D | | 3 |
| Semaphorin E | SEMAE | 3 |
| Semaphorin F | SEMA3/F | 3 |
| Semaphorin W | SEMAW | 3 |
| Serotonin N-acetyltransferase | SNAT | 1 |
| Serotonin receptor, 5HT1A | HTRIA | 5 |
| Serotonin receptor, 5HT1B | HTRIB | 5 |
| Serotonin receptor, 5HT1C | HTRLC | 5 |
| Serotonin receptor, 5HT1D | HTRL D | 5 |
| Serotonin receptor, 5HT1E | HTRI E | 5 |
| Serotonin receptor, 5HT1F | HTRL F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Signaling lymphocyte activation molecule | SLAM | 4 |
| Slug protein | | 6 |
| Small nuclear ribonucleoprotein polypeptide | SNRPNN | 3 |
| Sodium channel, non-voltage gated 1, alpha | SCNN1A | 5 |
| Sodium channel, non-voltage gated 1, beta | SCNN1B | 5 |
| Sodium channel, non-voltage gated 1, | SCNN1G | 5 |
| gamma | | |
| Sodium channel, voltage gated, type IV, | SCN4A | 5 |
| alpha polypeptide | | |
| Sodium channel, voltage-gated, type 1, beta | SCNLB | 5 |
| polypeptide | | |
| Solute carrier family 1 (amino acid | SLC1A6 | 2 |
| transporter), member 6 | | |
| Solute carrier family 1 (glial high affinity | SLC1A3 | 2 |
| glutamate transporter), member 3 | | |
| Solute carrier family 1 (glutamate | SLC1A1 | 2 |
| transporter), member 1 | | |
| Solute carrier family 1 (glutamate | SLC1A2 | 2 |
| transporter), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 18, member 3 | SLC18A3 | 2 |
| Solute carrier family 2 (facilitated glucose | SLC2A1 | 2 |
| transporter), member I | | |
| Solute carrier family 20, member 3 | SLC20A3 | 2 |
| Solute carrier family 25, member 12 | SLC25A12 | 2 |
| Solute carrier family 4 (anion exchangers, | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member I | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Solute carrier family 6, member 6 | SLC6A6 | 2 |
| Solute carrier family 7(amino acid | SLC7A1 | 2 |
| transporter), member 1 | | |
| Solute carrier family 7(amino acid | SLC7A2 | 2 |
| transporter), member 2 | | |
| Solute carrier family 7(amino acid | SLC7A7 | 2 |
| transporter), member 7 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Spastic paraplegia 7 | SPG7 | 6 |
| Spectrin beta | SPTB | 3 |
| Sphingomyelinase | SMPD1 | 1 |
| Spinocerebellar ataxia 8 gene | SCA8 | 5 |
| SRY-box 11 | SOX11 | 6 |
| Steroid 5 alpha reductase 1 | SRD5AI | 1 |
| Steroid 5 alpha reductase 2 | SRD5A2 | 1 |
| Steroid sulphatase | STS | 1 |
| Substance P | | 5 |
| Succinic semi-aldehyde dehydrogenase | SSADH | 1 |
| Sulfamidase | SGSH | 6 |
| Sulfite oxidase | SUOX | 1 |
| Superoxide dismutase 1 | SOD1 | 1 |
| Superoxide dismutase 3 | SOD3 | 1 |
| Surfeit 1 | SURF1 | 6 |
| Survival of motor neuron 1, telomeric | SMN1 | 2 |
| Synapsin 1a & 1b | SYN1 | 5 |
| Synapsin 2a & 2b | SYN2 | 5 |
| Synaptic vesicle amine transporter | SVAT | 5 |
| Synaptic vesicle protein 2 | SV2 | 5 |
| Synaptobrevin 1 | SYB1 | 5 |
| Synaptobrevin 2 | SYB2 | 5 |
| Synaptogyrin | | 5 |
| Synaptophysin | SYP | 5 |
| Synaptosomal-associated protein, 25 KD | SNAP25 | 5 |
| Synaptotagmin 1 | SYT1 | 5 |
| Synaptotagmin 2 | SYT2 | 5 |
| Syntaxin 1 | STX1 | 5 |
| Tachykinin receptor, NK1R | TACR1 | 5 |
| Tachykinin receptor, NK2R | TACR2 | 5 |
| Tachykinin receptor, NK3R | TACR3 | 5 |
| Talin | TLN | 6 |
| Tau protein | MAPT | 3 |
| TEK, tyrosine kinase, endothelial | TEK | 1 |
| Telomerase protein component | | 1 |
| Thiolase, perioxisomal | | 1 |
| Thrombin receptor | F2R | 4 |
| Thrombopoietin | THPO | 6 |
| Thromboxane A synthase 1 | TBXAS1 | 4 |
| Thromboxane A2 | TXA2 | 4 |
| Thromboxane A2 receptor | TBXA2R | 4 |
| Thy-1 T-cell antigen | THY1 | 4 |
| Thyroxin-binding globulin | TBG | 2 |
| Tocopherol (alpha) transfer protein | TTPA | 2 |
| Topoisomerase I | | 1 |
| Tofticollis, keloids, cryptorchidism and renal | TKCR | 6 |
| dysplasia gene | | |
| Transacylase | | 1 |
| Transferrin receptor | TFRC | 6 |
| Transforming growth factor, beta 2 | TGFB2 | 6 |
| Transforming growth factor, beta induced | TGFB1 | 6 |
| Transforming growth factor, beta receptor 2 | TGFBR2 | 6 |
| Transketolase-like 1 | TKTL1 | 1 |
| Transthyretin | TTR | 2 |
| Tremor, essential 1 | ETM1 | 5 |
| Tremor, essential 2 | ETM2 | 5 |
| Triosephosphate isomerase | TPI1 | 1 |
| Tropomyosin 3 (non-muscle) | TPM3 | 3 |
| Tryptophan hydroxylase | TPH | 1 |
| Tubby-like protein 1 | TULP1 | 6 |
| Tuberous sclerosis 1 | TSC1 | 6 |
| Tuberous sclerosis 2 | TSC2 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p73 | TP73 | 6 |
| Tyrosine aminotransferase | TAT | 1 |
| Tyrosine hydroxylase | TH | 1 |
| Ubiquitin | | 6 |
| Ubiquitin B | UBB | 6 |
| Ubiquitin C | UBC | 6 |
| Ubiquitin carboxyl-terminal esterase L1 | UCHL1 | 6 |
| UDP-glucuronosyltransferase 1 | ugt1d, UGTL | 1 |
| UDP-glucuronosyltransferase 2 | UGT2 | 1 |
| Urate oxidase | UOX | 1 |
| Uridinediphosphate(UDP)-galactose-4- | GALE | 1 |
| epimerase | | |
| Uroporphyrinogen III synthase | UROS | 1 |
| Usher syndrome 2A | USH2A | 3 |
| Vacuolar proton pump, subunit 1 | VPP1 | 5 |
| Vacuolar proton pump, subunit 3 | VPP3 | 5 |
| Vasoactive intestinal polypeptide | VIP | 5 |
| Vasoactive intestinal polypeptide receptor | VIPR | 5 |
| Vesicular monoamine transporter 1 | VMAT1 | 5 |
| Vesicular monoamine transporter 2 | VMAT2 | 5 |
| Vitamin B12-binding (R) protein | | 6 |
| Von Hippel-Lindau gene | VHL | 6 |
| Wolf-Hirschhorn syndrome candidate I gene | WHSC1 | 6 |
| Wolfram syndrome 1 gene | WFS1 | 3 |
| Xanthine dehydrogenase | XDH | 1 |
| Xeroderma pigmentosum, complementation | XPA | 1 |
| group A | | |
| Zinc finger protein 2 | ZIC2 | 3 |

that the genes associated with symptoms of aggression or behavioral disturbances are selected from:

| | HUGO | Protein- |
|---|---|---|
| List of genes | gene symbol | function |
| 11 beta Hydroxysteroid dehydrogenase 2 | HSD11B2 | 1 |
| 4-Hydroxyphenylpyruvate dioxygenase | HPD | 1 |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acety choline receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| alpha-Synuclein | SNCA | 5 |
| Amyloid beta A4 precursor protein | APP | 5 |
| Amyloid beta A4 precursor-like protein | APLP | 5 |
| Androgen binding protein | ABP | 2 |
| Androgen receptor | AR | 6 |
| Apolipoprotein E | APOE | 2 |
| Arginosuccinate synthetase | ASS | 1 |
| Ataxia telangiectasia gene, AT | ATM | 6 |
| beta-synuclein | SNCB | 5 |
| Ca(2+) transporting ATPase, slow twitch | ATP2A2 | 2 |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Catechol-o-methyltransferase | COMT | 1 |
| Cholecystokinin | CCK | 5 |
| Cholecystokinin B receptor | CCKBR | 5 |
| Choline acetyltransferase | CHAT | 1 |
| Ciliary neurotrophic factor (CNTF) | CNTF | 6 |
| Ciliary neurotrophic factor (CNTF) receptor | CNTFR | 6 |
| Corticotrophin-releasing hormone | CRH | 2 |
| Corticotrophin-releasing hormone receptor | CRHR1 | 2 |
| Cryptochrome 1 | CRY1 | 3 |
| Cryptochrome 2 | CRY2 | 3 |
| Cu2+ transporting ATPase beta polypeptide | ATP7B | 1 |
| Cyclic AMP-dependent protein kinase | PKA | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYPIA2 | CYPIA2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Dihydrolipoamide branched chain transacylase | DBT | 5 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Doublecortin, DCX | DCX | 3 |
| Enolase | EN01 | 1 |
| Flightless-II, Drosophila homolog of | FLII | 6 |
| Fragile site, folic acid type, rare, fra(X) A | FRAXA | 5 |
| Fragile site, folic acid type, rare, fra(X) E | FRAXE | 5 |
| Fragile site, folic acid type, rare, fra(X) F | FRAXF | 5 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| Galactose 1-phosphate uridyl-transferase | GALT | 1 |
| Geniospasm 1 | GSM1 | 6 |
| Glutathione | GSH | 2 |
| Glutathione S-transferase, GSTZ1 | GSTZ1 | 1 |
| Glyceraidehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| GM2 ganglioside activator protein, GM2A | GM2A | 1 |
| Gustducin, alpha (taste-specific G protein) | GDCA | 5 |
| Inositol monophosphatase | IMPA1 | 5 |
| IP3 kinase | | 1 |
| Mannosidase, beta A lysosomal | MAN1A | 1 |
| Melatonin receptor 1 A | MTNR1A | 5 |
| Melatonin receptor 1 B | MTNR1B | 5 |
| Monoamine oxidase A | MAOA | 1 |
| Monoamine oxidase B | MAOB | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| N-acetylglucosamine-6-sulfatase | GNS | 1 |
| reductase | | |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Neurotensin | NTS | 5 |
| Neurotensin receptor | NTSR1 | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Ocular albinism 1 | OA1 | 3 |
| Opioid receptor, delta | OPRD1 | 5 |
| Opioid receptor, kappa | OPRK1 | 5 |
| Opioid receptor, mu | OPRM1 | 5 |
| Orexin | OX | 6 |
| Orexin 1 receptor | OX1R | 6 |
| Orexin 2 receptor | OX2R | 6 |
| Phosphoglycerate kinase 1 | PGK1 | 1 |
| Potassium inwardly-rectifying channel J1 | KCNJ1 | 5 |
| Potassium voltage-gated channel E1 | KCNE1 | 5 |
| Potassium voltage-gated channel Q1 | KCNQ1 | 5 |
| Preproenkephalin | PENK | 5 |
| Preproglucagon | GCG, GLP1, | 6 |
| | GLP2 | |
| Prion protein | PRNP | 5 |
| Proline dehydrogenase | PRODH | 1 |
| Pro-melanin-concentrating hormone | PMCH | 6 |
| Proopiomelanocortin | POMC | 5 |
| Purine nucleoside phosphorylase | NP | 1 |
| RIGUI | RIGUI | 6 |
| Serotonin N-acetyltransferase | SNAT | |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Solute carrier family 18, member 3 | SLC18A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Synapsin 1a & 1 b | SYN1 | 5 |
| Synapsin 2a & 2b | SYN2 | 5 |
| Synaptogydn | | 5 |
| Synaptophysin | SYP | 5 |
| Synaptosomal-associated protein, 25 KD | SNAP25 | 5 |
| Syntaxin 1 | STx1 | 5 |
| Tachykinin receptor, NKIR | TACR1 | 5 |
| Tachykinin receptor, NK2R | TACR2 | 5 |
| Tachykinin receptor, NK3R | TACR3 | 5 |
| Tau protein | MAPT | 3 |
| Tryptophan hydroxylase | TPH | 1 |
| Tyrosine hydroxyiase | TH | 1 |
| Ubiquitin | | 6 |
| Ubiquitin B | UBB | 6 |
| Ubiquitin C | UBC | 6 |
| UDP-glucuronosyltransferase 1 | ugt1d, UGT1 | 1 |
| UDP-glucuronosyltransferase 2 | UGT2 | 1 |
| Vacuolar proton pump, subunit 1 | VPPI | 5 |
| Vacuolar proton pump, subunit 3 | VPP3 | 5 |
| Vasoactive intestinal polypeptide | VIP | 5 |
| Vasoactive intestinal polypeptide receptor | VIPR | 5, |

that the genes associated with the consequences of clinical, psychological and social consequences of a brain lesion are selected from:

| | HUGO | Protein- |
|---|---|---|
| List of genes | gene symbol | function |
| 2,3-bisphosphoglycerate mutase | BPGM | 1 |
| 3-beta hydroxysteroid dehydrogenase 2 | HSD3B2 | 1 |
| 4-hydroxyphenylpyruvate dioxygenase | HPD | 1 |
| 5,10-methylenetetrahydrofolate reductase | MTHFR | 1 |
| (NADPH) | | |
| 6-pyruvoyltetrahydropterin synthase | PTS | 1 |
| Acetoacetyl 2-CoA-thiolase | ACAT2 | 1 |
| Acetyl CoA acyltransferase | ACAA | 1 |
| Acetylcholine receptor, nicotinic, alpha Al | CHRNAL | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNBL | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Adducin, alpha | ADDI | 3 |
| Adducin, beta | ADD2 | 3 |
| Adenosine receptor Al | ADORAL | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alphal | ADRAL | 5 |
| Adrenergic receptor, alpha2 | ADRA2 | 5 |
| Adrenergic receptor, beta1 | ADRBI | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| Aldehyde dehydrogenase 10 | ALDH10 | 1 |
| Aldosterone receptor | MLR | 6 |
| Alpha 1 acid glycoprotein | AAG; AGP | 2 |
| Alpha 2 macroglobulin | A2M | 4 |
| alpha thalassemia gene | ATRX | 5 |
| alpha 1-antitrypsin | PI | 1 |
| alpha 2-antiplasmin | PLI | 1 |
| alpha-synuclein | SNCA | 5 |
| Aminomethyltransferase | AMT | 1 |
| Aminopeptidase P | XPNPEP2 | 1 |
| Amyloid beta (A4) precursor protein-binding, | APBB1 | 5 |
| APBB1 | | |
| Amyloid beta A4 precursor protein | APP | 5 |
| Amyloid beta A4 precursor-like protein | APLP | 5 |
| Angiopoietin 1 | ANGPT1 | 6 |
| Angiopoietin 2 | ANGPT2 | 6 |
| Angiotensin converting enzyme | ACE, DCPI | 1 |
| Angiotensin receptor 1 | AGTRL | 2 |
| Angiotensin receptor 2 | AGTR2 | 2 |
| Angiotensinogen | AGT | 1 |
| Annexin 1 | ANX 1 | 1 |
| Antidiuretic hormone receptor | ADHR | 2 |
| Antithrombin III | AT3 | 1 |
| Apolipoprotein A I | APOAL | 2 |
| Apolipoprotein A II | APOA2 | 2 |
| Apolipoprotein B | APOB | 2 |
| Apolipoprotein Cl | APOCI | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Apoptosis antigen 1 | APTI | 4 |
| Arginase | ARG1 | 1 |
| Arginine vasopressin | AVP | 5 |
| Arginine vasopressin receptor 1A | AVPR1A | 5 |
| Arginine vasopressin receptor 1 B | AVPR1B | 5 |
| Arginine vasopressin receptor 2 | AVPR2 | 5 |
| Arginosuccinate lyase | ASL | 1 |
| Arginosuccinate synthetase | ASS | 1 |
| Arylsulfatase A | ARSA | 1 |
| Arylsulfatase D | ARSD | 1 |
| Arylsulfatase E | ARSE | 1 |
| Arylsulfatase F | ARSF | 1 |
| Aspartoacylase | ASPA | 1 |
| Ataxia telangiectasia gene, AT | ATM | 6 |
| Atrial natriuretic peptide | ANP | 6 |
| Atrial natriuretic peptide receptor A | NPRI | 6 |
| Atdal natriuretic peptide receptor B | NPR2 | 6 |
| Atrial natriuretic peptide receptor C | NPR3 | 6 |
| Bagpipe homeobox, drosophila homolog of, 1 | BAPXL | 6 |
| beta-synuclein | SNCB | 5 |
| Bleomycin hydrolase | BLMH | 1 |
| Bradykinin receptor B1 | | 1 |
| Bradykinin receptor B2 | | 1 |
| Brain derived neurotrophic factor | BDNF | 6 |
| Brain derived neurotrophic factor (BDNF) | BDNFR | 6 |
| receptor | | |
| Butyrylcholinesterase | BCHE | 1 |
| Ca(2+) transporting ATPase, slow twitch | ATP2A2 | 2 |
| Cadherin E | CDHI | 6 |
| Cadherin EP | | 6 |
| Cadherin N | CDH2 | 6 |
| Cadherin P | CDH3 | 6 |
| Calbindin | CALB1 | 6 |
| Calbindin D9K | CALB3 | 6 |
| Calcineurin Al | CALNAL | 1 |
| Calcineurin A2 | CALNA2 | 1 |
| Calcineurin A3 | CALNA3 | 1 |
| Calcineurin B | | |
| Calcitonin/Calcitonin gene-related peptide | CALCA | 5 |
| alpha | | |
| Calcium channel, voltage-dependent, alpha | CACNA1F | 5 |
| 1F subunit | | |
| Calcium channel, voltage-dependent, Alpha- | CACNA1B | 5 |
| 1B (CACNL1A5) | | |
| Calcium channel, voltage-dependent, Alpha-1C | CACNA1C | 5 |
| Calcium channel, voltage-dependent, Alpha-1D | CACNA1D | 5 |
| Calcium channel, voltage-dependent, Alpha- | CACNA1E | 5 |
| 1E (CACNL1A6) | | |
| Calcium channel, voltage-dependent, Alpha- | CACNA2 | 5 |
| 2/delta | | |
| Calcium channel, voltage-dependent, Beta 1 | CACNBI | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase II | CAMK2A | 6 |
| Calnexin | CANX | 6 |
| Calpain | CAPN, | 1 |
| | CAPN3 | |
| Calretinin | CALB2 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Carnosinase | | 5 |
| Caspase 1 | CASP1 | 6 |
| Caspase 10 | CASP10 | 6 |
| Caspase 2 | CASP2 | 6 |
| Caspase 3 | CASP3 | 6 |
| Caspase 4 | CASP4 | 6 |
| Caspase 5 | CASP5 | 6 |
| Caspase 6 | CASP6 | 6 |
| Caspase 7 | CASP7 | 6 |
| Caspase 8 | CASP8 | 6 |
| Caspase 9 | CASP9 | 6 |
| Catechol-o-methyltransferase | COMT | 1 |
| CD1 | CD1 | 1 |
| CD4 | CD4 | 1 |
| Cell adhesion molecule, intercellular, LCAM | ICAM1 | 6 |
| Cell adhesion molecule, leukocyte- | LECAM1 | 6 |
| endothelial, LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM | 6 |
| PECAM | | |
| Cell adhesion molecule, vascular, VCAM | VCAM1 | 6 |
| Ceroid lipofuscinosis neuronal 2 | CLN2 | 5 |
| Ceroid lipofuscinosis neuronal 3 | CLN3 | 5 |
| Cerold lipofuscinosis neuronal 4 | CLN4 | 5 |
| Ceroid lipofuscinosis neuronal 5 | CLN5 | 5 |
| Ceroid lipofuscinosis neuronal 6 | CLN6 | 5 |
| Chemokine receptor CXCR4 | CXCR | 4 |
| Choline acetyltransferase | CHAT | 1 |
| Chymotrypsinogen | | 1 |
| Cockayne syndrome gene, CKN1 | CKN1 | 6 |
| Cofilin | | 3 |
| Collagen I alpha 1 | COL1A1 | 3 |
| Collagen I alpha 2 | COL1A2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, | 3 |
| | EDM2 | |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11A1 | 3 |
| Collagen XI alpha 2 | COL11A2 | 3 |
| Collagen XVII alpha 1 | COL17A1 | 3 |
| Corticotrophin-releasing hormone | CRH | 2 |
| Corticotrophin-releasing hormone receptor | CRHR1 | 2 |
| Creb binding protein | CREBBP | 6 |
| Cu2+ transporting ATPase beta polypeptide | ATP7B | 1 |
| Cyclic AMP-dependent protein kinase | PKA | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystatin B | CSTB | 2 |
| Cystatin C | CST3 | 2 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBPI | 4 |
| binding protein 1 | | |
| Cytokine-suppressive antiinflammatory drug- | CSBP2 | 4 |
| binding protein 2 | | |
| DAX1 nuclear receptor | DAX1 | |
| Deleted in malignant brain tumours 1 | DMBT1 | 6 |
| Delta-7-dehydrocholesterol reductase | DHCR7 | 1 |
| Dihydrolipoamide branched chain | DBT | 5 |
| transacylase | | |
| Dihydroxyacetonephosphate acyltransferase | DHAPAT | 1 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dystonia 9 | CSE | 3 |
| Dystrophia myotonica | DM, | 1 |
| | DMPK | |
| Dystrophia myotonica, atypical | DM2 | 1 |
| Dystrophin | DMD | 3 |
| Ectodermal Dysplasia 1 gene | ED1 | 3 |
| Empty spiracles (drosophila) homologue 1 | EMXL | 6 |
| Empty spiracles (drosophila) homologue 2 | EMX2 | 6 |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | ENOI | 1 |
| Epidermal growth factor | EGF | 6 |
| Epidermal growth factor receptor | EGFR | 6 |
| Epilepsy, benign neonatal 4 gene | ICCA | 1 |
| Epilepsy, female restricted | EFMR | 1 |
| Epilepsy, progressive myocionic 2 gene | EPM2A | 1 |
| Excision repair complementation group 4 | ERCC4 | 1 |
| protein | | |
| Factor 1 (No. one) | F1 | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & | 4 |
| | F13B | |
| Fanconi anemia, complementation group C | FANCC | 2 |
| Fanconi anemia, complementation group D | FANCD | 2 |
| Fibrinogen alpha | FGA | 3 |
| Fibrinogen beta | FGB | 3 |
| Fibrinogen gamma | FGG | 3 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Flightless-II, Drosophila homolog of | FLII | 6 |
| Follicle stimulating hormone receptor | FSHR, | 6 |
| | ODG1 | |
| Follicle stimulating hormone, FSH | FSHB | 6 |
| Formiminotransferase | | 1 |
| Fragile site, folic acid type, rare, fra(X) A | FRAXA | 5 |
| Fragile site, folic acid type, rare, fra(X) E | FRAXE | 5 |
| Fragile site, folic acid type, rare, fra(X) F | FRAXF | 5 |
| Frataxin | FRDA | 6 |
| Fukuyama type congenital muscular dystrophy | FCMD | 6 |
| Fumarase | FH | 1 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Galactosyltransferase 1 | GT1 | 6 |
| Galactosyltransferase, alpha 1,3 | GGTA1 | 6 |
| Galactosyltransferase, beta 3 | B3GALT | 6 |
| Galanin | GAL | 5 |
| Galanin receptor | GALNR1 | 5 |
| Gamma-glutamyltransferase I | GGT1 | 2 |
| Gastric Intrinsic factor, GIF | GIF | 1 |
| GDP dissociation inhibitor I | GDI1 | 6 |
| Glial-cell derived neurotrophic factor (GDNF) | | 5 |
| receptor | | |
| Gilal-cell derived neurotrophic factor, GDNF | GDNF | 5 |
| Glioma chloride ion channel, GCC | | 6 |
| Glutamate decarboxylase, GAD | GADI | 1 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutaryl-CoA dehydrogenase | GCDH | 1 |
| Glutathione | GSH | 2 |
| Glutathione S-transferase, GSTZ1 | GSTZ1 | 1 |
| Glutathione synthetase | GSS | 1 |
| Glyceraldehyde-3-phosphate | GAPDH | 1 |
| dehydrogenase, GAPDH | | |
| Glycerol kinase | GK | 1 |
| Glycinamide ribonucleotide (GAR) | GART | 1 |
| transformylase | | |
| Glycine dehydrogenase | GLDC | 1 |
| GM2 ganglioside activator protein, GM2A | GM2A | 1 |
| Gonadotropin releasing hormone receptor | GNRHR | 6 |
| GTP cylcohydrolase I | GCH1 | 6 |
| Guanine nucleotide-binding protein, alpha | GNAOI | 5 |
| activating activity polypeptide, GNAO | | |
| Guanylate cyclase 2D, membrane (retina- | GUCY2D | 1 |
| specific) | | |
| Guanylate cyclase activator 1A (retina) | GUCA1A | 1 |
| Guanylyl cyclase | | 1 |
| Haeme regulated inhibitor kinase | | 1 |
| Haemoglobin alpha 1 | HBA1 | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Heparan sulfamidase | | 1 |
| Heparin binding epidermal growth factor | HBEGF | 6 |
| Heparin Cofactor II | HCF2 | 4 |
| Hepatic lipase | LIPC | 1 |
| Hexosaminidase A | HEXA, TSD | 1 |
| Hexosaminidase B | HEXB | 1 |
| Histamine receptors, H1 | | 5 |
| Histamine receptors, H2 | | 5 |
| Histamine receptors, H3 | | 5 |
| Histidase | | 1 |
| HLA-B associated transcript 1 | BAT1 | |
| HMG-CoA reductase | HMGCR | 1 |
| Holocarboxylase synthetase | HLCS | 1 |
| Holoprosencephaly 1 | HPE1 | 6 |
| Holoprosencephaly 2 | HPE2 | 6 |
| Holoprosencephaly 3 | HPE3 | 6 |
| Holoprosenceohaly 4 | HPE4 | 6 |
| Hypoxia inducible factor 1 | HIF1A | 1 |
| Hypoxia inducible factor 2 | | 1 |
| IC7 A and B | | 4 |
| lnositol 1,4,5-triphosphate receptor 1 | ITPR1 | 6 |
| Inositol monophosphatase | IMPA1 | 5 |
| Insulin | INS | 6 |
| Insulin receptor | INSR | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| lntegrin beta 1 | ITGB1 | 6 |
| Integrin beta 2 | ITGB2 | 6 |
| Integrin beta 3 | ITGB3 | 6 |
| Integrin beta 4 | ITGB4 | 6 |
| Integrin beta 5 | ITGB5 | 6 |
| Integrin beta 6 | ITGB6 | 6 |
| Integrin beta 7 | ITGB7 | 6 |
| Integrin, alpha 1 | ITGAL | 6 |
| Integrin, alpha 2 | ITGA2 | 6 |
| Integrin, alpha 3 | ITGA3 | 6 |
| Integrin, alpha 4 | ITGA4 | 6 |
| lntegrin, alpha 5 | ITGA5 | 6 |
| Integrin, alpha 6 | ITGA6 | 6 |
| Integrin, alpha 7 | ITGA7 | 6 |
| lntegrin, alpha 8 | ITGA8 | 6 |
| Integrin, alpha 9 | ITGA9 | 6 |
| Integrin, alpha M | ITGAM | 6 |
| lntegrin, alpha X | ITGAX | 6 |
| Inter-alpha-trypsin inhibitor, IATI | | 1 |
| Interleukin(IL) 1 receptor | IL1R | 4 |
| Interleukin(IL) 1, alpha | IL1A | 4 |
| lnterleukin(IL) 1, beta | IL1B | 4 |
| lnterleukin(IL) 10 | IL10 | 4 |
| lnterleukin(IL) 10 receptor | IL10R | 4 |
| lnterieukin(IL) 11 | IL11 | 4 |
| lnterleukin(IL) 11 receptor | IL11R | 4 |
| lnterleukin(IL) 12 | IL12 | 4 |
| Interleukin(IL) 12 receptor, beta | IL12RB1 | 4 |
| Interleukin(IL) 13 | IL13 | 4 |
| lnterleukin(IL) 13 receptor | IL13R | 4 |
| Interleukin(IL) 2 | IL2 | 4 |
| lnterleukin(IL) 2 receptor, alpha | IL2RA | 4 |
| Interleukin(IL) 2 receptor, gamma | IL2RG | 4 |
| lnterleukin(IL) 3 | IL3 | 4 |
| Interleukin(IL) 3 receptor | IL3R | 4 |
| Interleukin(IL) 4 | IL4 | 4 |
| lnterleukin(IL) 4 receptor | IL4R | 4 |
| Interleukin(IL) 5 | IL5 | 4 |
| lnterleukin(IL) 5 receptor | IL5R | 4 |
| lnterleukin(IL) 6 | IL6 | 4 |
| lnterleukin(IL) 6 receptor | IL6R | 4 |
| Interleukin(IL) 7 | IL7 | 4 |
| Interleukin(IL) 7 receptor | IL7R | 4 |
| Interleukin(IL) 8 | IL8 | 4 |
| lnterleukin(IL) 8 receptor | IL8R | 4 |
| lnterleukin(IL) 9 | IL9 | 4 |
| lnterleukin(IL) 9 receptor | IL9R | 4 |
| lnterleukin(IL) receptor antagonist 1 | ILI RN, | 1 |
| IP3 kinase | IL1RA | |
| Kallikrein 3 | KAK3 | 4 |
| Kininogen, High molecular weight | KNG | 4 |
| Kynureninease | | 1 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta | LTBP2 | 6 |
| binding protein 2 | | |
| Leptin | LEP | 6 |
| Leptin receptor | LEPR | 6 |
| Leukin | | 4 |
| Leukocyte-specific transcript 1 | LST-1 | 4 |
| Leukotriene A4 hydrolase | | |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| LIM-Kinase I (LINK-1) | | 4 |
| Lipocortin 1 | ANX4 | 4 |
| Lipoprotein lipase | LPL | 4 |
| Lipoprotein receptor, Low Density | LDLR | 2 |
| Lipoprotein, High Density | HDLDT1 | 2 |
| Lipoprotein, Intermediate Density | | 2 |
| Lipoprotein, Low Density 1 | | 2 |
| Lipoprotein, Low Density 2 | | 2 |
| Lipoprotein, Very Low Density | VLDLR | 2 |
| Lipoprotein-associated coagulation factor | LACI | 4 |
| Low density lipoprotein receptor-related | LRP | 2 |
| protein precursor | | |
| Lymphoid enhancer-binding factor | LEF-1 | 6 |
| MAD (mothers against decapentaplegic, | MADH4 | 6 |
| Drosophila) homologue 4 | | |
| Malonyl CoA decarboxylase | | 1 |
| Mannosidase, alpha B lysosomal | MANB | 1 |
| Mannosidase, -beta A lysosomal | MANBA | 1 |
| Methionine synthase | MTR | 1 |
| Methylmalonyl-CoA mutase | MUT | 1 |
| Mevalonate kinase | MVK | 1 |
| Mismatch repair gene, PMSL2 | PMS2 | 6 |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Mucolipidoses | GNPTA | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Myelin basic protein | | 3 |
| N-acetylglucosamine-6-sulfatase | GNS | 1 |
| N-acetylglucosaminidase, alpha | NAGLU | 1 |
| reductase | | |
| NB6 | | 4 |
| Nerve growth factor | NGF | 6 |
| Nerve growth factor receptor | NGFR | 6 |
| Neurite inhibitory protein | | 5 |
| Neurofibromin 1 | NF1 | 6 |
| Neurofibromin 2 | NF2 | 6 |
| Neurofilament protein, NF68 | NF68 | 3 |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Notch 3 | NOTCH3 | 6 |
| Notch ligand-jagged 1 | JAG 1, AGS | 6 |
| Nuclear factor 1-kappa-B-like gene | IKBL | 4 |
| Nucieoside diphosphate kinase-A | NDPKA | 1 |
| Oncogene bcI2 | | 6 |
| Oncogene sis | PDGFB | 6 |
| Ornithine delta-aminotransferase | OAT | 1 |
| Ornithine transcarbamoylase | OTC, NME1 | 1 |
| Orthodenticle (Drosophila) homolog 1 | OTX1 | 6 |
| Orthodenticle (Drosophila) homolog 2 | OTX2 | 6 |
| Patched (Drosophila) homolog, PTCH | PTCH | 6 |
| Peroxisomal membrane protein 1 | PXMP1 | 3 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome receptor 1 | PXR1 | 2 |
| Persyn | | 3 |
| Phosphoglucose isomerase | GPI | 1 |
| Phosphoglycerate kinase 1 | PGK1 | 1 |
| Phospholipase A2, group 10 | PLA2G 10 | 1 |
| Phospholipase A2, group 1B | PLA2G 1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase 2 | PMM2 | 6 |
| Plasminogen | PLG | 1 |
| Plasminogen activator inhibitor 1 | PAI1 | 1 |
| Plasminogen activator inhibitor 2 | PAI2 | 1 |
| Plasminogen activator receptor, Urokinase | UPAR; | 3 |
| | PLAUR | |
| Plasminogen activator, Tissue | PLAT; TPA | 1 |
| Plasminogen activator, Urokinase | UPAPLAU | 1 |
| Platelet derived growth factor | PDGF | 6 |
| Platelet derived growth factor receptor | PDGFR | 6 |
| Platelet glycoprotein I b, alpha | GP1BA | 4 |
| Platelet glycoprotein I b, beta | GP1BB | 4 |
| Platelet glycoprotein I b, gamma | GPIBG | 4 |
| Platelet glycoprotein IX | GP9 | 4 |
| Platelet glycoprotein V | GP5 | 4 |
| Platelet-activating factor acetylhydrolase 2 | PAFAH2 | 4 |
| Platelet-activating factor receptor | PAFR | 4 |
| Plectin 1 | PLEC1 | 2 |
| Polycystin 1 | PKD1 | 2 |
| Polycystin 2 | PKD2 | 2 |
| Potassium inwardly-rectifying channel J1 | KCNJ1 | 5 |
| Potassium voltage-gated channel E1 | KCNE1 | 5 |
| Potassium voltage-gated channel Q1 | KCNQI | 5 |
| Potassium voltage-gated channel Q2 | KCN02 | 5 |
| Potassium voltage-gated channel Q3 | KCNQ3 | 5 |
| POU domain, class 1, transcription factor 1 | POU1F1 | 6 |
| (Pit1) | | |
| Prekallikrein | | |
| Prion protein | PRNP | 5 |
| Procollagen N-protease | | 1 |
| Proline dehydrogenase | PRODH | 1 |
| Proopiomelanocortin | POMC | 5 |
| Prostacyclin synthase | | 4 |
| Prostaglandin 15-OH dehydrogenase | HGPD, | 4 |
| | PGDH | |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin I2 receptor | | 2 |
| Prostaglandin IP receptor | | 4 |
| Protective protein for beta-galactosidase | PPGB | 1 |
| Protein C | PROC | 4 |
| Protein C inhibitor | PCI | 4 |
| Protein kinase C, alpha | PRKCA | 1 |
| Protein kinase C, gamma | PRKCG | 1 |
| Protein kinase G | | 1 |
| Protein phosphatase 1, regulatory (inhibitor) | PPPIR3 | 1 |
| subunit 3 | | |
| Protein S | PROS1 | 4 |
| Prothrombin precursor | F2 | 4 |
| Purine nucleoside phosphorylase | NP | 1 |
| Pyrroline-5-carboxylate synthetase | PYCS | 1 |
| Pyruvate carboxylase | PC | 1 |
| Ras-G-protein | RAS | 6 |
| Renin | REN | 1 |
| Replication factor C | RFC2 | 1 |
| RIGUI | RIGUI | 6 |
| S100 calcium-binding protein A1 | S100A1 | 5 |
| S100 calcium-binding protein A4 | S100A4 | 5 |
| S100 calcium-binding protein A5 | S100A5 | 5 |
| S100 calcium-binding protein A6 | S100A6 | 5 |
| S100 calcium-binding protein A7 | S100A7 | 5 |
| S100 calcium-binding protein A9 | S100A9 | 5 |
| S100 calcium-binding protein B | S100B | 5 |
| Secretase, alpha | | 5 |
| Secretase, beta | | 5 |
| Secretase, gamma | | 5 |
| Selectin E | SELE | 5 |
| Selectin L | SELL | 5 |
| Selectin P | SELP | 5 |
| Serotonin N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1 D | HTR1D | 5 |
| Serotonin receptor, 5HT1 E | HTR1E | 5 |
| Serotonin receptor, 5HT1 F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, | SCNN1G | 5 |
| gamma | | |
| Sodium channel, voltage-gated, type 1, beta | SCN B | 5 |
| polypeptide | | |
| Solute carder family 1 (glutamate | SLC1A1 | 2 |
| transporter), member 1 | | |
| Solute carrier family 1 (glutamate | SLC1A2 | 2 |
| transporter), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 18, member 3 | SLC18A3 | 2 |
| Solute carrier family 2 (facilitated glucose | SLC2A1 | 2 |
| transporter), member 1 | | |
| Solute carrier family 20, member 3 | SLC2OA3 | 2 |
| Solute carrier family 5 (sodium/glucose | SLC5AI | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member. | | |
| Solute carrier family 6 (neurotransmifter | SLC6A3 | 2 |
| transporter, dopamine), member 3 | | |
| Solute carrier family 6 (neurotransmifter | SLC6A2 | 2 |
| transporter, noradrenaline), member 2 | | |
| Solute carder family 6 (neurotransmifter | SLC6A4 | 2 |
| transporter, serotonin), member 4 | | |
| Solute carrier family 7(amino acid | SLC7A1 | 2 |
| transporter), member 1 | | |
| Solute carrier family 7(amino acid | SLC7A2 | 2 |
| transporter), member 2 | | |
| Solute carrier family 7(amino acid | SLC7A7 | 2 |
| transporter), member 7 | | |
| Sphingomyelinase | SMPD1 | 1 |
| Spinocerebellar ataxia 8 gene | SCA8 | 5 |
| Steroid 5 alpha reductase 1 | SRD5A1 | 1 |
| Steroid 5 alpha reductase 2 | SRD5A2 | 1 |
| Substance P | | 5 |
| Succinic semi-aldehyde dehydrogenase | SSADH | 1 |
| Sulfamidase | SGSH | 6 |
| Sulfite oxidase | SUOX | 1 |
| Superoxide dismutase 1 | SOD1 | 1 |
| Superoxide dismutase 3 | SOD3 | 1 |
| Surfeit 1 | SURF1 | 6 |
| Synapsin 1 a & 1 b | SYN1 | 5 |
| Synapsin 2a & 2b | SYN2 | 5 |
| Synaptic vesicle amine transporter | SVAT | 5 |
| Synaptobrevin 1 | SYB1 | 5 |
| Synaptobrevin 2 | SYB2 | 5 |
| Synaptogyrin | | 5 |
| Synaptophysin | SYP | 5 |
| Synaptotagmin 1 | SYT1 | 5 |
| Synaptotagmin 2 | SYT2 | 5 |
| Syntaxin 1 | STX1 | 5 |
| Talin | TLN | 6 |
| Tau protein | MAPT | 3 |
| TEK, tyrosine kinase, endothelial | TEK | 1 |
| Telomerase protein component | | 1 |
| Thrombin receptor | F2R | 4 |
| Thrombopoietin | THPO | 6 |
| Thromboxane A synthase I | TBXASI | 4 |
| Thromboxane A2 | TXA2 | 4 |
| Thromboxane A2 receptor | TBXA2R | 4 |
| Thyroxin-binding globulin | TBG | 2 |
| Topoisomerase I | | 1 |
| Transforming growth factor, beta 2 | TGFB2 | 6 |
| Transforming growth factor, beta receptor 2 | TGFBR2 | 6 |
| Tuberous sclerosis 1 | TSC1 | 6 |
| Tuberous sclerosis 2 | TSC2 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (‘I NF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tyrosine aminotransferase | TAT | 1 |
| Tyrosine hydroxylase | TH | 1 |
| Ubiquitin | | 6 |
| Ubiquitin B | UBB | 6 |
| Ubiquitin C | UBC | 6 |
| Ubiquitin carboxyl-terminal esterase L1 | UCHL1 | 6 |
| UDP-glucuronosyltransferase 1 | ugt1d, UGT1 | 1 |
| UDP-glucuronosyltransferase 2 | UGT2 | 1 |
| Undulin 1 | COL14A1 | 3 |
| Uridinediphosphate(UDP)-galactose-4- | GALE | 1 |
| epimerase | | |
| Uroporphyrinogen III synthase | UROS | |
| Vacuolar proton pump, subunit 1 | VPP1 | 5 |
| Vacuolar proton pump, subunit 3 | VPP3 | 5 |
| Vasoactive intestinal polypeptide | VIP | 5 |
| Vasoactive intestinal polypeptide receptor | VIPR | 5 |
| Von Hippel-Lindau gene | VHL | 6 |
| Wolf-Hirschhorn syndrome candidate 1 gene | WHSC1 | 6 |
| Xanthine dehydrogenase | XDH | 1 |
| Zinc finger protein 2 | ZIC2 | 3, |

that the genes associated with dementia and/or associated syndromes are selected from:

| | HUGO | Protein- |
|---|---|---|
| List of genes | gene symbol | function |
| 2,3-Bisphosphoglycerate mutase | BPGM | 1 |
| 3 beta Hydroxysteroid dehydrogenase 2 | HSD3B2 | 1 |
| 5,10-Methylenetetrahydrofolate reductase | MTHFR | 1 |
| (NADPH) | | |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Adducin, alpha | ADD1 | 3 |
| Adducin, beta | ADD2 | 3 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | AOCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| Aldosterone receptor | MLR | 6 |
| alpha 2 Macroglobulin | A2M | 4 |
| alpha1-Antitrypsin | PI | 1 |
| alpha2-Antiplasmin | PLI | 1 |
| alpha-Synuclein | SNCA | 5 |
| Aminopeptidase P | XPNPEP2 | 1 |
| Amyloid beta (A4) precursor protein-binding, | APBB1 | 5 |
| APBB1 | | |
| Amyloid beta A4 precursor protein | APP | 5 |
| Amyloid beta A4 precursor-like protein | APLP | 5 |
| Angiopoietin 1 | ANGPT1 | 6 |
| Angiopoietin 2 | ANGPT2 | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Antidiuretic hormone receptor | ADHR | 2 |
| Antithrombin III | AT3 | 1 |
| Apolipoprotein A 1 | APOA1 | 2 |
| Apoiipoprotein A 1 | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Apoptosis antigen 1 | APT1 | 4 |
| Arginase | ARG1 | 1 |
| Arginine vasopressin | AVP | 5 |
| Arginine vasopressin receptor 1A | AVPR1A | 5 |
| Arginine vasopressin receptor 1B | AVPRL1B | 5 |
| Arginine vasopressin receptor 2 | AVPR2 | 5 |
| Arginosuccinate lyase | ASL | 1 |
| Arginosuccinate synthetase | ASS | 1 |
| Ataxia telangiectasia gene, AT | ATM | 6 |
| ATP/ADP translocase | | 1 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Bagpipe homeobox, drosophila homolog of, 1 | BAPX1 | 6 |
| beta-synuclein | SNCB | 5 |
| Bleomycin hydrolase | BLMH | 1 |
| Bradykinin receptor B1 | | 4 |
| Bradykinin receptor B2 | | 4 |
| Brain derived neurotrophic factor | BDNF | 6 |
| Brain derived neurotrophic factor (BDNF) | BDNFR | 6 |
| receptor | | |
| Butyrylcholinesterase | BCHE | 1 |
| Cadherin E | CDH1 | 6 |
| Cadherin EP | | 6 |
| Cadherin N | CDH2 | 6 |
| Cadherin P | CDH3 | 6 |
| Calbindin 1 | CALB1 | 6 |
| Calbindin D9K | CALB3 | 6 |
| Calcineurin A1 | CALNA1 | 4 |
| Calcineurin A2 | CALNA2 | 4 |
| Calcineurin A3 | CALNA3 | 4 |
| Calcineurin B | | |
| Calcitonin/Calcitonin gene-related peptide | CALCA | 5 |
| alpha | | |
| Calcium channel, voltage-dependent, alpha 1 F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha-1 C | CACNA1C | 5 |
| Calcium channel, voltage-dependent, Alpha-1 D | CACNA1D | 5 |
| Calcium channel, voltage-dependent, Alpha- | CACNA1E | 5 |
| 1 E (CACNL1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNAL S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase II | CAMK2A | 6 |
| Calnexin | CANX | 6 |
| Calpain | CAPN, CAPN3 | 1 |
| Calretinin | CALB2 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Caspase 1 | CASP1 | 6 |
| Caspase 10 | CASP10 | 6 |
| Caspase 2 | CASP2 | 6 |
| Caspase 3 | CASP3 | 6 |
| Caspase 4 | CASP4 | 6 |
| Caspase 5 | CASP5 | 6 |
| Caspase 6 | CASP6 | 6 |
| Caspase 7 | CASP7 | 6 |
| Caspase 8 | CASP8 | 6 |
| Caspase 9 | CASP9 | 6 |
| Catechol-o-methyltransferase | COMT | 1 |
| CD1 | CD1 | |
| CD4 | CD4 | |
| Cell adhesion molecule, intercellular, ICAM | ICAM1 | 6 |
| Cell adhesion molecule, leukocyte-endothelial, | LECAM1 | 6 |
| LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cel adhesion molecule, platelet-endothelial, | PECAM1 | 6 |
| PECAM | | |
| Cell adhesion molecule, vascular, VCAM | VCAM1 | 6 |
| Chemokine receptor CXCR4 | CXCR4 | 4 |
| Choline acetyltransferase | CHAT | 1 |
| Chymotrypsinogen | | 1 |
| Cockayne syndrome gene, CKN1 | CKN1 | 6 |
| Cofilin | | 3 |
| Collagen I alpha 1 | COLIA1 | 3 |
| Collagen I alpha 2 | COLIA2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11A1 | 3 |
| Collagen XI alpha 2 | COL11A2 | 3 |
| Collagen XVII alpha 1 | COL17A1 | 3 |
| Corticotrophin releasing hormone | CRH | 2 |
| Corticotrophin releasing hormone receptor | CRHR1 | 2 |
| Cu2+ transporting ATPase beta polypeptide | ATP7B | 1 |
| Cyclic AMP-dependent protein kinase | PKA | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathione beta synthase | CBS | 1 |
| Cystatin C | CST3 | 2 |
| Cystinosin | CTNS | 2 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Dihydrolipoyl succinyltransferase | DLST | 1 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Doublecortin, DCX | DCX | 3 |
| Emerin | EMD | 2 |
| Endothelin 1 | DNI | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | ENO1 | 1 |
| Epidermal growth factor | EGF | 6 |
| Epidermal growth factor receptor | EGFR | 6 |
| Epilepsy, progressive myoclonic 2 gene | EPM2A | 1 |
| Excision repair complementation group 4 | ERCC4 | 1 |
| protein | | |
| Factor I (No. one) | F1 | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & F13B | 4 |
| Fanconi anemia, complementation group A | FANCA | 2 |
| Fibrinogen alpha | FGA | 3 |
| Fibrinogen beta | FGB | 3 |
| Fibrinogen gamma | FGG | 3 |
| Fibroblast growth factor | FGFI | 6 |
| Fibroblast growth factor receptor 1 | FGFRL | 6 |
| Fibroblast growth factor receptor 2 | FGFR2 | 6 |
| Fibroblast growth factor receptor 3 | FGFR3 | 6 |
| Fibronectin precursor | FN1 | 6 |
| Follicle stimulating hormone receptor | FSHR, ODG1 | 6 |
| Follicle stimulating hormone, FSH | FSHB | 6 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Galactosyltransferase 1 | GT1 | 6 |
| Galactosyltransferase, alpha 1, 3 | GGTA1 | 6 |
| Galactosyltransferase, beta 3 | B3GALT | 6 |
| Gastric Intrinsic factor, GIF | GIF | 1 |
| Glial-cell derived neurotrophic factor (GDNF) | | 5 |
| receptor | | |
| Glial-cell derived neurotrophic factor, GDNF | GDNF | 5 |
| Glutamate decarboxylase, GAD | GAD1 | 1 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutaryl-CoA dehydrogenase | GCDH | 1 |
| Glutathione | GSH | 2 |
| Glutathione S-transferase, GSTZ1 | GSTZ1 | 1 |
| Glyceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Gonadotropin releasing hormone receptor | GNRHR | 6 |
| Guanylyl cyclase | | 1 |
| Haemoglobin alpha 1 | HBAL | 2 |
| Haemoglobin alpha 2 | HBA2 | 2 |
| Haemoglobin beta | HBB | 2 |
| Haemoglobin delta | HBD | 2 |
| Haemoglobin gamma A | HBGL | 2 |
| Haemoglobin gamma B | HBG2 | 2 |
| Haemoglobin gamma G | HBGG | 2 |
| Heparan sulfamidase | | 1 |
| Heparin binding epidermal growth factor | HBEGF | 6 |
| Heparin Cofactor II | HCF2 | 4 |
| Hepatic lipase | LIPC | 1 |
| Hexosaminidase A | HEXA, TSD | 1 |
| Hexosaminidase B | HEXB | 1 |
| Hippocampal cholinergic neurostimulating peptide, HCNP | | 5 |
| Histamine receptors, H1 | | 5 |
| Histamine receptors, H2 | | 5 |
| Histamine receptors, H3 | | 5 |
| Histidase | | 1 |
| HLA-B associated transcript 1 | BAT1 | |
| HMG-CoA reductase | HMGCR | 1 |
| Holocarboxylase synthetase | HLCS | 1 |
| Hypoxia inducible factor 1 | HIF1A | 1 |
| Hypoxia inducible factor 2 | | 1 |
| IC7 A and B | | 4 |
| Inositol monophosphatase | IMPA1 | 5 |
| Insulin | INS | 6 |
| Insulin receptor | INSR | 6 |
| Integrin beta 1 | ITGB1 | 6 |
| Integrin beta 2 | ITGB2 | 6 |
| Integrin beta 3 | ITGB3 | 6 |
| Integrin beta 4 | ITGB4 | 6 |
| Integrin beta 5 | ITGB5 | 6 |
| Integrin beta 6 | ITGB6 | 6 |
| Integrin beta 7 | ITGB7 | 6 |
| Integrin, alpha 1 | ITGA1 | 6 |
| Integrin, alpha 2 | ITGA2 | 6 |
| Integrin, alpha 3 | ITGA3 | 6 |
| Integrin, alpha 4 | ITGA4 | 6 |
| lntegrin, alpha 5 | ITGA5 | 6 |
| Integrin, alpha 6 | ITGA6 | 6 |
| lntegrin, alpha 7 | ITGA7 | 6 |
| Integrin, alpha 8 | ITGA8 | 6 |
| lntegrin, alpha 9 | ITGA9 | 6 |
| Integrin, alpha M | ITGAM | 6 |
| Integrin, alpha, X | ITGAX | 6 |
| Interleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) II | IL11 | 4 |
| Interleukin(IL) I 1 receptor | IL11R | 4 |
| Interleukin(IL) 12 | IL12 | 4 |
| Interleukin(IL) 12 receptor, beta 1 | IL12RB1 | 4 |
| lnterleukin(IL) 13 | IL13 | 4 |
| Interleukin(IL) 13 receptor | IL13R | 4 |
| Interleukin(IL) 2 | IL2 | 4 |
| Interleukin(IL) 2 receptor, alpha | IL2RA | 4 |
| Interleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| lnterleukin(IL) 4 receptor | IL4R | 4 |
| Interleukin(IL) 5 | IL5 | 4 |
| Interleukin(IL) 5 receptor | IL5R | 4 |
| lnterleukin(IL) 6 | IL6 | 4 |
| Interleukin(IL) 6 receptor | IL6R | I′ |
| Interleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| Interleukin(IL) 9 | IL9 | 4 |
| lnterleukin(IL) 9 receptor | IL9R | 4 |
| Interleukin(IL) receptor antagonist 1 | IL1 RN, IL1RA | 4 |
| IP3 kinase | | 1 |
| Kallikrein 3 | KAK3 | 4 |
| Kininogen, High molecular weight | KNG | 4 |
| Kynureninease | | 1 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta binding | LTBP2 | 6 |
| protein 2 | | |
| Leptin | LEP | 6 |
| Leptin receptor | LEPR | 6 |
| Leukin | | 4 |
| Leukocyte-specific transcript 1 | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| LIM-Kinase I (LINK-1) | | 4 |
| Lipoprotein receptor, Low Density | LDLR | 2 |
| Lipoprotein, High Density | HDLDT1 | 2 |
| Lipoprotein, Intermediate Density | | 2 |
| Lipoprotein, Low Density I | | 2 |
| Lipoprotein, Low Density 2 | | 2 |
| Lipoprotein, Very Low Density | VLDLR | 2 |
| Low density lipoprotein receptor-related protein | LRP | 2 |
| precursor | | |
| Lymphoid enhancer-binding factor | LEF-1 | 6 |
| MAD (mothers against decapentaplegic, | MADH4 | 6 |
| Drosophila) homologue 4 | | |
| Mannosidase, alpha B lysosomal | MANB | 1 |
| Mannosidase, beta A lysosomal | MANBA | 1 |
| Methionine synthase | MTR | 1 |
| Mismatch repair gene, PMSL2 | PMS2 | 6 |
| Molybdenum cofactor synthesis 1 | MOSC1 | 1 |
| Molybdenum cofactor synthesis 2 | MOCS2 | 1 |
| Monoamine oxidase A | MAOA | 1 |
| Monoamine oxidase B | MAOB | 1 |
| Muscarinic receptor, M1 | CHRM1 | 5 |
| Muscadnic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Myelin basic protein | | 3 |
| N-Acetylglucosamine-6-sulfatase | GNS | 1 |
| N-Acetylglucosaminidase, alpha reductase NB6 | NAGLU | 1 |
| Nerve growth factor | NGF | 6 |
| Nerve growth factor receptor | NGFR | 6 |
| Neurite inhibitory protein | | 5 |
| Neuroendocrine convertase 1 | NEC1, PCSK1 | 1 |
| Neurofibromin 1 | NF1 | 6 |
| Neurofibromin 2 | NF2 | 6 |
| Neurofilament protein, NF68 | NF68 | 3 |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Notch 3 | NOTCH3 | 6 |
| Nuclear factor 1-kappa-B-like gene | IKB1 | 4 |
| Nucleoside diphosphate kinase-A | NDPKA | 1 |
| Oncogene bcI2 | | 6 |
| Oncogene sis | PDGFB | 6 |
| Omithine delta-aminotransferase | OAT | 1 |
| Omithine transcarbamoylase | OTC, NME1 | 1 |
| Parkin | PARK2 | 5 |
| Persyn | | 3 |
| Phosphoglucose isomerase | GPI | 1 |
| Phosphoglycerate kinase 1 | PGK1 | 1 |
| Phospholipase A2, group 10 | PLA2G 10 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Plasminogen | PLG | 1 |
| Plasminogen activator inhibitor 1 | PAI1 | 1 |
| Plasminogen activator inhibitor 2 | PAI2 | 1 |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Platelet-activating factor receptor | PAFR | 4 |
| Postsynaptic density-95 protein | PSD95 | 5 |
| Potassium inwardly-rectifying channel J1 | KCNJI | 5 |
| Potassium voltage-gated channel E1 | KCNE1 | 5 |
| Potassium voltage-gated channel Q1 | KCNQ1 | 5 |
| POU domain, class 1, transcription factor 1 (Pit1) | POU1F1 | 6 |
| Prekallikrein | | |
| Presenilin 1 | PSEN1 | 2 |
| Presenilin 2 | PSEN2 | 2 |
| Prion protein | PRNP | 5 |
| Procoliagen N-protease | | 1 |
| Proopiomelanocortin | POMC | 5 |
| Prostacyclin synthase | | 4 |
| Prostagiandin 15-OH dehydrogenase | HGPD; PGDH | 4 |
| Prostagiandin D - DP receptor | | 4 |
| Prostagiandin El receptor | | 4 |
| Prostaglandin E2 receptor | | 4 |
| Prostagiandin E3 receptor | | 4 |
| Prostaglandin F - FP receptor | | 4 |
| Prostaglandin 12 receptor | | 2 |
| Prostaglandin IP receptor | | 4 |
| Protective protein for beta-galactosidase | PPGB | 1 |
| Protein C | PROC | 4 |
| Protein C inhibitor | PCI | 4 |
| Protein kinase C, alpha | PRKCA | 1 |
| Protein kinase C, gamma | PRKCG | 1 |
| Protein kinase G | | 1 |
| Protein phosphatase 1, regulatory (inhibitor) | PPP1R3 | 1 |
| subunit 3 | | |
| Protein S | PROS1 | 4 |
| Prothrombin precursor | F2 | 4 |
| Purine nucleoside phosphorylase | NP | 1 |
| Pyruvate carboxylase | PC | 1 |
| Renin | REN | 1 |
| Replication factor C | RFC2 | 1 |
| RIGUI | RIGUI | 6 |
| S100 calcium-binding protein A3 | S 100A3 | 5 |
| S100 calcium-binding protein A4 | S 100A4 | 5 |
| S100 calcium-binding protein A5 | S 100A5 | 5 |
| S100 calcium-binding protein A7 | S 100A7 | 5 |
| S100 calcium-binding protein A8. | S 100A8 | 5 |
| S100 calcium-binding protein B | S 100B | 5 |
| S100 calcium-binding protein P | S 100P | 5 |
| Secretase, alpha | | 5 |
| Secretase, beta | | 5 |
| Secretase, gamma | | 5 |
| Selectin E | SELE | 5 |
| Selectin L | SELL | 5 |
| Selectin P | SELP | 5 |
| Serotonin N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage-gated, type 1, beta | SCN1B | 5 |
| polypeptide | | |
| Solute carrier family 1 (glutamate transporter), | SLC1A1 | 2 |
| member 1 | | |
| Solute carrier family 1 (glutamate transporter), | SLC1A2 | 2 |
| member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 8, member 3 | SLC18A3 | 2 |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5 member 3 | SLC5A3 | 2 |
| Solute carrier family 6 (GAMMA- | SLC6A | 2 |
| AMINOBUTYRIC ACID transporter), member 1 | | |
| Solute carrier family 6 (neurotransmifter | SLC6A3 | 2 |
| transporter, dopamine), member 3 | | |
| Solute carrier family 6 (neurotransmifter | SLC6A2 | 2 |
| transporter, noradrenaline), member 2 | | |
| Solute carrier family 6 (neurotransmifter | SLC6A4 | 2 |
| transporter, serotonin), member 4 | | |
| Sphingomyelinase | SMPD1 | 1 |
| Substance P | | 5 |
| Succinic semi-aidehyde dehydrogenase | SSADH | 1 |
| Sulfite oxidase | SUOX | 1 |
| Superoxide dismutase 1 | SOD1 | 1 |
| Superoxide dismutase 3 | SOD3 | 1 |
| Surfeit 1 | SURF1 | 6 |
| Synaptogyrin | | 5 |
| Synaptophysin | SYP | 5 |
| Syntaxin 1 | STXI | 5 |
| Talin | TLN | 6 |
| Tau protein | MAPT | 3 |
| TEK, tyrosine kinase, endothelial | TEK | 1 |
| Telomerase protein component | | 1 |
| Thrombin receptor | F2R | 4 |
| Thrombopoietin | THPO | 6 |
| Thromboxane A synthase 1 | TBXAS1 | 4 |
| Topoisomerase 1 | | 1 |
| Transforming growth factor, beta 2 | TGFB2 | 6 |
| Transforming growth factor, beta receptor 2 | TGFBR2 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tyrosine aminotransferase | TAT | 1 |
| Tyrosine hydroxylase | TH | 1 |
| Ubiquitin | | 66 |
| Ubiquitin B | UBB | 6 |
| Ubiquitin C | UBC | 6 |
| Ubiquitin carboxyl-terminal esterase L1 | UCHL1 | 6 |
| UDP-glucuronosyltransferase 1 | ugt1d, UGT1 | 1 |
| UDP-glucuronosyltransferase 2 | UGT2 | 1 |
| Uridinediphosphate(UDP)-galactose-4- | GALE | 1 |
| epimerase | | |
| Uroporphyrinogen III-synthase | UROS | 1 |
| Vacuolar proton pump, subunit 1 | VPP1 | 5 |
| Vacuolar proton pump, subunit 3 | VPP3 | 5 |
| Vasoactive intestinal polypeptide | VIP | 5 |
| Vasoactive intestinal polypeptide receptor | VIPR | 5 |
| Xanthine dehydrogenase | XDH | 1 |

that the genes associated with psychotic disturbances and personality disorders are selected from:

| | HUGO | Protein- |
|---|---|---|
| List of genes | gene symbol | function |
| 11 beta hydroxysteroid dehydrogenase 2 | HSD11B2 | 1 |
| 5,10-methylenetetrahydrofolate reductase | MTHFR | 1 |
| (NADPH) | | |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor. nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Adenosine receptor A1 | ADORA1 | 5 |
| Adenosine receptor A2A | ADORA2A | 5 |
| Adenosine receptor A2B | ADO 2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylosuccinate lyase | ADSL | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| alpha 1-antichymotrypsin | AACT | 1 |
| alpha-synuclein | SNCA | 5 |
| Amyloid beta A4 precursor protein | APP | 5 |
| Amyloid beta A4 precursor-like protein | APLP | 5 |
| Apolipoprotein A I | APOA1 | 2 |
| Apolipoprotein A II | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Arginosuccinate synthetase | ASS | 1 |
| Arylsuffatase A | ARSA | 1 |
| Ataxia telangiectasia gene, AT | ATM | 6 |
| ATP/ADP translocase | | 1 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Bagpipe homeobox, drosophila homolog of, 1 | BAPX1 | 6 |
| beta-synuclein | SNCB | 5 |
| Brain derived neurotrophic factor | BDNF | 6 |
| Brain derived neurotrophic factor (BDNF) | BDNFR | 6 |
| receptor | | |
| C1 inhibitor | | 1 |
| Ca(2+) transporting ATPase, slow twitch albindin | ATP2A2 | 2 |
| Calbindin D9K | CALB1 | 6 |
| Calcineurin A1 | CALB3 | 6 |
| Calcineurin A2 | CALNA1 | 4 |
| Calcineurin A3 | CALNA2 | 4 |
| Calcineurin B | CALNA3 | 4 |
| Calcitonin/Calcitonin gene-related peptide | CALCA | 5 |
| alpha | | |
| Calcium channel, voltage-dependent, alpha 1 F | CACNA 1F | 5 |
| subunit | | |
| Calcium channel, voltage-dependent, Alpha- | CACNA 1B | 5 |
| I B (CACNL1A5) | | |
| Calcium channel, voltage-dependent, Alpha- | CACNA 1C | 5 |
| I C | | |
| Calcium channel, voltage-dependent, Alpha- | CACNA 1D | 5 |
| 1 D | | |
| Calcium channel, voltage-dependent, Alpha- | CACNA 1E | 5 |
| I E (CACNL1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, | CACNG2 | 5 |
| Neuronal, Gamma | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase II | CAMK2A | 6 |
| Calnexin | CANX | 6 |
| Calpain | CAPN, | 1 |
| | CAPN3 | |
| Calretinin | CALB2 | 5 |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Caspase 1 | CASP1 | 6 |
| Catechol-o-methyltransferase | COMT | 1 |
| Ceroid lipofuscinosis neuronal 2 | CLN2 | 5 |
| Ceroid lipofuscinosis neuronal 3 | CLN3 | 5 |
| Ceroid lipofuscinosis neuronal 4 | CLN4 | 5 |
| Ceroid lipofuscinosis neuronal 5 | CLN5 | 5 |
| Ceroid lipofuscinosis neuronal 6 | CLN6 | 5 |
| Chemokine receptor CCR5 | CCR5 | 4 |
| Chemokine receptor CXCR4 | CXCR4 | 4 |
| Cholecystokinin | CCK | 5 |
| Cholecystokinin B receptor | CCKBR | 5 |
| Choline acetyltransferase | CHAT | 1 |
| Chymotrypsinogen | | 1 |
| Ciliary neurotrophic factor (CNTF) | CNTF | 6 |
| Ciliary neurotrophic factor (CNTF) receptor | CNTFR | 6 |
| Citrate synthase | | 1 |
| Colony-stimulating factor 2 | CSF2 | 6 |
| Colony-stimulating factor 2 alpha receptor | CSF2RA | 6 |
| Corticotrophin-releasing hormone | CRH | 2 |
| Corticotrophin-releasing hormone receptor | CRHR1 | 2 |
| Cu2+ transporting ATPase beta polypeptide | ATP7B | 1 |
| Cyclic AMP response element binding protein | CREB | 6 |
| Cyclic AMP-dependent protein kinase | PKA | 1 |
| Cyclic nucleotide phosphodiesterase 1 B | PDE1B | 1 |
| Cyclic nucleotide phosphodiesterase 1 B 1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Delta aminolevulinate dehydratase | ALAD | 1 |
| Delta-7-dehydrocholesterol reductase | DHCR7 | 1 |
| Dihydrolipoamide succinyltransferase | | 5 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | ENO1 | 1 |
| Epidermal growth factor | EGF | 6 |
| Epidermal growth factor receptor | EGFR | 6 |
| Excision repair complementation group 4 | ERCC4 | 1 |
| protein | | |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Flightless-II, Drosophila homolog of | FLII | 6 |
| Fragile site, folic acid type, rare, fra(X) A | FRAXA | 5 |
| Fragile site, folic acid type, rare, fra(X) E | FRAXE | 5 |
| Fragile site, folic acid type, rare, fra(X) F | FRAXF | 5 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRGI | 5 |
| GABA receptor, gamma 2 | GABRG2 | 5 |
| GABA receptor, gamma 3 | GABRG3 | 5 |
| GABA transaminase | ABAT | 1 |
| GDP dissociation inhibitor 1 | GDI 1 | 6 |
| Geniospasm 1 | GSM1 | 6 |
| Glial-cell derived neurotrophic factor (GDNF) | | 5 |
| receptor | | |
| Glial-cell derived neurotrophic factor, GDNF | GDNF | 5 |
| Glutamate decarboxylase, GAD | GAD1 | 1 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutaryl-CoA dehydrogenase | GCDH | 1 |
| Glutathione | GSH | 2 |
| Glutathione S-transferase, GSTZ1 | GSTZ1 | 1 |
| Glyceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Gonadotropin releasing hormone receptor | GNRHR | 6 |
| Guanidinoacetate N-methyltransferase | GAMT | 1 |
| Guanine nucleotide-binding protein, alpha | GNAO1 | 5 |
| activating activity polypeptide, GNAO | | |
| Guanine nucleotide-binding protein, alpha | GNAI1 | 5 |
| inhibiting activity polypeptide 1, GNAI1 | | |
| Guanine nucleotide-binding protein, alpha | GNAI2 | 5 |
| inhibiting activity polypeptide 2, GNAI2 | | |
| Guanine nucleotide-binding protein, alpha | GNAI3 | 5 |
| inhibiting activity polypeptide 3, GNAI3 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNAT1 | 5 |
| transducing activity polypeptide, GNAT1 | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, | GNAQ | 5 |
| polypeptide | | |
| Guanylate cyclase 2D, membrane (retina- | GUCY2D | 1 |
| specific) | | |
| Guanylate cyclase activator 1A (retina) | GUCA1A | 1 |
| Guanylyl cyclase | | 1 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Heparan sulfamidase | | 1 |
| Hepatic lipase | LIPC | 1 |
| Histamine receptors, H1 | | 5 |
| Histamine receptors, H2 | | 5 |
| Histamine receptors, H3 | | 5 |
| HMG-CoA reductase | HMGCR | 1 |
| Huntingtin | HD | 2 |
| Hypoxanthine-guanine | HPRT | 1 |
| phosphoribosyltransferase, HGPRT | | |
| Hypoxia inducible factor 1 | HIF1A | 1 |
| Hypoxia inducible factor 2 | | 1 |
| Inositol monophosphatase | IMPA1 | 5 |
| Insulin | INS | 6 |
| Insulin receptor | INSR | 6 |
| lnterleukin(IL) 1 receptor | IL1R | 4 |
| lnterleukin(IL) 1, alpha | IL1A | 4 |
| Interleukin(IL) 1, beta | IL1B | 4 |
| Interleukin(IL) 10 | IL10 | 4 |
| lnterleukin(IL) 10 receptor | IL10R | 4 |
| lnterleukin(IL) 11 | IL11 | 4 |
| Interleukin(IL) 11 receptor | IL11R | 4 |
| Interleukin(IL) 12 | IL12 | 4 |
| Interleukin(IL) 12 receptor, beta 1 | IL12RB1 | 4 |
| Interleukin(IL) 13 | IL13 | 4 |
| lnterleukin(IL) 13 receptor | IL13R | 4 |
| Interleukin(IL) 2 | IL2 | 4 |
| Interleukin(IL) 2 receptor, alpha | IL2RA | 4 |
| lnterleukin(IL) 2 receptor, gamma | IL2RG | 4 |
| Interleukin(IL) 3 | IL3 | 4 |
| lnterleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| lnterleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| lnterleukin(IL) 9 | IL9 | 4 |
| Interleukin(IL) 9 receptor | IL9R | 4 |
| lnterleukin(IL) receptor antagonist 1 | IL1 RN, | 4 |
| | IL1RA | |
| IP3 kinase | | 1 |
| Leukin | | 4 |
| Mismatch repair gene, PMSL2 | PMS2 | 6 |
| Monoamine oxidase A | MAOA | 1 |
| Monoamine oxidase B | MAOB | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | | |
| CHRM5 | | 5 |
| Myelin basic protein | | 3 |
| Myosin, light chain 3 | MYL3 | 3 |
| reductase | | |
| Nerve growth factor | NGF | 6 |
| Nerve growth factor receptor | NGFR | 6 |
| Neurite inhibitory protein | | 5 |
| Neurofibromin 1 | NF1 | 6 |
| Neurofibromin 2 | NF2 | 6 |
| Neurofilament protein, NF68 | NF68 | 3 |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Neurotensin | NTS | 5 |
| Neurotensin receptor | NTSR1 | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Nucleoside diphosphate kinase-A | NDPKA | 1 |
| Oncogene sis | PDGFB | 6 |
| Opioid receptor, delta | OPRD1 | 5 |
| Opioid receptor, kappa | OPRKI | 5 |
| Opioid receptor, mu | OPRMI | 5 |
| Ornithine delta-aminotransferase | OAT | 1 |
| Paraoxonase PON1 | PON1 | 1 |
| Parkin | PARK2 | 5 |
| Phospholipase A2, group 10 | PLA2G10 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Platelet derived growth factor | PDGF | 6 |
| Platelet derived growth factor receptor | PDGFR | 6 |
| Potassium inwardly-rectifying channel J1 | KCNJL | 5 |
| POU domain, class 1, transcription factor 1 | POU1F1 | 6 |
| (Pit1) | | |
| Presenilin 1 | PSEN1 | 2 |
| Presenilin 2 | PSEN2 | 2 |
| Prion protein | PRNP | 5 |
| Proline dehydrogenase | PRODH | 1 |
| Proopiomelanocortin | POMC | 5 |
| Prosaposin | PSAP | 5 |
| Protective protein for beta-galactosidase | PPGB | 1 |
| Protein kinase C, alpha | PRKCA | 1 |
| Protein kinase C, gamma | PRKCG | 1 |
| Protein kinase G | | 1 |
| Protein phosphatase 1, regulatory (inhibitor) | PPP1R3 | 1 |
| subunit 3 | | |
| Proteolipid protein | PLP | 5 |
| RIGUI | RIGUI | 6 |
| S100 calcium-binding protein A2 | S100A2 | 5 |
| S100 calcium-binding protein A3 | S100A3 | 5 |
| S100 calcium-binding protein A4 | S100A4 | 5 |
| S100 calcium-binding protein A5 | S100A5 | 5 |
| S100 calcium-binding protein A7 | S100A7 | 5 |
| S100 calcium-binding protein A8 | S100A8 | 5 |
| S100 calcium-binding protein A9 | S100A9 | 5 |
| S100 calcium-binding protein B | S100B | 5 |
| S100 calcium-binding protein P | S100P | 5 |
| Secretase, alpha | | 5 |
| Secretase, beta | | 5 |
| Secretase, gamma | | 5 |
| Serotonin N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage-gated, type 1, beta | SCN1B | 5 |
| polypeptide | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 4 (anion exchanger), | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A2 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Superoxide dismutase 1 | SOD1 | 1 |
| Superoxide dismutase 3 | SOD3 | 1 |
| Synapsin 1a & 1b | SYN1 | 5 |
| Synapsin 2a & 2b | SYN2 | 5 |
| Synaptic vesicle amine transporter | SVAT | 5 |
| Synaptogyrin | | 5 |
| Synaptophysin | SYP | 5 |
| Synaptosomal-associated protein, 25 KD | SNAP25 | 5 |
| Syntaxin 1 | STX1 | 5 |
| Tachykinin receptor, NK1R | TACR1 | 5 |
| Tachykinin receptor, NK2R | TACR2 | 5 |
| Tachykinin receptor, NK3R | TACR3 | 5 |
| Talin | TLN | 6 |
| TEK, tyrosine kinase, endothelial | TEK | 1 |
| Telomerase protein component | | 1 |
| Transcobalamin 1, TCN1 | | 2 |
| Transcobalamin 2, TCN2 | TCN2 | 2 |
| Transcription factor, TUPLE1 | TUPLE1 | 5 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Transthyretin | TTR | 2 |
| Trypsin inhibitor | | 1 |
| Tryptophan 2,3-dioxygenase | TDO2 | 5 |
| Tryptophan hydroxylase | TPH | 1 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tyrosinase | TYR | 1 |
| Tyrosine hydroxylase | TH | 1 |
| Ubiquitin | | 6 |
| Ubiquitin activating enzyme, E1 | | 1 |
| Ubiquitin B | UBB | 6 |
| Ubiquitin C | UBC | 6 |
| Ubiquitin protein ligase E3A | UBE3A | 1 |
| UDP-glucuronosyltransferase 1 | ugtld, UGTI | 1 |
| UDP-glucuronosyltransferase 2 | UGT2 | 1 |
| Uridinediphosphate(UDP)-galactose-4- | GALE | 1 |
| epimerase | | |
| Vacuolar proton pump, subunit 1 | VPP1 | 5 |
| Vacuolar proton pump, subunit 3 | VPP3 | 5 |
| Vesicular monoamine transporter 1 | VMAT1 | 5 |
| Vesicular monoamine transporter 2 | VMAT2 | 5, |

that the genes associated with cardiovascular disorder, malfunction or damage are selected from:

| | HUGO | |
|---|---|---|
| List of genes | gene symbol | Protein-function |
| 17beta hydroxysteroid oxidoreductase | | 1 |
| 2,3-bisphosphoglycerate mutase | BPGM | 1 |
| 3 beta hydroxysteroid dehydrogenase 2 | HSD3B2 | 1 |
| 3-oxoacid CoA transferase | OXCT | 1 |
| 5,10-methylenetetrahydrofolate reductase | MTHFR | 1 |
| (NADPH) | | |
| Acetoacetyl 1-CoA-thiolase | ACAT1 | 1 |
| Acetoacetyl 2-CoA-thiolase | ACAT2 | 1 |
| Acetyl CoA acyltransferase | ACAA | 1 |
| Acety[cholinesterase | ACHE | 1 |
| Acid phosphatase 2, lysosomal | ACP2 | 1 |
| Acidic amino acid transporter | | 2 |
| Actin, alpha, cardiac | ACTC | 3 |
| Actin, alpha, skeletal | ACTAL | 3 |
| Actin, alpha, smooth, aortic | ACTA2 | 3 |
| Activin A receptor, type 2B | ACVR2B | 6 |
| Acyl CoA dehydrogenase, long chain | ACADL | 1 |
| Acyl CoA dehydrogenase, very long chain | ACADVL | 1 |
| Adaptin, beta 3A | ADTB3A | 2 |
| Adducin, alpha | ADDI | 3 |
| Adducin, beta | ADD2 | 3 |
| Adenosine deaminase | ADA | 1 |
| Adenosine receptor A1 | ADORAL | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase I | ADCYI | 1 |
| Adenylate cyclase 2. | ADGY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylate kinase | AK1 | 1 |
| Adrenergic receptor, alphal | ADRAI | 5 |
| Adrenergic receptor, alpha2 | ADRA2 | 5 |
| Adrenergic receptor, betal | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) receptor | ACTHR | 6 |
| Alanine aminotransferase | | 2 |
| Alanine-glyoxylate aminotransferase | AGXT | 1 |
| Albumin, ALB | ALB | 2 |
| Alcohol dehydrogenase 1 | ADHL | 1 |
| Alcohol dehydrogenase 2 | ADH2 | 1 |
| Alcohol dehydrogenase 3 | ADH3 | F = |
| Alcohol dehydrogenase 5 | ADH5 | 1 |
| Alcohol dehydrogenase 6 | ADH6 | 1 |
| Alcohol dehydrogenase 7 | ADH7 | 1 |
| Aidehyde dehydrogenase 1 | ALDHL | 1 |
| Aidehyde dehydrogenase 10 | ALDH10 | 1 |
| Aidehyde dehydrogenase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldolase A | ALDOA | 1 |
| Aidolase B | ALDOB | 1 |
| Aldolase C | ALDOC | 1 |
| Aldosterone receptor | MLR | 6 |
| Alpha 1 acid glycoprotein | G; AGP | 2 |
| Alpha 2 macroglobulin | A2M | 4 |
| alphal-antitrypsin | Pi | 1 |
| alpha2-antiplasmin | PLI | 1 |
| alpha-actinin 2 | ACTN2 | 6 |
| alpha-actinin 3 | ACTN3 | 6 |
| alpha-Galactosidase A | GLA | 1 |
| alpha-L-Iduronidase | IDUA | 1 |
| Aminopeptidase P | XPNPEP2 | 1 |
| Amphiregulin | AREG | 6 |
| Amylo-1,6-glucosidase | AGL | 1 |
| Angiopoietin 1 | ANGPT1 | 6 |
| Angiopoietin 2 | ANGPT2 | 6 |
| Angiotensin converting enzyme | ACE, DCPL | 1 |
| Angiotensin receptor 1 | AGTRI | 2 |
| Angiotensi'n receptor 2 | AGTR2 | 2 |
| Angiotensinogen | AGT | 1 |
| Ankyrin 1 | ANKI | 3 |
| Ankyrin 2 | ANK2 | 3 |
| Ankyrin 3 | ANK3 | 3 |
| Annexin I | ANXI | 4 |
| Antidiuretic hormone receptor | ADHR | 2 |
| Antithrombin III | AT3 | 1 |
| Apolipoprotein (a) | LPA | 2 |
| Apolipoprotein A 4 | APOA4 | 2 |
| Apolipoprotein A I | APOAI | 2 |
| Apolipoprotein A 11 | APOA2 | 2 |
| Apolipoprotein B | APOB | 2 |
| Apolipoprotein C1 | APOCI | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Aquaporin I | AQPL | 2 |
| Aquaporin 2 | AQP2 | 2 |
| Arginine vasopressin | AVP | 5 |
| Arginine vasopressin receptor 1A | AVPRLA | 5 |
| Arginine vasopressin receptor 1B | AVPRLB | 5 |
| Arginine vasopressin receptor 2 | AVPR2 | 5 |
| Arginosuccinate lyase | ASL | 1 |
| Arylsuffatase B | ARSB | 1 |
| Aspartylglucosaminidase | AGA | 1 |
| Ataxia telangiectasia gene, AT | ATM | 6 |
| ATP/ADP transiocase | | 1 |
| ATP-binding cassette transporter 7 | ABC7 | 4 |
| Atrial natriuretic peptide | ANP | 6 |
| Atrial natriuretic peptide receptor A | NPRL | 6 |
| Atria natriuretic peptide receptor B | NPR2 | 6 |
| Atrial natriuretic peptide@ receptor C | NPR3 | 6 |
| Autoimmune regulator, AIRE | AIRE | 4 |
| BCL2-related protein A1 | BCL2AI | 6 |
| beta 2 microglob@ulin | B2M | |
| beta-endorphin receptor N | | |
| Bile acid coenzyme A: amino acid N- | BAAT | 1 |
| acyltransferase | | |
| Bile salt export pump | BSEP, PFIC2 | 2 |
| Bile salt-stimulated lipase | CEL | 1 |
| Bilirubin UDP-glucuronosyltransferase | | 1 |
| Bloom syndrome protein | BLM | 6 |
| Bradykinin receptor BI | | |
| Bradykinin receptor B2 | | |
| Butyrylcholinesterase | BCHE | 1 |
| Ca(2+) transporting ATPase, fast twitch | ATP2AI | 2 |
| Ca(2+) transporting ATPase, slow twitch | ATP2A2 | 2 |
| Cadherin E | CDHL | 6 |
| Cadherin EP | | 6 |
| Cadherin N | CDH2 | 6 |
| Cadherin P | CDH3 | 6 |
| Calbindin I | CALB1 | 6 |
| Calbindin D9K | CALB3 | 6 |
| Calcineurin A1 | CALNAL | 4 |
| Calcineurin A2 | CALNA2 | |
| Calcineurin A3 | CALNA3 | |
| Calcineurin B | | |
| Calcium channel, voltage-dependent, alpha | CACNAL F | 5 |
| 1 F subunit | | |
| Calcium channel, voltage-dependent, Alpha- | CACNAIB | 5 |
| 1B (CACNL1A5) | | |
| Calcium channel, voltage-dependent, Alpha-I c | CACNAL C | 5 |
| Calcium channel, voltage-dependent, Alpha-1 D | CACNAL D | 5 |
| Calcium channel, voltage-dependent, Alpha- | CACNAL E | 5 |
| 1E (CACNLIA6) | | |
| Calcium channel, voltage-dependent, Alpha- | CACNA2 | 5 |
| 2/delta | | |
| Calcium channel, voltage-dependent, Beta 1 | CACNBI | 5 |
| Calcium channel, voltage-dependent, Beta 3 | CACNB3 | 5 |
| Calcium channel, voltage-dependent, L type, | CACNAL S | 5 |
| alpha IS subunit | | |
| Calcium channel, voltage-dependent, | CACNG2 | 5 |
| Neuronal, Gamma | | |
| Calcium channel, voltage-dependent, P/Q | CACNALA | 5 |
| type, alpha 1A subunit | | |
| Calcium channel, voltage-dependent, T-type | | 5 |
| Calmodulin I | CALMI | 6 |
| Calmodutin 2 | CALM2 | 6 |
| Calmodulin 3 | CALM3 | 6 |
| Calmodulin-dependant protein kinase 11 | CAMK2A | 6 |
| Calpain | CAPNCAPN3 | 1 |
| Calretinin | CALB2 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carboxypeptidase | CPN | 1 |
| Cardiac-specific homeobox, CSX | csx | 6 |
| Carnitine acylcarnitine translocase | CACT | 1 |
| Carnitine transporter protein | CDSP, SCD | 2 |
| Cartilage-hair hypoplasia gene | CHH | 5 |
| Catechol-0-methyltransferase | COMT | 1 |
| Caveolin 3 | CAV3 | 1 |
| CD1 | CDI | |
| CD4 | CD4 | |
| Cdc 25 phosphatase | | 6 |
| Cell adhesion molecule, intercellular, ICAM | ICAMI | 6 |
| Cell adhesion molecule, leukocyte- | LECAM1 | 6 |
| endotheiial, LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM1 | 6 |
| PECAM | | |
| Cell adhesion molecule, vascular, VCAM | VCAM1 | 6 |
| Cellubrevin | CEB | 5 |
| Ceroid lipofuscinosis neuronal 3 | CLN3 | 5 |
| Ceruloplasmin precursor | CP | 1 |
| Chemokine receptor CCR2 | CCR2 | |
| Chemokine receptor CCR3 | CCR3 | |
| Chemokine receptor CCR5 | CCR5 | |
| Chemokine receptor CXCRI | CXCR1 | |
| Chemokine receptor CXCR2 | CXCR2 | |
| Chemokine receptor CXCR4 | CXCR4 | |
| Chloride channel KB | CLCNKB | 3 |
| Cholestasis, progressive familial intrahepatic | FICI | 6 |
| 1 gene | | |
| Cholesterol ester transfer protein | CETP | 2 |
| Choline acetyltransferase | CHAT | 1 |
| Chymase | CHYI | |
| Clathrin | | 2 |
| Cockayne syndrome gene, CKNI | CKNI | 6 |
| Collagen I alpha 1 | COLIAL | 3 |
| Collagen I alpha 2 | COLIA2 | 3 |
| Collagen 11 alpha 1 | COL2AI | 3 |
| Collagen III alpha 1 | COL3A1 | 3 |
| Collagen IV alpha 1 | COL4AI | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5AI | 3 |
| Collagen V alpha 2 | COL5A2 | 3 |
| Collagen VI alpha 1 | COL6Al | 3 |
| Collagen VI alpha 2 | COL6A2 | 3 |
| Collagen VI alpha 3 | COL6A3 | 3 |
| Collagen VII alpha 1 | COL7Al | 3 |
| Collagen X alpha I | COLlOAl | 3 |
| Collagen X alpha 1 | COLIlAl | 3 |
| Collagen Xi alpha 2 | COLL IA2 | 3 |
| Collagen XVII alpha I | COL17AI | 3 |
| Collagenic-like tail subunit of asymmetric | COLQ | 1 |
| acetylcholinesterase | | |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Corticosteroid binding globulin | CBG | 5 |
| Cortico-sterold binding protein | | 2 |
| Corticotrophin-releasing hormone | CRH | 2 |
| Corticotrophin-releasing hormone receptor | CRHR1 | 2 |
| Creb binding protein | CREBBP | 6 |
| Cu2+ transporting ATPase alpha polypeptide | ATP7A | 1 |
| Cu2+ transporting ATPase beta polypeptide | ATP7B | 1 |
| Cubilin | CUBN | 2 |
| Cyclic AMP-dependent protein kinase | PKA | 1 |
| Cyclin-dependent kinase 2 | CDK2 | 6 |
| Cyclin-dependent kinase inhibitor 1C (P57, KIP2) | CDKNIC | 6 |
| Cyclooxygenase I | coxi | 1 |
| Cyclooxygenase 2 | COX2 | 1 |
| CYPL 1A1 | CYPllA1 | 1 |
| CYPL 1 B1 | CYPllB1 | 1 |
| CYPI 1 B2 | CYPllB2 | 1 |
| CYP17 | CYP17 | 1 |
| CYP19 | cypig | 1 |
| CYPIAI | CYPlA1 | 1 |
| CYPlA2 | CYPIA2 | 1 |
| CYPIBL | CYPIBI | 1 |
| CYP21 | CYP21 | 1 |
| CYP24 | CYP24 | 1 |
| CYP27 | CYP27 | 1 |
| CYP27BI | PDDR | 1 |
| CYP2AI | CYP2AI | 1 |
| CYP2Al3 | CYP2Al3 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2Cl8 | CYP2Cl8 | F- |
| CYP2CI9 | CYP2Cl9 | 1 |
| CYP2C8 | CYP2C8 | 1 |
| CYP2C9 | CYP2C9 | 1 |
| CYP2El | CYP2El | 1 |
| CYP2Fl | CYP2Fl | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4AIl | CYP4All | 1 |
| CYP4B1 | CYP4BI | 1 |
| CYP4F2 | CYP4F2 | 1 |
| CYP4F3 | CYP4F3 | 1 |
| CYP51 | CYP51 | 1 |
| CYP5AI | CYP5Al | 1 |
| CYP7A | CYP7A | I |
| CYP8 | CYP8 | 1 |
| Cystathionase | CTH | 1 |
| Cystathione beta synthase | CBS | 1 |
| Cytidine deaminase | CDA | 1 |
| Cytidine-5-prime-triphosphate synthetase | CTPS | 1 |
| Cytochrome a | | 1 |
| Cytochrome b-5 | CYB5 | 1 |
| Cytochrome c | | 1 |
| Cytochrome c oxidase, MTCO | | 1 |
| DAX1 nuclear receptor | DAX1 | 4 |
| Delta aminolevulinate dehydratase | ALAD | 1 |
| Delta(4)-3-oxosteroid 5-beta-reductase | | 1 |
| Delta-7-dehydrocholesterol reductase | DHCR7 | 1 |
| Deoxycorticosterone (DOC) receptor | | 1 |
| Desmin | DES | 3 |
| Dihydrodiol dehydrogenase 1 | DDHL | 1 |
| Dihydrofolate reductase | DHFR | 1 |
| Dihydrolipoyl dehydrogenase | | 1 |
| Dihydrolipoyl dehydrogenase 2 | PDHA | 1 |
| Dihydrolipoyl transacetylase | PDHA | 1 |
| DM-Kinase | DMPK | 1 |
| DOPA decarboxylase | DDC | 1 |
| Dopamine beta hydroxylase | DBH | 1 |
| Dopamine receptors DI | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Duffy blood group | FY | 2 |
| Dynamin | DNMI | 6 |
| Dystrophia myotonica | DM, DMPK | 1 |
| Dystrophia myotonica, atypical | DM2 | 1 |
| Dystrophin | DMD | 3 |
| Elastin | ELN | 3 |
| Emerin | EMD | 2 |
| Endocardial fibroelastosis 2 gene | EFE2 | 3 |
| Endoglin | ENG | 3 |
| Endometrial bleeding-associated factor | EBAF | 6 |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | EN01 | 1 |
| Enoyl CoA isomerase | | 1 |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Erythrocyte membrane protein band 4.1 | EPB41 | 3 |
| Erythrocyte membrane protein band 4.2 | EPB42 | 3 |
| Erythrocyte membrane protein band 7.2 | EPB72 | 3 |
| Erythroid kruppel-like factor | EKLF | 6 |
| Erythropoietin | EPO | |
| Erythropoietin receptor | EPOR | |
| Estrogen receptor | ESR | 6 |
| Faciogenital dysplasia | FGD1, FGDY | 2 |
| Factor 1 (No. one) | F1 | |
| Factor B, properdin | | |
| Factor D | | |
| Factor H | HFI | |
| Factor I (letter 1) | IF | |
| Factor III | F3 | |
| Factor IX | F9 | |
| Factor V | F5 | |
| Factor VI I | F7 | 4 |
| Factor VI II | F8 | 4 |
| Factor X | F10 | 4 |
| Factor Xi | Fil | 4 |
| Factor XI I | F12 | 4 |
| Factor XIII A & B | F13A & F13B | 4 |
| Fanconi anemia, complementation group A | FANCA | 2 |
| Fanconi anemia, complementation group C | FANCC | 2 |
| Fanconi anemia, complementation group D | FANCD | 2 |
| Fatty acid binding proteins FABPI | | 2 |
| Fatty acid binding proteins FABP2 | FABP2 | 2 |
| Fatty acid binding proteins FABP3 | | 2 |
| Fatty acid binding proteins FABP4 | | 2 |
| Fatty acid binding proteins FABP5 | | 2 |
| Fatty acid binding proteins FABP6 | | 2 |
| Fc fragment of IgG, high affinity IA, receptor for | FCGRIA | 6 |
| Fc fragment of IgG, low affinity Ila, receptor | FCGR2A | 6 |
| for (CD32) | | |
| Fc fragment of IgG, low affinity llla, receptor | FCGR3A | 6 |
| for (CD 1 6) | | |
| Fibrillin 1 | FBNI | 6 |
| Fibrillin 2 | FBN2 | 6 |
| Fibrinogen alpha | FGA | 3 |
| Fibrinogen beta | FGB | 3 |
| Fibrinogen gamma | FGG | 3 |
| Fibroblast growth factor | FGFI | 6 |
| Fibroblast growth factor receptor 1 | FGFRI | 6 |
| Fibroblast growth factor receptor 2 | FGFR2 | 6 |
| Fibroblast growth factor receptor 3 | FGFR3 | 6 |
| Fibronectin precursor | FNI | 6 |
| Flightless-11, Drosophila homolog of | FLII | 6 |
| Follicle stimulating hormone receptor | FSHR, ODG1 | 6 |
| Follicle stimulating hormone, FSH | FSHB | 6 |
| Formiminotransferase | | 1 |
| Fragile site, folic acid type, rare, fra(X) A | FRAXA | 5 |
| Fucosidase alpha-L-2 | | 1 |
| Fucosyltransferase 2 | FUT2 | 2 |
| Fucosyltransferase 3 | FUT3 | 2 |
| Fucosyltransferase 6 | FUT6 | 2 |
| Fukuyama type congenital muscular dystrophy | FCMD | 6 |
| GABA receptor, alpha 1 | GABRAL | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta I | GABRB1 | 5 |
| GABA receptor, beta 2 | GABRB2 | 5 |
| GABA receptor, beta 3 | GABRB3 | 5 |
| GABA receptor, gamma 1 | GABRGI | 5 |
| GABA receptor, gamma 2 | GABRG2 | 5 |
| GABA receptor, gamma 3 | GABRG3 | 5 |
| GABA transaminase | ABAT | 1 |
| Galactose 1-phosphate uridyl-transferase | GALT | 1 |
| Galactosyltransferase I | GTI | 6 |
| Galactosyltransferase, alpha 1,3 | GGTAL | 6 |
| Galactosyltransferase, beta 3 | B3GALT | 6 |
| Galanin | GAL | 5 |
| Galanin receptor | GALNRI | 5 |
| Gamma-glutamyl carboxylase | GGCX | 2 |
| Gap junction protein alpha I | GJAL | 2 |
| Gap junction protein beta I | GJBL | 2 |
| Gap junction protein beta 2 | GJB2 | 2 |
| Glucocorticoid receptor | GRL | 6 |
| Glucosaminyl (N-acetyl) transferase 2, I- | GCNT2 | 1 |
| branching enzyme | | |
| Glucosidase, acid alpha | GAA | 1 |
| Glucosidase, acid beta | GBA | 1 |
| Glutamate decarboxylase, GAD | GAD1 | 1 |
| Glutamate receptor 1 | GLURI | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutamate-cysteine ligase | GLCLC | 1 |
| Glutaryi-CoA dehydrogenase | GCDH | 1 |
| Glutathione | GSH | 2 |
| Glutathione peroxidase, GPXI | GPXL | 1 |
| Glutathione reductase, GSR | GSR | 1 |
| Glutathione S-transferase, GSTZI | GSTZI | 1 |
| Glyceraldehyde-3-phosphate | GAPDH | 1 |
| dehydrogenase, GAPDH | | |
| Glycerol kinase | GK | 1 |
| Glycinamide ribonucleotide (GAR) transformylase | GART | 1 |
| Glycophorin A | GYPA | 3 |
| Glycophorin B | GYPB | 3 |
| Glycophorin C | GYPC | 3 |
| Glycosyltransferases, ABO blood group | ABO | 1 |
| Growth arrest-specific homeobox | GAX | 6 |
| Guanine nucleotide-binding protein, alpha | GNAOI | 5 |
| activating activity polypeptide, GNAO | | |
| Guanine nucleotide-binding protein, alpha | GNAll | 5 |
| inhibiting activity polypeptide 1, GNAII | | |
| Guanine nucleotide-binding protein, alpha | GNA 2 | 5 |
| inhibiting activity polypeptide 2, GNA12 | | |
| Guanine nucleotide-binding protein, alpha | GNA13 | 5 |
| inhibiting activity polypeptide 3, GNA13 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, q | GNAQ | 5 |
| polypeptide | | |
| Guanylyl cyclase | | 1 |
| H(+), K(+) - ATPase | ATP4B | 5 |
| Haemoglobin alpha I | HBAL | 2 |
| Haemoglobin alpha 2 | HBA2 | 2 |
| Haemoglobin beta | HBB | 2 |
| Haemoglobin delta | HBD | 2 |
| Haemoglobin epsilon | | 2 |
| Haemoglobin gamma A | HBGL | 2 |
| Haemoglobin gamma 8 | HBG2 | 2 |
| Haemoglobin gamma G | HBGG | 2 |
| Haptoglobin, alpha 1 | HPAI | |
| Haptoglobin, alpha 2 | HPA2 | |
| Haptoglobin, beta | HPB | |
| Heat shock protein, HSP60 | | |
| Heat shock protein, HSP70 | | |
| Heat shock protein, HSP90 | | |
| Heat shock protein, HSPAI | | |
| Heat shock protein, HSPA2 | | |
| Hemochromatosis | HFE | 2 |
| Hemopexin | HPX | 4 |
| Heparan sulfamidase | | 1 |
| Heparin binding epidermal growth factor | HBEGF | 6 |
| Heparin Cofactor 11 | HCF2 | 4 |
| Hepatic lipase | LIPC | 1 |
| Hermansky-pudiak syndrome gene | HPS | 2 |
| Hexokinase 1 | HKI | 1 |
| Hexosaminidase A | HEXA, TSD | 1 |
| Hexosaminidase B | HEXB | 1 |
| Histidine-rich glycoprotein | HRG | 2 |
| HLA-B associated transcript 1 | BATI | 4 |
| HLH transcription factor HAND1 | HAND1 | 6 |
| HLH transcription factor HAND2 | HAND2 | 6 |
| HMG-COA lyase | HMGCL | 1 |
| HMG-COA reductase | HMGCR | 1 |
| HMG-COA synthase | HMGCS2 | 1 |
| Homeobox (HOX) gene Al 3 | HOXA13 | 6 |
| Homeobox HB24 | HLXL | 6 |
| Hormone-sensitive lipase | HSL | 1 |
| Human chorionic gonadtrophin, hCG | CG | 6 |
| Human placental lactogen | CSHI | 6 |
| Hypoxanthine-guanine | HPRT | 1 |
| phosphoribosyltransferase, HGPRT | | |
| Hypoxia inducible factor I | HiFlA | 1 |
| Hypoxia inducible factor 2 | | 1 |
| IC7 A and B | | 4 |
| Iduronate 2 sulphatase | IDS | 1 |
| Indian hedgehog, ihh | IHH | 6 |
| lnosine triphosphatase | ITPA | 1 |
| lnositol 1,4,5-triphosphate receptor 1 | ITPRL | 6 |
| Inositol 1,4,5-triphosphate receptor 3 | ITPR3 | 6 |
| Inositol monophosphatase | IMPAL | 5 |
| Inositol polyphosphate 1-phosphatase | INPPI | 5 |
| Insulin | INS | 6 |
| Insulin receptor | INSR | 6 |
| Insulin receptor substrates | IRS1 | 6 |
| Insulin-like growth factor 1 | IGF1 | 6 |
| Insulin-like growth factor 1 receptor | IGF1 R | 6 |
| Insulin-like growth factor 2 | IGF2 | 6 |
| Insulin-like growth factor 2 receptor | IGF2R | 6 |
| Integrin beta 1 | ITGBL | 6 |
| lntegrin beta 2 | ITGB2 | 6 |
| Integrin beta 3 | ITGB3 | 6 |
| Integrin beta 4 | ITGB4 | 6 |
| Integrin beta 5 | ITGB5 | 6 |
| Integrin beta 6 | ITGB6 | 6 |
| Integrin beta 7 | ITGB7 | 6 |
| Integrin, alpha 1 | ITGAL | 6 |
| Integrin, alpha 2 | ITGA2 | 6 |
| Integrin, alpha 3 | ITGA3 | 6 |
| lntegrin, alpha 4 | ITGA4 | 6 |
| Integrin, alpha 5 | ITGA5 | 6 |
| lntegrin, alpha 6 | ITGA6 | 6 |
| lntegrin, alpha 7 | ITGA7 | 6 |
| lntegrin, alpha 8 | ITGA8 | 6 |
| lntegrin, alpha 9 | ITGA9 | 6 |
| lntegrin, alpha M | ITGAM | 6 |
| Integrin, alpha X | ITGAX | 6 |
| Inter-alpha-trypsin inhibitor, IATI | | 1 |
| Intercellular adhesion molecule 1 | ICAMI | |
| lntercellular adhesion molecule 2 | ICAM2 | |
| lntercellular adhesion molecule 3 | ICAM3 | |
| Interferon alpha | IFNAI | |
| Interferon beta | IFNB | |
| Interferon gamma | IFNG | |
| Interferon gamma receptor 1 | IFNGRL | |
| Interferon gamma receptor 2 | IFNGR2 | |
| Interleukin(IL) 1 receptor | ILl R | |
| Interieukin(IL) 1, alpha | ILLA | |
| lnterieukin(IL) 1, beta | ILLB | |
| lnterieukin(IL) 10 | ILIO | |
| lnterleukin(IL) 10 receptor | ILIOR | |
| Intedeukin(IL) 1 1 | ILl 1 | |
| Interieukin(IL) 1 1 receptor | ILIIR | |
| lntedeukin(IL) 12 | IL12 | |
| Intedeukin(IL) 12 receptor, beta 1 | IL12RB1 | 4 |
| Intedeukin(IL) 13 | IL13 | 4 |
| Interfeukin(IL) 13 receptor | IL13R | 4 |
| Intedeukin(IL) 2 | IL2 | 4 |
| Interleukin(IL) 2 receptor, alpha | IL2RA | 4 |
| lnterleukin(IL) 2 receptor, gamma | IL2RG | 4 |
| lnterleukin(IL) 3 | IL3 | |
| Interleukin(IL) 3 receptor | IL3R | |
| Interieukin(IL) 4 | IL4 | |
| Interieukin(IL) 4 receptor | IL4R | |
| Interieukin(IL) 5 | IL5 | |
| Interleukin(IL) 5 receptor | IL5R | |
| lntedeukin(IL) 6 | IL6 | |
| lnterieukin(IL) 6 receptor | IL6R | |
| Interleukin(IL) 7 | IL7 | |
| lnterleukin(IL) 7 receptor | IL7R | |
| Interieukin(IL) 8 | IL8 | |
| Intedeukin(IL) 8 receptor | IL8R | 4 |
| lnterfeukin(IL) 9 | IL9 | 4 |
| Interleukin(IL) 9 receptor | IL9R | |
| Interieukin(IL) receptor antagonist 1 | ILlRN, ILLRA | |
| IP3 kinase | | 1 |
| Isovaleric acid CoA dehydrogenase | IVD | 1 |
| Kallikrein 3 | KAK3 | 4 |
| Kell blood group precursor | XK, KEL | 2 |
| Ketohexokinase | KHK | 1 |
| Kininogen, High molecular weight | KNG | 4 |
| Kynureninease | | 1 |
| Lactate dehydrogenase, A | LDHA | 1 |
| Lactate dehydrogenase, B | LDHB | 1 |
| Lamin A/C | LMNA | 6 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMRI | 6 |
| Latent transforming growth factor-beta | LTBP2 | 6 |
| binding protein 2 | | |
| Lecithin-cholesterol acyltransferase | LCAT | |
| Lectin, mannose-binding 1 | LMANI | |
| Lectin, mannose-binding 2 | MBL2 | |
| Leptin | LEP | 6 |
| Leptin receptor | LEPR | 6 |
| Leukocyte-specific transcript 1 | LST-1 | 4 |
| Leukotriene A4 synthase | LTA4S | 1 |
| Leukotriene B4 receptor | | 4 |
| Leukotriene B4 synthase | LTB4S | 1 |
| Leukotriene C4 synthase | LTC4S | 1 |
| LIM homeobox protein I | LHXL | 6 |
| Lipocortin I | ANX4 | 4 |
| Lipoprotein lipase | LPL | 4 |
| Lipoprotein receptor, Low Density | LDLR | 2 |
| Lipoprotein, High Density | HDLDTI | 2 |
| Lipoprotein, Very Low Density | VLDLR | 2 |
| Lipoprotein-associated coagulation factor | LACI | 4 |
| Lipoxygenase | | 1 |
| Lipoxygenase 12 (platelets) | LOG12 | 4 |
| Long QT-type 2 potassium channels | LQT2, KCNH2 | 2 |
| Low density lipoprotein receptor-related | LRP | 2 |
| protein precursor | | |
| Lymphoid enhancer-binding factor | LEF-1 | 6 |
| Lysosomat acid lipase | LIPA | 1 |
| Macrophage inflammatory protein-2 | MIP2 | 4 |
| MAD (mothers against decapentaplegic, | MADH4 | 6 |
| Drosophila) homologue 4 | | |
| MADS box transcription-enhancer factor 2A | MEF2A | 6 |
| MADS box transcription-enhancer factor 2B | MEF2B | 6 |
| Mannosidase, alpha B lysosomal | MANB | 1 |
| Matrix Gla protein | MGP | 6 |
| Matrix metalloproteinase I | mmpl | 1 |
| Matrix metalloproteinase IO | mmplo | 1 |
| Matrix metalloproteinase 1 1 | mmpl 1 | 1 |
| Matrix metalloproteinase 12 | MMP12 | 1 |
| Matrix metalloproteinase 13 | MMP13 | 1 |
| Matrix metalloproteinase 4 | MMP14 | 1 |
| Matrix metalloproteinase 15 | MMP15 | 1 |
| Matrix metalloproteinase 16 | MMP16 | 1 |
| Matrix metalloproteinase 17 | MMP17 | 1 |
| Matrix metalloproteinase 18 | MMP18 | 1 |
| Matrix metalloproteinase 19 | m P19 | 1 |
| Matrix metalloproteinase 2 | MMP2 | 1 |
| Matrix metalloproteinase 3 | MMP3, STMY1 | 1 |
| Matrix metalloproteinase 4 | MMP4 | 1 |
| Matrix metalloproteinase 5 | MMP5 | 1 |
| Matrix metalloproteinase 6 | MMP6 | 1 |
| Matrix metalloproteinase 7 | MMP7 | 1 |
| Matrix metalloproteinase 8 | MMP8 | 1 |
| Matrix metalloproteinase 9 | MMP9 | 1 |
| Melanocort'in 2 receptor | MC2R | 2 |
| Melanocortin 4 receptor | MC4R | 2 |
| Methionine synthase | MTR | 1 |
| Methionine synthase reductase | MTRR | 1 |
| Methylmalonyl-CoA mutase | MUT | 1 |
| Mevalonate kinase | MVK | |
| MHC Class 1: A | | |
| MHC Class 1: B | | |
| MHC Class 1: C | | |
| MHC Class 1: LMP-2, LMP-7 | | |
| MHC Class 1: Tapl | ABCR, TAP1 | |
| MHC Class II: DP | HLA-DPBI | |
| MHC Class 11: DQ | | |
| MHC Class 11: DR | | |
| MHC Class II: Tap2 | TAP2, PSF2 | |
| MHC Class ll: Complementation group A | MHC2TA | |
| MHC Class ll: Complementation group B | rfxank | |
| MHC Class ll: Complementation group C | RFX5 | |
| MHC Class ll: Complementation group D | RFXAP | |
| Microsomal triglyceride transfer protein | MTP | 2 |
| Mismatch repair gene, PMSL2 | PMS2 | 6 |
| Mitochondrial trifunctional protein, alpha subunit | HADHA | 1 |
| Mitochondrial trifunctional protein, beta subunit | HADHB | 1 |
| Molybdenum cofactor synthesis 1 | mocsi | 1 |
| Molybdenum cofactor synthesis 2 | MOCS2 | 1 |
| Monoamine oxidase A | MAOA | 1 |
| Monoamine oxidase B | MAOB | 1 |
| Monocyte chemoaftractant protein 1 | mcpl | 4 |
| Mucolipidoses | GNPTA | 1 |
| Mulibrey nanism | MUL | 2 |
| Muscarinic receptor, M1 | CHRM1 | 5 |
| Musca nic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| MutS homolog 3 | MSH3 | 6 |
| Myoglobin | | 2 |
| Myosin, cardiac | MYH7 | 3 |
| Myosin, light chain 2 | MYL2 | 3 |
| Myosin, light chain 3 | MYL3 | 3 |
| Myosin-binding protein C, cardiac | MYBPC3 | 3 |
| Myotubularin | MTM1 | 3 |
| Na+, K+ ATPase, alpha | ATPIAL | 6 |
| Na+, K+ ATPase, beta 1 | ATPIBL | 6 |
| Na+, K+ ATPase, beta 2 | ATPIB2 | 6 |
| Na+, K+ ATPase, beta 3 | ATPIB3 | 6 |
| Na+/H+ exchanger 1 | NHEL | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| N-acetylglucosamine-6-sulfatase | GNS | 1 |
| NADPH oxidase | | 4 |
| reductase | | |
| NB6 | | |
| Nebulin | NEB | 3 |
| Nephronophthisis 1 | NPHP1 | 2 |
| Neuraminidase sialidase | NEU | 2 |
| Neuregulin | HGL | 6 |
| Neurite inhibitory protein | 5 | |
| Neuroendocrine convertase 1 | NEC1, PCSK1 | 1 |
| Neurokinin A | NKNA | 5 |
| Neurokinin B | NKNB | 5 |
| Neuropeptide Y | NPY | 5 |
| Neuropeptide Y receptor Yi | NPYIR | 5 |
| Neuropeptide Y receptor Y2 | NPY2R | 5 |
| Neutrophil cystolic factor 1 | NCF1 | |
| Neutrophil cystolic factor 2 | NCF2 | |
| Niemann-Pick disease protein | NPCL | 2 |
| Nitric oxide synthase 1, NOSI | NOSI | 1 |
| Nitric oxide synthase 2, NOS2 | NOS2 | 1 |
| Nitric oxide synthase 3, NOS3 | NOS3 | 1 |
| Notch ligand - jagged 1 | JAG 1, AG S | 6 |
| Nuclear factor 1-kappa-B-like gene | IKBL | 4 |
| Oncogene sis | PDGFB | 6 |
| Oncostatin M | osm | 6 |
| Oncostatin M receptor | OSMR | 6 |
| Osteonectin | ON | 6 |
| Osteopontin | OPN | 6 |
| Osteoprotegedn | OPG | 6 |
| Pancreatic lipase | PNLIP | 1 |
| Pancreatic lipase related protein 1 | PLRPI | 1 |
| Pancreatic lipase related protein 2 | PLRP2 | 1 |
| Paraoxonase PONI | PONI | 1 |
| Paraoxonase PON2 | PON2 | 1 |
| Paraoxonase PON3 | | 1 |
| Parvalbumin | PVALB | 6 |
| Patched (Drosophila) homolog, PTCH | PTCH | 6 |
| PCNA (proliferating cell nuclear antigen) | | 1 |
| Pepsinogen | | 1 |
| Peroxidase, salivary | SAPX | 1 |
| Peroxisomal membrane protein 1 | PXMPI | 3 |
| Peroxisomal membrane protein 3 | PXMP3 | 2 |
| Peroxisome biogenesis factor I | PEXI | 2 |
| Peroxisome biogenesis factor 1 9 | PEX19 | 2 |
| Peroxisome biogenesis factor 6 | PEX6 | 2 |
| Peroxisome biogenesis factor 7 | PEX7 | 2 |
| Peroxisome proliferative activated receptor, | PPARA | 2 |
| alpha | | |
| Peroxisome proliferative activated receptor, | PPARG | 2 |
| gamma | | |
| Peroxisome receptor 1 | PXRI | 2 |
| P-glycoprotein 3 | PGY3 | 2 |
| Phosphatidylinositol glycan, class A | PIGA | 6 |
| (paroxysmal nocturnal hemoglobinuria) | | |
| Phosphatidylinositol transfer protein | PITPN | 6 |
| Phosphofructokinase, muscle | PFKM | 1 |
| Phosphoglucose isomerase | GPI | 1 |
| Phospholipase A2, group IO | PLA2GIO | 4 |
| Phospholipase A2, group 1 B | PLA2G 1 B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG | 4 |
| Phosphomannomutase-2 | PMM2 | 2 |
| Phosphoribosyl pyrophosphate synthetase | PRPS1 | 1 |
| Phosphorylase kinase, alpha 2 | PHKA2 | 1 |
| Phytanoyi-CoA hydroxylase | PHYH | 6 |
| Plasminogen | PLG | 1 |
| Plasminogen activator inhibitor I | PAI 1 | 1 |
| Plasminogen activator inhibitor 2 | PA 2 | 1 |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Platelet glutaminase | GLS | 2 |
| Platelet glycoprotein I b, alpha | GPI BA | |
| Platelet glycoprotein I b, beta | GPIBB | |
| Platelet glycoprotein I b, gamma | GPLBG | |
| Platelet glycoprotein IX | GP9 | |
| Platelet glycoprotein V | GP5 | |
| Platelet monamine oxidase | | 2 |
| Platelet-activating factor acetylhydrolase 1 B | PAFAH1B1or | 4 |
| LISI | | |
| Platelet-activating factor acetylhydrolase 2 | PAFAH2 | 4 |
| Platelet-activating factor receptor | PAFR | 4 |
| Poly (ADP-ribose) synthetase | PARS | F |
| Polycystic kidney and hepatic disease 1 | PKHDL | 2 |
| Polycystin I | PKDL | 2 |
| Polycystin 2 | PKD2 | 2 |
| Potassium inwardly-rectifying channel Jl | KCNJL | 5 |
| Potassium inwardly-rectifong channel JI 1 | KCNJ I 1 | 5 |
| Potassium voltage-gated channel Al | KCNAL | 5 |
| Potassium voltage-gated channel El | KCNEI | 5 |
| Potassium voltage-gated channel Q1 | KCNQI | 5 |
| Potassium voltage-gated channel Q2 | KCNQ2 | 5 |
| Potassium voltage-gated channel Q3 | KCNQ3 | 5 |
| POU domain, class 1, transcription factor 1 (Piti) | POUlF1 | 6 |
| Prekallikrein | | |
| Procollagen N-protease | | 1 |
| receptor) | | |
| Pro-melanin-concentrating hormone | PMCH | 6 |
| Proopiomelanocortin | POMC | 5 |
| Prostagiandin (PG) D synthase, | PGDS | 1 |
| hematopoietic | | |
| Prostagiandin E2 receptor | | |
| Prostaglandin-endoperoxidase synthase 2 | PTGS2 | 6 |
| Protease inhibitor 1 | | 2 |
| Protease nexin 2 | PN2 | 1 |
| Protective protein for beta-galactosidase | PPGB | 1 |
| Protein C | PROC | |
| Protein C inhibitor | PCI | |
| Protein S | PROS1 | |
| Prothrombin precursor | F2 | |
| Protoporphyrinogen oxidase | PPOX | 1 |
| Purine nucleoside phosphorylase | NP | 1 |
| Purinergic receptor PlA1 | | 5 |
| Purinergic receptor PIA2 | | 5 |
| Purinergic receptor PIA3 | | 5 |
| Purinergic receptor P2X, 1 | P2RX1 | 5 |
| Purinergic receptor P2X, 2 | P2RX2 | 5 |
| Purinergic receptor P2X, 3 | P2RX3 | 5 |
| Purinergic receptor P2X, 4 | P2RX4 | 5 |
| Purinergic receptor P2X, 5 | P2RX5 | 5 |
| Purinergic receptor P2X, 6 | P12RX6 | 5 |
| Purinergic receptor P2X, 7 | P2RX7 | 5 |
| Purinergic receptor P2Y, 1 | P2RYI | 5 |
| Purinergic receptor P2Y, I 1 | P2RY1 1 | 5 |
| Purinergic receptor P2Y, 2 | P2RY2 | 5 |
| Pyruvate carboxylase | PC | 1 |
| Pyruvate decarboxylase | PDHA | 1 |
| Pyruvate kinase | PKLR | 1 |
| Radixin | RDX | 3 |
| Renin | REN | 1 |
| Replication factor C | RFC2 | 1 |
| Retinoic acid receptor, alpha | RARA | 6 |
| Retinoic acid receptor, beta | RARB | 6 |
| Retinoic acid receptor, gamma | RARG | 6 |
| Retinoid X receptor, alpha | RXRA | 6 |
| Retinoid X receptor, beta | RXRB | 6 |
| Retinoid X receptor, gamma | RXRG | 6 |
| Rhesus blood group, CcEe antigens | RHCE | 2 |
| Rhesus blood group, D antigen | RHD | 2 |
| Rhesus blood group-associlated glycoprotein | RHAG | 2 |
| Ribosomal protein S19 | RPS19 | 1 |
| RIGUI | RIGUI | 6 |
| SIOO calcium-binding protein A1 | SIOOAI | 5 |
| S 1 00 calcium-binding protein A2 | S I 0OA2 | 5 |
| SI OO calcium-binding protein A3 | S 1 0OA3 | 5 |
| SIOO calcium-binding protein A7 | S 1 0OA7 | 5 |
| SI OO calcium-binding protein A8 | S I 0OA8 | 5 |
| SIOO calcium-binding protein B | SIOOB | 5 |
| SIOO calcium-binding protein P | sloop | 5 |
| SA homolog | SAH | 6 |
| SAP (SLAM-associated protein) | SH2DlA | 4 |
| Secretase, alpha | | 5 |
| Secretase, beta | | 5 |
| Secretase, gamma | | 5 |
| Selectin E | SELE | 5 |
| Selectin L | SELL | 5 |
| Selectin P | SELP | 5 |
| Serotonin receptor, 5HTIA | HTRIA | 5 |
| Serotonin receptor, 5HTlB | HTRI B | 5 |
| Serotonin receptor, 5HTIC | HTRIC | 5 |
| Serotonin receptor, 5HT1 D | HTRID | 5 |
| Serotonin receptor, 5HTI E | HTRL E | 5 |
| Serotonin receptor, 5HT1 F | HTRL F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Serum amyloid A | SAA | 2 |
| Serum amyloid P | SAP | 2 |
| Sjoegren (Siogren) syndrome antigen A1 | SSAI | 4 |
| Sodium channel, non-voltage gated 1, alpha | SCNNLA | 5 |
| Sodium channel, non-voltage gated 1, beta | SCNN1 B | 5 |
| Sodium channel, non-voltage gated 1, | SCNNLG | 5 |
| gamma | | |
| Sodium channel, voltage gated, type IV, | SCN4A | 5 |
| alpha polypeptide | | |
| Sodium channel, voltage gated, type V, alpha SCN5A | | 5 |
| polypeptide | | |
| Sodiumchannel, voltage-gated, type1, beta SCNLB | | 5 |
| polypeptide | | |
| Solute carrier family 1 (glutamate | SLCIAL | 2 |
| transporter), member 1 | | |
| Solute carrier family 1 (glutamate | SLC1A2 | 2 |
| transporter), member 2 | | |
| Solute carrier family 1 0 (sodium/bile acid | SLC1OA1 | 2 |
| cotransporter family), member 1 | | |
| Solute carrier family IO (sodium/bile acid | SLC1OA2 | 2 |
| cotransporter family), member 2 | | |
| Solute carrier family 12, member I | SLC12A1 | 2 |
| Solute carrier family 12, member 2 | SLC12A2 | 2 |
| Solute carrier family 12, member 3 | SLC12A3 | 2 |
| Solute carrier family 2 (facilitated glucose | SLC2AI | 2 |
| transporter), member I | | |
| Solute carrier family 2 (facilitated glucose | SLC2A2 | 2 |
| transporter), member 2 | | |
| Solute carrier family 2 (facilitated glucose | SLC2A3 | 2 |
| transporter), member 3 | | |
| Solute carrier family 2 (facilitated glucose | SLC2A4 | 2 |
| transporter), member 4 | | |
| Solute carrier family 2 (facilitated glucose | SLC2A5 | 2 |
| transporter), member 5 | | |
| Solute carder family 21, member 2 | SLC21A2 | 2 |
| Solute carrier family 21, member 3 | SLC21A3 | 2 |
| Solute carrier family 22, member 5 | SLC22A5 | 2 |
| Solute carrier family 3 (facilitated glucose | SLC3AI | 2 |
| transporter), member I | | |
| Solute carrier family 4 (anion exchanger), member 1 | SLC4AI | 2 |
| Solute carrier family 4 (anion exchanger), member 2 | SLC4A2 | 2 |
| Solute carrier family 4 (anion exchanger), member 3 | SLC4A3 | 2 |
| Solute carrier family 5 (sodium/glucose | SLC5A1 | 2 |
| transporter), member I | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmifter | SLC6A3 | 2 |
| transporter, dopamine), member 3 | | |
| Solute carrier family 6 (neurotransmifter | SLC6A2 | 2 |
| transporter, noradrenaline), member 2 | | |
| Solute carrier family 6 (neurotransmifter | SLC6A4 | 2 |
| transporter, serotonin), member 4 | | |
| Solute carrier family 8 (sodium/calcium | SLC8A1 | 2 |
| exchanger), member 1 | | |
| Sonic hedgehog, SHH | SHH | 6 |
| So@cin | SRI | 2 |
| Spectrin alpha | SPTAI | 3 |
| Spectrin beta | SPTB | 3 |
| Sphingomyelinase | SMPD1 | 1 |
| Stem cell factor | SCF | 6 |
| Steroid 5 alpha reductase 1 | SRD5A1 | 1 |
| Steroid 5 alpha reductase 2 | SRD5A2 | 1 |
| Steroidogenic acute regulatory protein | STAR | 2 |
| Sterol carrier protein 2 | SCP2 | 2 |
| Succinate dehydrogenase I | SDHL | 1 |
| Succinate dehydrogenase 2 | SDH2 | 1 |
| Succinate thiokinase | | 1 |
| Superoxide dismutase 1 | SODI | 1 |
| Superoxide dismutase 3 | SOD3 | 1 |
| Surfeit I | SURF1 | 6 |
| Synapsin I a& b | SYNI | 5 |
| Synapsin 2a & 2b | SYN2 | 5 |
| Synaptic vesicle amine transporter | 8VAT | 5 |
| Synaptobrevin 1 | SYBI | 5 |
| Synaptobrevin 2 | SYB2 | 5 |
| Synaptogyrin | | 5 |
| Synaptophysin | SYP | 5 |
| Synaptosomal-associated protein, 25 KD | SNAP25 | 5 |
| Synaptotagmin I | SyTi | 5 |
| Synaptotagmin 2 | SYT2 | 5 |
| Syntaxin I | STxl | 5 |
| Talin | TLN | 6 |
| T-BOX 1 | TBXL | 6 |
| T-BOX 3 | TBX3 | 6 |
| TEK, tyrosine kinase, endothelial | TEK | 1 |
| Terminal deoxynucleotidyltransferase | TDT | 4 |
| Tetranectin | TNA | 2 |
| Thiolase, perioxisomal | | 1 |
| Thiopurine S-methyltransferase | TPMT | 1 |
| Thrombin receptor | F2R | |
| Thrombomodulin | THBD | |
| Thrombopoietin | THPO | 6 |
| Thrombospondin | THBSI | 6 |
| Thromboxane A synthase 1 | TBXASI | |
| Thromboxane A2 | TXA2 | |
| Thromboxane A2 receptor | TBXA2R | |
| Thy-I T-cell antigen | THYI | |
| Thymic humoral factor | | |
| Thymopoietin | TMPO | 6 |
| Thymosin | | 4 |
| Thyroid hormone receptor, alpha | THRA | 6 |
| Thyroid hormone receptor, beta | THRB | 6 |
| TIE receptor tyrosine kinase | TIE-1 | 6 |
| Tip-associated protein | TAP | 4 |
| Tissue inhibitor of metalloproteinase 1, | TIMP1 | 1 |
| TiMP1 | | |
| Tissue inhibitor of metalloproteinase 2, | TIMP2 | 1 |
| TIMP2 | | |
| Tissue inhibitor of metalloproteinase 3, | TIMP3 | 1 |
| TIMP3 | | |
| Tissue inhibitor of metalloproteinase 4, | TIMP4 | 1 |
| TIMP4 | | |
| Topoisomerase I | | 1 |
| Torticollis, keloids, cryptorchidism and renal | TKCR | 6 |
| dysplasia gene | | |
| Transcobalamin 2, TCN2 | TCN2 | 2 |
| Transcription factor 2, hepatic | TCF2 | 6 |
| Transferrin | TF | 6 |
| Transferdn receptor | TFRC | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Transtocation in renal carcinoma on | TRC8 | 6 |
| chromosome 8 gene | | |
| Transthyretin | TTR | 2 |
| Triosephosphate isomerase | TP11 | 1 |
| Tropomyosin 1 alpha | TPM1 | 3 |
| Troponin C | | 3 |
| Troponin I | TNN13 | 3 |
| Troponin T2, cardiac | TNNT2 | 3 |
| Tuberous sclerosis 1 | TSC1 | 6 |
| Tuberous sclerosis 2 | TSC2 | 6 |
| Tumour necrosis factor (TNF) receptor | TRAFI | |
| associated. factor 1 | | |
| Tumour necrosis factor (TNF) receptor | TRAF2. | |
| associated factor 2 | | |
| Tumour necrosis factor (TNF) receptor | TRAF3 | |
| associated factor 3 | | |
| Tumour necrosis factor (TNF) receptor | TRAF4 | |
| associated factor 4 | | |
| Tumour necrosis factor (TNF) receptor | TRAF5 | |
| associated factor 5 | | |
| Tumour necrosis factor (TNF) receptor | TRAF6 | |
| associated factor 6 | | |
| Tumour necrosis factor alpha | TNFA | |
| Tumour necrosis factor alpha receptor | TNFAR | |
| Tumour necrosis factor beta | TNFB | |
| Tumour necrosis factor beta receptor | TNFBR | |
| Tumour protein p53 | TP53, P53 | 6 |
| Tumour protein p63 | TP63 | 6 |
| Tyrosine hydroxylase | TH | 1 |
| Ubiquitin | | 6 |
| Ubiquitin B | UBB | 6 |
| Ubiquitin C | UBC | 6 |
| UDP-glucose pyrophosphorylase | | 1 |
| UDP-glucuronosyltransferase 1 | ugtld, UGTI | 1 |
| UDP-glucuronosyltransferase 2 | UGT2 | 1 |
| Uncoupling protein I | | 2 |
| Uncoupling protein 3 | UCP3 | 2 |
| Undulin I | COL14Al | 3 |
| Uridinediphosphate(UDP)-galactose-4- | GALE | 1 |
| epimerase | | |
| Uroporphyrinogen III synthase | UROS | 1 |
| Vacuolar proton pump, subunit 1 | VPPI | 5 |
| Vacuolar proton pump, subunit 3 | VPP3 | 5 |
| Vascular endothelial growth factor | VEGF | 6 |
| Vasoactive intestinal polypeptide | VIP | 5 |
| Vasoactive intestinal polypeptide receptor | VIPR | 5 |
| Vasoinhibitory peptide | | 6 |
| Vimentin | vim | 4 |
| Vinculin | | 3 |
| Vitamin D receptor | VDR | 6 |
| Von Hippel-Lindau gene | VHL | 6 |
| Von Willebrand factor | VWF | 2 |
| Werner syndrome helicase | WRN | 6 |
| Wiskoft-Aldrich syndrome protein | WASP, THC | 4 |
| Wolf-Hirschhorn syndrome candidate 1 gene | WHSCI | 6 |
| Wolfram syndrome I gene | WFS1 | 3 |
| Xanthine dehydrogenase | XDH | 1 |
| Zinc finger protein 3 | ZIC3 | 3, |

that the genes associated with malfunction, damage or disease of the gastrointestinal tract are selected from:

| List of genes | HUGO gene symbol | Protein-function |
|---|---|---|
| 11beta hydroxysteroid dehydrogenase 2 | HSD11B2 | 1 |
| 17beta hydroxysteroid dehydrogenase 1 | HSD17B1 | 1 |
| 17beta hydroxysteroid dehydrogenase 3 | HSD17B3 | 1 |
| 17beta hydroxysteroid dehydrogenase 4 | HSD17B4 | 1 |
| 17beta hydroxysteroid oxidoreductase | | 1 |
| 2,3-Bisphosphoglycerate mutase | BPGM | 1 |
| 3 beta Hydroxysteroid dehydrogenase 2 | HSD3B2 | 1 |
| 6-Phosphofructo-2-kinase | PFKFB1 | 1 |
| Acetoacetyl 1-CoA-thiolase | ACAT1 | 1 |
| Acetoacetyl 2-CoA-thiolase | ACAT2 | 1 |
| Acetyl CoA carboxylase | ACC | 1 |
| Acetyl CoA carboxylase alpha | ACACA | 1 |
| Acetylcholine receptor, nicotinic, gamma | CHRNG | 5 |
| Acetylcholinesterase | ACHE | 1 |
| Acid phosphatase 2, lysosomal | ACP2 | 1 |
| Actin, alpha, cardiac | ACTC | 3 |
| Actin, alpha, skeletal | ACTAL | 3 |
| Actin, alpha, smooth, aortic | ACTA2 | 3 |
| Actin, beta | ACTB | 3 |
| Actin, gamma 2 | ACTG2 | 3 |
| Acyl CoA dehydrogenase, long chain | ACADL | 1 |
| Acyl CoA dehydrogenase, medium chain | ACADM | 1 |
| Acyl CoA dehydrogenase, short chain | ACADS | 1 |
| Acyl CoA dehydrogenase, very long chain | ACADVL | 1 |
| Acyl CoA synthetase, long chain, 1 | LACS1 | 1 |
| Acyl CoA synthetase, long chain, 2 | LACS2 | 1 |
| Acyl CoA synthetase, long chain, 4 | ACS4 | 1 |
| Acyl malonyl condensing enzyme | | 1 |
| Acyl-CoA thioesterase | | 1 |
| Adaptin, beta 3A | ADTB3A | 2 |
| Adenine phosphoribosyltransferase | APRT | 2 |
| Adenomatous polyposis coli tumour supressor | APC | 6 |
| gene | | |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Alanine aminotransferase | | 2 |
| Alanine-glyoxylate aminotransferase | AGXT | 1 |
| Albumin, ALB | ALB | 2 |
| Alcohol dehydrogenase 1 | ADH1 | 1 |
| Alcohol dehydrogenase 2 | ADH2 | 1 |
| Alcohol dehydrogenase 3 | ADH3 | 1 |
| Alcohol dehydrogenase 4 | ADH4 | 1 |
| Alcohol dehydrogenase 5 | ADH5 | 1 |
| Alcohol dehydrogenase 6 | ADH6 | 1 |
| Alcohol dehydrogenase 7 | ADH7 | 1 |
| Aldehyde dehydrogenase 1 | ALDH1 | 1 |
| Aldehyde dehydrogenase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldolase A | ALDOA | 1 |
| Aldolase B | ALDOB | 1 |
| Aldolase C | ALDOC | 1 |
| Aldose reductase | | 2 |
| Aldosterone receptor | MLR | 6 |
| Alkaline phosphatase, liver/bone/kidney | ALPL | 2 |
| Alpha 2 macroglobulin | A2M | 4 |
| alpha1-Antitrypsin | PI | 1 |
| alpha2-Antiplasmin | PLI | 1 |
| alpha-Actinin 2 | ACTN2 | 6 |
| alpha-Actinin 3 | ACTN3 | 6 |
| alpha-Amylase | | 1 |
| alpha-Dextrinase | | 1 |
| alpha-Galactosidase A | GLA | 1 |
| alpha-Ketoglutarate dehydrogenase | | 1 |
| alpha-L-Iduronidase | IDUA | 1 |
| Aminomethyltransferase | AMT | 1 |
| Aminopeptidase P | XPNPEP2 | 1 |
| Amphiregulin | AREG | 6 |
| Amylo-1,6-glucosidase | AGL | 1 |
| Angiopoietin 1 | ANGPT1 | 6 |
| Angiopoietin 2 | ANGPT2 | 6 |
| Angiotensin converting enzyme | ACE, DCPI | 1 |
| Angiotensin receptor 1 | AGTR1 | 2 |
| Angiotensin receptor 2 | AGTR2 | 2 |
| Angiotensinogen | AGT | 1 |
| Antidiuretic hormone receptor | ADHR | 2 |
| Antithrombin III | AT3 | 1 |
| AP-2, alpha | TFAP2A | 6 |
| AP-2, beta | TFAP2B | 6 |
| AP-2, gamma | TFAP2C | 6 |
| Apolipoprotein A I | APOA1 | 2 |
| Apolipoprotein A II | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Aquaporin 1 | AQP1 | 2 |
| Aquaporin 2 | AQP2 | 2 |
| Arginine vasopressin | AVP | 5 |
| Arginine vasopressin receptor 1A | AVPR1A | 5 |
| Arginine vasopressin receptor 1B | AVPR1B | 5 |
| Arginine vasopressin receptor 2 | AVPR2 | 5 |
| Arginosuccinate lyase | ASL | 1 |
| Arginosuccinate synthetase | ASS | 1 |
| Aryl hydrocarbon receptor nuclear translocator | ARNT | 2 |
| Arylsulfatase A | ARSA | 1 |
| Arylsulfatase B | ARSB | 1 |
| Aspartate transaminase | | 2 |
| Aspartylglucosaminidase | AGA | 1 |
| Ataxia telangiectasia gene, AT | ATM | 6 |
| ATP/ADP translocase | | 1 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Autoimmune regulator, AIRE | AIRE | 4 |
| Azoospermia factor 1 | AZF1 | 6 |
| beta 2 Microglobulin | B2M | 4 |
| beta-Galactosidase | GLB1 | 1 |
| beta-Glucosidase, neutral | | 1 |
| beta-Glucuronidase | GUSB | 1 |
| beta-Ketoacyl reductase | | 1 |
| Bile acid coenzyme A: amino acid N- | BAAT | 1 |
| Acyltransferase | | |
| Bile salt export pump | BSEP, PFIC2 | 2 |
| Bile salt-stimulated lipase | CEL | 1 |
| Bilirubin UDP-glucuronosyltransferase | | 1 |
| Biliverdin reductase | | 2 |
| Bradykinin receptor B1 | | 4 |
| Bradykinin receptor B2 | | 4 |
| Branched chain keto acid dehydrogenase E1, | BCKDHA | 1 |
| alpha polypeptide | | |
| Branched chain keto acid dehydrogenase E1, | BCKDHB | 1 |
| beta polypeptide | | |
| Brush border guanylyl cyclase | | 1 |
| Ca(2+) transporting ATPase, fast twitch | ATP2AI | 2 |
| Ca(2+) transporting ATPase, slow twitch | ATP2A2 | 2 |
| Cadherin E | CDH1 | 6 |
| Cadherin EP | | 6 |
| Cadheein N | CDH2 | 6 |
| Cadherin P | CDH3 | 6 |
| Calcitonin/Calcitonin gene-related peptide | CALCA | 5 |
| alpha | | |
| Calcium channel, voltage-dependent, Alpha 1F | CACNA 1F | 5 |
| subunit | | |
| Calcium channel, voltage-dependent, Alpha- | CACNA 1B | 5 |
| 1 B (CACNL1A5) | | |
| Calcium channel, voltage-dependent, Alpha- | CACNA 1C | 5 |
| 1 C | | |
| Calcium channel, voltage-dependent, Alpha- | CACNA 1D | 5 |
| 1 D | | |
| Calcium channel, voltage-dependent, Alpha- | CACNA 1E | 5 |
| I E (CACNL1A6) | | |
| Calcium channel, voltage-dependent, Alpha- | CACNA2 | 5 |
| 2/delta | | |
| Calcium channel, voltage-dependent, Beta I | CACNB1 | 5 |
| Calcium channel, voltage-dependent, Beta 3 | CACNB3 | 5 |
| Calcium channel, voltage-dependent, | CACNG2 | 5 |
| Neuronal, Gamma | | |
| Calcium channel, voltage-dependent, T-type | | 5 |
| Calcium sensing receptor | CASR | 2 |
| Calmodulin 1 | CALM1 | 6 |
| Calmodulin 2 | CALM2 | 6 |
| Calmodulin 3 | CALM3 | 6 |
| Calmodulin dependent kinase | | 2 |
| Calmodulin-dependant protein kinase II | CAMK2A | 6 |
| Calnexin | CANX | 6 |
| Canalicular multispecific organic anion | CMOAT | 2 |
| transporter | | |
| Carbamoylphosphate synthetase 1 | CPS1 | 1 |
| Carbamoylphosphate synthetase 2 | CPS2 | 1 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carboxylesterase 1 | CES1 | 1 |
| Carboxypeptidase | CPN | 1 |
| Camitine acylcarnitine translocase | CACT | 1 |
| Carnitine palmitoyltransferase I | CPT1A | 1 |
| Carnitine palmitoyltransferase II | CPT2 | 1 |
| Carnitine transporter protein | CDSP, SCD | 2 |
| Cartilage-hair hypoplasia gene | CHH | 5 |
| Catalase | CAT | 4 |
| Cathepsin B | | 1 |
| Cathepsin D | | 1 |
| Cathepsin E | | 1 |
| Cathepsin G | CTSG | 1 |
| Cathepsin H | | 1 |
| Cathepsin K | CTSK | 1 |
| Cathepsin L | | 1 |
| Cathepsin S | | 1 |
| CD1 | CD1 | 4 |
| CD4 | CD4 | 4 |
| Cell adhesion molecule, intercellular, ICAM | ICAM1 | 6 |
| Cell adhesion molecule, leukocyte-endothelial, | LECAM1 | 6 |
| LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM1 | 6 |
| PECAM | | |
| Cell adhesion molecule, vascular, VCAM | VCAM1 | 6 |
| c-erbB2 | FRBB2 | 6 |
| c-erbB3 | ERBB3 | 6 |
| c-erbB4 | ERBB4 | 6 |
| Ceruloplasmin precursor | CP | 1 |
| Chemokine receptor CCR2 | CCR2 | 4 |
| Chemokine receptor CCR3 | CCR3 | 4 |
| Chemokine receptor CCR5 | CCR5 | 4 |
| Chemokine receptor CXCR4 | CXCR4 | 4 |
| Chitotriosidase | CHIT | 1 |
| Chloride channel 5 | CLCN5 | 3 |
| Chloride channel KB | CLCNKB | 3 |
| Cholecystokinin | CCK | 5 |
| Cholecystokinin B receptor | CCKBR | 5 |
| Cholestasis, progressive familial intrahepatic 1 | FIC1 | 6 |
| gene | | |
| Cholesterol ester hydroxylase | | 1 |
| Choline acetyltransferase | CHAT | 1 |
| Chromogranin A | CHGA | 6 |
| Chymotrypsinogen | | 1 |
| Citrate synthase | | 1 |
| Clathrin | | 2 |
| Clusterin | CLU | 6 |
| CoA transferase | | 1 |
| Cockayne syndrome gene, CKN1 | CKN1 | 6 |
| Collagen I alpha 1 | COL1A1 | 3 |
| Collagen I alpha 2 | COL1A2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11A1 | 3 |
| Collagen XI alpha 2 | COL11A2 | 3 |
| Collagen XVII alpha 1 | COL17A1 | 3 |
| Colony-stimulating factor 1 | CSF1 | 6 |
| Complement component C1 inhibitor | C1 NH | 4 |
| Complex I | | 1 |
| Complex II | | 1 |
| Complex III | | 1 |
| Corticotrophin-releasing hormone | CRH | 2 |
| Corticotrophin-releasing hormone receptor | CRHR1 | 2 |
| C-reactive protein CRP | | 4 |
| Creb binding protein | CREBBP | 6 |
| Cu2+ transporting ATPase beta polypeptide | ATP7B | 1 |
| Cubilin | CUBN | 2 |
| Cyclic AMP-dependent protein kinase | PKA | 1 |
| Cyclic nucleotide phosphodiesterase 1 B | PDE1B | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin F | CCNF | 6 |
| Cyclin-dependent kinase 2 | CDK2 | 6 |
| Cyclin-dependent kinase inhibitor 1C (P57, | CDKN 1C | 6 |
| KIP2) | | |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2FI | CYP2FI | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5AI | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cysteine-rich intestinal protein | | 2 |
| Cystic fibrosis transmembrane conductance | CFTR | 5 |
| regulator, CFTR | | |
| Cystinosin | CTNS | 2 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBPI | 4 |
| binding protein 1 | | |
| Cytokine-suppressive antiinflammatory drug- | CSBP2 | 4 |
| binding protein 2 | | |
| DAX1 nuclear receptor | DAX1 | 4 |
| Deleted in colorectal carcinoma | DCC | 6 |
| Delta aminolevulinate dehydratase | ALAD | 1 |
| Delta(4)-3-oxosteroid 5-beta-reductase | | 1 |
| Delta-7-dehydrocholesterol reductase | DHCR7 | 1 |
| Dihydrodiol dehydrogenase 1 | DDHI | 1 |
| Dihydrolipoamide branched chain transacylase | DBT | 5 |
| Dihydrolipoamide dehydrogenase | DLD | 5 |
| DNA glycosylases | | 1 |
| Dopamine beta hydroxylase | DBH | 1 |
| Dopamine receptors Dl | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dynamin | DNMI | 6 |
| Dynein | | 6 |
| Dystrophia myotonica | DM, DMPK | 1 |
| Dystrophia myotonica, atypical | DM2 | 1 |
| Dystrophin | DMD | 3 |
| EBI | | 6 |
| Elastase I | ELASI | 1 |
| Elastase 2 | ELAS2 | 1 |
| Electron-transfering-flavoprotein alpha | ETFA | 2 |
| Electron-transfering-flavoprotein beta | ETFB | 2 |
| Electron-transferring flavoprotein | ETFDH | 1 |
| dehydrogenase | | |
| Endothelin I | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | ENO1 | 1 |
| Enoyl CoA isomerase | | 1 |
| Enoyl CoA reductase | | 1 |
| Enteric lipase | | 2 |
| Enterokinase | PRSS7, ENTK | 1 |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Erythrocyte membrane protein band 4.1 | EPB41 | 3 |
| Erythropoietin | EPO | 4 |
| Excision repair complementation group 2 | ERCC2 | 1 |
| protein | | |
| Excision repair complementation group 2 | ERCC3 | 1 |
| protein | | |
| Eyes absent 1 | EYAI | 6 |
| Faciogenital dysplasia | FGD1, FGDY | 2 |
| Factor 1 (No. one) | F1 | 4 |
| Factor B, properdin | | 4 |
| Factor D | | 4 |
| Factor H | HF1 | 4 |
| Factor I (letter 1) | IF | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & F13B | 4 |
| FADH dehydrogenase | | 1 |
| Fanconi anemia, complementation group A | FANCA | 2 |
| Fanconi anemia, complementation group C | FANCC | 2 |
| Fanconi anemia, complementation group D | FANCD | 2 |
| Fatty acid binding proteins FABP1 | | 2 |
| Fatty acid binding proteins FABP2 | FABP2 | 2 |
| Fatty acid binding proteins FABP3 | | 2 |
| Fatty acid binding proteins FABP4 | | 2 |
| Fatty acid binding proteins FABP5 | | 2 |
| Fatty acid binding proteins FABP6 | | 2 |
| Ferritin, H subunit | | 2 |
| Ferritin, L subunit | FTL | 2 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Flavin-containing monooxygenase 1 | FMOL | 1 |
| Flavin-containing monooxygenase 2 | FMO2 | 1 |
| Flavin-containing monooxygenase 3 | FMO3 | 1 |
| Flavin-containing monooxygenase 4 | FMO4 | 1 |
| Folic acid receptor | FOLR | 6 |
| Follicle stimulating hormone receptor | FSHR, ODG1 | 6 |
| Follicle stimulating hormone, FSH | FSHB | 6 |
| Forkhead transcription factor I0 | FKHL10 | 6 |
| Forkhead transcription factor 14 | FKHL14 | 6 |
| Fragile site, folic acid type, rare, fra(X) A | FRAXA | 5 |
| Fructose-1,6-diphosphatase | FBP1 | 1 |
| Fucosidase alpha-L-1 | FUCA1 | 1 |
| Fucosidase alpha-L-2 | | 1 |
| Fucosyltransferase 2 | FUT2 | 2 |
| Fucosyltransferase 3 | FUT3 | 2 |
| Fumarase | FH | 1 |
| G/T mismatch binding protein | GTBP, MSH6 | 6 |
| Galactocerebrosidase | GALC | 1 |
| Galactose 1-phosphate uridyl-transferase | GALT | 1 |
| Galactosyltransferase 1 | GT1 | 6 |
| Galactosyltransferase, alpha 1,3 | GGTA1 | 6 |
| Galactosyltransferase, beta 3 | B3GALT | 6 |
| Galanin | GAL | 5 |
| Galanin receptor | GALNRL | 5 |
| Gamma-glutamyltransferase 1 | GGT1 | 2 |
| Gamma-glutamyltransferase 2 | GGT2 | 2 |
| Gap junction protein beta I | GJB1 | 2 |
| Gastric inhibitory polypeptide GIP | GIP | 2 |
| Gastric inhibitory polypeptide receptor, GIPR | GIPR | 2 |
| Gastric Intrinsic factor, GIF | GIF | 1 |
| Gastric lipase, LIPF | | 2 |
| Gastrin | GAS | 6 |
| Gastrin releasing peptide | GRP | 2 |
| Gastrin releasing peptide receptor | GRPR | 2 |
| Glial-cell derived neurotrophic factor (GDNF) | | 5 |
| receptor | | |
| Glial-cell derived neurotrophic factor, GDNF | GDNF | 5 |
| Glucagon receptor | GCGR | 6 |
| Glucagon synthase | | 2 |
| Glucagon-like peptide receptor 1 | GLP1R | 6 |
| Glucokinase | GCK | 1 |
| Glucose-6-phosphatase | G6PC | 1 |
| Glucose-6-phosphatase translocase | G6PT1 | 1 |
| Glucose-6-phosphate dehydrogenase | G6PD | 1 |
| Glucosidase, acid alpha | GAA | 1 |
| Glutamate dehydrogenase | GLUD1 | 1 |
| Glutamine synthase | | 1 |
| Glutamine transporter | | 2 |
| Glutathione | GSH | 2 |
| Glutathione peroxidase, GPX2 | GPX2 | 1 |
| Glutathione S-transferase, GSTZ1 | GSTZ1 | 1 |
| Glyceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Glycine dehydrogenase | GLDC | 1 |
| Glycogen branching enzyme | GBEI | 1 |
| Glycogen phosphorylase | PYGL | 1 |
| Glycogen synthase 1 (muscle) | GLYS1 | 1 |
| Glycogen synthase 2 (liver) | GYS2 | 1 |
| Glycosyltransferases, ABO blood group | ABO | 1 |
| Gonadotropin releasing hormone | GNRH | 6 |
| Goosecoid GSC | | 6 |
| Growth arrest-specific homeobox | GAX | 6 |
| Growth hormone receptor | GHR | 6 |
| Guanylin | GUCA2 | 2 |
| H(+), K(+) - ATPase | ATP4B | 5 |
| Haem oxygenase | | 2 |
| Haemoglobin alpha 1 | HBA1 | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Heparan sulfamidase | | 1 |
| Heparin binding epidermal growth factor | HBEGF | 6 |
| Heparin Cofactor 11 | HCF2 | 4 |
| Hepatic nuclear factor-3-beta | HNF3B | 1 |
| Hepatic nuclear factor-4-alpha | HNF4A | 1 |
| Hepatitis B virus integration site 1 | HVBS1 | |
| Hepatitis B virus integration site 2 | HVBS6 | |
| Hepatocyte growth factor | HGF | 6 |
| Hermansky-pudlak syndrome gene | HPS | 2 |
| Hexokinase 1 | HKI | 1 |
| Hexokinase 2 | HK2 | 1 |
| Hexosaminidase A | HEXA, TSD | 1 |
| Hexosaminidase B | HEXB | 1 |
| Histamine receptors, H1 | | 5 |
| Histamine receptors, H2 | | 5 |
| Histamine receptors, H3 | | 5 |
| Histatin 1 | | 4 |
| Histatin 2 | | 4 |
| Histatin 3 | HTN3 | 4 |
| HLA-B associated transcript I | BAT1 | 4 |
| HMG-CoA lyase | HMGCL | 1 |
| HMG-CoA reductase | HMGCR | 1 |
| HMG-CoA synthase | HMGCS2 | 1 |
| Holocarboxylase synthetase | HLCS | 1 |
| Hormone-sensitive lipase | HSL | 1 |
| Hydroxyacyl glutathione hydrolase | HAGH | 1 |
| Hypoxanthine-guanine | HPRT | 1 |
| phosphoribosyltransferase, HGPRT | | |
| IC7 A and B | | 4 |
| Iduronate 2 sulphatase | IDS | 1 |
| lmmunoglobulin E (IgE) reponsiveness gene | IGER | 4 |
| lmmunoglobulin E (IgE) serum concentration | IGES | 4 |
| regulator gene | | |
| lmmunoglobulin gamma (IgG) 2 | IGHG2 | 4 |
| lmmunoglobulin heavy mu chain | IGHM | 4 |
| Immunoglobulin J polypeptide | IGJ | 4 |
| Immunoglobulin kappa constant region | IGKC | 4 |
| Immunoglobulin kappa variable region | IGKV | 4 |
| lnhibin, alpha | INHA | 6 |
| lnhibin, beta A | INHBA | 6 |
| lnhibin, beta B | INHBB | 6 |
| Inhibin, beta C | INHBC | 6 |
| lnositol 1,4,5-triphosphate receptor 3 | ITPR3 | 6 |
| Insulin | INS | 6 |
| Insulin receptor | INSR | 6 |
| Insulin-like growth factor 1 | IGF1 | 6 |
| Insulin-like growth factor 1 receptor | IGFIR | 6 |
| Insulin-like growth factor 2 | IGF2 | 6 |
| Insulin-like growth factor 2 receptor | IGF2R | 6 |
| lntegrin beta 1 | ITGBL | 6 |
| Integrin beta 2 | ITGB2 | 6 |
| ntegrin beta 3 | ITGB3 | 6 |
| lntegrin beta 6 | ITGB6 | 6 |
| Integrin, alpha M | ITGAM | 6 |
| lntegrin, alpha X | ITGAX | 6 |
| Inter-alpha-trypsin inhibitor, IATI | | 1 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| Interieukin(IL) 1 receptor | IL1R | 4 |
| Interleukin(IL) 1, alpha | IL1A | 4 |
| lnterleukin(IL) 1, beta | IL1B | 4 |
| lnterleukin(IL) 10 | IL10 | 4 |
| Interleukin(IL) 10 receptor | IL10R | 4 |
| lnterleukin(IL) 11 | IL11 | 1 |
| lnterleukin(IL) 11 receptor | IL11R | 4 |
| Interleukin(IL) 12 | IL12 | 4 |
| Interleukin(IL) 12 receptor, beta 1 | IL12RBI | 4 |
| Interleukin(IL) 13 | IL13 | 4 |
| Interleukin(IL) 13 receptor | IL13R | 4 |
| Interleukin(IL) 2 | IL2 | 4 |
| lnterleukin(IL) 2 receptor, alpha | IL2RA | 4 |
| lnterleukin(IL) 2 receptor, gamma | IL2RG | 4 |
| lnterleukin(IL) 3 | IL3 | 4 |
| Interleukin(IL) 3 receptor | IL3R | 4 |
| lnterleukin(IL) 4 | IL4 | 4 |
| lnterleukin(IL) 4 receptor | IL4R | 4 |
| Interleukin(IL) 5 | IL5 | 4 |
| lnterleukin(IL) 5 receptor | IL5R | 4 |
| lnterleukin(IL) 6 | IL6 | 4 |
| lnterleukin(IL) 6 receptor | IL6R | 4 |
| Interleukin(IL) 7 | IL7 | 4 |
| lnterleukin(IL) 7 receptor | IL7R | 4 |
| Interleukin(IL) 8 | IL8 | 4 |
| Interleukin(IL) 8 receptor | IL8R | 4 |
| lnterleukin(IL) 9 | IL9 | 4 |
| lnterleukin(IL) 9 receptor | IL9R | 4 |
| Interleukin(IL) receptor antagonist 1 | IL1RN, IL1RA | 4 |
| Intestinal alkaline phosphatase IAP | | 2 |
| Islet amyloid polypeptide | IAPP | 5 |
| Isocitrate dehydrogenase | | 1 |
| lsovaleric acid CoA dehydrogenase | IVD | 1 |
| Kallikrein 3 | KAK3 | 4 |
| Kaliman syndrome gene 1 | KAL1 | 6 |
| Ketohexokinase | KHK | 1 |
| ketolase | | 1 |
| Kininogen, High molecular weight | KNG | 4 |
| Kynurenine hydroxylase | | 1 |
| Kynureninease | | 1 |
| Lactase | | 1 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta binding | LTBP2 | 6 |
| protein 2 | | |
| Lecithin-cholesterol acyltransferase | LCAT | 1 |
| Leptin | LEP | 6 |
| Leptin receptor | LEPR | 6 |
| Leukocyte-specific transcript 1 | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| LIM homeobox transcription factor 1, beta | LMX1B | 6 |
| Lipoamide dehydrogenase | OGDH | 1 |
| Lipoprotein lipase | LPL | 4 |
| Lipoprotein receptor, Low Density | LDLR | 2 |
| Lipoprotein, High Density | HDLDT1 | 2 |
| Lipoprotein, Intermediate Density | | 2 |
| Lipoprotein, Low Density 1 | | 2 |
| Lipoprotein, Low Density 2 | | 2 |
| Lipoprotein, Very Low Density | VLDLR | 2 |
| Low density lipoprotein receptor-related protein | LRP | 2 |
| precursor | | |
| Lymphoid enhancer-binding factor | LEF-1 | 6 |
| Lysosomal acid lipase | LIPA | 1 |
| Lysozyme | LYZ | 4 |
| MAD (mothers against decapentaplegic, | MADH4 | 6 |
| Drosophila) homologue 4 | | |
| MADS box transcription-enhancer factor 2A | MEF2A | 6 |
| MADS box transcription-enhancer factor 2B | MEF2B | 6 |
| MADS box transcription-enhancer factor 2C | MEF2C | 6 |
| MADS box transcription-enhancer factor 2D | MEF2D | 6 |
| Malonyl CoA decarboxylase | | 1 |
| Malonyl CoA transferase | | 1 |
| Maltase-glucoamylase | | 1 |
| Mannosidase, alpha B lysosomal | MANB | 1 |
| Marenostrin | MEFV | 2 |
| MAX-interacting protein 1 | mxi I | 6 |
| MEK kinase, MEKK | | 1 |
| Melanocortin 2 receptor | MC2R | 2 |
| Melanocortin 4 receptor | MC4R | 2 |
| Menin | MEN1 | 6 |
| Metallothionein | | 2 |
| Mevalonate kinase | MVK | 1 |
| MHC Class 1: A | | 4 |
| MHC Class 1: B | | 4 |
| MHC Class 1: C | | 4 |
| MHC Class 1: LMP-2, LMP-7 | | 4 |
| MHC Class 1: Tapl | ABCR, TAP1 | 4 |
| MHC Class II: DP | HLA-DPB1 | 4 |
| MHC Class II: DO | | 4 |
| MHC Class II: DR | | 4 |
| MHC Class II: Tap2 | TAP2, PSF2 | 4 |
| MHC Class II: Complementation group A | MHC2TA | 4 |
| MHC Class II: Complementation group B | RFXANK | 4 |
| MHC Class II: Complementation group C | RFX5 | 4 |
| MHC Class II: Complementation group D | RFXAP | 4 |
| Microsomal triglyceride transfer protein | MTP | 2 |
| Mitochondrial trifunctional protein, alpha | HADHA | 1 |
| subunit | | |
| Mitochondrial trifunctional protein, beta subunit | HADHB | 1 |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Motilin | MLN | 6 |
| Msh homeobox homolog 2 | MSX2 | 6 |
| Mucin 18 | MUC18 | 2 |
| Mucin, MUC2 | | 2 |
| Mucin, MUC5AC | | 2 |
| Mucin, MUC6 | | 2 |
| Mucolipidoses | GNPTA | 1 |
| Mulibrey nanism | MUL | 2 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Muscle phosphorylase | PYGM | 1 |
| Mutated in colorectal cancers, MCC | MCC | 6 |
| MutL homolog 1 | MLHI | 6 |
| MutS homolog 2 | MSH2 | 6 |
| MutS homolog 3 | MSH3 | 6 |
| Myoglobin | | 2 |
| Myosin 5A | MY05A | 3 |
| Myosin 6 | MY06 | 3 |
| Myosin 7A | MY07A | 3 |
| Myosin, cardiac | MYH7 | 3 |
| Myosin, light chain 2 | MYL2 | 3 |
| Myosin, light chain 3 | MYL3 | 3 |
| Myotubularin | MTM1 | 3 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+, K+ ATPase, beta 3 | ATP1B3 | 6 |
| Na+/H+ exchanger 1 | NHE1 | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| Na+coupled glucose/galactose transporter | | 2 |
| N-acetylgalactosamine-6-sulfate sulfatase | GALNS | 1 |
| N-acetylglucosamine-6-sulfatase | GNS | 1 |
| N-acetylglucosaminidase, alpha | NAGLU | 1 |
| NADH dehydrogenase | | 1 |
| NADH dehydrogenase (ubiquinone) Fe—S | NDUFS1 | 1 |
| protein I | | |
| NADH dehydrogenase (ubiquinone) Fe—S | NDUFS4 | 1 |
| protein 4 | | |
| NADH dehydrogenase (ubiquinone) | NDUFV1 | 1 |
| flavoprotein 1 | | |
| NADH-cytochrome b5 reductase | DIA1 | 1 |
| NB6 | | 4 |
| Nephrolithiasis 2 | NPHL2 | 2 |
| Nephronophthisis 1 | NPHP1 | 2 |
| Nephronophthisis 2 | NPHP2 | 2 |
| Nephrosis 1 | NPHS1 | 2 |
| Nerve growth factor | NGF | 6 |
| Nerve growth factor receptor | NGFR | 6 |
| Neuraminidase sialidase | NEU | 2 |
| Neurofibromin 1 | NF1 | 6 |
| Neurofibromin 2 | NF2 | 6 |
| Neurokinin A | NKNA | 5 |
| Neurokinin B | NKNB | 5 |
| Neurotensin | NTS | 5 |
| Neurotensin receptor | NTSR1 | 5 |
| Notch ligand-jagged I | JAG 1, AGS | 6 |
| Nuclear factor I-kappa-B-like gene | IKBL | 4 |
| Oncogene ERB | | 6 |
| Oncogene ERB2 | | 6 |
| Oncogene ERBA | | 6 |
| Oncogene ERBAL2 | | 6 |
| Oncogene GLI 1 | GLI | 6 |
| Oncogene GLI2 | GLI2 | 6 |
| Oncogene GLI3 | GLI3 | 6 |
| Oncogene met | MET | 6 |
| Oncogene myb | MYB | 6 |
| Oncogene myc | MYC | 6 |
| Oncogene n-myc | | 6 |
| Oncogene ret | RET | 6 |
| Oncogene r-myc | | 6 |
| Oncogene sis | PDGFB | 6 |
| Oncogene spil | | 6 |
| Oncogene src | | 6 |
| Oncogene v-Ki-ras2 | KRAS2 | 6 |
| Orexin | ox | 6 |
| Orexin 1 receptor | OX1R | 6 |
| Orexin 2 receptor | OX2R | 6 |
| Omithine transcarbamoylase | OTC, NME1 | 1 |
| Osteopontin | OPN | 6 |
| Paired box homeotic gene 2 | PAX2 | 6 |
| Paired box homeotic gene 3 | PAX3 | 6 |
| Paired box homeotic gene 6 | PAX6 | 6 |
| Paired box homeotic gene 8 | PAX8 | 6 |
| Palmitoyl-protein thioesterase | PPT | 2 |
| Pancreatic amylase | | 1 |
| Pancreatic colipase | | 2 |
| Pancreatic lipase | PNLIP | 1 |
| Pancreatic lipase related protein 1 | PLRP1 | 1 |
| Pancreatic lipase related protein 2 | PLRP2 | 1 |
| Paraoxonase PON1 | PON1 | 1 |
| Paraoxonase PON2 | PON2 | 1 |
| Paraoxonase PON3 | | 1 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Parvalbumin | PVALB | 6 |
| Patched (Drosophila) homolog, PTCH | PTCH | 6 |
| Pepsin | | 2 |
| Pepsinogen | | 1 |
| Peptidases A | | 2 |
| Peptidases B | | 2 |
| Peptidases C | | 2 |
| Peptidases D | PEPD | 2 |
| Peptidases E | | 2 |
| Peptidases S | | 2 |
| Peroxidase, salivary | SAPX | 1 |
| Peroxisomal membrane protein 1 | PXMP1 | 3 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome receptor 1 | PXR1 | 2 |
| Phenylalanine monooxygenase | | 1 |
| Phosphatase & tensin homolog | PTEN | 6 |
| Phosphate regulating gene with homologies to | PHEX | 6 |
| endopeptidases on the X chromosome | | |
| Phosphoenolpyruvate carboxykinase | PCK1 | 1 |
| Phosphofructokinase, liver | PFKL | 1 |
| Phosphofructokinase, muscle | PFKM | 1 |
| Phosphoglucomutase | | 1 |
| Phosphoglucose isomerase | GPI | 1 |
| Phosphoglycerate kinase 1 | PGKI | 1 |
| Phosphoglycerate mutase 2 | PGAM2 | 1 |
| Phospholipase A2, group 10 | PLA2G 10 | 4 |
| Phospholipase A2, group 1B | PLA2G 1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCGI | 4 |
| Phosphomannomutase 2 | PMM2 | 6 |
| Phosphomannomutase 2 | PMM2 | 2 |
| Phosphomannose isomerase 1, PMI1 | MPI | 2 |
| Phosphoribosyl pyrophosphate synthetase | PRPS1 | 1 |
| Phosphorylase kinase deficiency, liver | PHK | 1 |
| Phosphorylase kinase, alpha 1 (muscle) | PHKA1 | 1 |
| Phosphorylase kinase, alpha 2 | PHKA2 | 1 |
| Phosphorylase kinase, beta | PHKB | 1 |
| Phosphorylase kinase, delta | | 1 |
| Phosphorylase kinase, gamma 2 | PHKG2 | 1 |
| Plasminogen | PLG | 1 |
| Plasminogen activator inhibitor 1 | PAI 1 | 1 |
| Plasminogen activator inhibitor 2 | PA12 | 1 |
| Plasminogen activator receptor, Urokinase | UPAR, PLAUR | 3 |
| Plasminogen activator, Tissue | PLAT, TPA | 1 |
| Plasminogen activator, Urokinase | UPA, PLAU | 1 |
| Platelet derived growth factor | PDGF | 6 |
| Platelet derived growth factor receptor | PDGFR | 6 |
| Platelet monamine oxidase | | 2 |
| Platelet-activating factor receptor | PAFR | 4 |
| Polycystic kidney and hepatic disease 1 | PKHD1 | 2 |
| Polycystin 1 | PKD1 | 2 |
| Polycystin 2 | PKD2 | 2 |
| Polymorphonuclear elastase | | 2 |
| Potassium inwardly-rectifying channel J1 | KCNJ1 | 5 |
| Potassium inwardly-rectifying channel J11 | KCNJ 11 | 5 |
| Potassium voltage-gated channel E1 | KCNE1 | 5 |
| Prekallikrein | | 4 |
| Preproenkephalin | PENK | 5 |
| Preproglucagon | GCG, GLPL, GLP2 | 6 |
| Preproglucagon | | 2 |
| Preproinsulin | | 2 |
| Procoliagen N-protease | | 1 |
| Proline dehydrogenase | PRODH | 1 |
| Proline-rich protein BstNI subfamily 1 | PRB1 | 3 |
| Proline-rich protein BstNI subfamily 3 | PRB3 | 3 |
| Proline-rich protein BstNI subfamily 4 | PRB4 | 3 |
| Prolyl-4-hydroxylase | | 1 |
| Pro-melanin-concentrating hormone | PMCH | 6 |
| Proopiomelanocortin | POMC | 5 |
| Prosaposin | PSAP | 5 |
| Prostacyclin synthase | | 4 |
| Prostaglandin 15-OH dehydrogenase | HGPD, PGDH | 4 |
| Prostaglandin D - DP receptor | | 4 |
| Prostaglandin E1 receptor | | 4 |
| Prostagiandin E2 receptor | | 4 |
| Prostaglandin E3 receptor | | 4 |
| Prostaglandin F - FP receptor | | 4 |
| Prostagiandin F2 alpha receptor | | 4 |
| Prostaglandin 12 receptor | | 2 |
| Prostaglandin IP receptor | | 4 |
| Protease inhibitor 1 | | 2 |
| Protective protein for beta-galactosidase | PPGB | 1 |
| Protein C | PROC | 4 |
| Protein C inhibitor | PCI | 4 |
| Protein kinase B | PRKB | 4 |
| Protein S | PROSI | 4 |
| Protein tyrosine phosphatase, non-receptor | PTPNI2 | 6 |
| type 12 | | |
| Prothrombin precursor | F2 | 4 |
| Pterin-4-alpha-carbinolamine | PCBD | |
| Pyruvate carboxylase | PC | 1 |
| Pyruvate decarboxylase | PDHA | 1 |
| Pyruvate kinase | PKLR | 1 |
| Quinoid dihydropteridine reductase | QDPR | 1 |
| Renal glutaminase | | 2 |
| Renin | REN | 1 |
| Replication factor C | RFC2 | 1 |
| Retinoblastoma 1 | RB1 | 6 |
| Retinol binding protein 1 | | 2 |
| Retinol binding protein 2 | | 2 |
| Retinoschisis, X-linked, juvenile | RS | 6 |
| RIGUI | RIGUI | 6 |
| SA homolog | SAH | 6 |
| Salivary amylase, AMY1 | | 2 |
| SAP (SLAM-associated protein) | SH2D1A | 4 |
| Secretin | SCT | 2 |
| Secretin receptor, SCTR | SCTR | 2 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage-gated, type 1, beta | SCNLB | 5 |
| polypeptide | | |
| Solute carrier family 10 (sodium/bile acid | SLC10A1 | 2 |
| cotransporter family), member 1 | | |
| Solute carrier family 10 (sodium/bile acid | SLC10A2 | 2 |
| cotransporter family), member 2 | | |
| Solute carrier family 12, member 1 | SLC12A1 | 2 |
| Solute carder family 12, member 2 | SLC12A2 | 2 |
| Solute carrier family 12, member 3 | SLC12A3 | 2 |
| Solute carrier family 14, member 2 | SLC14A2 | 2 |
| Solute carrier family 15 (H+/peptide | SLC15A1 | 2 |
| transporter; intestinal), member 1 | | |
| Solute carrier family 15 (H+/peptide | SLC15A2 | 2 |
| transporter, kidney), member 2 | | |
| Solute carrier family 16 (monocarboxylate | SLC16A1 | 2 |
| transporter), member 1 | | |
| Solute carrier family 16 (monocarboxylate | SLC16A7 | 2 |
| transpofter), member 7 | | |
| Solute carrier family 17, member 1 | SLC17A1 | 2 |
| Solute carrier family 17, member 2 | SLC17A2 | 2 |
| Solute carrier family 2 (facilitated glucose | SLC2A1 | 2 |
| transporter), member 1 | | |
| Solute carrier family 2 (facilitated glucose | SLC2A2 | 2 |
| transporter), member 2 | | |
| Solute carrier family 2 (facilitated glucose | SLC2A3 | 2 |
| transporter), member 3 | | |
| Solute carrier family 2 (facilitated glucose | SLC2A4 | 2 |
| transporter), member 4 | | |
| Solute carrier family 2 (facilitated glucose | SLC2A5 | 2 |
| transpofter), member 5 | | |
| Solute carrier family 21, member 2 | SLC21A2 | 2 |
| Solute carrier family 21, member 3 | SLC21A3 | 2 |
| Solute carrier family 22, member 1 | SLC22A1 | 2 |
| Solute carrier family 22, member 2 | SLC22A2 | 2 |
| Solute carrier family 22, member 5 | SLC22A5 | 2 |
| Solute carrier family 3 (facilitated glucose | SLC3A1 | 2 |
| transporter), member 1 | | |
| Solute carrier family 4 (anion exchanger), | SLC4A1 | 2 |
| member I | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transpofter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmifter | SLC6A3 | 2 |
| transporter, dopamine), member 3 | | |
| Solute carrier family 6 (neurotransmifter | SLC6A2 | 2 |
| transporter, noradrenaline), member 2 | | |
| Solute carrier family 6, member 6 | SLC6A6 | 2 |
| Solute carrier family 7(amino acid transporters, | SLC7AI | 2 |
| member 1 | | |
| Solute carrier family 7(amino acid transporters, | SLC7A2 | 2 |
| member 2 | | |
| Solute carrier family 7(amino acid transporters, | SLC7A7 | 2 |
| member 7 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Sphingomyelinase | SMPD1 | 1 |
| Steroid 5 alpha reductase 1 | SRD5A1 | 1 |
| Steroid 5 alpha reductase 2 | SRD5A2 | 1 |
| Sterol carrier protein 2 | SCP2 | 2 |
| Substance P | | 5 |
| Succinyl CoA synthase | | 1 |
| Sucrase | | 1 |
| Sucrase-isomaltase | SI | 2 |
| Superoxide dismutase 1 | SOD1 | 1 |
| Surfeit I | SURF1 | 6 |
| Talin | TLN | 6 |
| Talin, TLN | | 3 |
| TATA binding protein | TBP | 6 |
| T-BOX 1 | TBXI | 6 |
| T-BOX 2 | TBX2 | 6 |
| T-BOX 3 | TBX3 | 6 |
| Thiolase, perioxisomal | | 1 |
| Thrombin receptor | F2R | 4 |
| Thrombopoietin | THPO | 6 |
| Thromboxane A synthase 1 | TBXAS1 | 4 |
| Tip-associated protein | TAP | 4 |
| Topoisomerase I | | 1 |
| Torticollis, keloids, cryptorchidism and renal | TKCR | 6 |
| dysplasia gene | | |
| Transacylase | | 1 |
| Transcobalamin 1, TCN1 | | 2 |
| Transcobalamin 2, TCN2 | TCN2 | 2 |
| Transcription factor 1, hepatic | TCF1 | 6 |
| Transcription factor 2, hepatic | TCF2 | 6 |
| Transferrin | TF | 6 |
| Transferrin receptor | TFRC | 6 |
| Transforming growth factor, beta 2 | TGFB2 | 6 |
| Transforming growth factor, beta induced | TGFB1 | 6 |
| Transforming growth factor, beta receptor 2 | TGFBR2 | 6 |
| Transglutaminase 4 | TGM4 | 6 |
| Transketolase | TKT | 1 |
| Transketolase-like I | TKTLI | 1 |
| Translocation in renal carcinoma on | TRC8 | 6 |
| chromosome 8 gene | | |
| Transthyretin | TTR | 2 |
| Trehalase | | 2 |
| Triosephosphate isomerase | TPI 1 | 1 |
| Trypsin inhibitor | | 1 |
| Trypsinogen 1 | TRY1 | 1 |
| Trypsinogen 2 | TRY2 | 1 |
| Trypsinogen activation peptide | | 2 |
| Tuberous sclerosis 1 | TSC1 | 6 |
| Tuberous sclerosis 2 | TSC2 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour suppresssor gene DRA | DRA | 4 |
| Tyrosinase | TYR | 1 |
| UDP-glucose pyrophosphorylase | | 1 |
| UDP-glucuronosyltransferase 1 | ugtld, UGT1 | 1 |
| UDP-glucuronosyltransferase 2 | UGT2 | 1 |
| Uridinediphosphate(UDP)-galactose-4- | GALE | 1 |
| epimerase | | |
| Uroporphyrinogen decarboxylase | UROD | 1 |
| Uroporphyrinogen III synthase | UROS | 1 |
| Vasoactive intestinal polypeptide | VIP | 5 |
| Vasoactive intestinal polypeptide receptor | VIPR | 5 |
| Vasoinhibitory peptide | | 6 |
| Villin | | 3 |
| Von Hippel-Lindau gene | VHL | 6 |
| Von Willebrand factor | VWF | 2 |
| Wiskoft-Aldrich syndrome protein | WASP, THC | 4 |
| Wolf-Hirschhom syndrome candidate 1 gene | WHSC1 | 6 |
| Wolfram syndrome 1 gene | WFS1 | 3 |
| Xanthine dehydrogenase | XDH | 1 |
| Xeroderma pigmentosum, complementation | XPA | 1 |
| group A | | |
| Xeroderma pigmentosum, complementation | XPB | 1 |
| group B | | |
| Xeroderma pigmentosum, complementation | XPC | 1 |
| group C | | |
| Xeroderma pigmentosum, complementation | | 1 |
| group D | | |
| Xeroderma pigmentosum, complementation | | 1 |
| group E | | |
| Xeroderma pigmentosum, complementation | XPF | 1 |
| group F | | |
| Xeroderma, pigmentosum, complementation | ERCC5 | 1 |
| group G | | |
| Zinc finger protein 3 | ZIC3 | 3, |

that the genes associated with malfunction, damage or disease of the respiratory system are selected from:

| | HUGO | Protein- |
|---|---|---|
| List of genes | gene symbol | function |
| 11 beta hydroxysteroid dehydrogenase 2 | HSD11B2 | 1 |
| 2,3-bisphosphoglycerate mutase | BPGM | 1 |
| 3 beta hydroxysteroid dehydrogenase 2 | HSD3B2 | 1 |
| Acetoacetyl 1-CoA-thiolase | ACAT1 | 1 |
| Acetoacetyl 2-CoA-thiolase | ACAT2 | 1 |
| Acetyl CoA synthase | | 1 |
| Acetylcholine receptor, nicotinic, gamma | CHRNG | 5 |
| Acetylcholinesterase | ACHE | 1 |
| Aconitase | | 1 |
| Acyl CoA dehydrogenase, long chain | ACADL | 1 |
| Acyl CoA dehydrogenase, medium chain | ACADM | 1 |
| Acyl CoA dehydrogenase, short chain | ACADS | 1 |
| Acyl CoA dehydrogenase, very long chain | ACADVL | 1 |
| Adaptin, beta 3A | ADTB3A | 2 |
| Adenosine deaminase | ADA | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic.receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| Alcohol dehydrogenase 1 | ADH1 | 1 |
| Alcohol dehydrogenase 2 | ADH2 | 1 |
| Alcohol dehydrogenase 3 | ADH3 | 1 |
| Alcohol dehydrogenase 4 | ADH4 | 1 |
| Alcohol dehydrogenase 5 | ADH5 | 1 |
| Alcohol dehydrogenase 6 | ADH6 | 1 |
| Alcohol dehydrogenase 7 | ADH7 | 1 |
| Aldolase A | ALDOA | 1 |
| Aldolase B | ALDOB | 1 |
| Aldolase C | ALDOC | 1 |
| Aldosterone receptor | MLR | 6 |
| Alpha 2 macroglobulin | A2M | 4 |
| Alpha1-antichymotrypsin | AACT | 1 |
| alpha1-Antitrypsin | PI | 1 |
| alpha2-Antiplasmin | PLI | 1 |
| alpha-Actinin 2 | ACTN2 | 6 |
| alpha-Actinin 3 | ACTN3 | 6 |
| alpha-Galactosidase A | GLA | 1 |
| alpha-ketoglutarate dehydrogenase | | 1 |
| Aminopeptidase P | XPNPEP2 | 1 |
| Amphiregulin | AREG | 6 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Annexin 1 | ANX 1 | 1 |
| Antidiuretic hormone receptor | ADHR | 2 |
| Antithrombin III | AT3 | 1 |
| Apolipoprotein E | APOE | 2 |
| Arginase | ARG1 | 1 |
| Arginine vasopressin | AVP | 5 |
| Arginine vasopressin receptor 1A | AVPR1A | 5 |
| Arginine vasopressin receptor 1 B | AVPR1B | 5 |
| Arginine vasopressin receptor 2 | AVPR2 | 5 |
| Arginosuccinate lyase | ASL | 1 |
| Arylsulfatase D | ARSD | 1 |
| Arylsulfatase E | ARSE | 1 |
| Arylsulfatase F | ARSF | 1 |
| Aspartate transaminase | | 2 |
| Ataxia telangiectasia gene, AT | ATM | 6 |
| ATP/ADP translocase | | 1 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| beta-galactosidase | GLB1 | 1 |
| beta-Glucuronidase | GUSB | 1 |
| Blotinidase | BTD | 1 |
| Bloom syndrome protein | BLM | 6 |
| Bradykinin receptor B1 | | |
| Bradykinin receptor B2 | | |
| Butyrylcholinesterase | BCHE | 1 |
| C1 inhibitor | | 1 |
| Cadherin E | CDH1 | 6 |
| Cadherin EP | | 6 |
| Cadherin N | CDH2 | 6 |
| Cadherin P | CDH3 | 6 |
| Calcitonin receptor/Calcitonin gene-related | CALCR | 5 |
| peptide receptor | | |
| Calcitonin/Calcitonin gene-related peptide | CALCA | 5 |
| alpha | | |
| Calcium channel, voltage-dependent, alpha | CACNA1F | 5 |
| 1F subunit | | |
| Calcium channel, voltage-dependent, Alpha- | CACNAL B | 5 |
| 1B (CACNL1A5) | | |
| Calcium channel, voltage-dependent, Alpha- | CACNA1C | 5 |
| 1 C | | |
| Calcium channel, voltage-dependent, Alpha- | CACNA1D | 5 |
| 1 D | | |
| Calcium channel, voltage-dependent, Alpha- | CACNA1E | 5 |
| E (CACNL1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, | CACNG2 | 5 |
| Neuronal, Gamma | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calnexin | CANX | 6 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carnitine acetyltransferase | CRAT | 1 |
| Camitine acylcarnitine translocase | CACT | 1 |
| Catalase | CAT | 4 |
| Cathepsin B | | 1 |
| Cathepsin D | | 1 |
| Cathepsin E | | 1 |
| Cathepsin G | CTSG | 1 |
| Cathepsin H | | 1 |
| Cathepsin K | CTSK | 1 |
| Cathepsin L | | 1 |
| Cathepsin S | | 1 |
| CD1 | CD1 | 4 |
| CD4 | CD4 | 4 |
| Cell adhesion molecule, intercellular, ICAM | ICAM1 | 6 |
| Cell adhesion molecule, leukocyte- | LECAM1 | 6 |
| endothelial, LECAM (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, PECAM1 | | 6 |
| PECAM | | |
| Cell adhesion molecule, vascular, VCAM | VCAM1 | 6 |
| Chemokine receptor CXCR4 | CXCR4 | 4 |
| Chitotriosidase | CHIT | 1 |
| Cholecystokinin | CCK | 5 |
| Cholecystokinin B receptor | CCKBR | 5 |
| Choline acetyltransferase | CHAT | 1 |
| Citrate synthase | | 1 |
| Coenzyme Q (CoQ)/ubiquinone | | 1 |
| Collagen I alpha 1 | COL1A1 | 3 |
| Collagen I alpha 2 | COL1A2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11A1 | 3 |
| Collagen XI alpha 2 | COL11A2 | 3 |
| Collagen XVII alpha 1 | COL17A1 | 3 |
| Colony-stimulating factor 1 | CSF1 | 6 |
| Colony-stimulating factor 1 receptor | CSF1R | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Complement component C1 inhibitor | C1NH | 4 |
| Complement component C1qa | C1QA | 4 |
| Complement component C1qb | C1QB | 4 |
| Complement component C1qg | C1QG | 4 |
| Complement component C1r | C1R | 4 |
| Complement component C1s | C1S | 4 |
| Complement component C2 | C2 | 4 |
| Complement component C3 | C3 | 4 |
| Complement component C4A | C4A | 4 |
| Complement component C4B | C4B | 4 |
| Complement component C5 | C5 | 4 |
| Complement component C6 | C6 | 4 |
| Complement component C7 | C7 | 4 |
| Complement component C8 | C8B | 4 |
| Complement component C9 | C9 | 4 |
| Complement component receptor 1 | CR1 | 4 |
| Complement component receptor 2 | CR2 | 4 |
| Complement component receptor 3 | CR3 | 4 |
| Complex I | | 1 |
| Complex II | | 1 |
| Complex III | | 1 |
| Complex III | | 1 |
| Complex V | MTATP6 | 1 |
| Coproporphyrinogen oxidase | CPO | 1 |
| Corticotrophin-releasing hormone | CRH | 2 |
| Corticotrophin-releasing hormone receptor | CRHR1 | 2 |
| Cortisol receptor | | 4 |
| C-reactive protein CRP | | 4 |
| Creatine kinase - B and m | CKBE | 1 |
| Creb binding protein | CREBBP | 6 |
| Cu2+ transporting ATPase alpha polypeptide | ATP7A | 1 |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin-dependent kinase 2 | CDK2 | 6 |
| Cyclin-dependent kinase inhibitor 2A (p 16) | CDKN2A | 6 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cytidine deaminase | CDA | 1 |
| Cytidine-5-prime-tdphosphate synthetase | CTPS | 1 |
| Cytochrome a | | 1 |
| Cytochrome b-5 | CYB5 | 1 |
| Cytochrome c | | 1 |
| Cytochrome c oxidase, MTCO | | 1 |
| Cytokine-suppressive antiinflammatory drug- | CSBP1 | 4 |
| binding protein I | | |
| Cytokine-suppressive antiinflammatory drug- | CSBP2 | 4 |
| binding protein 2 | | |
| DAX1 nuclear receptor | DAX1 | 4 |
| D-beta-hydroxybutyrate dehydrogenase | | 1 |
| Delta 4-5 alpha-reductase | | 1 |
| Desmin | DES | 3 |
| Dihydrolipoamide dehydrogenase | DLD | 5 |
| DNA glycosylases | | 1 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dystrophin | DMD | 3 |
| Elastase 1 | ELAS1 | 1 |
| Elastase 2 | ELAS2 | 1 |
| Elastin | ELN | 3 |
| Electron-transfering-flavoprotein alpha | ETFA | 2 |
| Electron-transfedng-flavoprotein beta | ETFB | 2 |
| Electron-transferring flavoprotein | ETFDH | 1 |
| dehydrogenase | | |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Enolase | EN01 | 1 |
| Enoyl CoA hydratase | | 1 |
| Enoyl CoA isomerase | | 1 |
| Enoyl CoA reductase | | 1 |
| Enterokinase | PRSS7, ENTK | 1 |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Epoxide hydrolase 1, microsomal | EPHX1 | 1 |
| Estrogen receptor | ESR | 6 |
| EWS RNA-binding protein | EWSR1 | 6 |
| Eyes absent 1 | EYA1 | 6 |
| Faciogenital dysplasia | FGD1, FGDY | 2 |
| Factor 1 (No. one) | F1 | 4 |
| Factor B, properdin | | 4 |
| Factor D | | 4 |
| Factor H | HF1 | 4 |
| Factor I (letter 1) | IF | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & F13B | 4 |
| Fc fragment of IgG, high affinity IA, receptor | FCG1A | 6 |
| for | | |
| Fc fragment of IgG, low affinity IIa, receptor | FCGR2A | 6 |
| for (CD32) | | |
| Fc fragment of IgG, low affinity IIIa, receptor | FCGR3A | 6 |
| for (CD 1 6) | | |
| Fibrillin 1 | FBN1 | 6 |
| Fibrinogen alpha | FGA | 3 |
| Fibrinogen beta | FGB | 3 |
| Fibrinogen gamma | FGG | 3 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FNI | 6 |
| Flightless-II, Drosophila homolog of | FLII | 6 |
| Follicle stimulating hormone receptor | FSHR, ODGI | 6 |
| Follicle stimulating hormone, FSH | FSHB | 6 |
| Forkhead rhabdomyosarcoma gene | FKHR | 6 |
| Fructose-1,6-diphosphatase | FBP1 | 1 |
| Furin | | 2 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRBI | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Galactocerebrosidase | GALC | 1 |
| Galactosyltransferase 1 | GT1 | 6 |
| Galactosyltransferase, alpha 1,3 | GGTA1 | 6 |
| Galactosyltransferase, beta 3 | B3GALT | 6 |
| Glucocorticoid receptor | GRL | 6 |
| Glucokinase | GCK | 1 |
| Glucosidase, acid alpha | GAA | 1 |
| Glutamate dehydrogenase | GLUD1 | 1 |
| Giutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutathione | GSH | 2 |
| Glutathione peroxidase, GPX1 | GPX1 | 1 |
| G utathione peroxidase, GPX2 | GPX2 | 1 |
| Glutathione reductase, GSR | GSR | 1 |
| Glutathione S-transferase mu 1, GSTM1 | GSTM1 | 1 |
| Glutathione S-transferase mu 4, GSTM4 | | 1 |
| Glutathione S-transferase theta 1, GSTT1 | GSTT1 | 1 |
| Glutathione S-transferase theta 2, GSTT2 | | 1 |
| Glutathione S-transferase, GSTP1 | GSTPI | 1 |
| Glutathione S-transferase, GSTZ1 | GSTZ1 | 1 |
| Glutathione synthetase | GSS | 1 |
| Glyceraldehyde-3-phosphate | GAPDH | 1 |
| dehydrogenase, GAPDH | | |
| Glycerol kinase | GK | 1 |
| Glycinamide ribonucleotide (GAR) | GART | 1 |
| transformylase | | |
| GM2 ganglioside activator protein, GM2A | GM2A | 1 |
| Growth arrest-specific homeobox | GAX | 6 |
| Guanylyl cyclase | | 1 |
| Haemoglobin alpha 1 | HBA1 | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Heparin binding epidermal growth factor | HBEGF | 6 |
| Heparin Cofactor II | HCF2 | 4 |
| Hermansky-pudlak syndrome gene | HPS | 2 |
| Hexokinase 1 | HK1 | 1 |
| Hexokinase 2 | HK2 | 1 |
| Hexosaminidase A | HEXA, TSD | 1 |
| Histamine receptors, H1 | | 5 |
| Histamine receptors, H2 | | 5 |
| Histamine receptors, H3 | | 5 |
| HMG-CoA lyase | HMGCL | 1 |
| HMG-CoA reductase | HMGCR | 1 |
| HMG-CoA synthase | HMGCS2 | 1 |
| Holocarboxylase synthetase | HLCS | 1 |
| Hyaluronidase | | 2 |
| Hypoxia inducible factor 1 | HIF1A | 1 |
| Hypoxia inducible factor 2 | | 1 |
| Immunoglobulin E (IgE) reponsiveness gene | IGER | 4 |
| Immunoglobulin E (IgE) serum concentration | IGES | 4 |
| regulator gene | | |
| Immunoglobulin gamma (IgG) 2 | IGHG2 | 4 |
| Insulin | INS | 6 |
| Insulin receptor | INSR | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| Integrin beta 1 | ITGB1 | 6 |
| Integrin beta 2 | ITGB2 | 6 |
| Integrin beta 5 | ITGB5 | 6 |
| Integrin beta 6 | ITGB6 | 6 |
| Integrin, alpha M | ITGAM | 6 |
| Inter-alpha-trypsin inhibitor | IATI | 1 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| Interleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) 11 | IL11 | 4 |
| Interleukin(IL) 11 receptor | IL11R | 4 |
| Interleukin(IL) 12 | IL12 | 4 |
| Interleukin(IL) 12 receptor, beta 1 | IL12RB1 | 4 |
| Interleukin(IL) 13 | IL13 | 4 |
| Interleukin(IL) 13 receptor | IL13R | 4 |
| Interleukin(IL) 2 | IL2 | 4 |
| Interleukin(IL) 2 receptor, alpha | IL2RA | 4 |
| Interleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| Interleukin(IL) 9 | IL9 | 4 |
| Interleukin(IL) 9 receptor | IL9R | 4 |
| Interleukin(IL) receptor antagonist 1 | IL1RN, IL1RA | 4 |
| Isocitrate dehydrogenase | | 1 |
| Kallikrein 3 | KAK3 | 4 |
| Kininogen, High molecular weight | KNG | 4 |
| Kynureninease | | 1 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta | LTBP2 | 6 |
| binding protein 2 | | |
| Lecithin-cholesterol acyltransferase | LCAT | 1 |
| Leptin | LEP | 6 |
| Leptin receptor | LEPR | 6 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| Lipoamide dehydrogenase | OGDH | 1 |
| Lipoprotein lipase | LPL | 4 |
| Lipoprotein receptor, Low Density | LDLR | 2 |
| Lipoprotein, High Density | HDLDT1 | 2 |
| Lipoprotein, Intermediate Density | | 2 |
| Lipoprotein, Low Density 1 | | 2 |
| Lipoprotein, Low Density 2 | | 2 |
| Lipoprotein, Very Low Density | VLDLR | 2 |
| Lipoxygenase | | 1 |
| Low density lipoprotein receptor-related | LRP | 2 |
| protein precursor | | |
| Lymphoid enhancer-binding factor | LEF-1 | 6 |
| Lysosomal acid lipase | LIPA | 1 |
| Lysozyme | LYZ | 4 |
| MAD (mothers against decapentaplegic, | MADH4 | 6 |
| Drosophila) homologue 4 | | |
| Malate dehydrogenase, mitochondrial | MDH2 | 1 |
| Malonyl CoA transferase | | 1 |
| Mannose binding protein | MBP | 4 |
| Mannosidase, alpha B lysosomal | MANB | 1 |
| Mannosidase, beta A lysosomal | MANBA | 1 |
| Matrix Gla protein | MGP | 6 |
| Matrix metalloproteinase 1 | MMP1 | 1 |
| Matrix metalloproteinase 10 | MMP10 | 1 |
| Matrix metalloproteinase 11 | MMP11 | 1 |
| Matrix metalloproteinase 12 | MMP12 | 1 |
| Matrix metalloproteinase 13 | MMP13 | 1 |
| Matrix metalloproteinase 14 | MMP14 | 1 |
| Matrix metalloproteinase 15 | MMP15 | 1 |
| Matrix metalloproteinase 16 | MMP16 | 1 |
| Matrix metalloproteinase 17 | MMP17 | 1 |
| Matrix metalloproteinase 18 | MMP18 | 1 |
| Matrix metalloproteinase 19 | MMP19 | 1 |
| Matrix metalloproteinase 2 | MMP2 | 1 |
| Matrix metalloproteinase 3 | MMP3, STMY1 | 1 |
| Matrix metalloproteinase 4 | MMP4 | 1 |
| Matrix metalloproteinase 5 | MMP5 | F |
| Matrix metalloproteinase 6 | MMP6 | 1 |
| Matrix metalloproteinase 7 | MMP7 | 1 |
| Matrix metalloproteinase 8 | MMP8 | 1 |
| Matrix metalloproteinase 9 | MMP9 | 1 |
| Methionine adenosyltransferase | MAT1A, MAT2A | 1 |
| Midline I | MID1 | 6 |
| Mitochondrial trifunctional protein, alpha | HADHA | 1 |
| subunit | | |
| Mitochondrial trifunctional protein, beta | HADHB | 1 |
| subunit | | |
| Monoamine oxidase A | MAOA | 1 |
| Monoamine oxidase B | MAOB | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Myoglobin | | 2 |
| Myotubularin | MTM1 | 3 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+, K+ ATPase, beta 3 | ATP1B3 | 6 |
| NADH dehydrogenase | | 1 |
| NADH dehydrogenase (ubiquinone) Fe—S | NDUFS1 | 1 |
| protein 1 | | |
| NADH dehydrogenase (ubiquinone) Fe—S | NDUFS4 | 1 |
| protein 4 | | |
| NADH dehydrogenase (ubiquinone) | NDUFV1 | 1 |
| flavoprotein 1 | | |
| NADH-cytochrome b5 reductase | DIA1 | 1 |
| reductase | | |
| Nebulin | NEB | 3 |
| Nephrosis 1 | NPHS1 | 2 |
| Nerve growth factor | NGF | 6 |
| Nerve growth factor receptor | NGFR | 6 |
| Neuraminidase sialidase | NEU | 2 |
| Neuregulin | HGL | 6 |
| Neurofibromin 1 | NF1 | 6 |
| Neurofibromin 2 | NF2 | 6 |
| Neurokinin A | NKNA | 5 |
| Neurokinin B | NKNB | 5 |
| Neuropeptide Y | NPY | 5 |
| Neuropeptide Y receptor Y1 | NPY1 R | 5 |
| Neuropeptide Y receptor Y2 | NPY2R | 5 |
| Nitric oxide synthase 1, NOS 1 | NOSI | 1 |
| Nitric oxide synthase 2, NOS2 | NOS2 | 1 |
| Nitric oxide synthase 3, NOS3 | NOS3 | 1 |
| Notch ligand - jagged 1 | JAG 1, AGS | 6 |
| Nucleoside diphosphate kinase-A | NDPKA | 1 |
| Oncogene ELK1 | ELK1 | 6 |
| Oncogene ELK2 | ELK2 | 6 |
| Oncogene sis | PDGFB | 6 |
| Omithine delta-aminotransferase | OAT | 1 |
| Paired box homeotic gene 6 | PAX6 | 6 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Patched (Drosophila) homolog, PTCH | PTCH | 6 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome receptor 1 | PXR1 | 2 |
| Phenylalanine hydroxylase | PAH | 1 |
| Phenylalanine monooxygenase | | 1 |
| Phenylethanolamine N-methyltransferase, | PNMT | 1 |
| PNMT | | |
| Phosphofructokinase, liver | PFKL | 1 |
| Phosphofructokinase, muscle | PFKM | 1 |
| Phosphoglucomutase | | 1 |
| Phosphoglucose isomerase | GPI | 1 |
| Phosphoglycerate kinase 1 | PGK1 | 1 |
| Phosphoglycerate mutase 2 | PGAM2 | 1 |
| Phospholipase A2, group 10 | PLA2G 10 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | P 2G5 | |
| Phospholipase A2, group 6 | PLA2G6 | 4 |
| Phospholipase C epsilon | | 4 |
| Pineolytic beta-receptors | | 1 |
| Plasminogen | PLG | 1 |
| Plasminogen activator inhibitor 1 | PAI 1 | 1 |
| Plasminogen activator inhibitor 2 | PA12 | 1 |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Platelet-activating factor receptor | PAFR | 4 |
| Potassium inwardly-rectifying channel J1 | KCNJ1 | 5 |
| Potassium voltage-gated channel E1 | KCNE1 | 5 |
| Prekallikrein | | 4 |
| Procollagen N-protease | | 1 |
| receptor) | | |
| Proliferin | PLF | 6 |
| Proopiomelanocortin | POMC | 5 |
| Properdin P factor, complement | PFC, PFD | 4 |
| Prosaposin | PSAP | 5 |
| Prostacyclin synthase | | 4 |
| Prostagiandin 15-OH dehydrogenase | HGPD; PGDH | 4 |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin F2 alpha receptor | | 4 |
| Prostaglandin 12 receptor | | 2 |
| Prostaglandin IP receptor | | 4 |
| Protein C | PROC | 4 |
| Protein C inhibitor | PCI | 4 |
| Protein phosphatase 2, regulatory subunit A, | PPP2R1B | 1 |
| beta isoform | | |
| Protein S | PROS1 | 4 |
| Prothrombin precursor | F2 | 4 |
| Pyruvate carboxylase | PC | 1 |
| Pyruvate decarboxylase | PDHA | 1 |
| Pyruvate kinase | PKLR | 1 |
| Quinoid dihydropteridine reductase | QDPR | 1 |
| Renin | REN | 1 |
| Replication factor C | RFC2 | 1 |
| Retinoblastoma 1 | RB1 | 6 |
| RIGUI | RIGUI | 6 |
| Salivary amylase, AMY1 | | 2 |
| Selectin E | SELE | 5 |
| Selectin L | SELL | 5 |
| Selectin P | SELP | 5 |
| Sedne hydroxymethyltransferase | SHMT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1 D | HTR1D | 5 |
| Serotonin receptor, 5HT1 E | HTR1E | 5 |
| Serotonin receptor, 5HT1 F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, | SCNN1G | 5 |
| gamma | | |
| Sodium channel, voltage gated, type IV, | SCN4A | 5 |
| alpha polypeptide | | |
| Sodium channel, voltage-gated, type 1, beta | SCN1B | 5 |
| polypeptide | | |
| Solute carrier family 21, member 2 | SLC21A2 | 2 |
| Solute carrier family 4 (anion exchanger), | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Somatostatin receptor, SSTR2 | SSTR2 | 6 |
| Sphingomyelinase | SMPD1 | 1 |
| Substance P | | 5 |
| Succinate dehydrogenase 2 | SDH2 | 1 |
| Succinate thiokinase | | 1 |
| Succinyl CoA synthase | | 1 |
| Superoxide dismutase 1 | SOD1 | 1 |
| Superoxide dismutase 3 | SOD3 | 1 |
| Surfactant pulmonary-associated protein A1 | SFTPA1 | 2 |
| Surfactant pulmonary-associated protein A2 | SFTPA2 | 2 |
| Surfactant pulmonary-associated protein B | SFTPB | 2 |
| Surfactant pulmonary-associated protein C | SFTPC | 2 |
| Surfactant puimonary-associated protein D | SFTPD | 2 |
| Surfeit 1 | SURF1 | 6 |
| Survival of motor neuron 1, telomeric | SMN1 | 2 |
| Talin | TLN | 6 |
| T-BOX 2 | TBX2 | 6 |
| T-BOX 3 | TBX3 | 6 |
| TEK, tyrosine kinase, endothelial | TEK | 1 |
| Telomerase protein component | | 1 |
| Thiolase, perioxisomal | | 1 |
| Thrombin receptor | F2R | 4 |
| Thrombomodulin | THBD | 4 |
| Thrombopoietin | THPO | 6 |
| Thrombospondin | THBS1 | 6 |
| Thromboxane A synthase 1 | TBXAS1 | 4 |
| Thromboxane A2 | TXA2 | 4 |
| Thromboxane A2 receptor | TBXA2R | 4 |
| Thyroglobulin | TG | 6 |
| Thyroid hormone receptor, alpha | THRA | 6 |
| Thyroid hormone receptor, beta | THRB | 6 |
| Thyroid peroxidase | TPO | 6 |
| Thyroid receptor auxiliary protein | TRAP | 6 |
| Thyroid-stimulating hormone receptor | TSHR | 6 |
| Thyroid-stimulating hormone, alpha | TSHA | 6 |
| Thyroid-stimulating hormone, beta | TSHB | 6 |
| Thyrotropin releasing hormone receptor | TRHR | 6 |
| Topoisomerase I | | 1 |
| Transacylase | | 1 |
| Transferrin | TF | 6 |
| Transferrin receptor | TFRC | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Transketolase | TKT | 1 |
| Transketolase-like 1 | TKTL1 | 1 |
| Triosephosphate isomerase | TPI 1 | 1 |
| Trypsin inhibitor | | 1 |
| Uncoupling protein I | | 2 |
| Uroporphyrinogen III synthase | UROS | 1 |
| Vasoactive intestinal polypeptide receptor | VIPR | 5 |
| Vasoinhibitory peptide | | 6 |
| Vitronectin receptor, alpha | VNRA | 2 |
| Von Hippel-Lindau gene | VHL | 6 |
| Wolf-Hirschhorn syndrome candidate 1 gene | WHSC1 | 6 |
| Xanthine dehydrogenase | XDH | 1 |
| | HUGO | Protein- |
|---|---|---|
| Liste der Gene | Gensymbol | funktion |
| 5,10-Methylenetetrahydrofolate reductase | MTHFR | 1 |
| (NADPH) | | |
| Acetylcholinesterase | ACHE | 1 |
| Acidic amino acid transporter | | 2 |
| Actin, alpha, cardiac | ACTC | 3 |
| Actin, alpha, skeletal | ACTAL | 3 |
| Actin, alpha, smooth, aortic | ACTA2 | 3 |
| Actin, beta | ACTB | 3 |
| Actin, gamma 2 | ACTG2 | 3 |
| ADAM (A disintegrin and metalloproteinase) | 1 ADAM 1 | 1 |
| ADAM (A disintegrin and metalloproteinase) | 10 ADAM | 1 |
| | 10 | |
| ADAM (A disintegrin and metalloproteinase) | 11 ADAM | 1 |
| | 11 | |
| ADAM (A disintegrin and metalloproteinase) | 12 ADAM | 1 |
| | 12 | |
| ADAM (A disintegrin and metalloproteinase) | 13 ADAM | 1 |
| | 13 | |
| ADAM (A disintegrin and metalloproteinase) | 14 ADAM | 1 |
| | 14 | |
| ADAM (A disintegrin and metalloproteinase) | 15 ADAM | 1 |
| | 15 | |
| ADAM (A disintegrin and metalloproteinase) | 16 ADAM | 1 |
| | 16 | |
| ADAM (A disintegrin and metalloproteinase) | 17 ADAM | 1 |
| | 17 | |
| ADAM (A disintegrin and metalloproteinase) | 18 ADAM | 1 |
| | 18 | |
| ADAM (A disintegrin and metalloproteinase) | 19 ADAM | 1 |
| | 19 | |
| ADAM (A disintegrin and metalloproteinase) | 2 ADAM 2 | 1 |
| ADAM (A disintegrin and metalloproteinase) | 3A ADAM | 1 |
| | 3A | |
| ADAM (A disintegrin and metalloproteinase) | 3B ADAM | 1 |
| | 3B | |
| ADAM (A disintegrin and metalloproteinase) | 4 ADAM 4 | 1 |
| ADAM (A disintegrin and metalloproteinase) | 5 ADAM 5 | 1 |
| ADAM (A disintegrin and metalloproteinase) | 6 ADAM 6 | 1 |
| ADAM (A disintegrin and metalloproteinase) | 7 ADAM 7 | 1 |
| ADAM (A disintegrin and metalloproteinase) | 8 ADAM 8 | 1 |
| ADAM (A disintegrin and metalloproteinase) | 9 ADAM 9 | 1 |
| Adducin, alpha | ADD1 | 3 |
| Adducin, beta | ADD2 | 3 |
| Adenosine deaminase | ADA | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate pyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Albumin, ALB | ALB | 2 |
| Aldosterone receptor | MLR | 6 |
| Alpha 1 acid glycoprotein | AAG, AGP | 2 |
| Alpha 2 macroglobulin | A2M | 4 |
| alpha1-antitrypsin | Pi | 1 |
| alpha2-antiplasmin | PLI | 1 |
| Alpha-fetoprotein | AFP | 6 |
| alpha-glucosidase, neutral AB | GANAB | 1 |
| alpha-glucosidase, neutral C | GANC | 1 |
| Aminopeptidase P | XPNPEP2 | 1 |
| Amylo-1,6-glucosidase | AGL | 1 |
| Amyloid beta A4 precursor protein | APP | 5 |
| Amyloid beta A4 precursor-like protein | APLP | 5 |
| Androgen binding protein | ABP | 2 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCPI | 1 |
| Angiotensin receptor 1 | AGTRL | 2 |
| Angiotensin receptor 2 | AGTR2 | 2 |
| Anglotensinogen | AGT | 1 |
| Annexin I | ANX 1 | 4 |
| Antidiuretic hormone receptor | ADHR | 2 |
| Anti-Mullerian hormone | AMH | 6 |
| Antithrombin III | AT3 | 1 |
| Apaf-1 | | 3 |
| Apolipoprotein E | APOE | 2 |
| Apoptosis antigen 1 | APT1 | 4 |
| Apoptosis antigen ligand 1 | APT1LG1 | 4 |
| Apoptosis-inducing factor | AIF | 4 |
| Arginosuccinate lyase | ASL | 1 |
| Aryl hydrocarbon receptor | AHR | 2 |
| Asparagine synthetase | AS | 1 |
| Aspartylglucosaminidase | AGA | 1 |
| Ataxia telangiectasia complementation group D | ATD, ATDC | 6 |
| Ataxia telangiectasia gene, AT | ATM | 6 |
| ATP-binding cassette transporter 7 | ABC7 | 4 |
| Attractin | | 4 |
| Autoimmune regulator, AIRE | AIRE | 4 |
| B-cell CLL/lymphoma 1 | BCL1 | 4 |
| B-cell CLL/lymphoma 10 | BCL10 | 4 |
| B-cell CLL/lymphoma 3 | BCL3 | 4 |
| B-cell CLL/lymphoma 4 | BCL4 | 4 |
| B-cell CLL/lymphoma 5 | BCL5 | 4 |
| B-cell CLL/lymphoma 6 | BCL6 | 4 |
| B-cell CLLUlymphoma 7 | BCL7 | 4 |
| B-cell CLL/lymphoma 8 | BCL8 | 4 |
| B-cell CLL/lymphoma 9 | BCL9 | 4 |
| BCL2-associated X protein | BAX | 6 |
| BCL2-related protein A1 | BCL2A1 | 6 |
| Beckwith-Wiedemann region 1A | BWR1A | 6 |
| beta 2 Microglobulin | B2M | 4 |
| Bleomycin hydrolase | BLMH | 1 |
| Bloom syndrome protein | BLM | 6 |
| Bradykinin receptor B1 | | |
| Bradykinin receptor B2 | | |
| Brain derived neurotrophic factor | BDNF | 6 |
| Brain derived neurotrophic factor (BDNF) | BDNFR | 6 |
| receptor | | |
| BRCAI-associated RING domain gene 1 | BARD1 | 6 |
| Breakpoint cluster region | BCR | 6 |
| Breast cancer I | BRCAL | 6 |
| Breast cancer 2 | BRCA2 | 6 |
| Breast cancer, ductal, 1 | BRCD1 | 6 |
| Breast cancer, ductal, 2 | BRCD2 | 6 |
| Butyrylcholinesterase | BCHE | 1 |
| C3 convertase | | 1 |
| Cadherin E | CDH1 | 6 |
| Cadherin EP | | 6 |
| Cadherin N | CDH2 | 6 |
| Cadherin P | CDH3 | 6 |
| Calbindin 1 | CALB1 | 6 |
| Calbindin D9K | CALB3 | 6 |
| Calcineurin A1 | CALNA1 | 4 |
| Calcineurin A2 | CALNA2 | 4 |
| Calcineurin A3 | CALNA3 | 4 |
| Calcineurin B | | 4 |
| Calcitonin receptor/Calcitonin gene-related | CALCR | 5 |
| peptide receptor | | |
| Calcitonin/Calcitonin gene-related peptide | CALCA | 5 |
| alpha | | |
| Calcium channel, voltage-dependent, Alpha 1F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha- | CACNA1C | 5 |
| 1C | | |
| Calcium channel, voltage-dependent, Alpha- | CACNA1D | 5 |
| 1D | | |
| Calcium channel, voltage-dependent, Alpha- | CACNA1E | 5 |
| 1E (CACNL1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calmodulin 1 | CALM1 | 6 |
| Calmodulin 2 | CALM2 | 6 |
| Calmodulin 3 | CALM3 | 6 |
| Calmodulin-dependant protein kinase II | CAMK2A | 6 |
| Calnexin | CANX | 6 |
| Calpain | CAPN, | 1 |
| | CAPN3 | |
| Calretinin | CALB2 | 5 |
| Canalicular multispecific organic anion | CMOAT | 2 |
| transporter | | |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carboxylesterase 1 | CES1 | 1 |
| Cardiac-specific homeobox, CSX | CSX | 6 |
| Cartilage-hair hypoplasia gene | CHH | 5 |
| Caspase 1 | CASP1 | 6 |
| Catalase | CAT | 4 |
| Cathepsin G | CTSG | 1 |
| CD1 | CD1 | 4 |
| CD10 | CD10 | 4 |
| CD100 | CD100 | 4 |
| CD112 | CD112 | 4 |
| CD113 | CD113 | 4 |
| CD114 | CD114 | 4 |
| CD115 | CD115 | 4 |
| CD116 | CD116 | 4 |
| CD117 | CD117 | 4 |
| CD118 | CD118 | 4 |
| CD119 | CD119 | 4 |
| CD12 | CD12 | 4 |
| CD13 | CD13 | 4 |
| CD14 | CD14 | 4 |
| CD15 | CD15 | 4 |
| CD17 | CD17 | 4 |
| CD19 | CD19 | 4 |
| CD2 | CD2 | 4 |
| CD20 | CD20 | 4 |
| CD22 | CD22 | 4 |
| CD23 | CD23 | 4 |
| CD24 | CD24 | 4 |
| CD25 | CD25 | 4 |
| CD26 | CD26 | 4 |
| CD27 | CD27 | 4 |
| CD28 | CD28 | 4 |
| CD3 | CD3 | 4 |
| CD30 | CD30 | 4 |
| CD31 | CD31 | 4 |
| CD33 | CD33 | 4 |
| CD34 | CD34 | 4 |
| CD36 | CD36 | 4 |
| CD37 | CD37 | 4 |
| CD38 | CD38 | 4 |
| CD39 | CD39 | 4 |
| CD4 | CD4 | 4 |
| CD40 | CD40 | 4 |
| CD41 | CD41 | 4 |
| CD42 | CD42 | 4 |
| CD43 | CD43 | 4 |
| CD44 | CD44 | 4 |
| CD45 | CD45 | 4 |
| CD46 | CD46 | 4 |
| CD47 | CD47 | 4 |
| CD48 | CD48 | 4 |
| CD5 | CD5 | 4 |
| CD50 | CD50 | 4 |
| CD52 | CD52 | 4 |
| CD53 | CD53 | 4 |
| CD55 | CD55 | 4 |
| CD57 | CD57 | 4 |
| CD58 | CD58 | 4 |
| CD59 | CD59 | 4 |
| CD6 | CD6 | 4 |
| CD60 | CD60 | 4 |
| CD63 | CD63 | 4 |
| CD65 | CD65 | 4 |
| CD66 | CD66 | 4 |
| CD67 | CD67 | 4 |
| CD68 | CD68 | 4 |
| CD69 | CD69 | 4 |
| CD7 | CD7 | 4 |
| CD70 | CD70 | 4 |
| CD71 | CD71 | 4 |
| CD72 | CD72 | 4 |
| CD73 | CD73 | 4 |
| CD74 | CD74 | 4 |
| CD75 | CD75 | 4 |
| CD76 | CD76 | 4 |
| CD77 | CD77 | 4 |
| CD78 | CD78 | 4 |
| CD79 | CD79 | 4 |
| CD8 | CD8 | 4 |
| CD80 | CD80 | 4 |
| CD81 | CD81 | 4 |
| CD83 | CD83 | 4 |
| CD84 | CD84 | 4 |
| CD85 | CD85 | 4 |
| CD86 | CD86 | 4 |
| CD88 | CD88 | 4 |
| CD89 | CD89 | 4 |
| CD9 | CD9 | 4 |
| CD90 | CD90 | 4 |
| CD91 | CD91 | 4 |
| CD92 | CD92 | 4 |
| CD93 | CD93 | 4 |
| CD94 | CD94 | 4 |
| CD96 | CD96 | 4 |
| CD97 | CD97 | 4 |
| CD98 | CD98 | 4 |
| CD99 | CD99 | 4 |
| Cell adhesion molecule, intercellular, ICAM | ICAM1 | 6 |
| Cell adhesion molecule, leukocyte-endothelial, | LECAM1 | 6 |
| LECAM (CD62) | | |
| Cell adhesion molecule, liver, ICAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM1 | 6 |
| PECAM | | |
| Cell adhesion molecule, vascular, VCAM | VCAM1 | 6 |
| Chediak-Higashi syndrome 1 gene | CHS1 | 2 |
| Chemokine MCAF | MCAF | 4 |
| Chemokine receptor CCR2 | CCR2 | 4 |
| Chemokine receptor CCR3 | CCR3 | 4 |
| Chemokine receptor CCR5 | CCR5 | 4 |
| Chemokine receptor CXCR1 | CXCR1 | 4 |
| Chemokine receptor CXCR2 | CXCR2 | 4 |
| Chemokine receptor CXCR4 | CXCR4 | 4 |
| Cholesteryiester hydrolase | | 4 |
| Chondritin Sulphate A - placental receptor | | 4 |
| Chromogranin A | CHGA | 6 |
| Chymase | CHY1 | |
| Clathrin | | 2 |
| CoA transferase | | 1 |
| Collagen I alpha 1 | COL1A1 | 3 |
| Collagen I alpha 2 | COL1A2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, | 3 |
| | EDM2 | |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen XI alpha 1 | COL11A1 | 3 |
| Collagen XI alpha 2 | COL11A2 | 3 |
| Collagen XVII alpha 1 | COLL 7A1 | 3 |
| Coliagenic-like tail subunit of asymmetric | COLQ | 1 |
| acetylcholinesterase | | |
| Colony-stimulating factor I | CSF1 | 6 |
| Colony-stimulating factor 1 receptor | CSF1R | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Complement component C1 inhibitor | Cl NH | 4 |
| Complement component Clqa | CIQA | 4 |
| Complement component Clqb | CLQB | 4 |
| Complement component C1qg | CIQG | 4 |
| Complement component C1r | C1R | 4 |
| Complement component Cls | C1S | 4 |
| Complement component C2 | C2 | 4 |
| Complement component C3 | C3 | 4 |
| Complement component C4A | C4A | 4 |
| Complement component C4B | C4B | 4 |
| Complement component C5 | C5 | 4 |
| Complement component C6 | C6 | 4 |
| Complement component C7 | C7 | 4 |
| Complement component C8 | C8B | 4 |
| Complement component C9 | C9 | 4 |
| Complement component receptor 1 | CR1 | 4 |
| Complement component receptor 2 | CR2 | 4 |
| Complement component receptor 3 | CR3 | 4 |
| Contactin | CNTN1 | 6 |
| Core-binding factor, alpha 1 | CBFA1 | 6 |
| Core-binding factor, alpha 2 | CBFA2 | 6 |
| Core-binding factor, beta | CBFB | 6 |
| Cortico-steroid binding protein | | 2 |
| Corticosteroid nuclear receptor | | 4 |
| Corticotrophin-releasing hormone | CRH | 2 |
| Corticotrophin-releasing hormone receptor | CRHR1 | 2 |
| Cortisol receptor | | |
| C-reactive protein | CRP | |
| c-src tyrosine kinase | CSK | 6 |
| Cyclic AMP response element binding protein | CREB | 6 |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin D | CCND1 | 6 |
| Cyclin-dependent kinase 1 | CDK1 | 6 |
| Cyclin-dependent kinase 10 | CDK10 | 6 |
| Cyclin-dependent kinase 2 | CDK2 | 6 |
| Cyclin-dependent kinase 3 | CDK3 | 6 |
| Cyclin-dependent kinase 4 | CDK4 | 6 |
| Cyclin-dependent kinase 5 | CDK5 | 6 |
| Cyclin-dependent kinase 6 | CDK6 | 6 |
| Cyclin-dependent kinase 7 | CDK7 | 6 |
| Cyclin-dependent kinase 8 | CDK8 | 6 |
| Cyclin-dependent kinase 9 | CDK9 | 6 |
| Cyclin-dependent kinase inhibitor 1A (P21, | CDKN1A | 6 |
| CIP1) | | |
| Cyclin-dependent kinase inhibitor 1 B (P27, | CDKN 1B | 6 |
| KIP1) | | |
| Cyclin-dependent kinase inhibitor 1 C (P57, | CDKN1C | 6 |
| KIP2) | | |
| Cyclin-dependent kinase inhibitor 2A (pl 6) | CDKN2A | 6 |
| Cyclin-dependent kinase inhibitor 3 | CDKN3 | 6 |
| Cyclooxygenase 1 | COX1 | 1 |
| Cyclooxygenase 2 | COX2 | 1 |
| Cyclophilin | | 4 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | |
| binding protein 1 | | |
| Cytokine-suppressive antiinflammatory drug- | CSBP2 | 4 |
| binding protein 2 | | |
| Defender against cell death 1 | DAD1 | 6 |
| Deleted in colorectal carcinoma | DCC | 6 |
| Deoxycorticosterone (DOC) receptor | | 1 |
| Deoxycytidine kinase | DCK | 1 |
| Dihydrolipoyl dehydrogenase 2 | PDHA | 1 |
| Dihydrolipayl transacetylase | PDHA | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Duffy blood group | FY | 2 |
| Dynamin | DNM1 | 6 |
| EB1 | | 6 |
| Elastase 1 | ELAS1 | 1 |
| Elastase 2 | ELAS2 | 1 |
| Endoglin | ENG | 3 |
| Endo-P-D-glucuronidase | | 4 |
| Enolase | ENO1 | 1 |
| Erythroid kruppel-like factor | EKLF | 6 |
| Erythropoietin | EPO | 4 |
| Erythropoietin receptor | EPOR | 4 |
| Estrogen receptor | ESR | 6 |
| EWS RNA-binding protein | EWSR1 | 6 |
| Factor 1 (No. one) | F1 | 4 |
| Factor B, properdin | | 4 |
| Factor D | | 4 |
| Factor H | HF1 | 4 |
| Factor I (letter I) | IF | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & | 4 |
| | F13B | |
| Fanconi anemia, complementation group C | FANCC | 2 |
| Fanconi anemia, complementation group D | FANCD | 2 |
| Fc fragment of IgG, low affinity IIa, receptor for | FCGR2A | 6 |
| (CD32) | | |
| Fc receptor | | 4 |
| Fibrinogen alpha | FGA | 3 |
| Fibrinogen beta | FGB | 3 |
| Fibrinogen gamma | FGG | 3 |
| Fibronectin precursor | FN1 | 6 |
| Follicle stimulating hormone receptor | FSHR, | 6 |
| | ODG1 | |
| Follicle stimulating hormone, FSH | FSHB | 6 |
| Follicular lymphoma variant translocation 1 | FVT1 | 4 |
| Forkhead rhabdomyosarcoma gene | FKHR | 6 |
| Forkhead transcription factor 7 | FKHL7 | 6 |
| Galactosyltransferase 1 | GT1 | 6 |
| Galactosyltransferase, alpha 1,3 | GGTA1 | 6 |
| Galactosyltransferase, beta 3 | B3GALT | 6 |
| Glial-cell derived neurotrophic factor (GDNF) | | 5 |
| receptor | | |
| Glial-cell derived neurotrophic factor, GDNF | GDNF | 5 |
| Glucosaminyl (N-acetyl) transferase 2, I- | GCNT2 | 1 |
| branching enzyme | | |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutamine synthase | | 1 |
| Glutathione | GSH | 2 |
| Glutathione peroxidase, GPX1 | GPX1 | 1 |
| Glutathione peroxidase, GPX2 | GPX2 | 1 |
| Glutathione S-transferase mu 1, GSTM1 | GSTM1 | 1 |
| Glutathione S-transferase mu 4, GSTM4 | | 1 |
| Glutathione S-transferase, GSTZ1 | GSTZ1 | 1 |
| Glyceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Glycophorin A | GYPA | 3 |
| Glycophorin B | GYPB | 3 |
| Glycophorin C | GYPC | 3 |
| Glycosyltransferases, ABO blood group | ABO | 1 |
| Glypican 3 | GPC3, | 6 |
| | SDYS | |
| Gonadotropin releasing hormone receptor | GNRHR | 6 |
| Growth-regulated protein precursor, GRO | GRO | 4 |
| Guanine nucleotide-binding protein, alpha | GNAI1 | 5 |
| inhibiting activity polypeptide 1, GNAI1 | | |
| Guanine nucleotide-binding protein, alpha | GNAI2 | 5 |
| inhibiting activity polypeptide 2, GNAI2 | | |
| Guanine nucleotide-binding protein, alpha | GNAI3 | 5 |
| inhibiting activity polypeptide 3, GNAI3 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, q | GNAQ | 5 |
| polypeptide | | |
| H(+), K(+) - ATPase | ATP4B | 5 |
| Haemoglobin alpha 1 | HBA1 | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Haptoglobin, alpha 1 | HPA1 | 4 |
| Haptoglobin, alpha 2 | HPA2 | 4 |
| Haptoglobin, beta | HPB | 4 |
| Hemochromatosis | HFE | 2 |
| Heparin binding epidermal growth factor | HBEGF | 6 |
| Heparin Cofactor 11 | HCF2 | 4 |
| Hepatitis B virus integration site 1 | HVBS1 | 4 |
| Hepatitis B virus integration site 2 | HVBS6 | 4 |
| High mobility group protein C | HMGIC | 6 |
| High mobility group protein Y | HMGIY | 6 |
| Histamine receptors, H1 | | 5 |
| Histamine receptors, H2 | | 5 |
| Histamine receptors, H3 | | 5 |
| Histatin 1 | | 4 |
| Histatin 2 | | 4 |
| Histatin 3 | HTN3 | 4 |
| HLA-B associated transcript 1 | BAT1 | 4 |
| Holocarboxylase synthetase | HLCS | 1 |
| Homeobox II | HOX11 | 6 |
| Homeobox HB24 | HLX1 | 6 |
| IC7 A and B | | 4 |
| Ikaros gene | IKAROS | 6 |
| Immunoglobulin alpha (IgA) | IGHA | 4 |
| Immunoglobulin delta (IgD) | IGHD | 4 |
| Immunoglobulin E (IgE) reponsiveness gene | IGER | 4 |
| Immunoglobulin E (IgE) serum concentration | IGES | 4 |
| regulator gene | | |
| Immunoglobulin epsilon (IgE) | IGHE | 4 |
| Immunoglobulin gamma (IgG) 2 | IGHG2 | 4 |
| Immunoglobulin heavy mu chain | IGHM | 4 |
| Immunoglobulin J polypeptide | IGJ | 4 |
| Immunoglobulin kappa constant region | IGKC | 4 |
| Immunoglobulin kappa variable region | IGKV | 4 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| Integrin beta 1 | ITGB1 | 6 |
| Integrin beta 2 | ITGB2 | 6 |
| Integrin beta 3 | ITGB3 | 6 |
| Integrin beta 4 | ITGB4 | 6 |
| Integrin beta 5 | ITGB5 | 6 |
| Integrin beta 6 | ITGB6 | 6 |
| Integrin beta 7 | ITGB7 | 6 |
| Integrin, alpha 1 | ITGAI | 6 |
| Integrin, alpha 2 | ITGA2 | 6 |
| Integrin, alpha 4 | ITGA4 | 6 |
| Integrin, alpha 5 | ITGA5 | 6 |
| Integrin, alpha 6 | ITGA6 | 6 |
| Integrin, alpha M | ITGAM | 6 |
| Intercellular adhesion molecule 1 | ICAM1 | 4 |
| Intercellular adhesion molecule 2 | ICAM2 | 4 |
| Intercellular adhesion molecule 3 | ICAM3 | 4 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| Interleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) 11 | IL11 | 4 |
| Interleukin(IL) 11 receptor | IL11R | 4 |
| Interleukin(IL) 12 | IL12 | 4 |
| Interleukin(IL) 12 receptor, beta 1 | IL12RB1 | 4 |
| Interleukin(IL) 13 | IL13 | 4 |
| Interleukin(IL) 13 receptor | IL13R | 4 |
| Interleukin(IL) 2 | IL2 | 4 |
| Interleukin(IL) 2 receptor, alpha | IL2RA | 4 |
| Interleukin(IL) 2 receptor, gamma | IL2RG | 4 |
| Interleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| Interleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| Interleukin(IL) 7 receptor | IL7R | 4 |
| Interleukin(IL) 8 | IL8 | 4 |
| Interleukin(IL) 8 receptor | IL8R | 4 |
| Interleukin(IL) 9 | IL9 | 4 |
| Interleukin(IL) 9 receptor | IL9R | 4 |
| Interleukin(IL) receptor antagonist 1 | IL1RN, | 4 |
| | IL1RA | |
| Janus kinase 1 | JAK1 | 6 |
| Janus kinase 2 | JAK2 | 6 |
| Janus kinase 3 | JAK3 | 6 |
| Kallikrein 3 | KAK3 | 4 |
| Kell blood group precursor | XK, KEL | 2 |
| Kininogen, High molecular weight | KNG | 4 |
| Kynureninease | | 1 |
| Lactotransferrin | LTF | 2 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta binding | LTBP2 | 6 |
| protein 2 | | |
| Lectin, mannose-binding 1 | LMAN1 | 4 |
| Lectin, mannose-binding 2 | MBL2 | 4 |
| Leptin | LEP | 6 |
| Leptin receptor | LEPR | 6 |
| Leukaemia inhibitory factor | LIF | 6 |
| Leukaemia inhibitory factor receptor | LIFR | 6 |
| Leukin | | 4 |
| Leukocyte-specific transcript I | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LIM homeobox protein 1 | LHX1 | 6 |
| LIM homeobox protein 2 | LHX2 | 6 |
| LIM homeobox protein 3 | LHX3 | 6 |
| LIM homeobox protein 4 | LHX4 | 6 |
| LIM-domain only protein 1 | LM01 | 6 |
| LIM-domain only protein 2 | LM02 | 6 |
| LIM-domain only protein 3 | LM03 | 6 |
| LIM-domain only protein 4 | LM04 | 6 |
| LIM-Kinase I (LINK-1) | | 4 |
| Lipocortin 1 | ANX4 | 4 |
| Lipoprotein-associated coagulation factor | LACI | 4 |
| Lipoxygenase 12 (platelets) | LOG12 | 4 |
| Lipoxygenase 5 (leukocytes) | | 4 |
| Lymphoblastic leukemia derived sequence 1 | LYLL | 4 |
| Lymphocyte-specific protein tyrosine kinase | LCK | 4 |
| Lymphoid enhancer-binding factor | LEF-1 | 6 |
| lymphotoxin | | 4 |
| Lysozyme | LYZ | 4 |
| Macrophage activating factor | MAF | 4 |
| Macrophage inflammatory protein-1 | MIP1 | 4 |
| Macrophage inflammatory protein-1 | receptor | 4 |
| Macrophage inflammatory protein-2 | MIP2 | 4 |
| Macrophage inflammatory protein-2 receptor | | |
| MAD (mothers against decapentaplegic, | MADH3 | 6 |
| Drosophila) homologue 3 | | |
| MAD (mothers against decapentaplegic, | MADH4 | 6 |
| Drosophila) homologue 4 | | |
| Malignant proliferation, eosinophil gene | MPE | 4 |
| Mannose binding protein | MBP | 4 |
| Mannosidase, alpha B lysosomal | MANB | 1 |
| Marenostrin | MEFV | 2 |
| Matrix metalloproteinase 1 | MMP1 | 1 |
| Matrix metalloproteinase 10 | NMP10 | 1 |
| Matrix metalloproteinase 11 | MMP11 | 1 |
| Matrix metalloproteinase 12 | MMP12 | 1 |
| Matrix metalloproteinase 13 | MMP13 | 1 |
| Matrix metalloproteinase 14 | MMP14 | 1 |
| Matrix metalloproteinase 15 | MMP15 | 1 |
| Matrix metalloproteinase 16 | MMP16 | 1 |
| Matrix metalloproteinase 17 | MMP17 | 1 |
| Matrix metalloproteinase 18 | MMP18 | 1 |
| Matrix metalloproteinase 19 | MMP19 | 1 |
| Matrix metalloproteinase 2 | MMP2 | 1 |
| Matrix metalloproteinase 3 | MMP3, | 1 |
| | STMY1 | |
| Matrix metalloproteinase 4 | MMP4 | 1 |
| Matrix metalloproteinase 5 | MMP5 | 1 |
| Matrix metalloproteinase 6 | MMP6 | 1 |
| Matrix metalloproteinase 7 | MMP7 | 1 |
| Matrix metalloproteinase 8 | MMP8 | 1 |
| Matrix metalloproteinase 9 | MMP9 | 1 |
| MHC Class I: A | | 4 |
| MHC Class I: B | | 4 |
| MHC Class I: C | | 4 |
| MHC Class I: LMP-2, LMP-7 | | 4 |
| MHC Class I: Tap1 | ABCR, | 4 |
| | TAP1 | |
| MHC Class II: DP | HLA-DPB1 | 4 |
| MHC Class II: DQ | | 4 |
| MHC Class II: DR | | 4 |
| MHC Class II: Tap2 | TAP2, PSF2 | 4 |
| MHC Class II: Complementation group A | MHC2TA | 4 |
| MHC Class II: Complementation group B | RFXANK | 4 |
| MHC Class II: Complementation group C | RFX5 | 4 |
| MHC Class II: Complementation group D | RFXAP | 4 |
| Monocyte chemoattractant protein I | MCP1 | |
| Mucin 18 | MUC18 | 2 |
| Mutated in colorectal cancers, MCC | MCC | 6 |
| MutL homolog 1 | MLH1 | 6 |
| MutS homolog 2 | MSH2 | 6 |
| MutS homolog 3 | MSH3 | 6 |
| Myeloid leukemia factor-1 | MLF1 | |
| Myeloperoxidase | MPO | |
| Myoglobin | | 2 |
| Myosin 5A | MY05A | 3 |
| N-acyl hydrolase | | 4 |
| NADPH oxidase | | 4 |
| reductase | | |
| Natural resistance-associated macrophage | NRAMP1 | 4 |
| protein 1 | | |
| NB6 | | 4 |
| Nerve growth factor | NGF | 6 |
| Nerve growth factor receptor | NGFR | 6 |
| Neurofibromin 1 | NF1 | 6 |
| Neurofibromin 2 | NF2 | 6 |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Neutral endopeptidase | | 1 |
| Neutrophil cystolic factor 1 | NCF1 | 4 |
| Neutrophil cystolic factor 2 | NCF2 | 4 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Norrie disease protein | NDP | 6 |
| Notch 3 | NOTCH3 | 6 |
| Notch ligand-jagged 1 | JAG 1, AGS | 6 |
| Nuclear factor 1-kappa-B-like gene | IKBL | 4 |
| Nuclear factor kappa beta | NFKB | 4 |
| Nuclear factor of activated T cells (NFAT) | NFATC | 6 |
| complex, cytosolic | | |
| Nuclear factor of activated T cells (NFAT) | NFATP | 6 |
| complex, preexisting component | | |
| Nucleoside diphosphate kinase-A | NDPKA | 1 |
| Oncogene bcI2 | | 6 |
| Oncogene ELK1 | ELK1 | 6 |
| Oncogene ELK2 | ELK2 | 6 |
| Oncogene ERG (early reponse gene) | | 6 |
| Oncogene GLI1 | GLI1 | 6 |
| Oncogene GLI2 | GLI2 | 6 |
| Oncogene GLI3 | GLI3 | 6 |
| Oncogene spi1 | | 6 |
| Oncogene TEL | ETV6 | 6 |
| Oncostatin M | OSM | 6 |
| Oncostatin M receptor | OSMR | 6 |
| Ornithine delta-aminotransferase | OAT | 1 |
| Osteonectin | ON | 6 |
| Osteopontin | OPN | 6 |
| Paired box homeotic gene 3 | PAX3 | 6 |
| Paired box homeotic gene 7 | PAX7 | 6 |
| Patched (Drosophila) homolog, PTCH | PTCH | 6 |
| Peanut-like 1 | PNUTL1 | 4 |
| Phagocytin | | 4 |
| Phenylethanolamine N-methyltransferase, | PNMT | 1 |
| PNMT | | |
| Phosphatidylinositol glycan, classA | PIGA | 6 |
| (paroxysmal nocturnal hemoglobinuria) | | |
| Phospholipase A2, group 10 | PLA2G10 | 4 |
| Phospholipase A2, group IB | PLA2GIB | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase-2 | PMM2 | 2 |
| Plakophilin 1 | PKP1 | 2 |
| Plasminogen | PLG | 1 |
| Plasminogen activator inhibitor 1 | PAI I | 1 |
| Plasminogen activator inhibitor 2 | PA12 | 1 |
| Plasminogen activator receptor, Urokinase | UPAR; | 3 |
| | PLAUR | |
| Plasminogen activator, Tissue | PLAT, TPA | 1 |
| Plasminogen activator, Urokinase | UPA; PLAU | 1 |
| Platelet glycoprotein lb, alpha | GPIBA | 4 |
| Platelet glycoprotein lb, beta | GPLBB | 4 |
| Platelet glycoprotein I b, gamma | GPLBG | 4 |
| Platelet glycoprotein IX | GP9 | 4 |
| Platelet glycoprotein V | GP5 | 4 |
| Platelet-activating factor acetylhydrolase 1 B | PAFAH1B1 | 4 |
| | or LIS1 | |
| Platelet-activating factor acetylhydrolase 2 | PAFAH2 | 4 |
| Platelet-activating factor receptor | PAFR | 4 |
| Poliovirus receptor | PVR, PVS | 4 |
| Potassium channel, calcium-activated, | KCNN4 | 5 |
| Potassium inwardly-rectifying channel J1 | KCNJ1 | 5 |
| Potassium inwardly-rectifying channel J11 | KCNJ11 | 5 |
| Potassium voltage-gated channel A1 | KCNA1 | 5 |
| Potassium voltage-gated channel E1 | KCNE1 | 5 |
| Potassium vo tage-gated channel Q1 | KCNQ1 | 5 |
| Potassium voltage-gated channel Q2 | KCNQ2 | 5 |
| Potassium voltage-gated channel Q3 | KCNQ3 | 5 |
| Prekallikrein | | 4 |
| Preproenkephalin | PENK | 5 |
| Procoliagen N-protease | | 1 |
| Promyelocytic leukemia gene | PML | 6 |
| Proopiomelanocortin | POMC | 5 |
| Properdin P factor, complement | PFC, PFD | 4 |
| Prostacyclin synthase | | 4 |
| Prostaglandin (PG) D synthase, hematopoietic | PGDS | 1 |
| Prostaglandin 15-OH dehydrogenase | HGPD; | 4 |
| | PGDH | |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin I2 receptor | | 2 |
| Prostaglandin IP receptor | | 4 |
| Prostaglandin isomerase | | 6 |
| Prostaglandin-endoperoxidase synthase 2 | PTGS2 | 6 |
| Protease inhibitor 1 | | 2 |
| Protein C | PROC | 4 |
| Protein C inhibitor | PCI | 4 |
| Protein kinase A | | 1 |
| Protein kinase C, alpha | PRKCA | 1 |
| Protein kinase C, gamma | PRKCG | 1 |
| Protein kinase DNA-activated | PRKDC | 1 |
| Protein kinase G | | 1 |
| Protein phosphatase 1, regulatory (inhibitor) | PPP1R3 | 1 |
| subunit 3 | | |
| Protein phosphatase 2, regulatory subunit A, | PPP2R1B | 1 |
| beta isoform | | |
| Protein S | PROS1 | 4 |
| Protein tyrosine phosphatase, non-receptor | PTPNI2 | 6 |
| type 12 | | |
| Proteinase 3 | | |
| Prothrombin precursor | F2 | 4 |
| Purine nucleoside phosphorylase | NP | 1 |
| Pyruvate decarboxylase | PDHA | 1 |
| Retinoblastoma 1 | RB1 | 6 |
| Retinol binding protein 4 | RBP4 | 2 |
| Rhesus blood group, CcEe antigens | RHCE | 2 |
| Rhesus blood group, D antigen | RHD | 2 |
| Rhesus blood group-associated glycoprotein | RHAG | 2 |
| Ribosomal protein S 1 9 | RPS19 | 1 |
| RIGUI | RIGUI | 6 |
| S100 calcium-binding protein A2 | S100A2 | 5 |
| S100 calcium-binding protein A3 | S100A3 | 5 |
| S100 calcium-binding protein A5 | S100A5 | 5 |
| S100 calcium-binding protein A6 | S100A6 | 5 |
| S100 calcium-binding protein A7 | S100A7 | 5 |
| S100 calcium-binding protein A8 | S100A8 | 5 |
| S100 calcium-binding protein P | S100P | 5 |
| SAP (SLAM associated protein) | SH2D1A | 4 |
| Selectin E | SELE | 5 |
| Selectin L | SELL | 5 |
| Selectin P | SELP | 5 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Severe combined immunodeficiency, type A | SCIDA | 4 |
| (Athabascan) | | |
| Signal transducer and activator of transcription 1 | STAT1 | 6 |
| Signal transducer and activator of transcription 2 | STAT2 | 6 |
| Signal transducer and activator of transcription 3 | STAT3 | 6 |
| Signal transducer and activator of transcription 4 | STAT4 | 6 |
| Signal transducer and activator of transcription 5 | STAT5 | 6 |
| Signaling lymphocyte activation molecule | SLAM | 4 |
| Sine oculis homeobox, drosophila, homolog 1 | SIX1 | 6 |
| Sine oculis homeobox, drosophila, homolog 2 | SIX2 | 6 |
| Sjoegren (Sjogren) syndrome antigen A1 | SSA1 | 4 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage gated, type V, alpha | SCN5A | 5 |
| polypeptide | | |
| Sodium channel, voltage-gated, type 1, beta | SCN1B | 5 |
| polypeptide | | |
| Solute carrier family 19 (folate transporter), | SLC19A1 | 2 |
| member 1 | | |
| Solute carrier family 20, member 1 | SLC20A1 | 2 |
| Solute carrier family 20, member 2 | SLC20A2 | 2 |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Sorcin | SRI | 2 |
| Sperm prolamine P1 | PRM1 | 6 |
| Sperm prolamine P2 | PRM2 | 6 |
| Stem cell factor | SCF | 6 |
| Stromal derived factor 1 | SDF1 | 6 |
| Succinate dehydrogenase 1 | SDHL | 1 |
| Succinate thiokinase | | 1 |
| Superoxide dismutase 1 | SOD1 | 1 |
| Superoxide dismutase 3 | SOD3 | 1 |
| SYK-related tyrosine kinase | SRK | 4 |
| Talin | TLN | 6 |
| Talin, TLN | | 3 |
| T-cell acute lymphocytic leukemia 1 | TAL1 | 4 |
| T-cell acute lymphocytic leukemia 2 | TAL2 | 4 |
| T-cell receptor, alpha | TCRA | 4 |
| T-cell receptor, delta | TCRD | 4 |
| Tenascin (cytotactin) | | 3 |
| Tenascin XA | TNXA | 3 |
| Terminal deoxynucleotidyltransferase | TDT | 4 |
| Terminal deoxynucleotidyltransferase, TDT | | 1 |
| Thrombin receptor | F2R | 4 |
| Thrombopoietin | THPO | 6 |
| Thrombospondin | THBSI | 6 |
| Thromboxane A synthase I | TBXAS1 | 4 |
| Thromboxane A2 | TXA2 | 4 |
| Thromboxane A2 receptor | TBXA2R | 4 |
| Thy-1 T-cell antigen | THY1 | 4 |
| Thymic humoral factor | | 4 |
| Thymopoietin | TMPO | 6 |
| Thymosin | | 4 |
| TIE receptor tyrosine kinase | TIE-1 | 6 |
| Tip-associated protein | TAP | 4 |
| Toll-like receptor 4 | TLR4 | 4 |
| Topoisomerase I | | 1 |
| Topoisomerase II | | 2 |
| Transcobalamin 2, TCN2 | TCN2 | 2 |
| Transcription factor 3 | TCF3 | 6 |
| Transcription factor binding to IGHM enhancer 3 | TFE3 | 6 |
| Transferrin | TF | 6 |
| Transferrin receptor | TFRC | 6 |
| Transforming growth factor, alpha | TGFA | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Tuberous sclerosis 1 | TSC1 | 6 |
| Tuberous sclerosis 2 | TSC2 | 6 |
| Tubulin | | 3 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour protein p73 | TP73 | 6 |
| Tumour protein, transiationally-controlled 1 | TPT1 | 6 |
| Tumour suppresssor gene DRA | DRA | 4 |
| Ubiquitin | | 6 |
| Ubiquitin activating enzyme, E1 | | 1 |
| Ubiquitin B | UBB | 6 |
| Ubiquitin C | UBC | 6 |
| Ubiquitin fusion degeneration I-like | UFD1L | 6 |
| Ubiquitin protein ligase E3A | UBE3A | 1 |
| Undulin 1 | COL14A1 | 3 |
| Uridine monophosphate kinase | UMPK | 4 |
| Uridine monophosphate synthetase | UMPS | 4 |
| Uroporphyrinogen III synthase | UROS | 1 |
| Vimentin | VIM | 4 |
| v-myc avian myelocytomatosis viral oncogene | MYC | 6 |
| homolog | | |
| Von Hippel-Lindau gene | VHL | 6 |
| Wemer syndrome helicase | WRN | 6 |
| Wilms tumour gene 1 | WT1 | 6 |
| Wilms tumour gene 2 | WT2 | 6 |
| Wilms tumour gene 4 | WT4 | 6 |
| Winged helix nude | WHN | 6 |
| Wiskott-Aldrich syndrome protein | WASP, THC | 4 |
| Xanthine dehydrogenase | XDH | 1 |
| X-ray repair gene | XRCC9 | 6 |
| Zinc finger protein HRX | ALL1 | 4, |

that the genes associated with malfunction, damage or disease of the body as a consequence of an abnormality in the development process are selected from:

| | HUGO | Protein- |
|---|---|---|
| List of genes | gene symbol | function |
| 17-Ketosteroid reductase | | 5 |
| 2,4-Dienoyl CoA reductase | DECR | 1 |
| 3 beta Hydroxysteroid dehydrogenase 2 | HSD3B2 | 1 |
| 3-Oxoacid CoA transferase | OXCT | 1 |
| 6-Pyruvoyltetrahydropterin synthase | PTS | 1 |
| Absent in melanoma 1 gene | AIM1 | 6 |
| Acetoacetyl 2-CoA-thiolase | ACAT2 | 1 |
| Acetyl CoA acyltransferase | ACAA | 1 |
| Acetyl CoA carboxylase alpha | ACACA | 1 |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Achromatopsia 2 | ACHM2 | 3 |
| Acid phosphatase 2, lysosomal | ACP2 | 1 |
| Acrosin | ACR | 6 |
| Actin, alpha, cardiac | ACTC | 3 |
| Actin, alpha, skeletal | ACTA1 | 3 |
| Actin, alpha, smooth, aortic | ACTA2 | 3 |
| Activin | | 6 |
| Activin A receptor, type 2B | ACVR2B | 6 |
| Activin A receptor, type 2-like kinase 1 | ACVRL1 | 6 |
| Acyl CoA dehydrogenase, short chain | ACADS | 1 |
| Acyl-CoA thioesterase | | 1 |
| ADAM (A disintegrin and metalloproteinase) 1 | ADAM1 | 1 |
| ADAM (A disintegrin and metalloproteinase) 10 | ADAM10 | 1 |
| ADAM (A disintegrin and metalloproteinase) 11 | ADAM11 | 1 |
| ADAM (A disintegrin and metalloproteinase) 12 | ADAM12 | 1 |
| ADAM (A disintegrin and metalloproteinase) 13 | ADAM13 | 1 |
| ADAM (A disintegrin and metalloproteinase) 14 | ADAM14 | 1 |
| ADAM (A disintegrin and metalloproteinase) 15 | ADAM15 | 1 |
| ADAM (A disintegrin and metalloproteinase) 16 | ADAM16 | 1 |
| ADAM (A disintegrin and metalloproteinase) 17 | ADAM17 | 1 |
| ADAM (A disintegrin and metalloproteinase) 18 | ADAM18 | 1 |
| ADAM (A disintegdn and metalloproteinase) 19 | ADAM19 | 1 |
| ADAM (A disintegrin and metalloproteinase) 2 | ADAM2 | 1 |
| ADAM (A disintegrin and metalloproteinase) | ADAM3A | 1 |
| 3A | | |
| ADAM (A disintegrin and metalloproteinase) | ADAM3B | 1 |
| 3B | | |
| ADAM (A disintegrin and metalloproteinase) 4 | ADAM4 | 1 |
| ADAM (A disintegrin and metalloproteinase) 5 | ADAM5 | 1 |
| ADAM (A disintegrin and metalloproteinase) 6 | ADAM6 | 1 |
| ADAM (A disintegrin and metalloproteinase) 7 | ADAM7 | 1 |
| ADAM (A disintegrin and metalloproteinase) 8 | ADAM8 | 1 |
| ADAM (A disintegrin and metalloproteinase) 9 | ADAM9 | 1 |
| Adducin, alpha | ADD1 | 3 |
| Adducin, beta | ADD2 | 3 |
| Adenomatous polyposis coli tumour supressor | APC | 6 |
| gene | | |
| Adenosine deaminase | ADA | 1 |
| Adenosine monophosphate deaminase | AMPD | 1 |
| Adenosine receptor A1 | ADORA1 | 5 |
| Adenosine receptor A2A | ADORA2A | 5 |
| Adenosine receptor A2B | ADORA2B | 5 |
| Adenosine receptor A3 | ADORA3 | 5 |
| Adenyl cyclase | | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylosuccinate lyase | ADSL | 1 |
| ADP-ribosyltransferase | ADPRT | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADR83 | 5 |
| Adrenocorticotrophic hormone (ACTH) | ACTHR | 6 |
| receptor | | |
| Adrenoleukodystrophy gene | ALD | 1 |
| Alanine-glyoxylate aminotransferase | AGXT | 1 |
| Albumin, ALB | ALB | 2 |
| Aldehyde dehydrogenase 1 | ALDH1 | 1 |
| Aldehyde dehydrogenase 10 | ALDH10 | 1 |
| Aldehyde dehydrogehase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldolase A | ALDOA | 1 |
| Aldolase B | ALDOB | 1 |
| Aldolase C | ALDOC | 1 |
| Aldosterone receptor | MLR | 6 |
| Alkaline phosphatase, liver/bone/kidney | ALPL | 2 |
| Alkaptonuris gene | AKU | 6 |
| Alkylglycerone phosphate synthase | AGPS | 1 |
| Alpha 2 macroglobulin | A2M | 4 |
| alpha tectorin | TECTA | 6 |
| alpha thalassemia gene | ATRX | 5 |
| alpha1-Antitrypsin | PI | 1 |
| alpha2-Antiplasmin | PLI | 1 |
| alpha-Actinin 2 | ACTN2 | 6 |
| alpha-Actinin 3 | ACTN3 | 6 |
| alpha-Amylase | | 1 |
| Alpha-fetoprotein | AFP | 6 |
| alpha-Galactosidase A | GLA | 1 |
| alpha-ketoglutarate dehydrogenase | | 1 |
| alpha-L-Iduronidase | IDUA | 1 |
| alpha-Synuclein | SNCA | 5 |
| Amelogenin | AMELX | 3 |
| Aminomethyltransferase | AMT | 1 |
| Aminopeptidase P | XPNPEP2 | 1 |
| Amphiregulin | AREG | 6 |
| Amylo-1,6-glucosidase | AGL | 1 |
| Amyloid beta (A4) precursor protein-binding, | APBB1 | 5 |
| APBB1 | | |
| Amyloid beta A4 precursor protein | APP | 5 |
| Amyloid beta A4 precursor-like protein | APLP | 5 |
| Androgen binding protein | ABP | 2 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCP1 | 1 |
| Angiotensinogen | AGT | 1 |
| Ankyrin 1 | ANK1 | 3 |
| Ankyrin 2 | ANK2 | 3 |
| Ankyrin 3 | ANK3 | 3 |
| Antidiuretic hormone receptor | ADHR | 2 |
| Anti-Mullerian hormone | AMH | 6 |
| Anti-Mullerian hormone type 2 receptor | AMHR2 | 6 |
| Antithrombin III | AT3 | 1 |
| AP-2, alpha | TFAP2A | 6 |
| AP-2, beta | TFAP2B | 6 |
| AP-2, gamma | TFAP2C | 6 |
| Apaf-1 | | 3 |
| Apical protein, xenopus laevis-like | APXL | 6 |
| Apolipoprotein A 4 | APOA4 | 2 |
| Apolipoprotein A I | APOA1 | 2 |
| Apolipoprotein A II | APOA2 | 2 |
| Apolipoprotein B | APOB | 2 |
| Apolipoprotein C1 | APOC1 | 2 |
| Apoiipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Apopain | CPP32 | 6 |
| Apoptosis antigen 1 | APT1 | 4 |
| Apoptosis antigen ligand 1 | APT1LG1 | 4 |
| Apoptosis-inducing factor | AIF | 4 |
| Apurinic endonuclease | APE | 1 |
| Archaete-scute homolog 1 | ASH1 | 6 |
| Archaete-scute homolog 2 | ASH2 | 6 |
| Arginosuccinate synthetase | ASS | 1 |
| Arrestin | SAG | 3 |
| Aryl hydrocarbon receptor | AHR | 2 |
| Aryl hydrocarbon receptor nuclear translocator | ARNT | 2 |
| Arylsulfatase A | ARSA | 1 |
| Arylsulfatase B | ARSB | 1 |
| Arylsulfatase C | ARSC1 | 1 |
| Arylsulfatase D | ARSD | 1 |
| Arylsulfatase E | ARSE | 1 |
| Arylsulfatase F | ARSF | 1 |
| Aspartate transaminase | | 2 |
| Aspartate transcarbamoylase | | 1 |
| Aspartoacylase | ASPA | 1 |
| Aspartylglucosaminidase | AGA | 1 |
| Astrotactin | ASTN | 6 |
| Ataxia telangiectasia complementation group D | ATD, ATDC | 6 |
| Ataxia telangiectasia gene, AT | ATM | 6 |
| Ataxin 1 | SCA1 | 6 |
| Ataxin 2 | SCA2 | 6 |
| Ataxin 3 | MJD | 6 |
| ATP-binding cassette transporter 7 | ABC7 | 4 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Atrophin 1 | DRPLA | 6 |
| Affractin | | |
| Autoimmune regulator, AIRE | AIRE | |
| Azoospermia factor 1 | AZF1 | 6 |
| Bagpipe homeobox, drosophila homolog of, 1 | BAPX1 | 6 |
| B-cell CLL/lymphoma 1 | BCL1 | 4 |
| B-cell CLL/lymphoma 10 | BCL10 | 4 |
| B-cell CLL/lymphoma 3 | BCL3 | 4 |
| B-cell CLL/lymphoma 4 | BCL4 | 4 |
| B-cell CLL/lymphoma 5 | BCL5 | 4 |
| B-cell CLL/lymphoma 6 | BCL6 | 4 |
| B-cell CLL/lymphoma 7 | BCL7 | 4 |
| B-cell CLL/lymphoma 8 | BCL8 | 4 |
| B-cell CLL/lymphoma 9 | BCL9 | 4 |
| BCL2-associated X protein | BAX | 6 |
| BCL2-related protein A1 | BCL2A1 | 6 |
| Beckwith-Wiedemann region 1A | BWR1A | 6 |
| Bestrophin | VMD2 | 2 |
| beta 2 Microglobulin | B2M | 4 |
| beta-Endorphin receptor | | 5 |
| beta-Glucuronidase | GUSB | 1 |
| beta-N-Acetylhexosaminidase, A | | 1 |
| beta-N-Acetylhexosaminidase, B | | 1 |
| Bilirubin UDP-glucuronosyltransferase | | 1 |
| Bleomycin hydrolase | BLMH | 1 |
| Bloom syndrome protein | BLM | 6 |
| Blue cone pigment | BCP | 3 |
| Bone morphogenetic protein, BMP1 | BMP1 | 6 |
| Bone morphogenetic protein, BMP2 | BMP2 | 6 |
| Bone morphogenetic protein, BMP3 | BMP3 | 6 |
| Bone morphogenetic protein, BMP4 | BMP4 | 6 |
| Bone morphogenetic protein, BMP5 | BMP5 | 6 |
| Bone morphogenetic protein, BMP6 | BMP6 | 6 |
| Bone morphogenetic protein, BMP7 | BMP7 | 6 |
| Bone morphogenetic protein, BMP8 | BMP8 | 6 |
| Brain derived neurotrophic factor | BDNF | 6 |
| Brain derived neurotrophic factor (BDNF) | BDNFR | 6 |
| receptor | | |
| Branched chain aminotransferase 1, cytosolic | BCAT1 | 1 |
| Branched chain aminotransferase 2, | BCAT2 | 1 |
| mitochondrial | | |
| BRCA1-associated RING domain gene 1 | BARD1 | 6 |
| Breakpoint cluster region | BCR | 6 |
| Breast cancer 1 | BRCA1 | 6 |
| Breast cancer 2 | BRCA2 | 6 |
| Breast cancer, ductal, 1 | BRCD1 | 6 |
| Breast cancer, ducta, 2 | BRCD2 | 6 |
| Bruton agammaglobulinaemia tyrosine kinase | BTK | 6 |
| Butyrylcholinesterase | BCHE | 1 |
| C3 convertase | | 1 |
| Ca(2+) transporting ATPase, fast twitch | ATP2A1 | 2 |
| Ca(2+) transporting ATPase, slow twitch | ATP2A2 | 2 |
| Cadherin E | CDHI | 6 |
| Cadherin EP | | 6 |
| Cadherin N | CDH2 | 6 |
| Cadherin P | CDH3 | 6 |
| Calbindin 1 | CALB1 | 6 |
| Calbindin D9K | CALB3 | 6 |
| Calcium channel, voltage-dependent, Alpha 1 F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha- | CACNA1C | 5 |
| 1 C | | |
| Calcium channel, voltage-dependent, Alpha- | CACNA1D | 5 |
| 1 D | | |
| Calcium channel, voltage-dependent, Alpha- | CACNA1E | 5 |
| 1 E (CACNL1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNA1S | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calcium sensing receptor | CASR | 2 |
| Calmodulin 1 | CALM1 | 6 |
| Calmodulin 2 | CALM2 | 6 |
| Calmodulin 3 | CALM3 | 6 |
| Calmodulin dependent kinase | | 2 |
| Calmodulin-dependant protein kinase II | CAMK2A | 6 |
| Calnexin | CANX | 6 |
| Calpain | CAPN, CAPN3 | 1 |
| Canalicular multispecific organic anion | CMOAT | 2 |
| transporter | | |
| Carbamoylphosphate synthetase 1 | CPS1 | 1 |
| Carbamoylphosphate synthetase 2 | CPS2 | 1 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Cardiac-specific homeobox, CSX | CSX | 6 |
| Carnitine acetyltransferase | CRAT | 1 |
| Carnitine acylcarnitine translocase | CACT | 1 |
| Carnitine transporter protein | CDSP, SCD | 2 |
| Cartilage oligomeric matrix protein | COMP, EDM1, | 5 |
| | PSACH | |
| Cartilage-hair hypoplasia gene | CHH | 5 |
| Caspase 1 | CASP1 | 6 |
| Caspase 10 | CASP10 | 6 |
| Caspase 2 | CASP2 | 6 |
| Caspase 3 | CASP3 | 6 |
| Caspase 4 | CASP4 | 6 |
| Caspase 5 | CASP5 | 6 |
| Caspase 6 | CASP6 | 6 |
| Caspase 7 | CASP7 | 6 |
| Caspase 8 | CASP8 | 6 |
| Caspase 9 | CASP9 | 6 |
| Catechol-o-methyltransferase | COMT | 1 |
| Catenin, alpha | CTNNA1 | 6 |
| Catenin, beta | CTNNB1 | 6 |
| Catenin, gamma | | 6 |
| Cathepsin K | CTSK | 1 |
| Caveolin 3 | CAV3 | 1 |
| CD1 | CD1 | 4 |
| CD44 | CD44 | 4 |
| Cdc 25 phosphatase | | 6 |
| Cdc2 | CDC2 | 6 |
| CDX1 | | 6 |
| CEA | | 6 |
| Cell adhesion molecule, intercellular, ICAM | ICAM1 | 6 |
| Cell adhesion molecule, leukocyte-endothelial, LECAM1 | | 6 |
| LECAM (CD62) | | |
| Cell adhesion molecule, liver, LCAM | LCA | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM | 6 |
| PECAM | | |
| Cell adhesion molecule, vascular, VCAM | VCAM1 | 6 |
| Cellubrevin | CEB | 5 |
| c-erbB1 | ERBB1 | 6 |
| c-erbB2 | ERBB2 | 6 |
| c-erbB3 | ERBB3 | 6 |
| c-erbB4 | ERBB4 | 6 |
| Ceroid lipofuscinosis neuronal 2 | CLN2 | 5 |
| Ceroid lipofuscinosis neuronal 3 | CLN3 | 5 |
| Ceroid lipofuscinosis neuronal 4 | CLN4 | 5 |
| Ceroid lipofuscinosis neuronal 5 | CLN5 | 5 |
| Ceroid lipofuscinosis neuronal 6 | CLN6 | 5 |
| Chediak-Higashi syndrome 1 gene | CHS1 | 2 |
| Chemokine MCAF | MCAF | 4 |
| Chemokine receptor CCR2 | CCR2 | 4 |
| Chemokine receptor CCR3 | CCR3 | 4 |
| Chemokine receptor CCR5 | CCR5 | 4 |
| Chemokine receptor CXCR1 | CXCR1 | 4 |
| Chemokine receptor CXCR2 | CXCR2 | 4 |
| Chemokine receptor CXCR4 | CXCR4 | 4 |
| Chloride channel 5 | CLCN5 | 3 |
| Cholestasis, progressive familial intrahepatic 1 | FIC1 | 6 |
| gene | | |
| Cholesterol ester transfer protein | CETP | 2 |
| Choline acetyltransferase | CHAT | 1 |
| Choroideremia gene | CHM | 3 |
| Chromogranin A | CHGA | 6 |
| Ciliary neurotrophic factor (CNTF) | CNTF | 6 |
| Ciliary neurotrophic factor (CNTF) receptor | CNTFR | 6 |
| c-kit receptor tyrosine kinase | | 6 |
| Clathrin | | 2 |
| Cleavage signal-1 protein | CS1 | 6 |
| Cleft palate gene | CPX | 6 |
| Clusterin | CLU | 6 |
| CoA transferase | | 1 |
| Cochlin | COCH | 4 |
| Cockayne syndrome gene, CKN1 | CKN1 | 6 |
| Collagen I alpha 1 | COL1A1 | 3 |
| Collagen I alpha 2 | COL1A2 | 8 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, EDM2 | 3 |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6A1 | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COL11A1 | 3 |
| Collagen XI alpha 2 | COL11A2 | 3 |
| Collagen XVII alpha 1 | COL17A1 | 3 |
| Collagenic-like tail subunit of asymmetric | COLQ | 1 |
| acetylcholinesterase | | |
| Collapsin | | 6 |
| Colony-stimulating factor 1 | CSF1 | 6 |
| Colony-stimulating factor 1 receptor | CSF1R | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Complex V | MTATP6 | 1 |
| Cone-rod homeobox-containing gene | CRX | 6 |
| Contactin | CNTN1 | 6 |
| Core-binding factor, alpha 1 | CBFA1 | 6 |
| Core-binding factor, alpha 2 | CBFA2 | 6 |
| Core-binding factor, beta | CBFB | 6 |
| Corticotrophin-releasing hormone | CRH | 2 |
| Corticotrophin-releasing hormone receptor | CRHR1 | 2 |
| Creatine kinase - B and m | CKBE | 1 |
| Creb binding protein | CREBBP | 6 |
| Cryptochrome 1 | CRY1 | 3 |
| Cryptochrome 2 | CRY2 | 3 |
| Crystallin, alpha A | CRYAA | 3 |
| Crystallin, alpha B | CRYAB | 3 |
| Crystallin, beta B2 | CRYBB2 | 3 |
| Crystallin, gamma A | CRYGA | 3 |
| c-src tyrosine kinase | CSK | 6 |
| Cu2+ transporting ATPase alpha polypeptide | ATP7A | 1 |
| Cu2+ transporting ATPase beta polypeptide | ATP7B | 1 |
| Cubilin | CUBN | 2 |
| Cyclic AMP response element binding protein | CREB | 6 |
| Cyclic AMP response element modulator | CREM | 6 |
| Cyclic AMP-dependent protein kinase | PKA | 1 |
| Cyclic nucleotide gated channel alpha 1, | CNGA1 | 5 |
| CNGA1 | | |
| Cyclic nucleotide gated channel alpha 3, | CNGA3 | 5 |
| CNGA3 | | |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin A | CCNA | 6 |
| Cyclin B | CCNB | 6 |
| Cyclin C | CCNC | 6 |
| Cyclin D | CCND1 | 6 |
| Cyclin E | CCNE | 6 |
| Cyclin F | CCNF | 6 |
| Cyclin-dependent kinase 1 | CDK1 | 6 |
| Cyclin-dependent kinase 10 | CDK10 | 6 |
| Cyclin-dependent kinase 2 | CDK2 | 6 |
| Cyclin-dependent kinase 3 | CDK3 | 6 |
| Cyclin-dependent kinase 4 | CDK4 | 6 |
| Cyclin-dependent kinase 5 | CDK5 | 6 |
| Cyclin-dependent kinase 6 | CDK6 | 6 |
| Cyclin-dependent kinase 7 | CDK7 | 6 |
| Cyclin-dependent kinase 8 | CDK8 | 6 |
| Cyclin-dependent kinase 9 | CDK9 | 6 |
| Cyclin-dependent kinase inhibitor 1A (P21, | CDKN1A | 6 |
| CIP1) | | |
| Cyclin-dependent kinase inhibitor 1B (P27, | CDKN1B | 6 |
| KIP1) | | |
| Cyclin-dependent kinase inhibitor 1C (P57, | CDKN1C | 6 |
| KIP2) | | |
| Cyclin-dependent kinase inhibitor 2A (p16) | CDKN2A | 6 |
| Cyclin-dependent kinase inhibitor 3 | CDKN3 | 6 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cystinosin | CTNS | 2 |
| Cytidine deaminase | CDA | 1 |
| Cytochrome b-5 | CYB5 | 1 |
| DAX1 nuclear receptor | DAX1 | 4 |
| Deafness autosomal dominant 5 | DFNA5 | 5 |
| Deafness dystonia peptide | DDP | 5 |
| Defender against cell death 1 | DAD1 | 6 |
| Deleted in azospermia | DAZ | 6 |
| Deleted in colorectal carcinoma | DCC | 6 |
| Deleted in malignant brain tumours 1 | DMBT1 | 6 |
| Delta aminolevulinate dehydratase | ALAD | 1 |
| Delta(4)-3-oxosteroid 5-beta-reductase | | 1 |
| Delta-7-dehydrocholesterol reductase | DHCR7 | 1 |
| Dentin sialophosphoprotein | DSPP | 6 |
| Deoxyuridine triphosphatase; dUTPase | | 1 |
| Desert hedgehog, dhh | | 6 |
| DHEA sulfotransferase | STD | 1 |
| Diaphanous 1 | DIAPH1 | 5 |
| Diaphanous 2 | DIAPH2 | 5 |
| Diastrophic dysplasia sulfate transporter | DTD | 2 |
| Dihydrolipoamide branched chain transacylase | DBT | 5 |
| Dihydrolipoamide dehydrogenase | DLD | 5 |
| Dihydrolipoyl dehydrogenase 2 | PDHA | 1 |
| Dihydrolipoyl transacetylase | PDHA | 1 |
| Dihydroorotase | | 1 |
| Dihydroxyacetonephosphate acyltransferase | DHAPAT | 1 |
| Disrupted meiotic cDNA 1, homolog | DMC1 | 6 |
| Distal-less homeobox 1 | DLX1 | 6 |
| Distal-less homeobox 2 | DLX2 | 6 |
| Distal-less homeobox 3 | DLX3 | 6 |
| Distal-less homeobox 4 | DLX4 | 6 |
| Distal-less homeobox 5 | DLX5 | 6 |
| Distal-less homeobox 6 | DLX6 | 6 |
| DNA damage binding protein, DDB1 | DDB1 | 3 |
| DNA damage binding protein, DDB2 | DDB2 | 3 |
| DNA directed polymerase, alpha | POLA | 1 |
| DNA glycosylases | | 1 |
| DNA helicases | | 1 |
| DNA Ligase 1 | LIG1 | 1 |
| DNA methyltransferase | DNMT | 1 |
| DNA polymerase 1 | | 1 |
| DNA polymerase 2 | | 1 |
| DNA polymerase 3 | | 1 |
| DNA primase | | 1 |
| DNA-damage-inducible transcript 3 | DDIT3 | 3 |
| DNA-dependant RNA polymerase | | 1 |
| DOPA decarboxylase | DDC | 1 |
| Doublecortin, DCX | DCX | 3 |
| Duffy blood group | FY | 2 |
| Dynamin | DNM1 | 6 |
| Dynein | | 6 |
| Dyskerin | DKC1 | 3 |
| Dystonia 1 | DYT1 | 3 |
| Dystonia 3 | DYT3 | 3 |
| Dystonia 6 | DYT6 | 3 |
| Dystonia 7 | DYT7 | 3 |
| Dystonia 9 | CSE | 3 |
| Dystrophia myotonica | DM, DMPK | 1 |
| Dystrophia myotonica, atypical | DM2 | 1 |
| Dystrophin | DMD | 3 |
| Dystrophin-associated glycoprotein 35 kD, | SGCD | 3 |
| SCGD | | |
| Dystrophin-associated glycoprotein 35 kD, | SGCG | 3 |
| SGSG | | |
| Dystrophin-associated glycoprotein 43 kD | SGCB | 3 |
| Dystrophin-associated glycoprotein 5OkD | SGCA | 3 |
| E74-like factor 1, ELF1 | ELF1 | 6 |
| EB1 | | 6 |
| Ectodermal Dysplasia 1 gene | ED1 | 3 |
| Electron-transfering-flavoprotein alpha | ETFA | 2 |
| Electron-transfering-flavoprotein beta | ETFB | 2 |
| Electron-transferring flavoprotein | ETFDH | 1 |
| dehydrogenase | | |
| Empty spiracles (drosophila) homologue 1 | EMX1 | 6 |
| Empty spiracles (drosophila) homologue 2 | EMX2 | 6 |
| Endobrevin | VAMP8 | 5 |
| Endocardial fibroelastosis 2 gene | EFE2 | 3 |
| Endometrial bleeding-associated factor | EBAF | 6 |
| Endothelin 1 | EDN1 | 5 |
| Endothelin 2 | EDN2 | 5 |
| Endothelin 3 | EDN3 | 5 |
| Endothelin converting enzyme | ECE1 | 5 |
| Endothelin receptor type A | EDNRA | 5 |
| Endothelin receptor type B | EDNRB | 5 |
| Engrailed-1 | ENI | 6 |
| Engrailed-2 | EN2 | 6 |
| Enolase | ENO1 | 1 |
| Enoyl CoA isomerase | | 1 |
| Enterokinase | PRSS7, ENTK | 1 |
| Ephrin receptor tyrosine kinase A | EPHA | 6 |
| Ephrin receptor tyrosine kinase B | EPHB | 6 |
| Ephrin-A | EFNA | 6 |
| Ephrin-B | EFNB | 6 |
| Epidermal growth factor | EGF | 6 |
| Epidermal growth factor receptor | EGFR | 6 |
| Epilepsy, benign neonatal 4 gene | ICCA | 1 |
| Epilepsy, female restricted | EFMR | 1 |
| Epilepsy, progressive myoclonic 2 gene | EPM2A | 1 |
| Erythrocyte membrane protein band 4.1 | EPB41 | 3 |
| Erythrocyte membrane protein band 4.2 | EPB42 | 3 |
| Erythrocyte membrane protein band 7.2 | EPB72 | 3 |
| Erythroid kruppel-like factor | EKLF | 6 |
| Erythropoietin | EPO | 4 |
| Erythropoietin receptor | EPOR | 4 |
| Estrogen receptor | ESR | 6 |
| Eukaryotic initiation translation factor | EIF4E | 6 |
| EWS RNA-binding protein | EWSR1 | 6 |
| Excision repair complementation group 1 | ERCC1 | 1 |
| protein | | |
| Excision repair complementation group 2 | ERCC2 | 1 |
| protein | | |
| Excision repair complementation group 2 | ERCC3 | 1 |
| protein | | |
| Excision repair complementation group 4 | ERCC4 | 1 |
| protein | | |
| Excision repair complementation group 6 | ERCC6 | 1 |
| protein | | |
| Exostosin 1 | EXT1 | 3 |
| Exostosin 2 | EXT2 | 3 |
| Exostosin 3 | EXT3 | 3 |
| Eyes absent 1 | EYA1 | 6 |
| Eyes absent 2 | EYA2 | 6 |
| Eyes absent 3 | EYA3 | 6 |
| Faciogenital dyspiasia | FGD1, FGDY | 2 |
| Factor 1 (No. one) | F1 | 4 |
| Factor B, properdin | | 4 |
| Factor D | | 4 |
| Factor H | HF1 | 4 |
| Factor I (letter 1) | IF | 4 |
| Factor III | F3 | 4 |
| Factor IX | F9 | 4 |
| Factor V | F5 | 4 |
| Factor VII | F7 | 4 |
| Factor VIII | F8 | 4 |
| Factor X | F10 | 4 |
| Factor XI | F11 | 4 |
| Factor XII | F12 | 4 |
| Factor XIII A & B | F13A & F13B | 4 |
| Fanconi anemia, complementation group A | FANCA | 2 |
| Fanconi anemia, complementation group C | FANCC | 2 |
| Fanconi anemia, complementation group D | FANCD | 2 |
| Fc fragment of IgG, high affinity IA, receptor for | FCGR1A | 6 |
| Fc fragment of IgG, low affinity IIa, receptor for | FCGR2A | 6 |
| (CD32) | | |
| Fc fragment of IgG, low affinity IIIa, receptor for | FCGR3A | 6 |
| (CD16) | | |
| Fc receptor | | 4 |
| Fertilin protein | FTNB | 6 |
| Fibrillin 1 | FBN1 | 6 |
| Fibrillin 2 | FBN2 | 6 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Flavin-containing monooxygenase 1 | FMO1 | 1 |
| Flavin-containing monooxygenase 2 | FMO2 | 1 |
| Flavin-containing monooxygenase 3 | FMO3 | 1 |
| Flavin-containing monooxygenase 4 | FMO4 | 1 |
| Flightless-II, Drosophila homolog of | FLII | 6 |
| Folic acid receptor | FOLR | 6 |
| Follicle stimulating hormone receptor | FSHR, ODG1 | 6 |
| Follicle stimulating hormone, FSH | FSHB | 6 |
| Follicular lymphoma variant translocation 1 | FVT1 | 4 |
| Follistatin | | 6 |
| Forkhead rhabdomyosarcoma gene | FKHR | 6 |
| Forkhead transcription factor 10 | FKHL10 | 6 |
| Forkhead transcription factor 14 | FKHL14 | 6 |
| Forkhead transcription factor 7 | FKHL7 | 6 |
| Formiminotransferase | | 1 |
| Fragile site, folic acid type, rare, fra(X) A | FRAXA | 5 |
| Fragile site, folic acid type, rare, fra(X) E | FRAXE | 5 |
| Fragile site, folic acid type, rare, fra(X) F | FRAXF | 5 |
| Frataxin | FRDA | 6 |
| Fringe secreted protein, lunatic | LFNG | 6 |
| Fringe secreted protein, manic | MFNG | 6 |
| Fringe secreted protein, radical | RFNG | 6 |
| Fructose-1,6-diphosphatase | FBP1 | 1 |
| Fucosyltransferase 6 | FUT6 | 2 |
| Fukuyama type congenital muscular dystrophy | FCMD | 6 |
| Fumarase | FH | 1 |
| Fumarylacetoacetase | FAH | 1 |
| G/T mismatch binding protein | GTBP, MSH6 | 6 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Gadd45 (growth arrest & DNA-damage-inducible protein) | | 1 |
| Galactocerebrosidase | GALC | 1 |
| Galactokinase | GALK1 | 1 |
| Galactose 1-phosphate uridyl-transferase | GALT | 1 |
| Galactosyltransferase 1 | GT1 | 6 |
| Galactosyltransferase, alpha 1,3 | GGTA1 | 6 |
| Galactosyltransferase, beta 3 | B3GALT | 6 |
| Galanin | GAL | 5 |
| Galanin receptor | GALNR1 | 5 |
| Gamma-glutamyl carboxylase | GGCX | 2 |
| Gap junction protein alpha 1 | GJA1 | 2 |
| Gap junction protein alpha 3 | GJA3 | 2 |
| Gap junction protein alpha 8 | GJA8 | 2 |
| Gap junction protein beta 1 | GJB1 | 2 |
| Gap junction protein beta 2 | GJB2 | 2 |
| Gap junction protein beta 3 | GJB3 | 2 |
| Gastric Intrinsic factor, GIF | GIF | 1 |
| Gastrin | GAS | 6 |
| Gastrin releasing peptide | GRP | 2 |
| Gastrulation brain homeobox 2 | GBX2 | 6 |
| GDP dissociation inhibitor I | GDI 1 | 6 |
| Gelsolin | GSN | 6 |
| Geniospasm 1 | GSM1 | 6 |
| Gephyrin | | 5 |
| Glial-cell derived neurotrophic factor (GDNF) | | 5 |
| receptor | | |
| Glial-cell derived neurotrophic factor, GDNF | GDNF | 5 |
| Glioma chloride ion channel, GCC | | 6 |
| Glucagon receptor | GCGR | 6 |
| Glucagon-like peptide receptor 1 | GLPLR | 6 |
| Glucocorticoid receptor | GRL | 6 |
| Glucose-6-phosphatase translocase | G6PT1 | 1 |
| Glucosidase, acid alpha | GAA | 1 |
| Glucosidase, acid beta | GBA | 1 |
| Glutamate decarboxylase, GAD | GAD1 | 1 |
| Glutamate-cysteine ligase | GLCLC | 1 |
| Glutathione | GSH | 2 |
| Glutathione peroxidase, GPX1 | GPX1 | 1 |
| Glutathione peroxidase, GPX2 | GPX2 | 1 |
| Glutathione reductase, GSR | GSR | 1 |
| Glutathione S-transferase mu 1, GSTM1 | GSTM1 | 1 |
| Glutathione S-transferase mu 4, GSTM4 | | 1 |
| Glutathione S-transferase theta 1, GSTT1 | GSTTI | 1 |
| Glutathione S-transferase theta 2, GSTT2 | | 1 |
| Glutathione S-transferase, GSTP1 | GSTP1 | 1 |
| Glutathione S-transferase, GSTZ1 | GSTZ1 | 1 |
| Glutathione synthetase | GSS | 1 |
| Glyceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Glycine dehydrogenase | GLDC | 1 |
| Glycine receptor, alpha | GLRA2 | 5 |
| Glycine receptor, beta | | 5 |
| Glycogen branching enzyme | GBE1 | 1 |
| Glycogen phosphorylase | PYGL | 1 |
| Glycogen synthase 1 (muscle) | GLYS1 | 1 |
| Glycogen synthase 2 (liver) | GYS2 | 1 |
| Glycosyltransferases, ABO blood group | ABO | 1 |
| Glypican 3 | GPC3, SDYS | 6 |
| GM2 ganglioside activator protein, GM2A | GM2A | 1 |
| Gonadotropin releasing hormone | GNRH | 6 |
| Gonadotropin releasing hormone receptor | GNRHR | 6 |
| Goosecoid | GSC | 6 |
| Green cone pigment | GCP | 3 |
| Growth arrest-specific homeobox | GAX | 6 |
| Growth factor receptor-bound protein 2 | GRB2 | 6 |
| Growth hormone 1 | GH1 | 6 |
| Growth hormone 2 (placental) | GH2 | 6 |
| Growth hormone receptor | GHR | 6 |
| Growth hormone releasing hormone (GHRH) | GHRH | 6 |
| Growth hormone releasing hormone receptor | GHRHR | 6 |
| Growth/differentiation factor 5 | GDF5 | 6 |
| Growth-regulated protein precursor, GRO | GRO | 4 |
| GTP cylcohydrolase 1 | GCH1 | 6 |
| GTPase-activating protein, GAP | RASA1 | 6 |
| Guanidinoacetate N-methyltransferase | GAMT | 1 |
| Guanine nucleotide-binding protein, alpha | GNA01 | 5 |
| activating activity polypeptide, GNAO | | |
| Guanine nucleotide-binding protein, alpha | GNA11 | 5 |
| inhibiting activity polypeptide 1, GNA11 | | |
| Guanine nucleotide-binding protein, alpha | GNA12 | 5 |
| inhibiting activity polypeptide 2, GNA12 | | |
| Guanine nucleotide-binding protein, alpha | GNA13 | 5 |
| inhibiting activity polypeptide 3, GNA13 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNAT1 | 5 |
| transducing activity polypeptide, GNAT1 | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, q | GNAQ | 5 |
| polypeptide | | |
| Guanylate cyclase 2D, membrane (retina- | GUCY2D | 1 |
| specific) | | |
| Guanylate cyclase activator 1A (retina) | GUCA1A | 1 |
| Guanylate kinase | | 1 |
| Gustducin, alpha (taste-specific G protein) | GDCA | 5 |
| Haeme regulated inhibitor kinase | | 1 |
| Haemoglobin epsilon | | 2 |
| Hairless | HR | 6 |
| Haptoglobin, alpha 1 | HPA1 | 4 |
| Haptoglobin, alpha 2 | HPA2 | 4 |
| Haptoglobin, beta | HPB | 4 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Hela tumor suppression gene | HTS1 | 6 |
| Hemochromatosis | HFE | 2 |
| Hemopexin | HPX | 4 |
| Heparan sulfamidase | | 1 |
| Heparin binding epidermal growth factor | HBEGF | 6 |
| Hepatic nuclear factor-3-beta | HNF3B | 1 |
| Hepatic nuclear factor-4-alpha | HNF4A | 1 |
| Hepatitis B virus integration site 1 | HVBS1 | 4 |
| Hepatitis B virus integration site 2 | HVBS6 | 4 |
| Hepatocyte growth factor | HGF | 6 |
| Hexosaminidase A | HEXA, TSD | 1 |
| Hexosaminidase B | HEXB | 1 |
| High mobility group protein 1 | HMG1 | 6 |
| High mobility group protein 2 | HMG2 | 6 |
| High mobility group protein C | HMGIC | 6 |
| High mobility group protein Y | HMGIY | 6 |
| Histone family H1 | H1 | 6 |
| Histone family H2 | H2 | 6 |
| Histone family H3 | H3 | 6 |
| Histone family H4 | H4 | 6 |
| HLA-B associated transcript 1 | BAT1 | 4 |
| HLH transcription factor HAND1 | HAND1 | 6 |
| HLH transcription factor HAND2 | HAND2 | 6 |
| HMG-CoA lyase | HMGCL | 1 |
| HMG-CoA reductase | HMGCR | 1 |
| HMG-CoA synthase | HMGCS2 | 1 |
| Holocarboxylase synthetase | HLCS | 1 |
| Holoprosencephaly 1 | HPE1 | 6 |
| Holoprosencephaly 2 | HPE2 | 6 |
| Holoprosencephaly 3 | HPE3 | 6 |
| Holoprosencephaly 4 | HPE4 | 6 |
| Homeobox (HOX) gene A1 | HOXA1 | 6 |
| Homeobox (HOX) gene A10 | HOXA10 | 6 |
| Homeobox (HOX) gene A11 | HOXA11 | 6 |
| Homeobox (HOX) gene A12 | HOXA12 | 6 |
| Homeobox (HOX) gene A13 | HOXA13 | 6 |
| Homeobox (HOX) gene A2 | HOXA2 | 6 |
| Homeobox (HOX) gene A3 | HOXA3 | 6 |
| Homeobox (HOX) gene A4 | HOXA4 | 6 |
| Homeobox (HOX) gene A5 | HOXA5 | 6 |
| Homeobox (HOX) gene A6 | HOXA6 | 6 |
| Homeobox (HOX) gene A7 | HOXA7 | 6 |
| Homeobox (HOX) gene A8 | HOXA8 | 6 |
| Homeobox (HOX) gene A9 | HOXA9 | 6 |
| Homeobox (HOX) gene B1 | HOXB1 | 6 |
| Homeobox (HOX) gene B2 | HOXB2 | 6 |
| Homeobox (HOX) gene B3 | HOXB3 | 6 |
| Homeobox (HOX) gene B4 | HOXB4 | 6 |
| Homeobox (HOX) gene B5 | HOXB5 | 6 |
| Homeobox (HOX) gene B6 | HOXB6 | 6 |
| Homeobox (HOX) gene B7 | HOXB7 | 6 |
| Homeobox (HOX) gene B8 | HOXB8 | 6 |
| Homeobox (HOX) gene B9 | HOXB9 | 6 |
| Homeobox (HOX) gene C13 | HOXC13 | 6 |
| Homeobox (HOX) gene C4 | HOXC4 | 6 |
| Homeobox (HOX) gene C8 | HOXC8 | 6 |
| Homeobox (HOX) gene C9 | HOXC9 | 6 |
| Homeobox (HOX) gene D1 | HOXD1 | 6 |
| Homeobox (HOX) gene D10 | HOXD10 | 6 |
| Homeobox (HOX) gene D12 | HOXD12 | 6 |
| Homeobox (HOX) gene D13 | HOXD13 | 6 |
| Homeobox (HOX) gene D3 | HOXD3 | 6 |
| Homeobox (HOX) gene D4 | HOXD4 | 6 |
| Homeobox (HOX) gene D8 | HOXD8 | 6 |
| Homeobox (HOX) gene D9 | HOXD9 | 6 |
| Homeobox 11 | HOX11 | 6 |
| Homeobox HB24 | HLX1 | 6 |
| Homeobox HB9 | HLXB9 | 6 |
| Homeobox, PROX1 | PROX1 | 6 |
| HSSB, replication protein | | 1 |
| Human atonal gene | ATOH1 | 6 |
| Human chorionic gonadtrophin, hCG | CG | 6 |
| Human placental factogen | CSH1 | 6 |
| Huntingtin | HD | 2 |
| Hypoxanthine-guanine | HPRT | 1 |
| phosphoribosyltransferase, HGPRT | | |
| Hypoxia inducible factor 1 | HIF1A | 1 |
| Hypoxia inducible factor 2 | | 1 |
| IC7 A and B | | 4 |
| Iduronate 2 sulphatase | IDS | 1 |
| Ikaros gene | IKAROS | 6 |
| Immunoglobulin alpha (IgA) | IGHA | 4 |
| Immunoglobulin delta (IgD) | IGHD | 4 |
| lmmunoglobulin E (Ig) reponsivigness gene | IGER | 4 |
| Immunoglobulin E (IgE) serum concentration | IGES | 4 |
| regulator gene | | |
| Immunoglobulin epsilon (IgE) | IGHE | 4 |
| Immunoglobulin gamma (IgG) 2 | IGHG2 | 4 |
| Immunoglobulin heavy mu chain | IGHM | 4 |
| Immunoglobulin J polypeptide | IGJ | 4 |
| Immunoglobulin kappa constant region | IGKC | 4 |
| lmmunoglobulin kappa variable region | IGKV | 4 |
| Indian hedgehog, ihh | IHH | 6 |
| Inhibin, alpha | INHA | 6 |
| Inhibin, beta A | INHBA | 6 |
| Inhibin, beta B | INHBB | 6 |
| Inhibin, beta C | INHBC | 6 |
| Inosine monophosphate dehydrogenase, | | 1 |
| IMPDH | | |
| Inositol 1,4,5-triphosphate receptor 1 | ITPR1 | 6 |
| Inositol 1,4,5-triphosphate receptor 3 | ITPR3 | 6 |
| Insulin | INS | 6 |
| Insulin promotor factor 1 | IPF1 | 6 |
| Insulin receptor | INSR | 6 |
| Insulin receptor substrate-1 | IRS1 | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| Integrin beta 1 | ITGB1 | 6 |
| Integrin beta 2 | ITGB2 | 6 |
| Integrin beta 3 | ITGB3 | 6 |
| Integrin beta 4 | ITGB4 | 6 |
| Integrin beta 5 | ITGB5 | 6 |
| Integrin beta 6 | ITGB6 | 6 |
| Integrin beta 7 | ITGB7 | 6 |
| Integrin, alpha 1 | ITGA1 | 6 |
| Integrin, alpha 2 | ITGA2 | 6 |
| Integrin, alpha 3 | ITGA3 | 6 |
| Integrin, alpha 4 | ITGA4 | 6 |
| Integrin, alpha 5 | ITGA5 | 6 |
| Integrin, alpha 6 | ITGA6 | 6 |
| Integrin, alpha 7 | ITGA7 | 6 |
| lntegrin, alpha 8 | ITGA8 | 6 |
| Integrin, alpha 9 | ITGA9 | 6 |
| Integrin, alpha M | ITGAM | 6 |
| Integrin, alpha X | ITGAX | 6 |
| Inter-alpha-trypsin inhibitor, IATI | | 1 |
| lntercellular adhesion molecule 1 | ICAM1 | 4 |
| Intercellular adhesion molecule 2 | ICAM2 | 4 |
| lntercellular adhesion molecule 3 | ICAM3 | 4 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| lnterleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| Interleukin(IL) 10 receptor | IL10R | 4 |
| Interleukin(IL) 11 | IL11 | 4 |
| Interleukin(IL) 11 receptor | IL11R | 4 |
| lnterleukin(IL) 12 | IL12 | 4 |
| Interleukin(IL) 12 receptor, beta 1 | IL12RB1 | 4 |
| Interleukin(IL) 13 | IL13 | 4 |
| Interleukin(IL) 13 receptor | IL13R | 4 |
| lnterleukin(IL) 2 | IL2 | 4 |
| lnterleukin(IL) 2 receptor, alpha | IL2RA | 4 |
| lnterleukin(IL) 2 receptor, gamma | IL2RG | 4 |
| Interleukin(IL) 8 | IL3 | 4 |
| lnterleukin(IL) 3 receptor | IL3R | 4 |
| lnterleukin(IL) 4 | IL4 | 4 |
| Interleukin(IL) 4 receptor | IL4R | 4 |
| lnterleukin(IL) 5 | IL5 | 4 |
| lnterleukin(IL) 5 receptor | IL5R | 4 |
| lnterleukin(IL) 6 | IL6 | 4 |
| Interleukin(IL) 6 receptor | IL6R | 4 |
| lnterleukin(IL) 7 | IL7 | 4 |
| Interleukin(IL) 7 receptor | IL7R | 4 |
| lnterleukin(IL) 8 | IL8 | 4 |
| lnterleukin(IL) 8 receptor | IL8R | 4 |
| lnterleukin(IL) 9 | IL9 | 4 |
| lnterleukin(IL) 9 receptor | IL9R | 4 |
| Interleukin(IL) receptor antagonist 1 | IL1RN, ILLRA | 4 |
| IP3 kinase | | 1 |
| Isocitrate dehydrogenase | | 1 |
| Isovaleric acid CoA dehydrogenase | IVD | 1 |
| Janus kinase 1 | JAKI | 6 |
| Janus kinase 2 | JAK2 | 6 |
| Janus kinase 3 | JAK3 | 6 |
| Kallman syndrome gene 1 | KAL1 | 6 |
| Kell blood group precursor | XK, KEL | 2 |
| Keratin 1 | KRT1 | 3 |
| Keratin 10 | KRT10 | 3 |
| Keratin 11 | KRT11 | 3 |
| Keratin 12 | KRT12 | 3 |
| Keratin 13 | KRT13 | 3 |
| Keratin 14 | KRT14 | 3 |
| Keratin 15 | KRT15 | 3 |
| Keratin 16 | KRT16 | 3 |
| Keratin 17 | KRT17, PCHC1 | 3 |
| Keratin 18 | KRT18 | 3 |
| Keratin 2 | KRT2 | 3 |
| Keratin 3 | KRT3 | 3 |
| Keratin 4 | KRT4 | 3 |
| Keratin 5 | KRT5 | 3 |
| Keratin 6 | KRT6 | 3 |
| Keratin 7 | KRT7 | 3 |
| Keratin 8 | KRT8 | 3 |
| Keratin 9 | KRT9 | 3 |
| Ketohexokinase | KHK | 1 |
| Kinectin | KTN1 | 6 |
| Kinesin, heavy chain | KNSL1 | 6 |
| Kinesin, light chain | KNS2 | 6 |
| L1 cell adhesion molecule | L1CAM | 5 |
| Lactotransferrin | LTF | 2 |
| Lamin A/C | LMNA | 6 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMR1 | 6 |
| Latent transforming growth factor-beta binding | LTBP2 | 6 |
| protein 2 | | |
| Leptin | LEP | 6 |
| Leptin receptor | LEPR | 6 |
| Leukaemia inhibitory factor | LIF | 6 |
| Leukaemia inhibitory factor receptor | LIFR | 6 |
| Leukin | | 4 |
| Leukocyte-specific transcript 1 | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| Leukotriene D4/E4 receptor | | 4 |
| LH/choriogonadotropin (CG) receptor | LHCGR | 6 |
| LIM homeobox protein 1 | LHX1 | 6 |
| LIM homeobox protein 2 | LHX2 | 6 |
| LIM homeobox protein 3 | LHX3 | 6 |
| LIM homeobox protein 4 | LHX4 | 6 |
| LIM homeobox transcription factor 1, beta | LMX1B | 6 |
| Limb girdle muscular dystrophy 1 A | LGMD1A | 6 |
| Limb girdle muscular dystrophy 1 B | LGMDI B | 6 |
| Limb girdle muscular dystrophy 2G | LGMD2G | 6 |
| Limb girdle muscular dystrophy 2H | LGMD2H | 6 |
| Limbic associated membrane protein | LAMP | 6 |
| LIM-domain only protein 1 | LM01 | 6 |
| LIM-domain only protein 2 | LM02 | 6 |
| LIM-domain only protein 3 | LM03 | 6 |
| LIM-domain only protein 4 | LM04 | 6 |
| Lipoma-preferred partner gene | LPP | 6 |
| Lipoprotein receptor, Low Density | LDLR | 2 |
| Lipoxygenase 12 (platelets) | LOG12 | 4 |
| Lipoxygenase 5 (leukocytes) | | 4 |
| Long QT-type 2 potassium channels | LQT2, KCNH2 | 2 |
| Loricrin | LOR | 3 |
| Low density lipoprotein receptor-related protein LRP | | 2 |
| precursor | | |
| Luteinizing hormone, beta chain | LHB | 6 |
| Lymphoblastic leukemia derived sequence 1 | LYL1 | 4 |
| Lymphocyte-specific protein tymsine kinase | LCK | 4 |
| Lymphoid enhancer-binding factor | LEF-1 | 6 |
| Lysosome-associated membrane protein 1 | LAMP1 | 6 |
| Lysosome-associated membrane protein 2 | LAMP2 | 6 |
| MAD (mothers against decapentaplegic, | MADH2 | 6 |
| Drosophila) homologue 2 | | |
| MAD (mothers against decapentaplegic, | MADH3 | 6 |
| Drosophila) homologue 3 | | |
| MAD (mothers against decapentaplegic, | MADH4 | 6 |
| Dmsophila) homologue 4 | | |
| MADS box transcription-enhancer factor 2A | MEF2A | 6 |
| MADS box transcription-enhancer factor 2B | MEF2B | 6 |
| MADS box transcription-enhancer factor 2C | MEF2C | 6 |
| MADS box transcription-enhancer factor 2D | MEF2D | 6 |
| Malate dehydrogenase, mitochondrial | MDH2 | 1 |
| Malignant proliferation, eosinophil gene | MPE | 4 |
| Malonyl CoA decarboxylase | | 1 |
| Malonyl CoA transferase | | 1 |
| Mannosidase, alpha B lysosomal | MANB | 1 |
| Mannosidase, beta A lysosomal | MANBA | 1 |
| MAPK kinase 1 | MAPKK1; MEK1 | 6 |
| MAPK kinase 4 | MAPKK4; MEK4; | 6 |
| | SERK1 | |
| MAPK kinase 6 | MAPKK6; MEK6 | 6 |
| MAPKK kinase | MAPKKK | 6 |
| Matrix Gla protein | MGP | 6 |
| Matrix metalloproteinase 1 | NMP1 | 1 |
| Matrix metalloproteinase 10 | NMP10 | 1 |
| Matrix metalloproteinase 11 | MMP11 | 1 |
| Matrix metalloproteinase 12 | MMP12 | 1 |
| Matrix metalloproteinase 13 | MMP13 | 1 |
| Matrix metalloproteinase 14 | MMP14 | 1 |
| Matrix metalloproteinase 15 | MMP15 | 1 |
| Matrix metalloproteinase 16 | MMP16 | 1 |
| Matrix metalloproteinase 17 | MMP17 | 1 |
| Matrix metalloproteinase 18 | MMP18 | 1 |
| Matrix metalloproteinase 19 | MMP19 | 1 |
| Matrix metalloproteinase 2 | MMP2 | 1 |
| Matrix metalloproteinase 3 | MMP3, STMY1 | 1 |
| Matrix metalloproteinase 4 | MMP4 | 1 |
| Matrix metalloproteinase 5 | MMP5 | 1 |
| Matrix metalloproteinase 6 | MMP6 | 1 |
| Matrix metalloproteinase 7 | MMP7 | 1 |
| Matrix metalloproteinase 8 | MMP8 | 1 |
| Matrix metalloproteinase 9 | MMP9 | 1 |
| MAX-interacting protein 1 | MXI1 | 6 |
| MEK kinase, MEKK | | 1 |
| Melanocortin 1 receptor | MC1R | 2 |
| Melanocortin 2 receptor | MC2R | 2 |
| Melanocortin 4 receptor | MC4R | 2 |
| Menin | MEN1 | 6 |
| Mesoderm-specific transcript | MEST | 6 |
| Methionine adenosyltransferase | MAT1A, MAT2A | 1 |
| Methionine synthase | MTR | 1 |
| Methionine synthase reductase | MTRR | 1 |
| Methylguanine-DNA methyltransferase | MGMT | 1 |
| Methylmalonyl-CoA mutase | MUT | 1 |
| Mevalonate kinase | MVK | 1 |
| MHC Class 1: A | | 4 |
| MHC Class 1: B | | 4 |
| MHC Class 1: C | | 4 |
| MHC Class 1: LMP-2, LMP-7 | | 4 |
| MHC Class 1: Tap1 | ABCR, TAP1 | 4 |
| MHC Class II: DP | HLA-DPB1 | 4 |
| MHC Class II: DQ | | 4 |
| MHC Class II: DR | | 4 |
| MHC Class II: Tap2 | TAP2, PSF2 | 4 |
| MHC Class II: Complementation group A | MHC2TA | 4 |
| MHC Class II: Complementation group B | RFXANK | 4 |
| MHC Class II: Complementation group C | RFX5 | 4 |
| MHC Class II: Complementation group D | RFXAP | 4 |
| Microphthalmia-associated transcription factor | MITF | 6 |
| Microsomal triglyceride transfer protein | MTP | 2 |
| Microtuble associated protein | MAP | 3 |
| Midline I | MID1 | 6 |
| Mismatch repair gene, PMSL1 | PMS1 | 6 |
| Mismatch repair gene, PMSL2 | PMS2 | 6 |
| Mitochondrial trifunctional protein, alpha | HADHA | 1 |
| subunit | | |
| Mitochondrial trifunctional protein, beta subunit | HADHB | 1 |
| Mitogen-activated protein (MAP) kinase | MAPK | 6 |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Monocyte chemoattractant protein 1 | MCP1 | 4 |
| Motilin | MLN | 6 |
| Msh homeobox homolog 1 | MSX1 | 6 |
| Msh homeobox homolog 2 | MSX2 | 6 |
| Mucolipidoses | GNPTA | 1 |
| Mulibrey nanism | MUL | 2 |
| Multidrug resistance associated protein | MRP | 6 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Muscle phosphorylase | PYGM | 1 |
| Mutated in colorectal cancers, MCC | MCC | 6 |
| MutL homolog 1 | MLH1 | 6 |
| MutS homolog 2 | MSH2 | 6 |
| MutS homolog 3 | MSH3 | 6 |
| Myelin protein peripheral 22 | PMP22 | 3 |
| Myelin protein zero | MPZ | 3 |
| Myelodysplasia syndrome 1 gene | MDS1 | 6 |
| Myeloid leukemia factor-1 | MLF1 | 4 |
| Myocilin | MYOC | 2 |
| Myogenic factor 3 | MYF3 | 6 |
| Myogenic factor 4 | MYF4 | 6 |
| Myogenic factor 5 | MYF5 | 6 |
| Myomesin 1 | MYOM1 | 3 |
| Myomesin 2 | MYOM2 | 3 |
| Myosin 15 | MYO15 | 3 |
| Myosin 6 | MY06 | 3 |
| Myosin 7A | MY07A | 3 |
| Myosin, cardiac | MYH7 | 3 |
| Myotubularin | MTM1 | 3 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+/H+ exchanger 1 | NHE1 | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| N-acetylgalactosamine-6-sulfate sulfatase | GALNS | 1 |
| N-acetylglucosamine-6-sulfatase | GNS | 1 |
| N-acetylglucosaminidase, alpha | NAGLU | 1 |
| N-acetyltransferase 1 | NAT1 | 1 |
| N-acetyltransferase 2 | NAT2 | 1 |
| NADH dehydrogenase | | 1 |
| NADH dehydrogenase (ubiquinone) Fe—S | NDUFS1 | 1 |
| protein 1 | | |
| NADH dehydrogenase (ubiquinone) Fe—S | NDUFS4 | 1 |
| protein 4 | | |
| NADH dehydrogenase (ubiquinone) | NDUFV1 | 1 |
| flavoprotein 1 | | |
| NADH-cytochrome b5 reductase | DIA1 | 1 |
| reductase | | |
| NADPH-dependent cytochrome P450 | POR | 1 |
| reductase | | |
| Natural resistance-associated macrophage | NRAMP1 | 4 |
| protein 1 | | |
| NB6 | | 4 |
| Necdin | NDN | 6 |
| Nephronophthisis 1 | NPHP1 | 2 |
| Nephronophthisis 2 | NPHP2 | 2 |
| Nephrosis 1 | NPHS1 | 2 |
| Nerve growth factor | NGF | 6 |
| Nerve growth factor receptor | NGFR | 6 |
| Neural retina-specific gene | NRL | 6 |
| Neuraminidase sialidase | NEU | 2 |
| Neuregulin | HGL | 6 |
| Neurite growth-promoting factor 2 | MDK | 5 |
| Neurite inhibitory protein | | 5 |
| Neuroendocrine convertase 1 | NEC1, PCSK1 | 1 |
| Neurofibromin 1 | NFI | 6 |
| Neurofibromin 2 | NF2 | 6 |
| Neurofliament protein, heavy | NFH | 3 |
| Neurofliament protein, NF68 | NF68 | 3 |
| Neuronal apoptosis inhibitory protein | NAIP | 4 |
| Neuronal molecule-1 | | 4 |
| Neuronal molecule-1 receptor | | 4 |
| Neuropeptide Y | NPY | 5 |
| Neuropeptide Y receptor Y1 | NPY1 R | 5 |
| Neuropeptide Y receptor Y2 | NPY2R | 5 |
| Neurotrophic tyrosine kinase receptor 1 | NTRK1 | 6 |
| Neurotrophin 3 | NTF3 or NT3 | 6 |
| Neurturin | NRTN | 6 |
| Neutral endopeptidase | | 1 |
| Neutrophil cystolic factor 1 | NCF1 | |
| Neutrophil cystolic factor 2 | NCF2 | |
| Niacin receptor | | 6 |
| Nibrin | NBS1 | 6 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Nodal | NODAL | 6 |
| Noggin | NOG | 6 |
| Norrie disease protein | NDP | 6 |
| Notch 1 | NOTCH1 | 6 |
| Notch 2 | NOTCH2 | 6 |
| Notch 3 | NOTCH3 | 6 |
| Notch ligand-jagged 1 | JAG 1, AGS | 6 |
| Nuclear factor I-kappa-B-like gene | IKBL | 4 |
| Nuclear factor kappa beta | NFKB | 4 |
| Nuclear factor of activated T cells (NFAT) | NFATC | 6 |
| complex, cytosolic | | |
| Nuclear factor of activated T cells (NFAT) | NFATP | 6 |
| complex, preexisting component | | |
| Nuclear mitotic apparatus protein 1 | NUMA1 | 6 |
| Nucleophosmin | NPM1 | 2 |
| Nucleoside diphosphate kinase-A | NDPKA | 1 |
| Ocular albinism 1 | OA1 | 3 |
| Oculocutaneous albinism II | OCA2 | 3 |
| Oligophrenin-1 | OPHN1 | 6 |
| Oncogene abl1 | ABL1 | 6 |
| Oncogene abl2 | | 6 |
| Oncogene akt1 | | 6 |
| Oncogene akt2 | AKT2 | 6 |
| Oncogene axI | AXL | 6 |
| Oncogene bcl2 | | 6 |
| Oncogene bcr/abl | | 6 |
| Oncogene B-lym | | 6 |
| Oncogene B-raf | | 6 |
| Oncogene clk1 | | 6 |
| Oncogene c-myc | | 6 |
| Oncogene cot | | 6 |
| Oncogene crk | | 6 |
| Oncogene crkI | | 6 |
| Oncogene ect2 | | 6 |
| Oncogene ELK1 | ELK1 | 6 |
| Oncogene ELK2 | ELK2 | 6 |
| Oncogene emsl | | 6 |
| Oncogene ERB | | 6 |
| Oncogene ERB2 | | 6 |
| Oncogene ERBA | | 6 |
| Oncogene ERBAL2 | | 6 |
| Oncogene ERG (early reponse gene) | | 6 |
| Oncogene ETS1 | | 6 |
| Oncogene ETS2 | | 6 |
| Oncogene EVI1 | EVI 1 | 6 |
| Oncogene fes | | 6 |
| Oncogene fgr | | 6 |
| Oncogene fos | FOS | 6 |
| Oncogene fps | | 6 |
| Oncogene GLI1 | GLI | 6 |
| Oncogene GLI2 | GLI2 | 6 |
| Oncogene GLI3 | GLI3 | 6 |
| Oncogene gro1 | | 6 |
| Oncogene gro2 | | 6 |
| Oncogene Ha-ras | HRAS | 6 |
| Oncogene hs1 | | 6 |
| Oncogene hst | FGF4 | 6 |
| Oncogene int1 | WNT1 | 6 |
| Oncogene int2 | FGF3 | 6 |
| Oncogene int3 | Notch4 | 6 |
| Oncogene int4 | WNT3 | 6 |
| Oncogene jun | JUN | 6 |
| Oncogene KIT | KIT, PBT | 6 |
| Oncogene LCO | LCO | 6 |
| Oncogene I-myc | | 6 |
| Oncogene Ipsa | | 6 |
| Oncogene lyn | | 6 |
| Oncogene maf | | 6 |
| Oncogene mas1 | | 6 |
| Oncogene mcf2 | | 6 |
| Oncogene mdm2 | MDM2 | 6 |
| Oncogene meI | | 6 |
| Oncogene met | MET | 6 |
| Oncogene mos | | 6 |
| Oncogene mpI | | 6 |
| Oncogene MUM1 | MUM1 | 6 |
| Oncogene myb | MYB | 6 |
| Oncogene myc | MYC | 6 |
| Oncogene n-myc | | 6 |
| Oncogene N-ras (neuroblastoma v-ras) | NRAS | 6 |
| Oncogene ovc | | 6 |
| Oncogene pimi | | 6 |
| Oncogene pti-1sea | | 6 |
| Oncogene pvt1 | | 6 |
| Oncogene raf | RAF | 6 |
| Oncogene ralb | | 6 |
| Oncogene reI | | 6 |
| Oncogene ret | RET | 6 |
| Oncogene r-myc | | 6 |
| Oncogene ros | | 6 |
| Oncogene R-ras | | 6 |
| Oncogene sis | PDGFB | 6 |
| Oncogene ski | | 6 |
| Oncogene sno | | 6 |
| Oncogene spi1 | | 6 |
| Oncogene src | | 6 |
| Oncogene tc21 | | 6 |
| Oncogene TEL | ETV6 | 6 |
| Oncogene tim | | 6 |
| Oncogene vavtrk | | 6 |
| Oncogene v-Ki-ras2 | KRAS2 | 6 |
| Oncogene yes | | 6 |
| Oncogene yuasa | | 6 |
| Oncostatin M | OSM | 6 |
| Oncostatin M receptor | OSMR | 6 |
| Orexin | OX | 6 |
| Orexin 1 receptor | OXIR | 6 |
| Orexin 2 receptor | OX2R | 6 |
| Ornithine delta-aminotransferase | OAT | 1 |
| Ornithine transcarbamoylase | OTC, NME1 | 1 |
| Orthodenticle (Drosophila) homolog 1 | OTX1 | 6 |
| Orthodenticle (Drosophila) homolog 2 | OTX2 | 6 |
| Osteocalcin | | 3 |
| Osteonectin | ON | 6 |
| Osteopontin | OPN | 6 |
| Osteoprotegerin | OPG | 6 |
| Otoferlin | OTOF | 5 |
| Oxytocin | OXT | 5 |
| Oxytocin receptor | OXTR | 5 |
| p21-activated kinase 3 | PAK3 | 6 |
| Paired box homeotic gene 1 | PAX1 | 6 |
| Paired box homeotic gene 2 | PAX2 | 6 |
| Paired box homeotic gene 3 | PAX3 | 6 |
| Paired box homeotic gene 6 | PAX6 | 6 |
| Paired box homeotic gene 7 | PAX7 | 6 |
| Paired box homeotic gene 8 | PAX8 | 6 |
| Paired-like homeodomain transcription factor 2 | PITX2 | 6 |
| Paired-like homeodomain transcription factor 3 | PITX3 | 6 |
| Palmitoyl-protein thioesterase | PPT | 2 |
| Pancreatic amylase | | 1 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Parvalbumin | PVALB | 6 |
| Patched (Drosophila) homolog, PTCH | PTCH | 6 |
| PCNA (proliferating cell nuclear antigen) | | 1 |
| Peanut-like 1 | PNUTL1 | 4 |
| Pendrin, PDS | PDS | 2 |
| Peptidylglycine alpha-amidating | PAM | 1 |
| monooxygenase | | |
| Peripherin, PRPH | | 3 |
| Peroxisomal membrane protein 1 | PXMP1 | 3 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome proliferative activated receptor, | PPARA | 2 |
| alpha | | |
| Peroxisome proliferative activated receptor, | PPARG | 2 |
| gamma | | |
| Peroxisome receptor 1 | PXR1 | 2 |
| Phenylethanolamine N-methyltransferase, | PNMT | 1 |
| PNMT | | |
| Phosphatase & tensin homolog | PTEN | 6 |
| Phosphate regulating gene with homologies to | PHEX | 6 |
| endopeptidases on the X chromosome | | |
| Phosphatidylinositol glycan, class A | PIGA | 6 |
| (paroxysmal nocturnal hemoglobinuria) | | |
| Phosphatidylinositol transfer protein | PITPN | 6 |
| Phosphodiesterase 1/nucleotide | PDNP1 | 6 |
| pyrophosphatase 1 | | |
| Phosphodiesterase 1/nucleotide | PDNP2 | 6 |
| pyrophosphatase 2 | | |
| Phosphodiesterase 1/nucleotide | PDNP3 | 6 |
| pyrophosphatase 3 | | |
| Phosphofructokinase, liver | PFKL | 1 |
| Phosphofructokinase, muscle | PFKM | 1 |
| Phosphoglucose isomerase | GPI | 1 |
| Phosphoglycerate kinase 1 | PGKL | 1 |
| Phosphoglycerate mutase 2 | PGAM2 | 1 |
| Phospholipase A2, group 10 | PLA2G10 | 4 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase 1 | PMM1 | 6 |
| Phosphomannomutase 2 | PMM2 | 6 |
| Phosphomannomutase 2 | PMM2 | 2 |
| Phosphorylase kinase deficiency, liver | PHK | 1 |
| Phosphorylase kinase, alpha 2 | PHKA2 | 1 |
| Phytanoyl-CoA hydroxylase | PHYH | 6 |
| Plakophilin 1 | PKP1 | 2 |
| Plasminogen | PLG | 1 |
| Plasminogen activator inhibitor 1 | PAI 1 | 1 |
| Plasminogen activator inhibitor 2 | PAI 2 | 1 |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Plectin 1 | PLEC1 | 2 |
| Poly (ADP-ribose) synthetase | PARS | 1 |
| Poly(A) binding protein 2 | PABP2 | 6 |
| Postsynaptic density-95 protein | PSD95 | 5 |
| Potassium inwardly-rectifying channel J1 | KCNJL | 5 |
| Potassium inwardly-rectifying channel J11 | KCNJL11 | 5 |
| Potassium voltage-gated channel A1 | KCNA1 | 5 |
| Potassium voltage-gated channel E1 | KCNE1 | 5 |
| Potassium voltage-gated channel Q1 | KCNQ1 | 5 |
| Potassium voltage-gated channel Q2 | KCNQ2 | 5 |
| Potassium voltage-gated channel Q3 | KCNQ3 | 5 |
| Potassium voltage-gated channel Q4 | KCNQ4 | 5 |
| POU domain, class 1, transcription factor 1 | POU1F1 | 6 |
| (Piti) | | |
| POU domain, class 3, transcription factor 4 | POU3F4 | 6 |
| POU domain, class 4, transcription factor 3 | POU4F3 | 6 |
| Pre-B-cell leukemia transcription factor 1 | PBX1 | 6 |
| Preproglucagon | GCG; GLPL; GLP2 | 6 |
| Procoliagen N-protease | | 1 |
| Procollagen peptidase | | 1 |
| Profibrinolysin | | 6 |
| receptor) | | |
| Prohibitin | PHB | 6 |
| Prolactin | PRL | 6 |
| Prolactin receptor | PRLR | 6 |
| Prolactin releasing hormone | PRH | 6 |
| Proliferin | PLF | 6 |
| Proline dehydrogenase | PRODH | 1 |
| Pro-melanin-concentrating hormone | PMCH | 6 |
| Promyelocytic leukemia gene | PML | 6 |
| Proopiomeianocortin | POMC | 5 |
| Prophet of Pit1 | PROP1 | 6 |
| Propionyl-CoA carboxylase, alpha | PCCA | 1 |
| Propionyl-CoA carboxylase, beta | PCCB | 1 |
| Prosaposin | PSAP | 5 |
| Prostaglandin (PG) D synthase, hematopoietic | PGDS | 1 |
| Prostaglandin isomerase | | 6 |
| Prostaglandin-endoperoxidase synthase 2 | PTGS2 | 6 |
| Prostate cancer anti-metastasis gene KAI 1 | KAI 1 | 6 |
| Protease nexin 2 | PN2 | 1 |
| Protective protein for beta-galactosidase | PPGB | 1 |
| Protein C | PROC | 4 |
| Protein kinase A | | 1 |
| Protein kinase B | PRKB | |
| Protein kinase C, alpha | PRKCA | 1 |
| Protein kinase C, gamma | PRKCG | 1 |
| Protein kinase DNA-activated | PRKDC | 1 |
| Protein kinase G | | 1 |
| Protein phosphatase 1, regulatory (inhibitor) | PPP1R3 | 1 |
| subunit 3 | | |
| Protein phosphatase 2, regulatory subunit A, | PPP2R1B | 1 |
| beta isoform | | |
| Protein tyrosine phosphatase, non-receptor | PTPN12 | 6 |
| type 12 | | |
| Protoporphyrinogen oxidase | PPOX | 1 |
| Pterin-4-alpha-carbinolamine | PCBD | |
| Purine nucleoside phosphorylase | NP | 1 |
| Purinergic receptor P1A1 | | 5 |
| Purinergic receptor P1A2 | | 5 |
| Purinergic receptor P1A3 | | 5 |
| Purinergic receptor P2X, 1 | P2RX1 | 5 |
| Purinergic receptor P2X, 2 | P2RX2 | 5 |
| Purinergic receptor P2X, 3 | P2RX3 | 5 |
| Purinergic receptor P X, 4 | P2RX4 | 5 |
| Purinergic receptor P2X, 5 | P2RX5 | 5 |
| Purinergic receptor P9X, 6 | P2RX6 | 5 |
| Purinergic receptor P2X, 7 | P2RX7 | 5 |
| Purinergic receptor P2Y, 1 | P2RY1 | 5 |
| Purinergic receptor P2Y, 11 | P2RY11 | 5 |
| Purinergic receptor P2Y, 2 | P2RY2 | 5 |
| Pyrroline-5-carboxylate synthetase | PYCS | 1 |
| Pyruvate carboxylase | PC | 1 |
| Pyruvate decarboxylase | PDHA | 1 |
| Pyruvate kinase | PKLR | 1 |
| RAD51, DNA repair protein | RAD51 | 6 |
| RAD52, DNA repair protein | RAD52 | 6 |
| RAD54, DNA repair protein | RAD54 | 6 |
| RAD55, DNA repair protein | RAD55 | 6 |
| RAD57, DNA repair protein | RAD57 | 6 |
| Ras-G-protein | RAS | 6 |
| Rathke pouch homeobox, RPX | RPX | 6 |
| Receptor tyrosine kinase (RTK), Nsk2 | NSK2 | 6 |
| Recombination activating gene 1 | RAG1 | 6 |
| Recombination activating gene 2 | RAG2 | 6 |
| Red cone pigment | RCP | 3 |
| Relaxin H1 | RLN1 | 6 |
| Relaxin H2 | RLN2 | 6 |
| Replication factor A | | 1 |
| Replication factor C | RFC2 | 1 |
| Retinal pigment epithelium specific protein | RPE65 | 3 |
| (65 kD) | | |
| Retinitis pigmentosa gene 1 | RP1 | 3 |
| Retinitis pigmentosa gene 2 | RP2 | 3 |
| Retinitis pigmentosa gene 3 | RP3 | 3 |
| Retinitis pigmentosa gene 6 | RP6 | 3 |
| Retinitis pigmentosa gene 7 | RP7, RDS | 3 |
| Retinoblastoma 1 | RB1 | 6 |
| Retinoic acid receptor, alpha | RARA | 6 |
| Retinoic acid receptor, beta | RARB | 6 |
| Retinoic acid receptor, gamma | RARG | 6 |
| Retinoid X receptor, alpha | RXRA | 6 |
| Retinoid X receptor, beta | RXRB | 6 |
| Retinoid X receptor, gamma | RXRG | 6 |
| Retinoschisis, X-linked, juvenile | RS | 6 |
| Rhabdoid tumors | SMARCB1 | 6 |
| Rhodopsin | RHO | 3 |
| Ribonucleotide reductase, RRM | | 1 |
| Ribosomal protein L13A | RPL13A | 6 |
| Ribosomat protein L17 | RPL17 | 6 |
| Ribosomal protein S19 | RPS19 | 1 |
| Ribosomal protein S4, X-linked | RPS4X | 1 |
| Ribosomat protein S6 kinase | RPS6KA3 | 1 |
| Ribosomal protein S9 | RPS9 | 6 |
| RIGUI | RIGUI | 6 |
| Rod outer segment membrane protein 1 | ROM1 | 3 |
| Ryanodine receptor 1, skeletal | RYR1 | 6 |
| SA homolog | SAH | 6 |
| Sal-like 1 | SALL1 | 6 |
| Secretin | SCT | 2 |
| Semaphorin A4 | SEMA4 | 3 |
| Semaphorin A5 | SEMA5 | 3 |
| Semaphorin D | | 3 |
| Semaphorin E | SEMAE | 3 |
| Semaphorin F | SEMA3/F | 3 |
| Semaphorin W | SEMAW | 3 |
| Serine/threonine kinase 11 | STK11 | 6 |
| Serine/threonine kinase 2 | STK2 | 6 |
| Serotonin N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Serum amyloid A | SAA | 2 |
| Serum amyloid P | SAP | 2 |
| Sex determining region Y, SRY | SRY | 6 |
| Short stature homeobox | SHOX | 6 |
| Sialoprotein, bone | BSP | 6 |
| Signal transducer and activator of transcription 1 | STAT1 | 6 |
| Signal transducer and activator of transcription 2 | STAT2 | 6 |
| Signal transducer and activator of transcription 3 | STAT3 | 6 |
| Signal transducer and activator of transcription 4 | STAT4 | 6 |
| Signal transducer and activator of transcription 5 | STAT5 | 6 |
| Signaling lymphocyte activation molecule | SLAM | 4 |
| Sine oculis homeobox, drosophila, homolog 1 | SIX1 | 6 |
| Sine oculis homeobox, drosophila, homolog 2 | SIX2 | 6 |
| Sine oculis homeobox, drosophila, homolog 5 | SIX5 | 6 |
| Sjoegren (Siogren) syndrome antigen A1 | SSAL | 4 |
| Slug protein | | 6 |
| Small nuclear ribonucleoprotein polypeptide N | SNRPN | 3 |
| Smoothelin | SMTN | 6 |
| Smoothened (Drosophila) homolog | SMOH | 6 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage gated, type IV, alpha | SCN4A | 5 |
| polypeptide | | |
| Sodium channel, voltage gated, type V, alpha | SCN5A | 5 |
| polypeptide | | |
| Sodium channel, voltage-gated, type 1, beta | SCN1B | 5 |
| polypeptide | | |
| Solute carrier family 1 (amino acid transporter), | SLC1A6 | 2 |
| member 6 | | |
| Solute carrier family 1 (glial high affinity | SLC1A3 | 2 |
| glutamate transporter), member 3 | | |
| Solute carrier family 1 (glutamate transporter), | SLC1A1 | 2 |
| member 1 | | |
| Solute carrier family 1 (glutamate transporter), | SLC1A2 | 2 |
| member 2 | | |
| Solute carrier family 1 (neutral amino acid | SLC1A4 | 2 |
| transporter), member 4 | | |
| Solute carrier family 10 (sodium/bile acid | SLC10A1 | 2 |
| cotransporter family), member 1 | | |
| Solute carrier family 10 (sodium/bile acid | SLC10A2 | 2 |
| cotransporter family), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 14, member 2 | SLC14A2 | 2 |
| Solute carrier family 15 (H+/peptide | SLC15A1 | 2 |
| transporter, intestinal), member 1 | | |
| Solute carrier family 15 (H+/peptide | SLC15A2 | 2 |
| transporter, kidney), member 2 | | |
| Solute carrier family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 17, member 1 | SLC17A1 | 2 |
| Solute carrier family 17, member 2 | SLC17A2 | 2 |
| Solute carrier family 18, member 3 | SLC18A3 | 2 |
| Solute carrier family 19 (folate transporter), | SLC19A1 | 2 |
| member 1 | | |
| Solute carrier family 2 (facilitated glucose | SLC2A1 | 2 |
| transporter), member 1 | | |
| Solute carrier family 2 (facilitated glucose | SLC2A2 | 2 |
| transporter), member 2 | | |
| Solute carrier family 2 (facilitated glucose | SLC2A3 | 2 |
| transporter), member 3 | | |
| Solute carrier family 2 (facilitated glucose | SLC2A4 | 2 |
| transporter), member 4 | | |
| Solute carrier family 2 (facilitated glucose | SLC2A5 | 2 |
| transporter), member 5 | | |
| Solute carrier family 20, member 1 | SLC20A1 | 2 |
| Solute carrier family 20, member 2 | SLC20A2 | 2 |
| Solute carrier family 20, member 3 | SLC20A3 | 2 |
| Solute carder family 21, member 2 | SLC21A2 | 2 |
| Solute carrier family 21, member 3 | SLC21A3 | 2 |
| Solute carrier family 22, member 1 | SLC22A1 | 2 |
| Solute carrier family 22, member 2 | SLC22A2 | 2 |
| So ute carrier family 22, member 5 | SLC22A5 | 2 |
| Solute carrier family 25, member 12 | SLC25A12 | 2 |
| Solute carrier family 3 (facilitated glucose | SLC3A1 | 2 |
| transporter), member 1 | | |
| Solute carrier family 4 (anion exchanger), | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carder family 6 (neurotransmitter | SLC6A4 | 2 |
| transporter, serotonin), member 4 | | |
| Solute carrier family 6, member 10 | SLC6A10 | 2 |
| Solute carrier family 6, member 6 | SLC6A6 | 2 |
| Solute carrier family 6, member 8 | SLC6A8 | 2 |
| Solute carrier family 7(amino acid transporter), | SLC7A1 | 2 |
| member 1 | | |
| Solute carrier family 7(amino acid transporter), | SLC7A2 | 2 |
| member 2 | | |
| Solute carrier family 7(amino acid transporter), | SLC7A7 | 2 |
| member 7 | | |
| So ute carrier family 8 (sodium/calcium | SLC8A1 | 2 |
| exchanger), member 1 | | |
| Somatostatin receptor, SSTR2 | SSTR2 | 6 |
| Somatotrophin | | 6 |
| Sonic hedgehog, SHH | SHH | 6 |
| Sorbitol dehydrogenase | SORD | 1 |
| Sorcin | SRI | 2 |
| SOSI guanine nucleotide exchange factor | SOS1 | 6 |
| Spastic paraplegia 7 | SPG7 | 6 |
| Spectrin alpha | SPTAL | 3 |
| Spectdn beta | SPTB | 3 |
| Sperm adhesion molecule | SPAM1 | 6 |
| Sperm prolamine P1 | PRM1 | 6 |
| Sperm prolamine P2 | PRM2 | 6 |
| Sphingomyelinase | SMPDI | 1 |
| Spinocerebellar ataxia 8 gene | SCA8 | 5 |
| Split hand/foot malformation gene | DSS1 | 6 |
| SRY-box 10 | SOX10 | 6 |
| SRY-box 11 | SOX11 | 6 |
| SRY-box 3 | SOX3 | 6 |
| SRY-box 4 | SOX4 | 6 |
| SRY-box 9 | SOX9 | 6 |
| Stem cell factor | SCF | 6 |
| Steroid 5 alpha reductase 1 | SRD5A1 | 1 |
| Steroid 5 alpha reductase 2 | SRD5A2 | 1 |
| Steroid hormone receptor responsive DNA | | 6 |
| elements | | |
| Steroid sulphatase | STS | 1 |
| Steroidogenic acute regulatory protein | STAR | 2 |
| Stromal derived factor 1 | SDF1 | 6 |
| Succinate dehydrogenase 1 | SDH1 | 1 |
| Succinate dehydrogenase 2 | SDH2 | 1 |
| Succinate thiokinase | | 1 |
| Succinic semi-aldehyde dehydrogenase | SSADH | 1 |
| Suffamidase | SGSH | 6 |
| Sulfite oxidase | SUOX | 1 |
| Sulfonylurea receptor | SUR | 6 |
| Suppression of tumorigenicity 3 gene | ST3 | 6 |
| Suppression of tumorigenicity 8 gene | ST8 | 6 |
| Surfactant pulmonary-associated protein A1 | SFTPA1 | 2 |
| Surfactant pulmonary-associated protein A2 | SFTPA2 | 2 |
| Surfactant pulmonary-associated protein B | SFTPB | 2 |
| Surfactant pulmonary-associated protein C | SFTPC | 2 |
| Surfactant pulmonary-associated protein D | SFTPD | 2 |
| Surfeit 1 | SURF1 | 6 |
| Survival of motor neuron 1, telomeric | SMN1 | 2 |
| SYK-related tyrosine kinase | SRK | 4 |
| Syndecan 1 | SYND1 | 6 |
| Syndecan 2 | SYND2 | 6 |
| Syndecan 3 | SYND3 | 6 |
| Syndecan 4 | SYND4 | 6 |
| Synovial sarcoma gene 1 | SSX1 | 6 |
| Synovial sarcoma gene 2 | SSX2 | 6 |
| Talin | TLN | 6 |
| TATA binding protein | TBP | 6 |
| TATA binding protein associated factor 2A | TAF2A | 6 |
| TATA binding protein associated factor 2C2 | TAF2C2 | 6 |
| TATA binding protein associated factor 2D | TAF2E | 6 |
| TATA binding protein associated factor 2F | TAF2F | 6 |
| TATA binding protein associated factor 2H | TAF2H | 6 |
| TATA binding protein associated factor 2I | TAF21 | 6 |
| TATA binding protein associated factor 2J | TAF2J | 6 |
| TATA binding protein associated factor 2K | TAF2K | 6 |
| Tau protein | MAPT | 3 |
| T-BOX 1 | TBX1 | 6 |
| T-BOX 2 | TBX2 | 6 |
| T-BOX 3 | TBX3 | 6 |
| T-BOX 4 | TBX4 | 6 |
| T-BOX 5 | TBX5 | 6 |
| T-BOX 6 | TBX6 | 6 |
| T-cell acute lymphocytic leukemia 1 | TAL1 | 4 |
| T-cell acute lymphocytic leukemia 2 | TAL2 | 4 |
| T-cell receptor, alpha | TCRA | 4 |
| T-cell receptor, delta | TCRD | 4 |
| Telomerase protein component | | 1 |
| Tenascin (cytotactin) | | 3 |
| Tenascin XA | TNXA | 3 |
| Terminal deoxynucleotidyltransferase, TDT | | 1 |
| Testis-specific protein Y | TSPY | 6 |
| Thiolase, perioxisomal | | 1 |
| Thiopurine S-methyltransferase | TPMT | 1 |
| Thrombopoietin | THPO | 6 |
| Thrombospondin | THBS1 | 6 |
| Thromboxane A synthase 1 | TBXAS1 | 4 |
| Thromboxane A2 | TXA2 | 4 |
| Thromboxane A2 receptor | TBXA2R | 4 |
| Thy-1 T-cell antigen | THY1 | 4 |
| Thymidylate synthase | TYMS | 1 |
| Thymopoietin | TMPO | 6 |
| Thyroglobulin | TG | 6 |
| Thyroid hormone receptor, alpha | THRA | 6 |
| Thyroid hormone receptor, beta | THRB | 6 |
| Thyroid peroxidase | TPO | 6 |
| Thyroid receptor auxiliary protein | TRAP | 6 |
| Thyroid-stimulating hormone receptor | TSHR | 6 |
| Thyroid-stimulating hormone, alpha | TSHA | 6 |
| Thyroid-stimulating hormone, beta | TSHB | 6 |
| Thyrotroph embryonic factor | TEF | 6 |
| Thyrotropin releasing hormone | TRH | 6 |
| Thyrotropin releasing hormone receptor | TRHR | 6 |
| Thyroxin-binding globulin | TBG | 2 |
| TIE receptor tyrosine kinase | TIE-1 | 6 |
| Tip-associated protein | TAP | 4 |
| Tissue inhibitor of metalloproteinase 1, TIMP1 | TIMP1 | 1 |
| Tissue inhibitor of metalloproteinase 2, TIMP2 | TIMP2 | 1 |
| Tissue inhibitor of metalloproteinase 3, TIMP3 | TIMP3 | 1 |
| Tissue inhibitor of metalloproteinase 4, TIMP4 | TIMP4 | 1 |
| Tissue non-specific alkaline phosphatase | | 1 |
| TNSAP | | |
| Titin | TTN | 3 |
| Tocopherol (alpha) transfer protein | TTPA | 2 |
| Toll-like receptor 4 | TLR4 | 4 |
| Topoisomerase I | | 1 |
| Topoisomerase II | | 1 |
| Torticollis, keloids, cryptorchidism and renal | TKCR | 6 |
| dysplasia gene | | |
| Transacylase | | 1 |
| Transcobalamin 1, TCN1 | | 2 |
| Transcobalamin 2, TCN2 | TCN2 | 2 |
| Transcription factor 1, hepatic | TCF1 | 6 |
| Transcription factor 2, hepatic | TCF2 | 6 |
| Transcription factor 3 | TCF3 | 6 |
| Transcription factor binding to IGHM enhancer 3 | TFE3 | 6 |
| Transcription factor, TUPLE1 | TUPLE1 | 5 |
| Transcription termination factor, RNA | TTF1 | 6 |
| polymerase 1 | | |
| Transcription termination factor, RNA | TTF2 | 6 |
| polymerase 2 | | |
| Transcription termination factor, RNA | TTF3 | 6 |
| polymerase 3 | | |
| Transferrin | TF | 6 |
| Transferrin receptor | TFRC | 6 |
| Transforming growth factor, alpha | TGFA | 6 |
| Transforming growth factor, beta 2 | TGFB2 | 6 |
| Transforming growth factor, beta induced | TGFB1 | 6 |
| Transforming growth factor, beta receptor 2 | TGFBR2 | 6 |
| Transglutaminase 1 | TGMI | 6 |
| Transglutaminase 2 | TGM2 | 6 |
| Transglutaminase 4 | TGM4 | 6 |
| Transketolase | TKT | 1 |
| Transketolase-like 1 | TKTL1 | 1 |
| Translocation in renal carcinoma on | TRC8 | 6 |
| chromosome 8 gene | | |
| Transthyretin | TTR | 2 |
| Treacle gene | TCOF1 | 6 |
| Triosephosphate isomerase | TPI 1 | 1 |
| Tropomyosin 1 alpha | TPM1 | 3 |
| Tropomyosin 3 (non-muscle) | TPM3 | 3 |
| Troponin C | | 3 |
| Troponin I | TNNI3 | 3 |
| Troponin T2, cardiac | TNNT2 | 3 |
| Trypsin inhibitor | | 1 |
| Trypsinogen 1 | TRY1 | 1 |
| Trypsinagen 2 | TRY2 | 1 |
| Tryptophan hydroxylase | TPH | 1 |
| Tubby-like protein 1 | TULP1 | 6 |
| Tuberous sclerosis 1 | TSC1 | 6 |
| Tuberous sclerosis 2 | TSC2 | 6 |
| Tubulin | | 3 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour protein p73 | TP73 | 6 |
| Tumour protein, transiationally-controlled 1 | TPT1 | 6 |
| Tumour suppresssor gene DRA | DRA | 4 |
| Twist (Drosophila) homolog | TWIST | 6 |
| Tyrosinase | TYR | 1 |
| Tyrosinase-related protein 1 | TYRP1 | 1 |
| Tyrosine aminotransferase | TAT | 1 |
| Tyrosine hydroxylase | TH | 1 |
| Ubiquitin | | 6 |
| Ubiquitin activating enzyme, E1 | | 1 |
| Ubiquitin B | UBB | 6 |
| Ubiquitin C | UBC | 6 |
| Ubiquitin carboxyl-terminal esterase L1 | UCHL1 | 6 |
| Ubiquitin fusion degeneration I-like | UFD1L | 6 |
| Ubiquitin protein ligase E3A | UBE3A | 1 |
| UDP-glucose pyrophosphorylase | | 1 |
| UDP-glucuronosyltransferase 1 | ugt1d, UGT1 | 1 |
| UDP-glucuronosyltransferase 2 | UGT2 | 1 |
| Uncoupling protein 1 | | 2 |
| Uncoupling protein 3 | UCP3 | 2 |
| Undulin 1 | COL14A1 | 3 |
| Uridine monophosphate kinase | UMPK | 4 |
| Uridine monophosphate synthetase | UMPS | 4 |
| Uridinediphosphate(UDP)-galactose-4- | GALE | 1 |
| epimerase | | |
| Uroporphyrinogen decarboxylase | UROD | 1 |
| Uroporphyrinogen III synthase | UROS | 1 |
| Usher syndrome 2A | USH2A | 3 |
| Vascular endothelial growth factor | VEGF | 6 |
| Vasoinhibitory peptide | | 6 |
| Vitamin B12-binding (R) protein | | 6 |
| Vitamin D receptor | VDR | 6 |
| Vitelliform macular dystrophy, atypical gene | VMD1 | 2 |
| v-myc avian myelocytomatosis viral oncogene | MYC | 6 |
| homolog | | |
| Von Hippel-Lindau gene | VHL | 6 |
| Werner syndrome helicase | WRN | 6 |
| Wilms tumour gene 1 | WT1 | 6 |
| Wilms tumour gene 2 | WT2 | 6 |
| Wilms tumour gene 4 | WT4 | 6 |
| Winged helix nude | WHN | 6 |
| Wingless family, wnt2 | WNT2 | 6 |
| Wingless family, wnt4 | WNT4 | 6 |
| Wingless family, wnt5 | WNT5 | 6 |
| Wingless family, wnt7 | WNT7 | 6 |
| Wingless family, wnt8 | WNT8 | 6 |
| Wiskoft-Aldrich syndrome protein | WASP, THC | 4 |
| Wnt inhibitory factor, WIF-1 | WIF1 | 6 |
| Wolf-Hirschhorn syndrome candidate 1 gene | WHSC1 | 6 |
| Wolfram syndrome 1 gene | WFS1 | 3 |
| X (inactive)-specific transcript | XIST | 6 |
| Xanthine dehydrogenase | XDH | 1 |
| Xeroderma pigmentosum, complementation | XPA | 1 |
| group A | | |
| Xeroderma pigmentosum, complementation | XPB | 1 |
| group B | | |
| Xeroderma pigmentosum, complementation | XPC | 1 |
| group C | | |
| Xeroderma pigmentosum, complementation | | 1 |
| group D | | |
| Xeroderma pigmentosum, complementation | | 1 |
| group E | | |
| Xeroderma pigmentosum, complementation | XPF | 1 |
| group F | | |
| Xeroderma pigmentosum, complementation | ERCC5 | 1 |
| group G | | |
| X-ray repair gene | XRCC9 | 6 |
| Xylitol dehydrogenase | | 6 |
| YY1 transcription factor | YY1 | 6 |
| Zinc finger protein 2 | ZIC2 | 3 |
| Zinc finger protein 3 | ZIC3 | 3 |
| Zinc finger protein HRX | ALL1 | 4 |
| Zona pellucida glycoprotein I | ZP1 | 6 |
| Zona pellucida glycoprotein 2 | ZP2 | 6 |
| Zona pellucida glycoprotein 3 | ZP3 | 6 |
| Zona pellucida receptor tyrosine kinase | ZRK | 6 |
| Zonadhesin | ZAN | 6 |

that the genes associated with a malfunction, damage or disease of the skin, the muscles, the connective tissue or the bones are selected from:

| | HUGO | Protein- |
|---|---|---|
| List of genes | gene symbol | function |
| 17beta Hydroxysteroid oxidoreductase | | 1 |
| 5,10-methylenetetrahydrofolate reductase | MTHFR | 1 |
| (NADPH) | | |
| 6-Phosphofructo-2-kinase | PFKFB1 | 1 |
| Acetoacetyl 2-CoA-thiolase | ACAT2 | 1 |
| Acetylcholine receptor, nicotinic, alpha A1 | CHRNA1 | 5 |
| Acetylcholine receptor, nicotinic, alpha A2 | CHRNA2 | 5 |
| Acetylcholine receptor, nicotinic, alpha A3 | CHRNA3 | 5 |
| Acetylcholine receptor, nicotinic, alpha A4 | CHRNA4 | 5 |
| Acetylcholine receptor, nicotinic, alpha A5 | CHRNA5 | 5 |
| Acetylcholine receptor, nicotinic, alpha A6 | CHRNA6 | 5 |
| Acetylcholine receptor, nicotinic, alpha A7 | CHRNA7 | 5 |
| Acetylcholine receptor, nicotinic, beta 1 | CHRNB1 | 5 |
| Acetylcholine receptor, nicotinic, beta 2 | CHRNB2 | 5 |
| Acetylcholine receptor, nicotinic, beta 3 | CHRNB3 | 5 |
| Acetylcholine receptor, nicotinic, beta 4 | CHRNB4 | 5 |
| Acetylcholine receptor, nicotinic, epsilon | CHRNE | 5 |
| Acetylcholine receptor, nicotinic, gamma | CHRNG | 5 |
| Acetylcholinesterase | ACHE | 1 |
| Achromatopsia 2 | ACHM2 | 3 |
| Acid phosphatase 2, lysosomal | ACP2 | 1 |
| Actin, alpha, cardiac | ACTC | 3 |
| Actin, alpha, skeletal | ACTA1 | 3 |
| Actin, alpha, smooth, aortic | ACTA2 | 3 |
| Actin, beta | ACTB | 3 |
| Actin, gamma 2 | ACTG2 | 3 |
| Activin | | 6 |
| Acyl CoA dehydrogenase, short chain | ACADS | 1 |
| Acyl-CoA thioesterase | | 1 |
| Adaptin, beta 3A | ADTB3A | 2 |
| Adducin, alpha | ADD1 | 3 |
| Adducin, beta | ADD2 | 3 |
| Adenosine deaminase | ADA | 1 |
| Adenosine monophosphate deaminase | AMPD | 1 |
| Adenosine receptor A1 | ADORA1 | 5 |
| Adenosine receptor A2A | ADORA2A | 5 |
| Adenosine receptor A26 | ADORA2B | 5 |
| Adenosine receptor A3 | ADORA3 | 5 |
| Adenyl cyclase | | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | AOCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylate kinase | AK1 | 1 |
| Adenylosuccinate lyase | ADSL | 1 |
| Adrenergic receptor, alphal | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenocorticotrophic hormone (ACTH) | ACTHR | 6 |
| receptor | | |
| Adrenoleukodystrophy gene | ALD | 1 |
| Alanine aminotransferase | | 2 |
| Alanine-glyoxylate aminotransferase | AGXT | 1 |
| Albumin, ALB | ALB | 2 |
| Alcohol dehydrogenase 1 | ADH1 | 1 |
| Alcohol dehydrogenase 2 | ADH2 | 1 |
| Alcohol dehydrogenase 3 | ADH3 | 1 |
| Alcohol dehydrogenase 4 | ADH4 | 1 |
| Alcohol dehydrogenase 5 | ADH5 | 1 |
| Alcohol dehydrogenase 6 | ADH6 | 1 |
| Alcohol dehydrogenase 7 | ADH7 | 1 |
| Aldehyde dehydrogenase 1 | ALDH1 | 1 |
| Aldehyde dehydrogenase 10 | ALDH10 | 1 |
| Aldehyde dehydrogenase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldolase A | ALDOA | 1 |
| Aldolase B | ALDOB | 1 |
| Aldolase C | ALDOC | 1 |
| Aldosterone receptor | MLR | 6 |
| Alkaline phosphatase, liver/bone/kidney | ALPL | 2 |
| Alkaptonuria gene | AKU | 6 |
| Alkylglycerone phosphate synthase | AGPS | 1 |
| alpha tectorin | TECTA | 6 |
| alpha thalassemia gene | ATRX | 5 |
| alpha 1-antichymotrypsin | AACT | 1 |
| alpha1-antitrypsin | PI | 1 |
| alpha2-antiplasmin | PLI | 1 |
| alpha-actinin 2 | ACTN2 | 6 |
| alpha-actinin 3 | ACTN3 | 6 |
| alpha-Galactosidase A | GLA | 1 |
| Alpha-galactosidase B, GALB | NAGA | 1 |
| alpha-synuclein | SNCA | 5 |
| Amelogenin | AMELX | 3 |
| Aminopeptidase P | XPNPEP2 | 1 |
| Amphiregulin | AREG | 6 |
| Amylo-1,6-glucosidase | AGL | 1 |
| Amyloid beta A4 precursor protein | APP | 5 |
| Amyloid beta A4 precursor-like protein | APLP | 5 |
| Androgen binding protein | ABP | 2 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCPI | 1 |
| Angiotensinogen | AGT | 1 |
| Antidiuretic hormone receptor | ADHR | 2 |
| Anti-Mulledan hormone | AMH | 6 |
| Apolipoprotein A 4 | APOA4 | 2 |
| Apolipoprotein A I | APOA1 | 2 |
| Apolipoprotein A II | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Arginine vasopressin | AVP | 5 |
| Arginine vasopressin receptor 1A | AVPR1A | 5 |
| Arginine vasopressin receptor 1B | AVPR1B | 5 |
| Arginine vasopressin receptor 2 | AVPR2 | 5 |
| Arrestin | SAG | 3 |
| Aryl hydrocarbon receptor nuclear | ARNT | 2 |
| translocator | | |
| Arylsuffatase A | ARSA | 1 |
| Arylsulfatase B | ARSB | 1 |
| Arylsulfatase C | ARSCI | 1 |
| Arylsulfatase D | ARSD | 1 |
| Arylsulfatase E | ARSE | 1 |
| Arylsulfatase F | ARSF | 1 |
| Aspartate receptor | | 5 |
| Aspartoacylase | ASPA | 1 |
| Aspartylglucosaminidase | AGA | 1 |
| Ataxia telangiectasia complementation group D | ATD, ATDC | 6 |
| Ataxia telangiectasia gene, AT | ATM | 6 |
| ATP cobalamin adenoxyltransferase | | 1 |
| ATP sulphurylase | atpsk2 | 1 |
| ATP/ADP translocase | | 1 |
| Aftractin | | 4 |
| Autoimmune regulator, AIRE | AIRE | 4 |
| BCL2-related protein A1 | BCL2A1 | 6 |
| Benzodiazepine receptor | | 5 |
| Bestrophin | VMD2 | 2 |
| beta 2 Microglobulin | B2M | 4 |
| beta-endorphin receptor | | 5 |
| beta-galactosidase | GLBI | 1 |
| beta-Glucuronidase | GUSB | 1 |
| beta-synuclein | SNCB | 5 |
| Bilirubin UDP-glucuronosyltransferase | | 1 |
| Bloom syndrome protein | BLM | 6 |
| Blue cone pigment | BCP | 3 |
| Bone morphogenetic protein, BMP1 | BMP1 | 6 |
| Bone morphogenetic protein, BMP2 | BMP2 | 6 |
| Bone morphogenetic protein, BMP3 | BMP3 | 6 |
| Bone morphogenetic protein, BMP4 | BMP4 | 6 |
| Bone morphogenetic protein, BMP5 | BMP5 | 6 |
| Bone morphogenetic protein, BMP6 | BMP6 | 6 |
| Bone morphogenetic protein, BMP7 | BMP7 | 6 |
| Bone morphogenetic protein, BMP8 | BMP8 | 6 |
| Bradykinin receptor B1 | | 4 |
| Bradykinin receptor B2 | | 4 |
| Branched chain aminotransferase 1, cytosolic | BCAT1 | 1 |
| Branched chain aminotransferase 2, | BCAT2 | 1 |
| mitochondrial | | |
| Breast cancer, ductal, 1 | BRCD1 | 6 |
| Breast cancer, ductal, 2 | BRCD2 | 6 |
| Butyrylcholinesterase | BCHE | 1 |
| Ca(2+) transporting ATPase, fast twitch | ATP2A1 | 2 |
| Ca(2+) transporting ATPase, slow twitch | ATP2A2 | 2 |
| Cadherin E | CDH1 | 6 |
| Cadherin EP | | 6 |
| Cadherin N | CDH2 | 6 |
| Cadherin P | CDH3 | 6 |
| Calbindin 1 | CALB1 | 6 |
| Calbindin D9K | CALB3 | 6 |
| Calcitonin receptor/Calcitonin gene-related | CALCR | 5 |
| peptide receptor | | |
| Calcitonin/Calcitonin gene-related peptide | CALCA | 5 |
| alpha | | |
| Calcium channel, voltage-dependent, L type, | CACNA1S | 5 |
| alpha 1S subunit | | |
| Calcium channel, voltage-dependent, P/Q | CACNA1A | 5 |
| type, alpha 1A subunit | | |
| Calmodulin 1 | CALM1 | 6 |
| Calmodulin 2 | CALM2 | 6 |
| Calmodulin 3 | CALM3 | 6 |
| Calnexin | CANX | 6 |
| Calpain | CAPN, | 1 |
| | CAPN3 | |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Camitine acetyltransferase | CRAT | 1 |
| Camitine acylcarnitine translocase | CACT | 1 |
| Carnitine palmitoyltransferase I | CPT1A | 1 |
| Carnitine paimitoyltransferase II | CPT2 | 1 |
| Camitine transporter protein | CDSP, SCD | 2 |
| Cartilage oligomeric matrix protein | COMP, | 5 |
| | EDM1, | |
| | PSACH | |
| Cartilage-hair hypoplasia gene | CHH | 5 |
| Catenin, beta | CTNNB1 | 6 |
| Cathepsin K | CTSK | 1 |
| Caveolin 3 | CAV3 | 1 |
| CD1 | CD1 | |
| C04 | CD4 | |
| Ceroid lipofuscinosis neuronal 3 | CLN3 | 5 |
| Ce loplasmin precursor | CP | 1 |
| Chemokine MCAF | MCAF | 4 |
| Chloride channel 1, skeletal muscle | CLCN1 | 3 |
| Cholecystokinin | CCK | 5 |
| Cholecystokinin B receptor | CCKBR | 5 |
| Cholesterol ester hydroxylase | | 1 |
| Choline acetyltransferase | CHAT | 1 |
| Choroideremia gene | CHM | 3 |
| Citrate synthase | | 1 |
| Clathdn | | 2 |
| Cleft palate gene | CPX | 6 |
| Cockayne syndrome gene, CKN1 | CKN1 | 6 |
| Coenzyme Q (CoQ)/ubiquinone | | 1 |
| Collagen I alpha 1 | COL1A1 | 3 |
| Collagen I alpha 2 | COL1A2 | 3 |
| Collagen II alpha 1 | COL2A1 | 3 |
| Collagen III alpha 1 | COL3A1 | 3 |
| Collagen IV alpha 1 | COL4A1 | 3 |
| Collagen IV alpha 2 | COL4A2 | 3 |
| Collagen IV alpha 3 | COL4A3 | 3 |
| Collagen IV alpha 4 | COL4A4 | 3 |
| Collagen IV alpha 5 | COL4A5 | 3 |
| Collagen IV alpha 6 | COL4A6 | 3 |
| Collagen IX alpha 2 | COL9A2, | 3 |
| | EDM2 | |
| Collagen IX alpha 3 | COL9A3 | 3 |
| Collagen receptor | COLR | 3 |
| Collagen V alpha 1 | COL5A1 | 3 |
| Collagen V alpha 2 | COL5A2 | 3 |
| Collagen VI alpha 1 | COL6AI | 3 |
| Collagen VI alpha 2 | COL6A2 | 3 |
| Collagen VI alpha 3 | COL6A3 | 3 |
| Collagen VII alpha 1 | COL7A1 | 3 |
| Collagen X alpha 1 | COL10A1 | 3 |
| Collagen X alpha 1 | COLL11A1 | 3 |
| Collagen XI alpha 2 | COLL11A2 | 3 |
| Collagen XVI I alpha 1 | COL17A1 | 3 |
| Collagenic-like tail subunit of asymmetric | COLQ | 1 |
| acetylcholinesterase | | |
| Collapsin | | 6 |
| Colony-stimulating factor 1 | CSF1 | 6 |
| Colony-stimulating factor 1 receptor | CSF1R | 6 |
| Colony-stimulating factor 2 | CSF2 | 6 |
| Colony-stimulating factor 2 alpha receptor | CSF2RA | 6 |
| Colony-stimulating factor 2 beta receptor | CSF2RB | 6 |
| Colony-stimulating factor 3 | CSF3 | 6 |
| Colony-stimulating factor 3 receptor | CSF3R | 6 |
| Complement component C1 inhibitor | C1NH | 4 |
| Complement component C1QA | C1QA | 4 |
| Complement component C1QB | C1QB | 4 |
| Complement component C1QG | C1QG | 4 |
| Complement component C1r | C1R | 4 |
| Complement component C1s | C1S | 4 |
| Complement component C2 | C2 | 4 |
| Complement component C3 | C3 | 4 |
| Complement component C4A | C4A | 4 |
| Complement component C4B | C4B | 4 |
| Complement component C5 | C5 | 4 |
| Complement component C6 | C6 | 4 |
| Complement component C7 | C7 | 4 |
| Complement component C8 | C8B | 4 |
| Complement component C9 | C9 | 4 |
| Complement component receptor 1 | CR1 | 4 |
| Complement component receptor 2 | CR2 | 4 |
| Complement component receptor 3 | CR3 | 4 |
| Complex | | 1 |
| Complex II | | 1 |
| Complex III | | 1 |
| Complex III | | 1 |
| Complex V | MTATP6 | 1 |
| Cone-rod homeobox-containing gene | CRX | 6 |
| Coproporphyrinogen oxidase | CPO | 1 |
| Core-binding factor, alpha 1 | CBFAL | 6 |
| Core-binding factor, alpha 2 | CBFA2 | 6 |
| Core-binding factor, beta | CBFB | 6 |
| Corticosteroid binding globulin | CBG | 5 |
| Corticosteroid binding protein | | 2 |
| Corticotrophin-releasing hormone | CRH | 2 |
| Corticotrophin-releasing hormone receptor | CRHR1 | 2 |
| Cortisol receptor | | 4 |
| C-reactive protein CRP | | 4 |
| Creatine kinase - B and m | CKBE | 1 |
| Creb binding protein | CREBBP | 6 |
| Crystallin, alpha A | CRYAA | 3 |
| Crystallin, alpha B | CRYAB | 3 |
| Crystallin, beta B2 | CRYBB2 | 3 |
| Crystallin, gamma A | CRYGA | 3 |
| c-src tyrosine kinase | CSK | 6 |
| Cu2+ transporting ATPase alpha polypeptide | ATP7A | 1 |
| Cu2+ transporting ATPase beta polypeptide | ATP7B | 1 |
| Cyclic AMP response element binding protein | | 6 |
| CREB | | |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin-dependent kinase 2 | CDK2 | 6 |
| Cyclin-dependent kinase inhibitor IC (P57, | CDKN1C | 6 |
| KIP2) | | |
| Cyclin-dependent kinase inhibitor 2A (p16) | CDKN2A | 6 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cystinosin | CTNS | 2 |
| Cytidine deaminase | CDA | 1 |
| Cytidine-5-prime-triphosphate synthetase | CTPS | 1 |
| Cytochrome a | | 1 |
| Cytochrome b-5 | CYB5 | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| DAX1 nuclear receptor | DAX1 | 4 |
| Deafness dystonia peptide | DDP | 5 |
| Delta-4,5-alpha-reductase | | 1 |
| Delta aminolevulinate dehydratase | ALAD | 1 |
| Delta(4)-3-oxosteroid 5-beta-reductase | | 1 |
| Delta-7-dehydrocholesterol reductase | DHCR7 | 1 |
| Dentin sialophosphoprotein | DSPP | 6 |
| Desmin | DES | 3 |
| DHEA sulfotransferase | STD | 1 |
| Diastrophic dysplasia sulfate transporter | DTD | 2 |
| Dihydrolipoamide dehydrogenase | DLD | 5 |
| Dihydroxyacetonephosphate acyltransferase | DHAPAT | 1 |
| DNA damage binding protein, DDB1 | DDB1 | 3 |
| DNA damage binding protein, DDB2 | DDB2 | 3 |
| DNA methyltransferase | DNMT | 1 |
| DNA-damage-inducible transcript 3 | DDIT3 | 3 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dynamin | DNM1 | 6 |
| Dynorphin receptor | | 5 |
| Dyskehn | DKC1 | 3 |
| Dystonia 1 | DYT1 | 3 |
| Dystonia 3 | DYT3 | 3 |
| Dystonia 6 | DYT6 | 3 |
| Dystonia 7 | DYT7 | 3 |
| Dystrophia myotonica | DM, DMPK | 1 |
| Dystrophia myotonica, atypical | DM2 | 1 |
| Dystrophin | DMD | 3 |
| Dystrophin-associated glycoprotein 35 kD, | SGCD | 3 |
| SCGD | | |
| Dystrophin-associated glycoprotein 35 kD, | SGCG | 3 |
| SGSG | | |
| Dystrophin-associated glycoprotein 43 kD | SGCB | 3 |
| Dystrophin-associated glycoprotein 50 kD | SGCA | 3 |
| Ectodermal Dysplasia 1 gene | ED1 | 3 |
| Elastase 1 | ELAS1 | 1 |
| Elastase 2 | ELAS2 | 1 |
| Elastin | ELN | 3 |
| Electron-transfering-flavoprotein alpha | ETFA | 2 |
| Electron-transfe ng-flavoprotein beta | ETFB | 2 |
| Electron-transferdng flavoprotein | ETFDH | 1 |
| dehydrogenase | | |
| Emerin | EMD | 2 |
| Endocardial fibroelastosis 2 gene | EFE2 | 3 |
| Endometrial bleeding-associated factor | EBAF | 6 |
| Endothelin 1 | EDN1 | 5 |
| Endothelin 2 | EDN2 | 5 |
| Endothelin 3 | ED 3 | 5 |
| Endothelin converting enzyme | ECE1 | 5 |
| Endothelin receptor type A | EDNRA | 5 |
| Endothelin receptor type 8 | EDNRB | 5 |
| Engrailed-1 | EN1 | 6 |
| Engrailed-2 | EN2 | 6 |
| Enolase | ENO1 | 1 |
| Enoyl CoA hydratase | | 1 |
| Enoyl CoA isomerase | | 1 |
| Enoyl CoA reductase | | 1 |
| Enterokinase | PRSS7, | 1 |
| | ENTK | |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Erythrocyte membrane protein band 4.1 | EPB41 | 3 |
| Erythropoietin | EPO | 4 |
| Erythropoietin receptor | EPOR | 4 |
| Estrogen receptor | ESR | 6 |
| Exostosin 1 | EXT1 | 3 |
| Exostosin 2 | EXT2 | 3 |
| Exostosin 3 | EXT3 | 3 |
| Eye colour gene 3 (brown) | EYCL3 | 3 |
| Eyes absent 1 | EYA1 | 6 |
| Faciogenital dysplasia | FGDI, | 2 |
| | FGDY | |
| Factor I (No. one) | F1 | |
| Factor B, properdin | | |
| Factor D | | |
| Factor H | HF1 | |
| Factor X | F10 | |
| Fanconi anemia, complementation group A | FANCA | 2 |
| Fanconi anemia, complementation group C | FANCC | 2 |
| F nconi anemia, complementation group D | | 2 |
| FANCD | | |
| Fc fragment of IgG, high affinity IA, receptor | FCGR1A | 6 |
| for | | |
| Fc fragment of IgG, low affinity IIa, receptor | FCGR2A | 6 |
| for (CD32) | | |
| Ferritin, H subunit | | 2 |
| Ferritin, L subunit | FTL | 2 |
| Fibrillin 1 | FBN1 | 6 |
| Fibrillin 2 | FBN2 | 6 |
| Fibrinogen alpha | FGA | 3 |
| Fibrinogen beta | FGB | 3 |
| Fibrinogen gamma | FGG | 3 |
| Fibroblast growth factor | FGF1 | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Fibronectin precursor | FN1 | 6 |
| Flightless-II, Drosaphila homolog of | FLII | 6 |
| Folic acid receptor | FOLR | 6 |
| Follicle stimulating hormone receptor | FSHR, | 6 |
| | ODG1 | |
| Follicle stimulating hormone, FSH | FSHB | 6 |
| Forkhead transcription factor 10 | FKHL10 | 6 |
| Forkhead transcription factor 14 | FKHL14 | 6 |
| Forkhead transcription factor 7 | FKHL7 | 6 |
| Fragile site, folic acid type, rare, fra(X) A | FRAXA | 5 |
| Frataxin | FRDA | 6 |
| Fringe secreted protein, lunatic | LFNG | 6 |
| Fringe secreted protein, manic | MFNG | 6 |
| Fringe secreted protein, radical | RFNG | 6 |
| Fructose-1,6-diphosphatase | FBP1 | 1 |
| Fucosidase alpha-L-1 | FUCA1 | 1 |
| Fucosidase alpha-L-2 | | 1 |
| Fukuyama type congenital muscular | FCMD | 6 |
| dystrophy | | |
| Fumarase | FH | 1 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma I | GABRG1 | 5 |
| GABA receptor, gamma 2 | GABRG2 | 5 |
| GABA receptor, gamma 3 | GABRG3 | 5 |
| Galactocerebrosidase | GALC | 1 |
| Galactokinase | GALK1 | 1 |
| Galactose I-phosphate uridyl-transferase | GALT | 1 |
| Gamma-glutamyl carboxylase | GGCX | 2 |
| Gap junction protein alpha 3 | GJA3 | 2 |
| Gap junction protein alpha 8 | GJA8 | 2 |
| Gap junction protein beta 3 | GJB3 | 2 |
| Gastrulation brain homeobox 2 | GBX2 | 6 |
| Glucosidase, acid alpha | GAA | 1 |
| Glucosidase, acid beta | GBA | 1 |
| Glutamate receptor 1 | GLUR1 | 5 |
| Glutamate receptor 2 | GLUR2 | 5 |
| Glutamate receptor 3 | GLUR3 | 5 |
| Glutamate receptor 4 | GLUR4 | 5 |
| Glutamate receptor 5 | GLUR5 | 5 |
| Glutamate receptor 6 | GLUR6 | 5 |
| Glutamate receptor 7 | GLUR7 | 5 |
| Glutamate receptor, ionotropic, NMDA 1 | NMDAR1 | 5 |
| Glutamate receptor, ionotropic, NMDA 2A | NMDAR2A | 5 |
| Glutamate receptor, ionotropic, NMDA 2B | NMDAR2B | 5 |
| Glutamate receptor, ionotropic, NMDA 2C | NMDAR2C | 5 |
| Glutamate receptor, ionotropic, NMDA 2D | NMDAR2D | 5 |
| Glutathione | GSH | 2 |
| Glutathione peroxidase, GPX1 | GPX1 | 1 |
| Glutathione S-transferase, GSTZ1 | GSTZ1 | 1 |
| Glyceraldehyde-3-phosphate | GAPDH | 1 |
| dehydrogenase, GAPDH | | |
| Glycerol kinase | GK | 1 |
| Glycinamide ribonucleotide (GAR) | GART | 1 |
| transformylase | | |
| Glycine receptor, alpha | GLRA2 | 5 |
| Glycine receptor, beta | | 5 |
| Glycine transporter | GLYT | 5 |
| Glycogen phosphorylase | PYGL | 1 |
| Glycosyltransferases, ABO blood group | ABO | 1 |
| GM2 ganglioside activator protein, GM2A | GM2A | 1 |
| Green cone pigment | GCP | 3 |
| Growth arrest-specific homeobox | GAX | 6 |
| Growth factor receptor-bound protein 2 | GRB2 | 6 |
| Growth hormone 1 | GH1 | 6 |
| Growth hormone 2 (placental) | GH2 | 6 |
| Growth hormone receptor | GHR | 6 |
| Growth hormone releasing hormone (GHRH) | GHRH | 6 |
| Growth hormone releasing hormone receptor | GHRHR | 6 |
| Growth/differentiation factor 5 | GDF5 | 6 |
| GTP cylcohydrolase 1 | GCH1 | 6 |
| GTPase-activating protein, GAP | RASAL | 6 |
| Guanidinoacetate N-methyltransferase | GAMT | 1 |
| Guanine nucleotide-binding protein, alpha | GNAO1 | 5 |
| activating activity polypeptide, GNAO y | | |
| Guanine nucleotide-binding protein, alpha | GNAI1 | 5 |
| inhibiting activity polypeptide 1, GNA[L | | |
| Guanine nucleotide-binding protein, alpha | GNAI2 | 5 |
| inhibiting activity polypeptide 2, GNA 2 | | |
| Guanine nucleotide-binding protein, alpha | GNAI3 | 5 |
| inhibiting activity polypeptide 3, GNA[3 | | |
| Guanine nucleotide-binding protein, alpha | GNAS1 | 5 |
| stimulating activity polypeptide, GNAS I | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNAT1 | 5 |
| transducing activity polypeptide, GNATI | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, q | GNAQ | 5 |
| polypeptide | | |
| Guanylate cyclase 2D, membrane (retina- | GUCY2D | 1 |
| specific) | | |
| Guanylate cyclase activator 1A (retina) | GUCALA | 1 |
| H(+), K(+) - ATPase | ATP4B | 5 |
| Haeme regulated inhibitor kinase | 1 | |
| Haemoglobin alpha 1 | HBAL | 2 |
| Haemoglobin alpha 2 | HBA2 | 2 |
| Haemoglobin beta | HBB | 2 |
| Haemoglobin delta | HBD | 2 |
| Haemoglobin gamma A | HBG1 | 2 |
| Haemoglobin gamma B | HBG2 | 2 |
| Haemoglobin gamma G | HBGG | 2 |
| Hairless | HR | 6 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Heparan sulfamidase | | 1 |
| Heparin binding epidermal growth factor | HBEGF | 6 |
| Heparin Cofactor II | HCF2 | 4 |
| Hepatocyte growth factor | HGF | 6 |
| Hermansky-pudiak syndrome gene | HPS | 2 |
| Hexokinase 2 | HK2 | 1 |
| Hexosaminidase A | HEXA, TSD | 1 |
| Hexosaminidase B | HEXB | 1 |
| Histamine receptors, H1 | | 5 |
| Histamine receptors, H2 | | 5 |
| Histamine receptors, H3 | | 5 |
| HLA-B associated transcript 1 | BAT1 | 4 |
| Holocarboxylase synthetase | HLCS | 1 |
| Holoprosencephaly 1 | HPEI | 6 |
| Holoprosencephaly 2 | HPE2 | 6 |
| Holoprosencephaly 3 | HPE3 | 6 |
| Holoprosencephaly 4 | HPE4 | 6 |
| Homeobox (HOX) gene A1 | HOXA1 | 6 |
| Homeobox (HOX) gene A10 | HOXA10 | 6 |
| Homeobox (HOX) gene A11 | HOXA11 | 6 |
| Homeobox (HOX) gene A12 | HOXA12 | 6 |
| Homeobox (HOX) gene A13 | HOXA13 | 6 |
| Homeobox (HOX) gene A2 | HOXA2 | 6 |
| Homeobox (HOX) gene A3 | HOXA3 | 6 |
| Homeobox (HOX) gene A4 | HOXA4 | 6 |
| Homeobox (HOX) gene A5 | HOXA5 | 6 |
| Homeobox (HOX) gene A6 | HOXA6 | 6 |
| Homeobox (HOX) gene A7 | HOXA7 | 6 |
| Homeobox (HOX) gene A8 | HOXA8 | 6 |
| Homeobox (HOX) gene A9 | HOXA9 | 6 |
| Homeobox (HOX) gene Bl | HOXBL | 6 |
| Homeobox (HOX) gene B2 | HOXB2 | 6 |
| Homeobox (HOX) gene B3 | HOXB3 | 6 |
| Homeobox (HOX) gene B4 | HOXB4 | 6 |
| Homeobox (HOX) gene B5 | HOXB5 | 6 |
| Homeobox (HOX) gene B6 | HOXB6 | 6 |
| Homeobox (HOX) gene B7 | HOXB7 | 6 |
| Homeobox (HOX) gene B8 | HOXB8 | 6 |
| Homeobox (HOX) gene B9 | HOXB9 | 6 |
| Homeobox (HOX) gene C13 | HOXC13 | 6 |
| Homeobox (HOX) gene C4 | HOXC4 | 6 |
| Homeobox (HOX) gene C8 | HOXC8 | 6 |
| Homeobox (HOX) gene C9 | HOXC9 | 6 |
| Homeobox (HOX) gene D1 | HOXD1 | 6 |
| Homeobox (HOX) gene D10 | HOXD10 | 6 |
| Homeobox (HOX) gene D12 | HOXD12 | 6 |
| Homeobox (HOX) gene D13 | HOXD13 | 6 |
| Homeobox (HOX) gene D3 | HOXD3 | 6 |
| Homeobox (HOX) gene D4 | HOXD4 | 6 |
| Homeobox (HOX) gene D8 | HOXD8 | 6 |
| Homeobox (HOX) gene D9 | HOXD9 | 6 |
| Homeobox 11 | HOX11 | 6 |
| Homeobox HB24 | HLX1 | 6 |
| Homeobox HB9 | HLXB9 | 6 |
| Homeobox, PROX1 | PROX1 | 6 |
| Homogentisate 1,2 dioxygenase | HGD | 1 |
| Human placental lactogen | CSH1 | 6 |
| Hypoxia inducible factor 1 | HIF1A | 1 |
| Hypoxia inducible factor 2 | | 1 |
| IC7 A and B | | 4 |
| Immunoglobulin E (IgE) reponsiveness gene | IGER | 4 |
| Indian hedgehog, ihh | IHH | 6 |
| Inhibin, alpha | INHA | 6 |
| Inhibin, beta A | INHBA | 6 |
| Inhibin, beta B | INHBB | 6 |
| Inhibin, beta C | INHBC | 6 |
| Inositol 1,4,5-triphosphate receptor 3 | ITPR3 | 6 |
| Insulin promotor factor 1 | IPF1 | 6 |
| Insulin-like growth factor 1 | IGF1 | 6 |
| Insulin-like growth factor 1 receptor | IGF | 6 |
| Insulin-like growth factor 2 | IGF2 | 6 |
| Insulin-like growth factor 2 receptor | IGF2R | 6 |
| Integrin beta 1 | ITGBL | 6 |
| Integrin beta 3 | ITGB3 | 6 |
| Integrin beta 4 | ITGB4 | 6 |
| Integrin, alpha 5 | ITGA5 | 6 |
| Integrin, alpha 7 | ITGA7 | 6 |
| Inter-alpha-trypsin inhibitor, IATI | | 1 |
| Interferon alpha | IFNA1 | 4 |
| Interferon beta | IFNB | 4 |
| Interferon gamma | IFNG | 4 |
| Interferon gamma receptor 1 | IFNGR1 | 4 |
| Interferon gamma receptor 2 | IFNGR2 | 4 |
| Interferon regulatory factor 1 | IRF1 | 4 |
| Interferon regulatory factor 4 | IRF4 | 4 |
| Interleukin(IL) 1 receptor | IL1R | 4 |
| Interleukin(IL) 1, alpha | IL1A | 4 |
| Interleukin(IL) 1, beta | IL1B | 4 |
| Interleukin(IL) 10 | IL10 | 4 |
| interleukin(IL) 10 receptor | IL10R | 4 |
| Interleukin(IL) 11 | IL11 | 4 |
| Interleukin(IL) 11 receptor | IL11R | 4 |
| Interleukin(IL) 12 | IL12 | 4 |
| Interleukin(IL) 12 receptor, beta 1 | IL12RB1 | 4 |
| lnterleukin(IL) 13 | IL13 | 4 |
| lnterleukin(IL) 13 receptor | IL13R | 4 |
| lnterleukin(IL) 2 | IL2 | 4 |
| Interleukin(IL) 2 receptor, alpha | IL2RA | 4 |
| Interleukin(IL) 2 receptor, gamma | IL2RG | 4 |
| lnterleukin(IL) 3 | IL3 | 4 |
| Interleukin(IL) 3 receptor | IL3R | 4 |
| Interleukin(IL) 4 | IL4 | 4 |
| Interleukin(IL) 4 receptor | IL4R | 4 |
| Interleukin(IL) 5 | IL5 | 4 |
| lnterleukin(IL) 5 receptor | IL5R | 4 |
| Interleukin(IL) 6 | IL6 | 4 |
| Interleukin(IL) 6 receptor | IL6R | 4 |
| Interleukin(IL) 7 | IL7 | 4 |
| lnterleukin(IL) 7 receptor | IL7R | 4 |
| lnterleukin(IL) 8 | IL8 | 4 |
| Interleukin(IL) 8 receptor | IL8R | 4 |
| lnterleukin(IL) 9 | IL9 | 4 |
| Interleukin(IL) 9 receptor | IL9R | 4 |
| lnterleukin(IL) receptor antagonist I | 1L1RN, | 4 |
| | IL1RA | |
| lsocitrate dehydrogenase | | 1 |
| Kaliman sypdrome gene 1 | KAL1 | 6 |
| Keratin 1 | KRT1 | 3 |
| Keratin 10 | KRT10 | 3 |
| Keratin 11 | KRT11 | 3 |
| Keratin 12 | KRT12 | 3 |
| Keratin 13 | KRT13 | 3 |
| Keratin 14 | KRT14 | 3 |
| Keratin 15 | KRTI 5 | 3 |
| Keratin 16 | KRT16 | 3 |
| Keratin 17 | KRT17, | 3 |
| | PCHC1 | |
| Keratin 18 | KRT18 | 3 |
| Keratin 2 | KRT2 | 3 |
| Keratin 3 | KRT3 | 3 |
| Keratin 4 | KRT4 | 3 |
| Keratin 5 | KRT5 | 3 |
| Keratin 6 | KRT6 | 3 |
| Keratin 7 | KRT7 | 3 |
| Keratin 8 | KRT8 | 3 |
| Keratin 9 | KRT9 | 3 |
| Keratin, hair acidic 1 | KRTHA1 | 3 |
| Keratin, hair basic 2 | KRTHB1 | 3 |
| Keratin, hair basic 6 | KRTHB6 | 3 |
| Kininogen, High molecular weight | KNG | 4 |
| Lactate dehydrogenase, A | LDHA | 1 |
| Lactate dehydrogenase, B | LDHB | 1 |
| Lamin A/C | LMNA | 6 |
| Laminin 5, alpha 3 | LAMA3 | 6 |
| Laminin 5, beta 3 | LAMB3 | 6 |
| Laminin 5, gamma 2 | LAMC2 | 6 |
| Laminin M | LAMM | 6 |
| Laminin receptor 1 | LAMRI | 6 |
| Latent transforming growth factor-beta | LTBP2 | 6 |
| binding protein 2 | | |
| Leukocyte-specific transcript 1 | LST-1 | 4 |
| Leukotriene A4 hydrolase | | 4 |
| Leukotriene A4 synthase | LTA4S | 1 |
| Leukotriene B4 receptor | | 4 |
| Leukotriene B4 synthase | LTB4S | 1 |
| Leukotriene C4 receptor | | 4 |
| Leukotriene C4 synthase | LTC4S | 1 |
| LIM homeobox transcription factor 1, beta | LMX1B | 6 |
| Limb girdle muscular dystrophy 1A | LGMD1A | 6 |
| Limb girdle muscular dystrophy 1B | LGMD1B | 6 |
| Limb girdle muscular dystrophy 2G | LGMD2G | 6 |
| Limb girdle muscular dystrophy 2H | LGMD2H | 6 |
| Limbic associated membrane protein | LAMP | 6 |
| Lipoprotein receptor, Low Density | LDLR | 2 |
| Lipoxygenase 12 (platelets) | LOG12 | 4 |
| Loricrin | LOR | 3 |
| Low density lipoprotein receptor-related | LRP | 2 |
| protein precursor | | |
| Luteinizing hormone-releasing hormone | | 5 |
| Luteinizing hormone-releasing hormone | | 5 |
| receptor | | |
| Lymphotoxin | | 4 |
| Lysosome-associated membrane protein 1 | LAMP1 | 6 |
| Lysosome-associated membrane protein 2 | LAMP2 | 6 |
| Lysozyme | LYZ | 4 |
| Lysyl hydroxylase | PLOD | 1 |
| Lysyl oxidase | LOX | 1 |
| Macrophage activating factor | MAF | 4 |
| Macrophage inflammatory protein-1 | MIP1 | 4 |
| Macrophage inflammatory protein-1 | receptor | 4 |
| Macrophage inflammatory protein-2 | MIP2 | 4 |
| Macrophage inflammatory protein-2 receptor | | 4 |
| MADS box transcription-enhancer factor 2A | MEF2A | 6 |
| MADS box transcription-enhancer factor 2B | MEF2B | 6 |
| MADS box transcription-enhancer factor 2C | MEF2C | 6 |
| MADS box transcription-enhancer factor 2D | MEF2D | 6 |
| Mannose binding protein | MBP | 4 |
| Mannosidase, alpha B lysosomal | MANB | 1 |
| Mannosidase, beta A lysosomal | MANBA | 1 |
| Marenostrin | MEFV | 2 |
| Matrix Gla protein | MGP | 6 |
| Matrix metalloproteinase 1 | MMP1 | 1 |
| Matrix metalloproteinase 10 | MMP10 | 1 |
| Matrix metalloproteinase 11 | MMP11 | 1 |
| Matrix metalloproteinase 12 | MMP12 | 1 |
| Matrix metalloproteinase 13 | MMP13 | 1 |
| Matrix metalloproteinase 14 | MMP14 | 1 |
| Matrix metalloproteinase 15 | MMPI 5 | 1 |
| Matrix metalloproteinase 16 | MMP16 | 1 |
| Matrix metalloproteinase 17 | MP17 | 1 |
| Matrix metalloproteinase 18 | MMP18 | 1 |
| Matrix metalloproteinase 19 | MMP19 | 1 |
| Matrix metalloproteinase 2 | MMP2 | 1 |
| Matrix metalloproteinase 3 | MMP3, | 1 |
| | STMYJ | |
| Matrix metalloproteinase 4 | MMP4 | 1 |
| Matrix metalloproteinase 5 | MMP5 | 1 |
| Matrix metalloproteinase 6 | MMP6 | 1 |
| Matrix metalloproteinase 7 | MMP7 | 1 |
| Matrix metalloproteinase 8 | MMP8 | 1 |
| Matrix metalloproteinase 9 | MMP9 | 1 |
| MEK kinase, MEKK | | 1 |
| Melanocortin 1 receptor | MC1R | 2 |
| Melanocortin 2 receptor | MC2R | 2 |
| Melanocortin 4 receptor | MC4R | 2 |
| Mesoderm-specific transcript | MEST | 6 |
| Methylguanine-DNA methyltransferase | MGMT | 1 |
| Methylmalonyl-CoA mutase | MUT | 1 |
| Mevalonate kinase | MVK | 1 |
| MHC Class 1: A | | 4 |
| MHC Class 1: B | | 4 |
| MHC Class 1: C | | 4 |
| MHC Class 1: LMP-2, LMP-7 | | 4 |
| MHC Class 1: Tap1 | ABCR, | 4 |
| | TAP1 | |
| MHC Class II: DP | HLA-DPB1 | 4 |
| MHC Class II: DQ | | 4 |
| MHC Class II: DR | | 4 |
| MHC Class II: Tap2 | TAP2, PSF2 | 4 |
| MHC Class II: Complementation group A | MHC2TA | 4 |
| MHC Class II: Complementation group B | RFXANK | 4 |
| MHC Class II: Complementation group C | RFX5 | 4 |
| MHC Class II: Complementation group D | RFXAP | 4 |
| Microphthalmia-associated transcription | MITF | 6 |
| factor | | |
| Midline 1 | MID1 | 6 |
| Mitochondrial trifunctional protein, alpha | HADHA | 1 |
| subunit | | |
| Mitochondrial trifunctional protein, beta | HADHB | 1 |
| subunit | | |
| Moesin, MSN | | 3 |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Monocyte chemoattractant protein 1 | MCP1 | 4 |
| Mucolipidoses | GNPTA | 1 |
| Mulibrey nanism | MUL | 2 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Muscle phosphoylase | PYGM | 1 |
| Mutated in colorectal cancers, MCC | MCC | 6 |
| MutS homolog 3 | MSH3 | 6 |
| Myeloperoxidase | MPO | 4 |
| Myocilin | MYOC | 2 |
| Myogenic factor 3 | MYF3 | 6 |
| Myogenic factor 4 | MYF4 | 6 |
| Myogenic factor 5 | MYF5 | 6 |
| Myoglobin | | 2 |
| Myomesin 1 | MYOM1 | 3 |
| Myomesin 2 | MYOM2 | 3 |
| Myopia 1 | MYP1 | 2 |
| Myopia 2 | MYP2 | 2 |
| Myosin 15 | MYO15 | 3 |
| Myosin 5A | MYO5A | 3 |
| Myosin 6 | MYO6 | 3 |
| Myosin 7A | MYO7A | 3 |
| Myosin, cardiac | MYH7 | 3 |
| Myosin, light chain 2 | MYL2 | 3 |
| Myosin, light chain 3 | MYL3 | 3 |
| Myotubuladn | MTM1 | 3 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+, K+ ATPase, beta 3 | ATP1B3 | 6 |
| Na+/H+ exchanger 1 | NHEI | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| N-acetylgalactosamine-6-sulfate sulfatase | GALNS | 1 |
| N-acetylglucosamine-6-suffatase | GNS | 1 |
| N-acetylglucosaminidase, alpha | NAGLU | 1 |
| NADH dehydrogenase | | 1 |
| NADH-cytochrome b5 reductase | DIA1 | 1 |
| reductase | | |
| NB6 | | 4 |
| Nebulin | EB | 3 |
| Nephrosis 1 | NPHS1 | 2 |
| Neural retina-specific gene | NRL | 6 |
| Neuraminidase sialidase | NEU | 2 |
| Neuregulin | HGL | 6 |
| Neurexin | | 5 |
| Neuroendocrine convertase 1 | NEC1, | 1 |
| | PCSK1 | |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Neurotensin | NTS | 5 |
| Neurotensin receptor | NTSR1 | 5 |
| Nibrin | NBS1 | 6 |
| Noggin | NOG | 6 |
| Notch ligand - jagged 1 | JAG 1, AGS | 6 |
| Nuclear factor 1-kappa-B-like gene | IKBL | 4 |
| Nuclear factor kappa beta | NFKB | 4 |
| Nuclear factor of activated T cells (NFAT) | NFATC | 6 |
| complex, cytosolic | | |
| Nuclear factor of activated T cells (NFAT) | NFATP | 6 |
| complex, preexisting component | | |
| Ocular albinism 1 | OA1 | 3 |
| Oculocutaneous albinism 11 | OCA2 | 3 |
| Oncogene ERG (early reponse gene) | | 6 |
| Oncogene fos | FOS | 6 |
| Oncogene GLI1 | GLI | 6 |
| Oncogene GLI2 | GLI2 | 6 |
| Oncogene GLI3 | GLI3 | 6 |
| Oncogene sis | PDGFB | 6 |
| Oncogene src | | 6 |
| Opioid receptor, delta | OPRD1 | 5 |
| Opioid receptor, kappa | OPRK1 | 5 |
| Opioid receptor, mu | OPRM1 | 5 |
| Ornithine delta-aminotransferase | OAT | 1 |
| Osteocalcin | | 3 |
| Osteonectin | ON | 6 |
| Osteopontin | OPN | 6 |
| Osteoprotegerin | OPG | 6 |
| Oxytocin | OXT | 5 |
| Oxytocin receptor | OXTR | 5 |
| p21-activated kinase 3 | PAK3 | 6 |
| Paired box homeotic gene 1 | PAX1 | 6 |
| Paired box homeotic gene 2 | PAX2 | 6 |
| Paired box homeotic gene 3 | PAX3 | 6 |
| Paired box homeotic gene 6 | PAX6 | 6 |
| Paired box homeotic gene 7 | PAX7 | 6 |
| Paired box homeotic gene 8 | PAX8 | 6 |
| Paired-like homeodomain transcription factor 2 | PITX2 | 6 |
| Paired-like homeodomain transcription factor 3 | PITX3 | 6 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Patched (Drosophila) homolog, PTCH | PTCH | 6 |
| Peanut-like 1 | PNUTL1 | 4 |
| Peripherin, PRPH | | 3 |
| Peroxisomal membrane protein 1 | PXMP1 | 3 |
| Peroxisoma membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome receptor 1 | PXR1 | 2 |
| Phenylethanolamine N-methyltransferase, | PNMT | 1 |
| PNMT | | |
| Phosphate regulating gene with homologies | PHEX | 6 |
| to endopeptidases on the X chromosome | | |
| Phosphodiesterase 1/nucleotide | PDNP1 | 6 |
| pyrophosphatase 1 | | |
| Phosphodiesterase 1/nucleotide | PDNP2 | 6 |
| pyrophosphatase 2 | | |
| Phosphodiesterase 1/nucleotide | PDNP3 | 6 |
| pyrophosphatase 3 | | |
| Phosphofructokinase, muscle | PFKM | 1 |
| Phosphoglucose isomerase | GPI | 1 |
| Phosphoglycerate kinase 1 | PGK1 | 1 |
| Phosphoglycerate mutase 2 | PGAM2 | 1 |
| Phospholipase A2, group 10 | PLA2G10 | 4 |
| Phospholipase A2, group 1B | PLA2G1B | |
| Phospholipase A2, group 2A | PLA2G2A | |
| Phospholipase A2, group 2B | PLA2G2B | 4 |
| Phospholipase A2, group 4A | PG4A | 4 |
| Phospholipase A2, group 4C | P 2G4C | 4 |
| Phospholipase A2, group 5 | PLA2G5 | 4 |
| Phospholipase A2, group 6 | PLA2G6 | 4 |
| Phosphomannomutase 2 | PMM2 | 6 |
| Phosphoribosyl pyrophosphate synthetase | PRPS1 | 1 |
| Phosphorylase kinase, alpha 1 (muscle) | PHKA1 | 1 |
| Phosphorylase kinase, beta | PHKB | 1 |
| Phosphorylase kinase, delta | | 1 |
| Phosphorylase kinase, gamma 2 | PHKG2 | 1 |
| Phytanoyi-CoA hydroxylase | PHYH | 6 |
| Pineolytic beta-receptors | | 1 |
| Plakophilin 1 | PKP1 | 2 |
| Plasminogen | PLG | 1 |
| Platelet derived growth factor | PDGF | 6 |
| Platelet derived growth factor receptor | PDGFR | 6 |
| Plectin 1 | PLEC1 | 2 |
| Potassium inwardly-rectifying channel J1 | KCNJ1 | 5 |
| Potassium voltage-gated channel E1 | KCNE1 | 5 |
| Potassium voltage-gated channel Q1 | KCNQ1 | 5 |
| Potassium voltage-gated channel Q2 | KCNQ2 | 5 |
| Potassium voltage-gated channel Q3 | KCN03 | 5 |
| POU domain, class 3, transcription factor 4 | POU3F4 | 6 |
| POU domain, class 4, transcription factor 3 | POU4F3 | 6 |
| Prion protein | PRNP | 5 |
| Procollagen N-protease | | 1 |
| Procollagen peptidase | | 1 |
| Prodynorphin | | 5 |
| Profibrinolysin | | 6 |
| Prolactin receptor | PRLR | 6 |
| Prolactin releasing hormone | PRH | 6 |
| Proliferin | PLF | 6 |
| Proopiomelanocortin | POMC | 5 |
| Properdin P factor, complement | PFC, PFD | 4 |
| Prophet of Pit1 | PROP1 | 6 |
| Propionyl-CoA carboxylase, alpha | PCCA | 1 |
| Prosaposin | PSAP | 5 |
| Prostacyclin synthase | | 4 |
| Prostaglandin 15-OH dehydrogenase | HGPD; | 4 |
| | PGDH | |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin F2 alpha receptor | | 4 |
| Prostaglandin I2 receptor | | 2 |
| Prostaglandin IP receptor | | 4 |
| Prostaglandin isomerase | | 6 |
| Protease nexin 2 | PN2 | 1 |
| Protective protein for beta-galactosidase | PPGB | 1 |
| Protein C | PROC | 4 |
| Proteinase 3 | | 4 |
| Purine nucieoside phosphorylase | NP | 1 |
| Purinergic receptor P1A1 | | 5 |
| Purinergic receptor P1A2 | | 5 |
| Purinergic receptor P1A3 | | 5 |
| Purinergic receptor P2X, 1 | P2RX1 | 5 |
| Purinergic receptor P2X, 2 | P2RX2 | 5 |
| Purinergic receptor P2X, 3 | P2RX3 | 5 |
| Pudnergic receptor P2X, 4 | P2RX4 | 5 |
| Purinergic receptor P2X, 5 | P2RX5 | 5 |
| Purinergic receptor P2X, 6 | P2RX6 | 5 |
| Purinergic receptor P2X, 7 | P2RX7 | 5 |
| Purinergic receptor P2Y, 1 | P2RY1 | 5 |
| Purinergic receptor P2Y, 11 | P2RY11 | 5 |
| Purinergic receptor P2Y, 2 | P2RY2 | 5 |
| Pyrroline-5-carboxylate synthetase | PYCS | 1 |
| Pyruvate kinase | PKLR | 1 |
| Rabphilin | | 5 |
| Radixin | RDX | 3 |
| RAS-associated protein, RAB3A | RAB3A | 5 |
| Rathke pouch homeobox, RPX | RPX | 6 |
| Receptor tyrosine kinase (RTK), NSK2 | NSK2 | 6 |
| Retina pigment epithelium specific protein | RPE65 | 3 |
| (65 kD) | | |
| Retinitis pigmentosa gene 1 | RP1 | 3 |
| Retinitis pigmentosa gene 2 | RP2 | 3 |
| Retinitis pigmentosa gene 3 | RP3 | 3 |
| Retinitis pigmentosa gene 6 | RP6 | 3 |
| Retinitis pigmentosa gene 7 | RP7, RDS | 3 |
| Retinoblastoma 1 | RB1 | 6 |
| Retinoic acid receptor, alpha | RARA | 6 |
| Retinoic acid receptor, beta | RARB | 6 |
| Retinoic acid receptor, gamma | RARG | 6 |
| Retinoid X receptor, alpha | RXRA | 6 |
| Retinoid X receptor, beta | RXRB | 6 |
| Retinoid X receptor, gamma | RXRG | 6 |
| Retinol binding protein 4 | RBP4 | 2 |
| Rhodopsin | RHO | 3 |
| RIGUI | RIGUI | 6 |
| Rim | | 5 |
| Rod outer segment membrane protein 1 | ROM1 | 3 |
| Ryanodine receptor 1, skeletal | RYR1 | 6 |
| Serotonin N-acetyltransferase | SNAT | 1 |
| Serotonin receptor, 5HT1A | HTR1A | 5 |
| Serotonin receptor, 5HT1B | HTRI1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sex hormone binding globulin, SHBG | | 2 |
| Sialoprotein, bone | BSP | 6 |
| Signal transducer and activator of | STAT1 | 6 |
| transcription 1 | | |
| Signaling lymphocyte activation molecule | SLAM | 4 |
| Sine oculis homeobox, drosophila, homolog 1 | SIX1 | 6 |
| Sine oculis homeobox, drosophila, homolog 2 | SIX2 | 6 |
| Sine oculis homeobox, drosophila, homolog 5 | SIX5 | 3 |
| Sjoegren (Sjogren) syndrome antigen A1 | SSA1 | 4 |
| Slug protein | | 6 |
| Small nuclear ribonucleoprotein polypeptide N | SNRPN | 3 |
| Smoothelin | SMTN | 6 |
| Smoothened (Drosophila) homolog | SMOH | 6 |
| Sodium channel, non-voltage gated 1, alpha | SCNN1A | 5 |
| Sodium channel, non-voltage gated 1, beta | SCNN1B | 5 |
| Sodium channel, non-voltage gated 1, | SCNN1G | 5 |
| gamma | | |
| Sodium channel, voltage gated, type IV, | SCN4A | 5 |
| alpha polypeptide | | |
| Sodium channel, voltage gated, type V, alpha | SCN5A | 5 |
| polypeptide | | |
| Sodium channel, voltage-gated, type 1, beta | SCN1B | 5 |
| polypeptide | | |
| Solute carrier family 1 (glutamate | SLC1A1 | 2 |
| transporter), member 1 | | |
| Solute carrier family 1 (glutamate | SLC1A2 | 2 |
| transporter), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 17, member 1 | SLC17A1 | 2 |
| Solute carrier family 17, member 2 | SLC17A2 | 2 |
| Solute carder family 19 (folate transporter), | SLC19A1 | 2 |
| member 1 | | |
| Solute carrier family 21, member 2 | SLC21A2 | 2 |
| Solute carrier family 21, member 3 | SLC21A3 | 2 |
| Solute carrier family 25, member 12 | SLC25A12 | 2 |
| Solute carrier family 6 (GAMMA- | SLC6A1 | 2 |
| AMINOBUTYRIC ACID transporter), member 1 | | |
| Solute carrier family 6 (neurotransmifter | SLC6A3 | 2 |
| transporter, dopamine), member 3 | | |
| Solute carrier family 6 (neurotransmifter | SLC6A2 | 2 |
| transporter, noradrenaline), member 2 | | |
| Solute carrier family 6, member IO | SLC6A10 | 2 |
| Solute carrier family 6, member 8 | SLC6A8 | 2 |
| Solute carrier family 7(amino acid | SLC7A1 | 2 |
| transporter), member 1 | | |
| Solute carrier family 7(amino acid | SLC7A2 | 2 |
| transpo er), member 2 | | |
| Solute carrier family 7(amino acid | SLC7A7 | 2 |
| transporter), member 7 | | |
| Solute carrier family 8 (sodium/calcium | SLC8A1 | 2 |
| exchanger), member 1 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Sonic hedgehog, SHH | SHH | 6 |
| Sorbitol dehydrogenase | SORD | 1 |
| Sorcin | SRI | 2 |
| Spectrin alpha | SPTA1 | 3 |
| Spectrin beta | SPTB | 3 |
| Sperm adhesion molecule | SPAM1 | 6 |
| Sperm prolamine P1 | PRM1 | 6 |
| Sperm prolamine P2 | PRM2 | 6 |
| Sphingomyelinase | SMPD1 | 1 |
| Split hand/foot malformation gene | DSS1 | 6 |
| SRY-box 10 | SOX10 | 6 |
| SRY-box 11 | SOX11 | 6 |
| SRY-box 3 | SOX3 | 6 |
| SRY-box 4 | SOX4 | 6 |
| SRY-box 9 | SOX9 | 6 |
| Steroid 5 alpha reductase 1 | SRD5A1 | 1 |
| Steroid 5 alpha reductase 2 | SRD5A2 | 1 |
| Steroid sulphatase | STS | 1 |
| Substance P | | 5 |
| Succinate dehydrogenase 1 | SDH1 | 1 |
| Succinate dehydrogenase 2 | SDH2 | 1 |
| Sulfamidase | SGSH | 6 |
| Superoxide dismutase 1 | SOD1 | 1 |
| Superoxide dismutase 3 | SOD3 | 1 |
| Survival of motor neuron 1, telomeric | SMN1 | 2 |
| Synapsin I a & I b | SYN1 | 5 |
| Synapsin 2a & 2b | SYN2 | 5 |
| Synaptic vesicle protein 2 | SV2 | 5 |
| Synaptobrevin 1 | SYB1 | 5 |
| Synaptobrevin 2 | SYB2 | 5 |
| Synaptogyrin | | 5 |
| Synaptophysin | SYP | 5 |
| Synaptosomal-associated protein, 25 KD | SNAP25 | 5 |
| Synaptotagmin 1 | SYT1 | 5 |
| Synaptotagmin 2 | SYT2 | 5 |
| Synovial sarcoma gene 1 | SSX1 | 6 |
| Synovial sarcoma gene 2 | SSX2 | 6 |
| Syntaxin 1 | STX1 | 5 |
| Tachykinin receptor, NK1R | TACR1 | 5 |
| Tachykinin receptor, NK2R | TACR2 | 5 |
| Tachykinin receptor, NK3R | TACR3 | 5 |
| Talin, TLN | | 3 |
| T-BOX 1 | TBX1 | 6 |
| T-BOX 2 | TBX2 | 6 |
| T-BOX 3 | TBX3 | 6 |
| T-BOX 4 | TBX4 | 6 |
| T-BOX 5 | TBX5 | 6 |
| T-BOX 6 | TBX6 | 6 |
| TEK, tyrosine kinase, endothelial | TEK | 1 |
| Telomerase protein component | | 1 |
| Tetranectin | TNA | 2 |
| Thrombospondin | THBS1 | 6 |
| Thromboxane A synthase 1 | TBXAS1 | 4 |
| Thromboxane A2 | TXA2 | 4 |
| Thromboxane A2 receptor | TBXA2R | 4 |
| Thymosin | | 4 |
| Thyrotropin releasing hormone | TRH | 5 |
| Thyrotropin releasing hormone | TRH | 6 |
| Thyrotropin releasing hormone receptor | TRHR | 5 |
| Tip-associated protein | TAP | 4 |
| Tissue non-specific alkaline phosphatase | | 1 |
| TNSAP | | |
| Titin | TTN | 3 |
| Tocopherol (alpha) transfer protein | TTPA | 2 |
| Torticollis, keloids, cryptorchidism and renal | TKCR | 6 |
| dysplasia gene | | |
| Transforming growth factor, alpha | TGFA | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Transglutaminase 1 | TGM1 | 6 |
| Transglutaminase 2 | TGM2 | 6 |
| Transglutaminase 4 | TGM4 | 6 |
| Transthyretin | TTR | 2 |
| Treacle gene | TCOF1 | 6 |
| Triosephosphate isomerase | TPI 1 | 1 |
| Tropomyosin 1 alpha | TPM1 | 3 |
| Tropomyosin 3 (non-muscle) | TPM3 | 3 |
| Troponin C | | 3 |
| Troponin I | TNNI3 | 3 |
| Troponin T2, cardiac | TNNT2 | 3 |
| Tryypsinogen 1 | TRYI | 1 |
| Trypsinogen 2 | TRY2 | 1 |
| Tubby-like protein | TULP1 | 6 |
| Tuberous sclerosis 1 | TSC1 | 6 |
| Tuberous sclerosis 2 | TSC2 | 6 |
| Tumor susceptibility gene 101 | TSG101 | 6 |
| Tumour necrosis factor (TNF) receptor | TRAF1 | 4 |
| associated factor 1 | | |
| Tumour necrosis factor (TNF) receptor | TRAF2 | 4 |
| associated factor 2 | | |
| Tumour necrosis factor (TNF) receptor | TRAF3 | 4 |
| associated factor 3 | | |
| Tumour necrosis factor (TNF) receptor | TRAF4 | 4 |
| associated factor 4 | | |
| Tumour necrosis factor (TNF) receptor | TRAF5 | 4 |
| associated factor 5 | | |
| Tumour necrosis factor (TNF) receptor | TRAF6 | 4 |
| associated factor 6 | | |
| Tumour necrosis factor alpha | TNFA | 4 |
| Tumour necrosis factor alpha receptor | TNFAR | 4 |
| Tumour necrosis factor beta | TNFB | 4 |
| Tumour necrosis factor beta receptor | TNFBR | 4 |
| Tumour protein p53 | TP53, P53 | 6 |
| Tumour protein p63 | TP63 | 6 |
| Tumour protein p73 | TP73 | 6 |
| Tumour protein, translationally-controlled 1 | TPT1 | 6 |
| Tumour suppresssor gene DRA | DRA | 4 |
| Tyrosinase | TYR | 1 |
| Tyrosinase-related protein 1 | TYRP1 | 1 |
| Tyrosine aminotransferase | TAT | 1 |
| Ubiquitin activating enzyme, E1 | | 1 |
| Ubiquitin protein ligase E3A | UBE3A | 1 |
| Uncoupling protein 3 | UCP3 | 2 |
| Undulin 1 | COL14AI | 3 |
| Uroporphyrinogen decarboxylase | UROD | 1 |
| Usher syndrome 2A | USH2A | 3 |
| Vacuolar proton pump, subunit 1 | VPP1 | 5 |
| Vacuolar proton pump, subunit 3 | VPP3 | 5 |
| Vascular endothelial growth factor | VEGF | 6 |
| Vasoactive intestinal polypeptide | VIP | 5 |
| Vasoactive intestinal polypeptide receptor | VIPR | 5 |
| Villin | | 3 |
| Vinculin | | 3 |
| Vitamin D receptor | VDR | 6 |
| Vitelliform macular dystrophy, atypical gene | VMD1 | 2 |
| Von Hippel-Lindau gene | VHL | 6 |
| Von Willebrand factor | VWF | 2 |
| Werner syndrome helicase | WRN | 6 |
| Winged helix nude | WHN | 6 |
| Wingless family, wnt2 | WNT2 | 6 |
| Wingless family, wnt4 | WNT4 | 6 |
| Wingless family, wnt5 | WNT5 | 6 |
| Wingless family, wnt7 | WNT7 | 6 |
| Wingless family, wnt8 | WNT8 | 6 |
| Wiskoft-Aldrich syndrome protein | WASP, THC | 4 |
| Wnt inhibitory factor, WIF-1 | WIF1 | 6 |
| Wolf-Hirschhorn syndrome candidate 1 gene | WHSC1 | 6 |
| Wolfram syndrome 1 gene | WFS1 | 3 |
| Xeroderma pigmentosum, complementation | XPA | 1 |
| group A | | |
| Xeroderma pigmentosum, complementation | XPB | 1 |
| group B | | |
| Xeroderma pigmentosum, complementation | XPC | 1 |
| group C | | |
| Xeroderma pigmentosum, complementation | | 1 |
| group D | | |
| Xeroderma pigmentosum, complementation | | 1 |
| group E | | |
| Xeroderma pigmentosum, complementation | XPF | 1 |
| group F | | |
| Xeroderma pigmentosum, complementation | ERCC5 | 1 |
| group G | | |
| X-ray repair gene | XRCC9 | 6, |

that the genes associated with endocrine and metabolic malfunction, damage or disease are selected from:

| | HUGO | |
|---|---|---|
| List of genes | gene symbol | Protein-function |
| 17beta Hydroxysteroid dehydrogenase 1 | HSD17B1 | 1 |
| 17beta Hydroxysteroid dehydragenase 3 | HSD17B3 | 1 |
| 17beta Hydroxysteroid dehydrogenase 4 | HSD17B4 | 1 |
| 17beta Hydroxysteroid oxidoreductase | | 1 |
| 17-Ketosteroid reductase | | 5 |
| 18-Hydroxysteroid oxidoreductase | | 1 |
| 2,3-Bisphosphoglycerate mutase | BPGM | 1 |
| 2,4-Dienoyl CoA reductase | DECR | 1 |
| 3 beta Hydroxysteroid dehydrogenase 2 | HSD3B2 | 1 |
| 3-Oxoacid CoA transferase | OXCT | 1 |
| 5-Adenosyl homocysteine hydrolase | | 1 |
| 6-Phosphofructo-2-kinase | PFKFBL | 1 |
| 6-Pyruvoyltetrahydropterin synthase | PTS | 1 |
| Acetoacetyl 1-CoA-thiolase | ACAT1 | 1 |
| Acetyl CoA acyltransferase | ACAA | 1 |
| Acetyl CoA carboxylase | ACC | 1 |
| Acetyl CoA carboxylase alpha | ACACA | 1 |
| Acetylcholinesterase | ACHE | 1 |
| Acid phosphatase 2, lysosomal | ACP2 | 1 |
| Actin, alpha, cardiac | ACTC | 3 |
| Actin, alpha, skeletal | ACTA1 | 3 |
| Actin, alpha, smooth, aortic | ACTA2 | 3 |
| Activin | | 6 |
| Activin A receptor, type 2B | ACVR2B | 6 |
| Activin A receptor, type 2-like kinase 1 | ACVRL1 | 6 |
| Acyl CoA dehydrogenase, long chain | ACADL | 1 |
| Acyl CoA dehydrogenase, medium chain | ACADM | 1 |
| Acyl CoA dehydrogenase, short chain | ACADS | 1 |
| Acyl CoA dehydrogenase, very long chain | ACADVL | 1 |
| Acyl CoA synthetase, long chain, 1 | LACS1 | 1 |
| Acyl CoA synthetase, long chain, 2 | LACS2 | 1 |
| Acyl CoA synthetase, long chain, 4 | ACS4 | 1 |
| Acyl malonyl condensing enzyme | | 1 |
| Adenomatous polyposis coli tumour | APC | 6 |
| supressor gene | | |
| Adenosine deaminase | ADA | 1 |
| Adenosine monophosphate deaminase | AMPD | 1 |
| Adenosine receptor A1 | ADORA1 | 5 |
| Adenosine receptor A2A | ADORA2A | 5 |
| Adenosine receptor A28 | ADORA2B | 5 |
| Adenosine receptor A3 | ADORA3 | 5 |
| Adenyl cyclase | | 5 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adenylate transferase | | 1 |
| ADP-ribosyltransferase | ADPRT | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenoleukodystrophy gene | ALD | 1 |
| Albumin, ALB | ALB | 2 |
| Alcohol dehydrogenase | ADH | 1 |
| Alcohol dehydrogenase 2 | ADH2 | 1 |
| Alcohol dehydrogenase 3 | ADH3 | 1 |
| Alcohol dehydrogenase 4 | ADH4 | 1 |
| Alcohol dehydrogenase 5 | ADH5 | 1 |
| Alcohol dehydrogenase 6 | ADH6 | 1 |
| Alcohol dehydrogenase 7 | ADH7 | 1 |
| Aldehyde dehydrogenase 1 | ALDH1 | 1 |
| Aldehyde dehydrogehase 10 | ALDH10 | 1 |
| Aldehyde dehydrogenase 2 | ALDH2 | 1 |
| Aldehyde dehydrogenase 5 | ALDH5 | 1 |
| Aldehyde dehydrogenase 6 | ALDH6 | 1 |
| Aldehyde dehydrogenase 7 | ALDH7 | 1 |
| Aldolase A | ALDOA | 1 |
| Aldolase B | ALDOB | 1 |
| Aldolase C | ALDOC | 1 |
| Aldosterone receptor | MLR | 6 |
| Alkaline phosphatase, liver/bone/kidney | ALPL | 2 |
| Alkylglycerone phosphate synthase | AGPS | 1 |
| Alpha 1 acid glycoprotein | AAG; AGP | 2 |
| alpha1-antitrypsin | PI | 1 |
| alpha-actinin 2 | ACTN2 | 6 |
| alpha-actinin 3 | ACTN3 | 6 |
| alpha-amino adipic semialdehyde synthase | | 1 |
| alpha-glucosidase, neutral AB | GANAB | 1 |
| alpha-glucosidase, neutral C | GANC | 1 |
| alpha-ketoglutarate dehydrogenase | | 1 |
| Aminomethyltransferase | AMT | 1 |
| Aminopeptidase P | XPNPEP2 | 1 |
| Amphiregulin | AREG | 6 |
| Amylo-1,6-glucosidase | AGL | 1 |
| Androgen receptor | AR | 6 |
| Angiopoietin 1 | ANGPT1 | 6 |
| Angiopoietin 2 | ANGPT2 | 6 |
| Angiotensin converting enzyme | ACE, DCPL | 1 |
| Angiotensin receptor 1 | AGTR1 | 2 |
| Angiotensin receptor 2 | AGTR2 | 2 |
| Anglotensinogen | AGT | 1 |
| Anti-Multerian, hormone | AMH | 6 |
| Anti-Mullerian hormone type 2 receptor | AMHR2 | 6 |
| Apolipoprotein A I | APOA1 | 2 |
| Apolipoprotein A II | APOA2 | 2 |
| Apolipoprotein B | APOB | 2 |
| Apolipoprotein C1 | APOC1 | 2 |
| Apolipoprotein C2 | APOC2 | 2 |
| Apolipoprotein C3 | APOC3 | 2 |
| Apolipoprotein D | APOD | 2 |
| Apolipoprotein E | APOE | 2 |
| Apolipoprotein H | APOH | 2 |
| Aquaporin 1 | AQP1 | 2 |
| Aquaporin 2 | AQP2 | 2 |
| Arginine vasopressin | AVP | 5 |
| Arginine vasopressin receptor 1A | AVPR1A | 5 |
| Arginine vasopressin receptor 2B | AVPR1B | 5 |
| Arginine vasopressin receptor 2 | AVPR2 | 5 |
| Asparagine synthetase | AS | 1 |
| Aspartate transcarbamoylase | | 1 |
| Ataxia telangiectasia complementation group D | ATD, ATDC | 6 |
| Ataxia telangiectasia gene, AT | ATM | 6 |
| ATP cobalamin adenoxyltransferase | | 1 |
| Atrial natriuretic peptide | ANP | 6 |
| Atrial natriuretic peptide receptor A | NPR1 | 6 |
| Atrial natriuretic peptide receptor B | NPR2 | 6 |
| Atdal natriuretic peptide receptor C | NPR3 | 6 |
| Attractin | | |
| Autoimmune regulator, AIRE | AIRE | |
| beta-endorphin receptor | | 5 |
| beta-galactosidase | GLB I | 1 |
| beta-ketoacyl reductase | | 1 |
| Bile acid coenzyme A: amino acid N- | BAAT | 1 |
| acyltransferase | | |
| Bile salt export pump | BSEP, PFIC2 | 2 |
| Bile salt-stimulated lipase | CEL | 1 |
| Bilirubin UDP-glucuronosyltransferase | | 1 |
| Bloom syndrome protein | BLM | 6 |
| Bradykinin receptor B1 | | 4 |
| Bradykinin receptor B2 | | 4 |
| Branched chain aminotransferase 1, cytosolic | BCAT1 | 1 |
| Branched chain aminotransferase 2, | BCAT2 | 1 |
| mitochondrial | | |
| Branched chain keto acid dehydrogenase E1, | BCKDHA | 1 |
| alpha polypeptide | | |
| Branched chain keto acid dehydrogenase E1, | BCKDHB | 1 |
| beta polypeptide | | |
| Butyrylcholinesterase | BCHE | 1 |
| C17-20 desmolase | | 1 |
| C3 convertase | | 1 |
| Calbindin 1 | CALB1 | 6 |
| Calbindin DgK | CALB3 | 6 |
| Calcineurin A1 | CALNA1 | 4 |
| Calcineurin A2 | CALNA2 | 4 |
| Calcineurin A3 | CALNA3 | 4 |
| Calcineurin B | | 4 |
| Calcitonin receptor/Calcitonin gene-related | CALCR | 5 |
| peptide receptor | | |
| Calcitonin/Calcitonin gene-related peptide alpha | CALCA | 5 |
| Calcium channel, voltage-dependent, alpha | CACNA1F | 5 |
| I F subunit | | |
| Calcium channel, voltage-dependent, Alpha- | CACNA1B | 5 |
| I B (CACNL1A5) | | |
| Calcium channel, voltage-dependent, Alpha-1 C | CACNA1C | 5 |
| Calcium channel, voltage-dependent, Alpha-I D | CACNA1D | 5 |
| Calcium channel, voltage-dependent, Alpha- | CACNA1E | 5 |
| 1 E (CACN1A6) | | |
| Calcium channel, voltage-dependent, Alpha- | CACNA2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, L type, | CACNALS | 5 |
| alpha 1S subunit | | |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calcium sensing receptor | CASR | 2 |
| Calmodulin 1 | CALM1 | 6 |
| Calmodutin 2 | CALM2 | 6 |
| Calmodulin 3 | CALM3 | 6 |
| Calmodulin-dependant protein kinase II | CAMK2A | 6 |
| Calnexin | CANX | 6 |
| Calpain | CAPN, CAPN3 | 1 |
| Calretinin | CALB2 | 5 |
| Canalicular multispecific organic anion | CMOAT | 2 |
| transporter | | |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Carboxylesterase 1 | CES1 | 1 |
| Cardiac-specific homeobox, CSX | CSX | 6 |
| Carnitine acetyltransferase | CRAT | 1 |
| Carnitine acylcarnitine translocase | CACT | 1 |
| Camitine palmitoyltransferase I | CPT1A | 1 |
| Carnitine palmitoyltransferase II | CPT2 | 1 |
| Camitine transporter protein | CDSP, SCD | 2 |
| Carnosinase | | 5 |
| Cartilage-hair hypoplasia gene | CHH | 5 |
| Catechol-o-methyltransferase | COMT | 1 |
| Cell adhesion molecule, intercellular, ICAM | ICAM1 | 6 |
| Cell adhesion molecule, leukocyte- | LECAM1 | 6 |
| endothelial, LECA (CD62) | | |
| Cell adhesion molecule, liver, LCAM | LCAM | 6 |
| Cell adhesion molecule, neural, NCAM1 | NCAM1 | 6 |
| Cell adhesion molecule, neural, NCAM2 | NCAM2 | 6 |
| Cell adhesion molecule, platelet-endothelial, | PECAM1 | 6 |
| PECAM | | |
| Cell adhesion molecule, vascular, VCAM | VCAM1 | 6 |
| c-erbB2 | ERBB2 | 6 |
| c-erbB3 | ERBB3 | 6 |
| c-erbB4 | ERBB4 | 6 |
| Chitotriosidase | CHIT | 1 |
| Cholecystokinin | CCK | 5 |
| Cholecystokinin B receptor | CCKBR | 5 |
| Cholesterol ester hydroxylase | | 1 |
| Cholesterol ester transfer protein | CETP | 2 |
| Choline acetyltransferase | CHAT | 1 |
| Chromogranin A | CHGA | 6 |
| Chymase | CHY1 | |
| Citrate synthase | | 1 |
| Clathrin | | 2 |
| Clusterin | CLU | 6 |
| CoA transferase | | 1 |
| Collagen IV alpha 5 | COL4A5 | 3 |
| Collagen IV alpha 6 | COL4A6 | 3 |
| Complex III | | 1 |
| Complex V | MTATP6 | 1 |
| Corticosteroid binding globulin | CBG | 5 |
| Corticotrophin-releasing hormone | CRH | 2 |
| Corticotrophin-releasing hormone receptor | CRHR1 | 2 |
| Cortisol receptor | | 4 |
| Cubilin | CUBN | 2 |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclin-dependent kinase inhibitor 1C (P57, | CDKN1C | 6 |
| KIP2) | | |
| Cyclin-dependent kinase inhibitor 2A (pl 6) | CDKN2A | 6 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYPIA2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4AII | CYP4AII | 1 |
| CYP4BI | CYP4BI | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cystic fibrosis transmembrane conductance | CFTR | 5 |
| regulator, CFTR | | |
| Cystinosin | CTNS | 2 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| DAX1 nuclear receptor | DAX1 | 4 |
| D-beta-hydroxybutyrate dehydrogenase | | 1 |
| Dehydratase | | 1 |
| Delta 4-5 oxosteroid isomerase | | 1 |
| Delta aminolevulinate synthase 1 | ALAS1 | 1 |
| Delta aminolevuiinate synthase 2 | ALAS2 | 1 |
| Deoxycorticosterone (DOC) receptor | | 1 |
| Deoxyuridine triphosphatase; dUTPase | | 1 |
| DHEA sulfotransferase | STD | 1 |
| Dihydrodiol dehydrogenase 1 | DDH1 | 1 |
| Dihydrolipoamide branched chain | DBT | 5 |
| transacylase | | |
| Dihydrolipoamide dehydrogenase | DLD | 5 |
| Dihydrolipoyl dehydrogenase 2 | PDHA | 1 |
| Dihydrolipoyl transacetylase | PDHA | 1 |
| Dihydroorotase | | 1 |
| Dihydropyramidinase | DPYS | 1 |
| Dihydroxyacetonephosphate acyltransferase | DHAPAT | 1 |
| Dihyropyrimidine dehydrogenase | DPYD | 1 |
| DNA glycosylases | | 1 |
| DNA helicases | | 1 |
| DNA Ligase 1 | LIG1 | 1 |
| DNA methyltransferase | DNMT | 1 |
| DOPA decarboxylase | DDC | 1 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dynamin | DNM1 | 6 |
| Electron-transfering-flavoprotein alpha | ETFA | 2 |
| Electron-transfering-flavoprotein beta | ETFB | 2 |
| Electron-transferring flavoprotein | ETFDH | 1 |
| dehydrogenase | | |
| Endometrial bleeding-associated factor | EBAF | 6 |
| Endothelin converting enzyme | ECEI | 5 |
| Endothelin receptor type A | EDNRA | 5 |
| Endothelin receptor type B | EDNRB | 5 |
| Enolase | ENO1 | 1 |
| Enoyl CoA reductase | | 1 |
| Enterokinase | PRSS7, ENTK | 1 |
| Ephrin receptor tyrosine kinase A | EPHA | 6 |
| Ephrin receptor tyrosine kinase B | EPHB | 6 |
| Epidermal growth factor | EGF | 6 |
| Epidermal growth factor receptor | EGFR | 6 |
| Erythropoietin | EPO | 4 |
| Estrogen receptor | ESR | 6 |
| Excision repair complementation, group 1 | ERCC1 | 1 |
| protein | | |
| Factor 1 (No. one) | F1 | 4 |
| FADH dehydrogenase | | 1 |
| Fatty acid binding proteins FABP2 | FABP2 | 2 |
| Fc fragment of IgG, high affinity IA, receptor | FCGRIA | 6 |
| for | | |
| Fc fragment of IgG, low affinity IIa, receptor | FCGR2A | 6 |
| for (CD32) | | |
| Fc fragment of IgG, low affinity IIIa, receptor | FCGR3A | 6 |
| for (CDI 6) | | |
| Ferritin, H subunit | | 2 |
| Ferritin, L subunit | FTL | 2 |
| Fibrinogen alpha | FGA | 3 |
| Fibrinogen beta | FGB | 3 |
| Fibrinogen gamma | FGG | 3 |
| Fibroblast growth factor | FGFI | 6 |
| Fibroblast growth factor receptor 1 | FGFR1 | 6 |
| Fibroblast growth factor receptor 2 | FGFR2 | 6 |
| Fibroblast growth factor receptor 3 | FGFR3 | 6 |
| Flavin-containing monooxygenase 1 | FMOL | 1 |
| Flavin-containing monooxygenase 2 | FMO2 | 1 |
| Flavin-containing monooxygenase 3 | FMO3 | 1 |
| Flavin-containing monooxygenase 4 | FMO4 | 1 |
| Follicle stimulating hormone receptor | FSHR, ODG1 | 6 |
| Follicle stimulating hormone, FSH | FSHB | 6 |
| Follistatin | | 6 |
| Frataxin | FRDA | 6 |
| Fructose-1,6-diphosphatase | FBP1 | 1 |
| Fumarase | FH | 1 |
| Fumarylacetoacetase | FAH | 1 |
| GABA receptor, alpha 1 | GABRA1 | 5 |
| GABA receptor, alpha 2 | GABRA2 | 5 |
| GABA receptor, alpha 3 | GABRA3 | 5 |
| GABA receptor, alpha 4 | GABRA4 | 5 |
| GABA receptor, alpha 5 | GABRA5 | 5 |
| GABA receptor, alpha 6 | GABRA6 | 5 |
| GABA receptor, beta 1 | GABRB1 | 5 |
| GABA receptor, beta 2 | GABRB2 | 5 |
| GABA receptor, beta 3 | GABRB3 | 5 |
| GABA receptor, gamma 1 | GABRG1 | 5 |
| GABA receptor, gamma 2 | GABRG2 | 5 |
| GABA receptor, gamma 3 | GABRG3 | 5 |
| GABA transaminase | ABAT | 1 |
| Galactocerebrosidase | GALC | 1 |
| Galactokinase | GALK1 | 1 |
| Galactose 1-phosphate uridyl-transferase | GALT | 1 |
| Galanin | GAL | 5 |
| Galanin receptor | GALNRI | 5 |
| Gamma-glutamyl carboxylase | GGCX | 2 |
| Gamma-glutamyltransferase 1 | GGT1 | 2 |
| Gamma-glutamyltransferase 2 | GGT2 | 2 |
| Gap junction protein beta 1 | GJB1 | 2 |
| Gap junction protein beta 3 | GJB3 | 2 |
| Gastric inhibitory polypeptide GIP | GIP | 2 |
| Gastric inhibitory polypeptide receptor, GIPR | GIPR | 2 |
| Gastric Intrinsic factor, GIF | GIF | 1 |
| Gastric lipase, LIPF | | 2 |
| Gastrin | GAS | 6 |
| Gastrin releasing peptide | GRP | 2 |
| Gastrin releasing peptide receptor | GRPR | 2 |
| Glucagon receptor | GCGR | 6 |
| Glucagon synthase | | 2 |
| Glucagon-like peptide receptor 1 | GLP1 R | 6 |
| Glucocorticoid receptor | GRL | 6 |
| Glucokinase | GCK | 1 |
| Glucosaminyl (N-acetyl) transferase 2, I- | GCNT2 | 1 |
| branching enzyme | | |
| Glucose-6-phosphatase | G6PC | 1 |
| Glucose-6-phosphatase translocase | G6PT1 | 1 |
| Glucose-6-phosphate dehydrogenase | G6PD | 1 |
| Glucosidase, acid beta | GBA | 1 |
| Glutamate decarboxylase, GAD | GAD1 | 1 |
| Glutamate dehydrogenase | GLUD1 | 1 |
| Glutamine phosphoribosylpyrophosphate | | 1 |
| amidotransferase/PRPP amidotransferase | | |
| Glutamine synthase | | 1 |
| Glutathione | GSH | 2 |
| Glutathione peroxidase, GPX2 | GPX2 | 1 |
| Glutathione reductase, GSR | GSR | 1 |
| Glutathione S-transferase, GSTZ1 | GSTZ1 | 1 |
| Glutathione synthetase | GSS | 1 |
| Glyceraldehyde-3-phosphate dehydmgenase, GAPDH | | 1 |
| GAPDH | | |
| Glycerol kinase | GK | 1 |
| Glycerophosphate d hydrogenase 2 | GPD2 | 1 |
| Glycinamide ribonucleotide (GAR) | GART | 1 |
| transformylase | | |
| Glycine dehydrogenase | GLDC | 1 |
| Glycogen branching enzyme | GBE1 | 1 |
| Glycogen phosphorylase | PYGL | 1 |
| Glycogen synthase 1 (muscle) | GLYS1 | 1 |
| Glycogen synthase 2 (liver) | GYS2 | 1 |
| Glycosyltransferases, ABO blood group | ABO | 1 |
| Gonadotropin releasing hormone | GNRH | 6 |
| Gonadotropin releasing hormone receptor | GNRHR | 6 |
| Growth arrest-specific homeobox | GAX | 6 |
| Growth hormone 1 | GH1 | 6 |
| Growth hormone 2 (placental) | GH2 | 6 |
| Growth hormone receptor | GHR | 6 |
| Growth hormone releasing hormone (GHRH) | GHRH | 6 |
| Growth hormone releasing hormone receptor | GHRHR | 6 |
| GTP cylcohydrolase I | GCHI | 6 |
| GTPase-activating protein, GAP | RASAI | 6 |
| Guanidinoacetate N-methyltransferase | GAMT | 1 |
| Guanine nucleotide-binding protein, alpha | GNA01 | 5 |
| activating activity polypeptide, GNAO | | |
| Guanine nucleotide-binding protein, alpha | GNA11 | 5 |
| inhibiting activity polypeptide 1, GNA11 | | |
| Guanine nucleotide-binding protein, alpha | GNA12 | 5 |
| inhibiting activity polypeptide 2, GNA12 | | |
| Guanine nucleotide-binding protein, alpha | GNA13 | 5 |
| inhibiting activity polypeptide 3, GNA13 | | |
| Guanine nucleotide-binding protein, alpha | GNAS1 | 5 |
| stimulating activity polypeptide, GNAS1 | | |
| Guanine nucleotide-binding protein, alpha | GNAS2 | 5 |
| stimulating activity polypeptide, GNAS2 | | |
| Guanine nucleotide-binding protein, alpha | GNAS3 | 5 |
| stimulating activity polypeptide, GNAS3 | | |
| Guanine nucleotide-binding protein, alpha | GNAS4 | 5 |
| stimulating activity polypeptide, GNAS4 | | |
| Guanine nucleotide-binding protein, alpha | GNAT1 | 5 |
| transducing activity polypeptide, GNAT1 | | |
| Guanine nucleotide-binding protein, alpha | GNAT2 | 5 |
| transducing activity polypeptide, GNAT2 | | |
| Guanine nucleotide-binding protein, beta | GNB3 | 5 |
| polypeptide 3 | | |
| Guanine nucleotide-binding protein, gamma | GNG5 | 5 |
| polypeptide 5 | | |
| Guanine nucleotide-binding protein, q | GNAQ | 5 |
| polypeptide | | |
| Guanylate cyclase 2D, membrane (retina- | GUCY2D | 1 |
| specific) | | |
| Guanylate cyclase activator 1A (retina) | GUCA1A | 1 |
| Guanylate kinase | | 1 |
| Guanylin | GUCA2 | 2 |
| Guanylyl cyclase | | 1 |
| Heat shock protein, HSP60 | | 4 |
| Heat shock protein, HSP70 | | 4 |
| Heat shock protein, HSP90 | | 4 |
| Heat shock protein, HSPA1 | | 4 |
| Heat shock protein, HSPA2 | | 4 |
| Hemopexin | HPX | 4 |
| Heparin binding epidermal growth factor | HBEGF | 6 |
| Hepatic lipase | LIPC | 1 |
| Hepatic nuclear factor-3-beta | HNF3B | 1 |
| Hepatic nuclear factor-4-alpha | HNF4A | 1 |
| Hexokinase 1 | HK1 | 1 |
| Hexokinase 2 | HK2 | 1 |
| Hexosaminidase A | HEXA, TSD | 1 |
| Hexosaminidase B | HEXB | 1 |
| Histamine receptors, H1 | | 5 |
| Histamine receptors, H2 | | 5 |
| Histamine receptors, H3 | | 5 |
| HMG-CoA lyase | HMGCL | 1 |
| HMG-CoA reductase | HMGCR | 1 |
| HMG-CoA synthase | HMGCS2 | 1 |
| Holocarboxylase synthetase | HLCS | 1 |
| Holoprosencephaly 1 | HPE1 | 6 |
| Holoprosencephaly 2 | HPE2 | 6 |
| Holoprosencephaly 3 | HPE3 | 6 |
| Holoprosencephaly 4 | HPE4 | 6 |
| Homeobox (HOX) gene A13 | HOXA13 | 6 |
| Hormone-sensitive lipase | HSL | 1 |
| HSSB, replication protein | | 1 |
| Human chorionic gonadtrophin, hCG | CG | 6 |
| Human placental lactogen | CSH1 | 6 |
| Hydroxyacyl glutathione hydrolase | HAGH | 1 |
| Hypoxanthine-guanine | HPRT | 1 |
| phosphoribosyltransferase, HGPRT | | |
| Hypoxia inducible factor 1 | HIF1A | 1 |
| Hypoxia inducible factor 2 | | 1 |
| Iduronate 2 sulphatase | IDS | 1 |
| Immunoglobulin E (IgE) reponsiveness gene | IGER | 4 |
| Immunoglobulin E (IgE) serum concentration | IGES | 4 |
| regulator gene | | |
| Immunoglobulin gamma (IgG) 2 | IGHG2 | |
| Indian hedgehog, ihh | IHH | 6 |
| Inhibin, alpha | INHA | 6 |
| Inhibin, beta A | INHBA | 6 |
| Inhibin, beta B | INHBB | 6 |
| Inhibin, beta C | INHBC | 6 |
| Inosine monophosphate dehydrogenase, | | 1 |
| IMPDH | | |
| Inosine triphosphatase | ITPA | 1 |
| Inositol 1,4,5-triphosphate receptor I | ITPR1 | 6 |
| lnositol monophosphatase | IMPA1 | 5 |
| Inositol polyphosphate 1-phosphatase | INPP1 | 5 |
| Insulin | INS | 6 |
| Insulin receptor | INSR | 6 |
| Insulin receptor substrate-1 | IRS1 | 6 |
| Insulin-like growth factor 1 | IGF1 | 6 |
| Insulin-like growth factor 1 receptor | IGF1R | 6 |
| Insulin-like growth factor 2 | IGF2 | 6 |
| Insulin-like growth factor 2 receptor | IGF2R | 6 |
| lntegrin beta 1 | TGB1 | 6 |
| Integrin beta 2 | ITGB2 | 6 |
| lnterleukin(IL) 1 receptor | IL1R | |
| lnterleukin(IL) 1, alpha | IL1A | |
| Interleukin(IL) 1, beta | IL1B | |
| Interleukin(IL) 10 | IL10 | |
| Interleukin(IL) 10 receptor | IL10R | |
| Interleukin(IL) 11 | IL11 | |
| Interleukin(IL) 11 receptor | IL11R | |
| Interleukin(IL) 12 | IL12 | |
| Interleukin(IL) 12 receptor, beta 1 | IL12RB1 | |
| Interleukin(IL) 13 | IL13 | |
| lnterleukin(IL) 13 receptor | IL13R | |
| Interleukin(IL) 2 | IL2 | |
| Interleukin(IL) 2 receptor, alpha | IL2RA | |
| Interleukin(IL) 2 receptor, gamma | IL2RG | |
| Interleukin(IL) 3 | IL3 | |
| Interleukin(IL) 3 receptor | IL3R | |
| Interleukin(IL) 4 | IL4 | |
| Interleukin(IL) 4 receptor | IL4R | |
| lnterleukin(IL) 5 | IL5 | |
| Interleukin(IL) 5 receptor | IL5R | |
| Interleukin(IL) 6 | IL6 | |
| Interleukin(IL) 6 receptor | IL6R | |
| lnterleukin(IL) 7 | IL7 | |
| lnterleukin(IL) 7 receptor | IL7R | |
| Interleukin(IL) 8 | IL8 | |
| Interleukin(IL) 8 receptor | IL8R | |
| Interleukin(IL) 9 | IL9 | |
| Interleukin(IL) 9 receptor | IL9R | |
| Interleukin(IL) receptor antagonist 1 | IL1RN, IL1RA | 4 |
| Iodothyronine-5′-deiodinase, type 1 and 2 | | 1 |
| IP3 kinase | | 1 |
| Islet amyloid polypeptide | IAPP | 5 |
| Isocitrate dehydrogenase | | 1 |
| Isovaleric acid CoA dehydrogenase | IVD | 1 |
| Janus kinase 1 | JAK1 | 6 |
| Janus kinase 2 | JAK2 | 6 |
| Janus kinase 3 | JAK3 | 6 |
| Kallman syndrome gene | KAL1 | 6 |
| Ketohexokinase | KHK | 1 |
| Ketolase | | 1 |
| Lactase | | 1 |
| Lactotransferrin | LTF | 2 |
| Laminin 5, alpha 3 | LAMA3 | 6 |
| Laminin 5, beta 3 | LAMB3 | 6 |
| Laminin receptor 1 | LAMR1 | 6 |
| Lecithin-cholesterol acyltransferase | LCAT | 1 |
| Leptin | LEP | 6 |
| Leptin receptor | LEPR | 6 |
| Leukotriene C4 synthase | LTC4S | 1 |
| LH/choriogonadotropin (CG) receptor | LHCGR | 6 |
| Lipoamide dehydrogenase | OGDH | 1 |
| Lipoprotein lipase | LPL | 4 |
| Lipoprotein, High Density | HDLDT1 | 2 |
| Lipoprotein, Intermediate Density | | 2 |
| Lipoprotein, Low Density 1 | | 2 |
| Lipoprotein, Low Density 2 | | 2 |
| Lipoprotein, Very Low Density | VLDLR | 2 |
| Lipoprotein-associated coagulation factor | LACI | 4 |
| Lipoxygenase | | 1 |
| Lipoxygenase 12 (platelets) | LOG12 | |
| Lipoxygenase 5 (leukocytes) | | |
| Luteinizing hormone, beta chain | LHB | 6 |
| Lymphocyte-specific protein tyrosine kinase | LCK | 4 |
| Lysosomal acid lipase | LIPA | 1 |
| MAD (mothers against decapentaplegic, | MADH2 | 6 |
| Drosophila) homologue 2 | | |
| Malate dehydrogenase, mitochondrial | MDH2 | 1 |
| Malonyl CoA decarboxylase | | 1 |
| Malonyl CoA transferase | | 1 |
| Maltase-glucoamylase | | 1 |
| Mannosidase, alpha B lysosomal | MANB | 1 |
| Mannosyl (alpha-1,6-)-glycoprotein beta-1,2- | MGAT2 | 2 |
| N-acetylglucosaminyltransferase | | |
| Marenostrin | MEFV | 2 |
| Matrix Gla protein | MGP | 6 |
| MEK kinase, MEKK | | 1 |
| Melanocortin 2 receptor | MC2R | 2 |
| Melanocortin 4 receptor | MC4R | 2 |
| Menin | MEN1 | 6 |
| Methionine adenosyltransferase | MAT1A, MAT2A | 1 |
| Methionine synthase | MTR | 1 |
| Methionine synthase reductase | MTRR | 1 |
| Methylguanine-DNA methyltransferase | MGMT | 1 |
| Methylmalonyl-CoA mutase | MUT | 1 |
| Mitochondrial trifunctional protein, alpha | HADHA | 1 |
| subunit | | |
| Mitochondrial trifunctional protein, beta | HADHB | 1 |
| subunit | | |
| Molybdenum cofactor synthesis 1 | MOCS1 | 1 |
| Molybdenum cofactor synthesis 2 | MOCS2 | 1 |
| Monoamine oxidase A | MAOA | 1 |
| Monoamine oxidase B | MAOB | 1 |
| Multidrug resistance associated protein | MRP | 6 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Muscle phosphorylase | PYGM | 1 |
| Na+, K+ ATPase, alpha | ATP1A1 | 6 |
| Na+, K+ ATPase, beta 1 | ATP1B1 | 6 |
| Na+, K+ ATPase, beta 2 | ATP1B2 | 6 |
| Na+, K+ ATPase, beta 3 | ATP1B3 | 6 |
| Na+/H+ exchanger 1 | NHE1 | 2 |
| Na+/H+ exchanger 2 | NHE2 | 2 |
| Na+/H+ exchanger 3 | NHE3 | 2 |
| Na+/H+ exchanger 4 | NHE4 | 2 |
| Na+/H+ exchanger 5 | NHE5 | 2 |
| N-acetyltransferase 1 | NAT1 | 1 |
| N-acetyltransferase 2 | NAT2 | 1 |
| NADH dehydrogenase (ubiquinone) Fe—S | NDUFS1 | 1 |
| protein 1 | | |
| NADH dehydrogenase (ubiquinone) Fe—S | NDUFS4 | 1 |
| protein 4 | | |
| NADH dehydrogenase (ubiquinone) | NDUFV1 | 1 |
| avoprotein 1 | | |
| NADH-cytochrome b5 reductase | DIA1 | 1 |
| reductase | | |
| NADPH-dependent cytochrome P450 reductase | POR | 1 |
| Nephronophthisis 1 | NPHP1 | 2 |
| Nephrosis 1 | NPHS1 | 2 |
| Nerve growth factor | NGF | 6 |
| Nerve growth factor receptor | NGFR | 6 |
| Neuraminidase sialidase | NEU | 2 |
| Neuregulin | HGL | 6 |
| Neuroendocrine convertase 1 | NEC1, PCSK1 | 1 |
| Neurofibromin 1 | NF1 | 6 |
| Neurofibromin 2 | NF2 | 6 |
| Neuropeptide Y | NPY | 5 |
| Neuropeptide Y receptor Y1 | NPYI R | 5 |
| Neuropeptide Y receptor Y2 | NPY2R | 5 |
| Neurotensin | NTS | 5 |
| Neurotensin receptor | NTSR1 | 5 |
| Neurotrophin 3 | NTF3 or NT3 | 6 |
| Neutral endopeptidase | | 1 |
| Niemann-Pick disease protein | NPC1 | 2 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Notch ligand-jagged | JAG1, AGS | 6 |
| Nucleoside diphosphate kinase-A | NDPKA | 1 |
| Oncogene ret | RET | 6 |
| Oncogene sis | PDGFB | 6 |
| Orexin | OX | 6 |
| Orexin 1 receptor | OX1R | 6 |
| Orexin 2 receptor | OX2R | 6 |
| Omithine delta-aminotransferase | OAT | 1 |
| Ornithine transcarbamoylase | OTC, NME1 | 1 |
| Oxytocin | OXT | 5 |
| Oxytocin receptor | OXTR | 5 |
| Paired box homeotic gene 6 | PAX6 | 6 |
| Paired box homeotic gene 8 | PAX8 | 6 |
| Palmitoyi-protein thioesterase | PPT | 2 |
| Pancreatic lipase | PNLIP | 1 |
| Paraoxonase PON1 | PON1 | 1 |
| Paraoxonase PON2 | PON2 | 1 |
| Paraoxonase PON3 | | 1 |
| Parathyroid hormone | PTH | 6 |
| Parathyroid hormone receptor | PTHR1 | 6 |
| Parathyroid hormone related-peptide | PTHRP | 6 |
| Parathyroid hormone-like hormone | PTHLH | 6 |
| Peanut-like 1 | PNUTL1 | 4 |
| Peptidylglycine alpha-amidating | PAM | 1 |
| monooxygenase | | |
| Peroxidase, salivary | SAPX | 1 |
| Peroxisomal membrane protein 3 | PXMP3 | 2 |
| Peroxisome biogenesis factor 1 | PEX1 | 2 |
| Peroxisome biogenesis factor 19 | PEX19 | 2 |
| Peroxisome biogenesis factor 6 | PEX6 | 2 |
| Peroxisome biogenesis factor 7 | PEX7 | 2 |
| Peroxisome proliferative activated receptor, | PPARA | 2 |
| alpha | | |
| Peroxisome proliferative activated receptor, | PPARG | 2 |
| gamma | | |
| P-glycoprotein I | PGY1 | 2 |
| P-glycoprotein 3 | PGY3 | 2 |
| Phenylaianine hydroxylase | PAH | 1 |
| Phenylatanine monooxygenase | | 1 |
| Phenylethanolamine N-methyltransferase, | PNMT | 1 |
| PNMT | | |
| Phosphodiesterase 1/nucleotide | PDNP1 | 6 |
| pyrophosphatase 1 | | |
| Phosphodiesterase 1/nucleotide | PDNP2 | 6 |
| pyrophosphatase 2 | | |
| Phosphodiesterase 1/nucleotide | PDNP3 | 6 |
| pyrophosphatase 3 | | |
| Phosphoenolpyruvate carboxykinase | PCK1 | 1 |
| Phosphofructokinase, liver | PFKL | 1 |
| Phosphofructokinase, muscle | PFKM | 1 |
| Phosphoglucomutase | | 1 |
| Phosphoglucose isomerase | GPI | 1 |
| Phosphoglycerate kinase 1 | PGK1 | 1 |
| Phosphoglycerate mutase 2 | PGAM2 | 1 |
| Phospholipase A2, group 10 | PLA2G10 | 4 |
| Phospholipase A2, group 1B | PLA2G 1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Phosphomannomutase 2 | PMM2 | 6 |
| Phosphomannomutase 2 | PMM2 | 2 |
| Phosphomannose isomerase-1, PMI | MPI | 2 |
| Phosphoribosyl pyrophosphate synthetase | PRPS1 | 1 |
| Phosphorylase kinase deficiency, liver | PHK | 1 |
| Phosphorylase kinase, alpha 1 (muscle) | PHKA1 | 1 |
| Phosphorylase kinase, alpha 2 | PHKA2 | 1 |
| Phosphorylase kinase, beta | PHKB | 1 |
| Phosphorylase kinase, delta | | 1 |
| Phosphorylase kinase, gamma 2 | PHKG2 | 1 |
| Phytanoyl-CoA hydroxylase | PHYH | 6 |
| Pineolytic beta-receptors | | 1 |
| Pituitary adenylate cyclase activating peptide | PACAP | 5 |
| Pituitary adenylate cyclase activating peptide | PACAPIR | 5 |
| receptor | | |
| Plasminogen activator receptor, Urokinase | UPAR; PLAUR | 3 |
| Plasminogen activator, Tissue | PLAT; TPA | 1 |
| Plasminogen activator, Urokinase | UPA; PLAU | 1 |
| Platelet derived growth factor | PDGF | 6 |
| Platelet derived growth factor receptor | PDGFR | 6 |
| Poly (ADP-ribose) synthetase | PARS | 1 |
| Polycystin 1 | PKD1 | 2 |
| Polycystin 2 | PKD2 | 2 |
| Porphobilinogen deaminase | HMBS | 1 |
| Potassium inwardly-rectifying channel J1 | KCNJ1 | 5 |
| Potassium inwardly-rectifying channel J11 | KCNJ11 | 5 |
| Potassium voltage-gated channel A1 | KCNA1 | 5 |
| Potassium voltage-gated channel E1 | KCNE1 | 5 |
| Potassium voltage-gated channel Q1 | KCNQ1 | 5 |
| Preproenkephalin | PENK | 5 |
| Preproglucagon | GCG; GLP1; GLP2 | 6 |
| Preproglucagon | | 2 |
| Preproinsulin | | 2 |
| Profibrinolysin | | 6 |
| receptor) | | |
| Prolactin | PRL | 6 |
| Prolactin receptor | PRLR | 6 |
| Prolactin releasing hormone | PRH | 6 |
| Proliferin | PLF | 6 |
| Proline dehydrogenase | PRODH | 1 |
| Proline-rich protein BstNI subfamily 1 | PRB1 | 3 |
| Proline-rich protein BstNI subfamily 3 | PRB3 | 3 |
| Proline-rich protein BstNI subfamily 4 | PRB4 | 3 |
| Pro-melanin-concentrating hormone | PMCH | 6 |
| Proopiomelanocortin | POMC | 5 |
| Prophet of Pit1 | PROP1 | 6 |
| Prostacyclin synthase | | 4 |
| Prostaglandin (PG) D synthase, | PGDS | 1 |
| hematopoietic | | |
| Prostaglandin 15-OH dehydrogenase | HGPD; PGDH | 4 |
| Prostaglandin D - DP receptor | | 4 |
| Prostaglandin E1 receptor | | 4 |
| Prostaglandin E2 receptor | | 4 |
| Prostaglandin E3 receptor | | 4 |
| Prostaglandin F - FP receptor | | 4 |
| Prostaglandin I2 receptor | | 2 |
| Prostaglandin IP receptor | | 4 |
| Prostaglandin isomerase | | 6 |
| Prostasin, PRSS8 | PRSS8 | 1 |
| Protease nexin 2 | PN2 | 1 |
| Protein kinase B | PRKB | |
| Protein kinase C, alpha | PRKCA | 1 |
| Protein S | PROS | 4 |
| Protoporphyrinogen oxidase | PPOX | 1 |
| Pterin-4-alpha-carbinolamine | PCBD | |
| Pyrroline-5-carboxylate synthetase | PYCS | 1 |
| Pyruvate carboxylase | PC | 1 |
| Pyruvate decarboxylase | PDHA | 1 |
| Pyruvate kinase | PKLR | 1 |
| Quinoid dihydropteridine reductase | QDPR | 1 |
| Rathke pouch homeobox, RPX | RPX | 6 |
| Relaxin H1 | RLN1 | 6 |
| Relaxin H2 | RLN2 | 6 |
| Renin | REN | 1 |
| Replication factor C | RFC2 | 1 |
| Retinal pigment epithelium specific protein | RPE65 | 3 |
| (65 kD) | | |
| Retinaldehyde binding protein 1 | RLBP1 | 2 |
| Retinoic acid receptor, alpha | RARA | 6 |
| Retinoic acid receptor, beta | RARB | 6 |
| Retinoic acid receptor, gamma | RARG | 6 |
| Retinoid X receptor, alpha | RXRA | 6 |
| Retinoid X receptor, beta | RXRB | 6 |
| Retinoid X receptor, gamma | RXRG | 6 |
| Retinol binding protein 1 | | 2 |
| Retinol binding protein 2 | | 2 |
| Ribosephosphate pyrophosphokinase | | 1 |
| RIGUI | RIGUI | 6 |
| Ryanodine receptor 1, skeletal | RYRI | 6 |
| S100 calcium-binding protein A3 | S100A3 | 5 |
| S100 calcium-binding protein A4 | S100A4 | 5 |
| S100 calcium-binding protein A7 | S100A7 | 5 |
| S100 calcium-binding protein A8 | S100A8 | 5 |
| S100 calcium-binding protein A9 | S100A9 | 5 |
| S100 calcium-binding protein B | S100B | 5 |
| S100 calcium-binding protein P | S100P | 5 |
| S-Adenosylmethionine decarboxylase, AMD | | 1 |
| Salivary amylase, AMY1 | | 2 |
| Secretin | SCT | 2 |
| Secretin receptor, SCTR | SCTR | 2 |
| Serine hydroxymethyltransferase | SHMT | 1 |
| Serotonin N-acetyltransferase | SNAT | 1 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Serum amyloid A | SAA | 2 |
| Serum amyloid P | SAP | 2 |
| Sex determining region Y, SRY | SRY | 6 |
| Sex hormone binding globulin, SHBG | | 2 |
| Sodium channel, non-voltage gated 1, alpha | SCNN1A | 5 |
| Sodium channel, non-voltage gated 1, beta | SCNN1B | 5 |
| Sodium channel, non-voltage gated 1, | SCNN1G | 5 |
| gamma | | |
| Sodium channel, voltage-gated, type 1, beta | SCN1B | 5 |
| polypeptide | | |
| Solute carrier family 1 (amino acid | SLC1A6 | 2 |
| transporter), member 6 | | |
| Solute carrier family 1 (neutral amino acid | SLC1A4 | 2 |
| transporter), member 4 | | |
| Solute carrier family 10 (sodium/bile acid | SLC1OAL | 2 |
| cotransporter family), member 1 | | |
| Solute carrier family 10 (sodium/bile acid | SLC1OA2 | 2 |
| cotransporter family), member 2 | | |
| Solute carrier family 12, member 1 | SLC12A1 | 2 |
| Solute carrier family 12, member 2 | SLC12A2 | 2 |
| Solute carrier family 12, member 3 | SLC12A3 | 2 |
| Solute carrier family 14, member 2 | SLC14A2 | 2 |
| Solute carrier family 15 (H+/peptide | SLC15A1 | 2 |
| transporter, intestinal), member 1 | | |
| Solute carrier family 15 (H+/peptide | SLC15A2 | 2 |
| transporter, kidney), member 2 | | |
| Solute carder family 16 (monocarboxylate | SLC16A1 | 2 |
| transporter), member 1 | | |
| Solute carrier family 16 (monocarboxylate | SLC16A7 | 2 |
| transporter), member 7 | | |
| Solute carrier family 17, member 1 | SLC17A1 | 2 |
| Solute carrier family 17, member 2 | SLC 17A2 | 2 |
| Solute carrier family 2 (facilitated glucose | SLC2A1 | 2 |
| transporter), member 1 | | |
| Solute carrier family 2 (facilitated glucose | SLC2A2 | 2 |
| transporter), member 2 | | |
| Solute carrier family 2 (facilitated glucose | SLC2A3 | 2 |
| transporter), member 3 | | |
| Solute carrier family 2 (facilitated glucose | SLC2A4 | 2 |
| transporter), member 4 | | |
| Solute carrier family 2 (facilitated glucose | SLC2A5 | 2 |
| transporter), member 5 | | |
| Solute carrier family 20, member 3 | SLC2OA3 | 2 |
| Solute carrier family 21, member 2 | SLC21 | 2 |
| Solute carrier family 21, member 3 | SLC2lA3 | 2 |
| Solute carrier family 22, member 1 | SLC22A1 | 2 |
| Solute carrier family 22, member 2 | SLC22A2 | 2 |
| Solute carrier family 22, member 5 | SLC22A5 | 2 |
| Solute carrier family 3 (facilitated glucose | SLC3A1 | 2 |
| transporter), member 1 | | |
| Solute carrier family 4 (anion exchanger), | SLC4A1 | 2 |
| member 1 | | |
| Solute carrier family 4 (anion exchanger), | SLC4A2 | 2 |
| member 2 | | |
| Solute carrier family 4 (anion exchanger), | SLC4A3 | 2 |
| member 3 | | |
| Solute carrier family 5 (sodium/glucose | SLC5A1 | 2 |
| transporter), member 1 | | |
| Solute carrier family 5 (sodium/glucose | SLC5A2 | 2 |
| transporter), member 2 | | |
| Solute carrier family 5 (sodium/glucose | SLC5A5 | 2 |
| transporter), member 5 | | |
| Solute carrier family 5, member 3 | SLC5A3 | 2 |
| Solute carrier family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Solute carrier family 6 (neurotransmitter | SLC6A4 | 2 |
| transporter, serotonin), member 4 | | |
| Solute carrier family 6, member 10 | SLC6A10 | 2 |
| Solute carrier family 6, member 6 | SLC6A6 | 2 |
| Solute carrier family 6, member 8 | SLC6A8 | 2 |
| Solute carrier family 7(amino acid | SLC7A | 2 |
| transporter), member 1 | | |
| Solute carrier family 7(amino acid | SLC7A2 | 2 |
| transporter), member 2 | | |
| Solute carrier family 7(amino acid | SLC7A7 | 2 |
| transporter), member 7 | | |
| Solute carrier family 8 (sodium/calcium | SLC8A1 | 2 |
| exchanger), member 1 | | |
| Somatostatin | SST | 5 |
| Somatostatin receptor, SSTR1 | SSTR1 | 5 |
| Somatostatin receptor, SSTR2 | SSTR2 | 6 |
| Somatostatin receptor, SSTR3 | SSTR3 | 5 |
| Somatostatin receptor, SSTR4 | SSTR4 | 5 |
| Somatostatin receptor, SSTR5 | SSTR5 | 5 |
| Somatotrophin | | 6 |
| Sorcin | SRI | 2 |
| SOS1 guanine nucleotide exchange factor | SOS1 | 6 |
| Sperm prolamine P1 | PRM1 | 6 |
| Sperm prolamine P2 | PRM2 | 6 |
| Sphingomyelinase | SMPD1 | 1 |
| SRY-box 10 | SOX10 | 6 |
| SRY-box 11 | SOX11 | 6 |
| SRY-box 3 | SOX3 | 6 |
| SRY-box 4 | SOX4 | 6 |
| SRY-box 9 | SOX9 | 6 |
| Steroid sulphatase | STS | 1 |
| Steroidogenic acute regulatory protein | STAR | 2 |
| Substance P | | 5 |
| Succinyl CoA synthase | | 1 |
| Sucrase | | 1 |
| Sulfonylurea receptor | SUR | 6 |
| Superoxide dismutase 1 | SOD1 | 1 |
| Superoxide dismutase 3 | SOD3 | 1 |
| Surfeit | SURF1 | 6 |
| T-BOX 1 | TBX1 | 6 |
| T-BOX 3 | TBX3 | 6 |
| Thiolase, perioxisomal | | 1 |
| Thiopurine S-methyltransferase | TPMT | 1 |
| Thrombospondin | THBS1 | 6 |
| Thromboxane A synthase 1 | TBXAS1 | 4 |
| Thromboxane A2 | TXA2 | 4 |
| Thromboxane A2 receptor | TBXA2R | 4 |
| Thymopoietin | TMPO | 6 |
| Thymosin | | 4 |
| Thyroglobulin | TG | 6 |
| Thyroid hormone receptor, alpha | THRA | 6 |
| Thyroid hormone receptor, beta | THRB | 6 |
| Thyroid peroxidase | TPO | 6 |
| Thyroid receptor auxiliary protein | TRAP | 6 |
| Thyroid-stimulating hormone receptor | TSHR | 6 |
| Thyroid-stimulating hormone, alpha | TSHA | 6 |
| Thyroid-stimulating hormone, beta | TSHB | 6 |
| Thyrotropin releasing hormone | TRH | 6 |
| Thyrotropin releasing hormone receptor | TRHR | 6 |
| Thyroxin-binding globulin | TBG | 2 |
| Transacylase | | 1 |
| Transcobalamin 2, TCN2 | TCN2 | 2 |
| Transcription factor 1, hepatic | TCF1 | 6 |
| Transcription factor 2, hepatic | TCF2 | 6 |
| Transcription termination factor, RNA | TTF1 | 6 |
| polymerase 1 | | |
| Transferrin | TF | 6 |
| Transferrin receptor | TFRC | 6 |
| Transforming growth factor, beta 2 | TGFB2 | 6 |
| Transforming growth factor, beta induced | TGFBI | 6 |
| Transforming growth factor, beta receptor 2 | TGFBR2 | 6 |
| Transketolase | TKT | 1 |
| Transketolase-like 1 | TKTL1 | 1 |
| Transthyretin | TTR | 2 |
| Tubby-like protein 1 | TULP1 | 6 |
| Tuberous sclerosis 1 | TSC1 | 6 |
| Tuberous sclerosis 2 | TSC2 | 6 |
| Tyrosinase | TYR | 1 |
| Tyrosinase-related protein 1 | TYRPI | 1 |
| Tyrosine aminotransferase | TAT | 1 |
| Tyrosine hydroxylase | TH | 1 |
| Ubiquitin activating enzyme, E1 | | 1 |
| Ubiquitin protein ligase E3A | UBE3A | 1 |
| UDP-glucose pyrophosphorylase | | 1 |
| UDP-glucuronosyltransferase 1 | ugt1d, UGT1 | 1 |
| UDP-glucuronosyltransferase 2 | UGT2 | 1 |
| Uncoupling protein 1 | | 2 |
| Uncoupling protein 3 | UCP3 | 2 |
| Urate oxidase | UOX | 1 |
| Ureidopropionase | | 1 |
| Uridine monophosphate kinase | UMPK | 4 |
| Uddine monophosphate synthetase | UMPS | 4 |
| Uridinediphosphate(UDP)-galactose-4- | GALE | 1 |
| epimerase | | |
| Uroporphyrinogen decarboxylase | UROD | 1 |
| Uteroglobin | UGB | 2 |
| Vasoactive intestinal polypeptide | VIP | 5 |
| Vasoactive intestinal polypeptide receptor | VPR | 5 |
| Vasoinhibitory peptide | | 6 |
| Von Hippel-Lindau gene | VHL | 6 |
| Werner syndrome helicase | WRN | 6 |
| Wolfram syndrome I gene | WFS1 | 3 |
| Xylito dehydrogenase | | 1, |

that the genes associated with headaches are selected from:

| List of genes | HUGO gene symbol | Protein-function |
|---|---|---|
| Acetylcholinesterase | ACHE | 1 |
| Adenyfate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Angiopoietin 1 | ANGPT1 | 6 |
| Angiopoietin 2 | ANGPT2 | 6 |
| Angiotensin converting enzyme | ACE, DCPL | 1 |
| Angiotensin receptor 1 | AGTR1 | 2 |
| Angiotensin receptor 2 | AGTR2 | 2 |
| Angiotensinogen | AGT | 1 |
| Arginase | ARG1 | 1 |
| Arginine vasopressin | AVP | 5 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Calcitonin/Calcitonin gene-related peptide alpha | CALCA | 5 |
| Calcium channel, voltage-dependent, alpha 1F | CACNA1F | 5 |
| subunit | | |
| Calcium channel, voltage-dependent, Alpha- | CACNA1B | 5 |
| 1B (CACNL1A5) | | |
| Calcium channel, voltage-dependent, Alpha-I C | CACNA1C | 5 |
| Calcium channel, voltage-dependent, Alpha-I D | CACNA1D | 5 |
| Calcium channel, voltage-dependent, Alpha- | CACNA1E | 5 |
| I E (CACNL1A6) | | |
| Calcium channel, voltage-dependent, Alpha-2/delta | CACNA2 | 5 |
| Calcium channel, voltage-dependent, Beta 1 | CACNB1 | 5 |
| Calcium channel, voltage-dependent, Beta 3 | CACNB3 | 5 |
| Calcium channel, voltage-dependent, | CACNG2 | 5 |
| Neuronal, Gamma | | |
| Calcium channel, voltage-dependent, P/Q | CACNA1A | 5 |
| type, alpha 1A subunit | | |
| Calcium channel, voltage-dependent, T-type | | 5 |
| Calnexin | CANX | 6 |
| Cannabinoid receptor | CNR1 | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Catechol-o-methyltransferase | COMT | 1 |
| Choline acetyltransferase | CHAT | 1 |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesterase 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDE4C | 1 |
| Cyclic nucleotide phosphodiesterase 5A | PDE5A | 1 |
| Cyclic nucleotide phosphodiesterase 6A | PDE6A | 1 |
| Cyclic nucleotide phosphodiesterase 68 | PD 6B | 1 |
| Cyclic nucleotide phosphodiesterase 7 | PDE7 | 1 |
| Cyclic nucleotide phosphodiesterase 8 | PDE8 | 1 |
| Cyclic nucleotide phosphodiesterase 9A | PDE9A | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2FI | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Dystonia 9 | CSE | 3 |
| Endothelin 1 | EDN1 | 5 |
| Endothelin 2 | EDN2 | 5 |
| Endothelin 3 | EDN3 | 5 |
| Endothelin converting enzyme | ECEI | 5 |
| Endothelin receptor type A | EDNRA | 5 |
| Endothelin receptor type B | EDNRB | 5 |
| Enolase | ENO1 | 1 |
| Epidermal growth factor | EGF | 6 |
| Epidermal growth factor receptor | EGFR | 6 |
| Erythropoietin receptor | EPOR | 4 |
| Glutathione | GSH | 2 |
| Glutathione S-transferase, GSTZ1 | GSTZ1 | 1 |
| Glyceraldehyde-3-phosphate dehydrogenase, GAPDH | GAPDH | 1 |
| Glycerol kinase | GK | 1 |
| Glycinamide ribonucleotide (GAR) | GART | 1 |
| transformylase | | |
| Hexosaminidase B | HEXB | 1 |
| Histamine receptors, H1 | | 5 |
| Histamine receptors, H2 | | 5 |
| Histamine receptors, H3 | | 5 |
| Hypoxia inducible factor 1 | HIF1A | 1 |
| Hypoxia inducible factor 2 | | 1 |
| Insulin | INS | 6 |
| Insulin receptor | INSR | 6 |
| Interleukin(IL) 1, alpha | IL1A | 4 |
| Interleukin(IL) 1, beta | IL1B | 4 |
| Interleukin(IL) receptor antagonist 1 | IL1RN, IL1RA | 4 |
| IP3 kinase | | 1 |
| Marenostrin | MEFV | 2 |
| Methylmalonyl-CoA mutase | MUT | 1 |
| Monoamine oxidase A | MAOA | 1 |
| Monoamine oxidase B | MAOB | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| Myogenic factor 3 | MYF3 | 6 |
| Myogenic factor 4 | MYF4 | 6 |
| Myogenic factor 5 | MYF5 | 6 |
| NADH dehydrogenase reductase | | 1 |
| Neurokinin A | NKNA | 5 |
| Neurokinin B | NKNB | 5 |
| Neuropeptide Y | NPY | 5 |
| Neuropeptide Y receptor Y1 | NPY1R | 5 |
| Neuropeptide Y receptor Y2 | NPY2R | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Phospho ipase A2, group 10 | PLA2G10 | 1 |
| Phospholipase A2, group 1B | PLA2G1B | 4 |
| Phospholipase A2, group 2A | PLA2G2A | 4 |
| Phospholipase A2, group 2B | PLA2G2B | 4 |
| Phospholipase A2, group 4A | PLA2G4A | 4 |
| Phospholipase A2, group 4C | PLA2G4C | 4 |
| Phospholipase A2, group 5 | PLA2G5 | 4 |
| Phospholipase A2, group 6 | PLA2G6 | 4 |
| Phospholipase C alpha | | 4 |
| Phospholipase C beta | | 4 |
| Phospholipase C delta | PLCD1 | 4 |
| Phospholipase C epsilon | | 4 |
| Phospholipase C gamma | PLCG1 | 4 |
| Potassium inwardly-rectifying channel J1 | KCNJ1 | 5 |
| Potassium voltage-gated channel E1 | KCNE1 | 5 |
| Potassium voltage-gated channel Q1 | KCNQ1 | 5 |
| Pmopiometanocortin | POMC | 5 |
| RIGUI | RIGUI | 6 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage-gated, type 1, beta | SCN1B | 5 |
| polypeptide | | |
| Solute carrier family 5, member 3 | SLC5A3 | 2 |
| Solute carder family 6 (GAMMA- | SLC6A1 | 2 |
| AMINOBUTYRIC ACID transporter), member 1 | | |
| Solute carrier family 6 (neurotransmitter | SLC6A3 | 2 |
| transporter, dopamine), member 3 | | |
| Solute carrier family 6 (neurotransmitter | SLC6A2 | 2 |
| transporter, noradrenaline), member 2 | | |
| Substance P | | 5 |
| Tyrosine hydroxylase | TH | 1 |
| UDP-glucuronosyltransferase 1 | ugt1d, UGT1 | 1 |
| UDP-glucuronosyltransferase 2 | UGT2 | 1 |
| Vasoactive intestinal polypeptide | VIP | 5 |
| Vasoactive intestinal polypeptide receptor | VIPR | 5 |

that the genes associated with seaual malfunctions are selected from:

| | HUGO | Protein- |
|---|---|---|
| List of genes | gene symbol | function |
| 11 beta Hydroxysteroid dehydrogenase 2 | HSD11B2 | 1 |
| Acetylcholinesterase | ACHE | 1 |
| Activin | | 6 |
| Adenylate cyclase 1 | ADCY1 | 1 |
| Adenylate cyclase 2 | ADCY2 | 1 |
| Adenylate cyclase 3 | ADCY3 | 1 |
| Adenylate cyclase 4 | ADCY4 | 1 |
| Adenylate cyclase 5 | ADCY5 | 1 |
| Adenylate cyclase 6 | ADCY6 | 1 |
| Adenylate cyclase 7 | ADCY7 | 1 |
| Adenylate cyclase 8 | ADCY8 | 1 |
| Adenylate cyclase 9 | ADCY9 | 1 |
| Adrenergic receptor, alpha1 | ADRA1 | 5 |
| Adrenergic receptor, alpha2 | ADRA2 | 5 |
| Adrenergic receptor, beta1 | ADRB1 | 5 |
| Adrenergic receptor, beta2 | ADRB2 | 5 |
| Adrenergic receptor, beta3 | ADRB3 | 5 |
| Adrenoleukodystrophy gene | ALD | 1 |
| alpha thalassemia gene | ATRX | 5 |
| Androgen binding protein | ABP | 2 |
| Angiopoietin 1 | ANGPT1 | 6 |
| Angiopoietin 2 | ANGPT2 | 6 |
| Angiotensin converting enzyme | ACE, DCP1 | 1 |
| Angiotensin receptor 1 | AGTR1 | 2 |
| Angiotensin receptor 2 | AGTR2 | 2 |
| Angiotensinogen | AGT | 1 |
| Anti-Mullerian hormone | AMH | 6 |
| Anti-Mullerian hormone type 2 receptor | AMHR2 | 6 |
| Arginase | ARG1 | 1 |
| Arginine vasopressin | AVP | 5 |
| Arginine vasopressin receptor 1A | AVPR1A | 5 |
| Arginine vasopressin receptor 1B | AVPR1B | 5 |
| Arginine vasopressin receptor 2 | AVPR2 | 5 |
| Atrial natriuretic peptide | ANP | 6 |
| Atrial natriuretic peptide receptor A | NPR1 | 6 |
| Atrial natriuretic peptide receptor B | NPR2 | 6 |
| Atrial natriuretic peptide receptor C | NPR3 | 6 |
| Autoimmune regulator, AIRE | AIRE | 4 |
| BCL2-associated X protein | BAX | 6 |
| Bloom syndrome protein | BLM | 6 |
| Calcium channel, voltage-dependent, alpha 1F | CACNA 1F | 5 |
| subunit | | |
| Calcium channel, voltage-dependent, Alpha- | CACNA 1B | 5 |
| 1 B (CACNL1A5) | | |
| Calcium channel, voltage-dependent, Alpha- | CACNA 1C | 5 |
| 1 C | | |
| Calcium channel, voltage-dependent, Alpha- | CACNA 1D | 5 |
| 1 D | | |
| Calcium channel, voltage-dependent, Alpha- | CACNA 1E | 5 |
| 1 E (CACNL1A6) | | |
| Calcium channel, voltage-dependent, Alpha- | CACNA 2 | 5 |
| 2/delta | | |
| Calcium channel, voltage-dependent, Beta 1 | CACNB1 | 5 |
| Calcium channel, voltage-dependent, Beta 3 | CACNB3 | 5 |
| Calcium channel, voltage-dependent, | CACNG2 | 5 |
| Neuronal, Gamma | | |
| Calcium channel, voltage-dependent, T-type | | 5 |
| Carbonic anhydrase 3 | CA3 | 1 |
| Carbonic anhydrase 4 | CA4 | 1 |
| Carbonic anhydrase, alpha | CA1 | 1 |
| Carbonic anhydrase, beta | CA2 | 1 |
| Catechol-o-methyltransferase | COMT | 1 |
| Choline acetyltransferase | CHAT | 1 |
| Cyclic AMP response element modulator | CREM | 6 |
| Cyclic AMP-dependent protein kinase | PKA | 1 |
| Cyclic nucleotide phosphodiesterase 1B | PDE1B | 1 |
| Cyclic nucleotide phosphodiesterase 1B1 | PDE1B1 | 1 |
| Cyclic nucleotide phosphodiesteease 2A3 | PDE2A3 | 1 |
| Cyclic nucleotide phosphodiesterase 3A | PDE3A | 1 |
| Cyclic nucleotide phosphodiesterase 3B | PDE3B | 1 |
| Cyclic nucleotide phosphodiesterase 4A | PDE4A | 1 |
| Cyclic nucleotide phosphodiesterase 4C | PDF4C | 1 |
| Cyclic nucleotide phosphodiesterase 5A | PDE5A | 1 |
| Cyclic nucteotide phosphodiesterase 6A | PDE6A | 1 |
| Cyclic nucleotide phosphodiesterase 6B | PDE6B | 1 |
| Cyclic nucleotide phosphodiesterase 7 | PDE7 | 1 |
| Cyclic nucleotide phosphodiesterase 8 | PDF8 | 1 |
| Cyclic nucleotide phosphodiesterase 9A | PDE9A | 1 |
| Cyclooxygenase 1 | COX1 | 1 |
| Cyclooxygenase 2 | COX2 | 1 |
| CYP11A1 | CYP11A1 | 1 |
| CYP11B1 | CYP11B1 | 1 |
| CYP11B2 | CYP11B2 | 1 |
| CYP17 | CYP17 | 1 |
| CYP19 | CYP19 | 1 |
| CYP1A1 | CYP1A1 | 1 |
| CYP1A2 | CYP1A2 | 1 |
| CYP1B1 | CYP1B1 | 1 |
| CYP21 | CYP21 | 1 |
| CYP24 | CYP24 | 1 |
| CYP27 | CYP27 | 1 |
| CYP27B1 | PDDR | 1 |
| CYP2A1 | CYP2A1 | 1 |
| CYP2A13 | CYP2A13 | 1 |
| CYP2A3 | CYP2A3 | 1 |
| CYP2A6V2 | CYP2A6V2 | 1 |
| CYP2A7 | CYP2A7 | 1 |
| CYP2B6 | CYP2B6 | 1 |
| CYP2C18 | CYP2C18 | 1 |
| CYP2C19 | CYP2C19 | 1 |
| CYP2C8 | CYP2C8 | 1 |
| CYP2C9 | CYP2C9 | 1 |
| CYP2D6 | CYP2D6 | 1 |
| CYP2E1 | CYP2E1 | 1 |
| CYP2F1 | CYP2F1 | 1 |
| CYP2J2 | CYP2J2 | 1 |
| CYP3A3 | CYP3A3 | 1 |
| CYP3A4 | CYP3A4 | 1 |
| CYP3A5 | CYP3A5 | 1 |
| CYP3A7 | CYP3A7 | 1 |
| CYP4A11 | CYP4A11 | 1 |
| CYP4B1 | CYP4B1 | 1 |
| CYP4F2 | CYP4F2 | 1 |
| CYP4F3 | CYP4F3 | 1 |
| CYP51 | CYP51 | 1 |
| CYP5A1 | CYP5A1 | 1 |
| CYP7A | CYP7A | 1 |
| CYP8 | CYP8 | 1 |
| Cystathionase | CTH | 1 |
| Cystathione beta synthase | CBS | 1 |
| Cytidine deaminase | CDA | 1 |
| Cytidine-5-prime-triphosphate synthetase | CTPS | 1 |
| Cytochrome a | | 1 |
| Cytochrome c | | 1 |
| Cytochrome c oxidase, MTCO | | 1 |
| Cytokine-suppressive antiinflammatory drug- | CSBP1 | 4 |
| binding protein 1 | | |
| Cytokine-suppressive antiinflammatory drug- | CSBP2 | 4 |
| binding protein 2 | | |
| DAX1 nuclear receptor | DAX1 | |
| Deleted in azoospermia | DAZ | 6 |
| Diaphanous 2 | DIAPH2 | 5 |
| Disrupted meiotic CDNA 1, homolog | DMC1 | 6 |
| Dopamine beta hydroxylase | DBH | 1 |
| Dopamine receptors D1 | DRD1 | 5 |
| Dopamine receptors D2 | DRD2 | 5 |
| Dopamine receptors D3 | DRD3 | 5 |
| Dopamine receptors D4 | DRD4 | 5 |
| Dopamine receptors D5 | DRD5 | 5 |
| Electron-transfering-flavoprotein alpha | ETFA | 2 |
| Electron-transfering-flavoprotein beta | ETFB | 2 |
| Electron-transferring flavoprotein | ETFDH | 1 |
| dehydrogenase | | |
| Endometrial bleeding-associated factor | EBAF | 6 |
| Endothelin 1 | EDN1 | 5 |
| Endothelin 2 | EDN2 | 5 |
| Endothelin 3 | EDN3 | 5 |
| Endothelin converting enzyme | ECEI | 5 |
| Endothelin receptor type A | EDNRA | 5 |
| Endothelin receptor type B | EDNRB | 5 |
| Enolase | ENO1 | 1 |
| Enoyl CoA isomerase | | 1 |
| Enterokinase | PRSS7, ENTK | 1 |
| Epidermal growth factor | EGF | 6 |
| Epidermal growth factor receptor | EGFR | 6 |
| Faciogenital dysplasia | FGD1, FGDY | 2 |
| Factor XIll A & B | F13A & F13B | 4 |
| Fanconi anemia, complementation group A | FANCA | 2 |
| Fertilin protein | FTNB | 6 |
| Flightless-II, Drosophila homolog of | FLII | 6 |
| Folic acid receptor | FOLR | 6 |
| Glutathione | GSH | 2 |
| Glutathione 5-transferase, GSTZ1 | GSTZ1 | 1 |
| G yceraldehyde-3-phosphate dehydrogenase, | GAPDH | 1 |
| GAPDH | | |
| Glycerol kinase | GK | 1 |
| Glycinamide ribonucleotide (GAR) | GART | 1 |
| transformylase | | |
| Glycogen phosphorylase | PYGL | 1 |
| Gonadotropin releasing hormone | GNRH | 6 |
| Gonadotropin releasing hormone receptor | GNRHR | 6 |
| Guanine nucleotide-binding protein, aloha | GNA11 | 5 |
| inhibiting activity polypeptide 1, GNA11 | | |
| Guanine nucleotide-binding protein, alpha | GNA12 | 5 |
| inhibiting activity polypeptide 2, GNA12 | | |
| Guanine nucleotide-binding protein, alpha | GNA13 | 5 |
| inhibiting activity polypeptide 3, GNA13 | | |
| Hexosaminidase B | HEXB | 1 |
| Holoprosencephaly 1 | HPE1 | 6 |
| Holoprosencephaly 2 | HPE2 | 6 |
| Holoprosencephaly 3 | HPE3 | 6 |
| Holoprosencephaly 4 | HPE4 | 6 |
| Human placental lactogen | CSH1 | 6 |
| Inhibin, alpha | INHA | 6 |
| inhibin, beta A | INHBA | 6 |
| Inhibin, beta B | INHBB | 6 |
| Inhibin, beta C | NHBC | 6 |
| Insulin | INS | 6 |
| Insulin receptor | INSR | 6 |
| IP3 kinase | | 1 |
| Kallman syndrome gene 1 | KAL1 | 6 |
| Laminin 5, alpha 3 | LAMA3 | 6 |
| Laminin 5, beta 3 | LAMB3 | 6 |
| Laminin receptor 1 | LAMR1 | 6 |
| Long QT-type 2 potassium channels | LQT2, KCNH2 | 2 |
| Luteinizing hormone, beta chain | LHB | 6 |
| MAD (mothers against decapentaplegic, | MADH2 | 6 |
| Drosophila) homologue 2 | | |
| Methylmalonyl-CoA mutase | MUT | 1 |
| Monoamine oxidase A | MAOA | 1 |
| Monoamine oxidase B | MAOB | 1 |
| Muscarinic receptor, M1 | CHRM1 | 5 |
| Muscarinic receptor, M2 | CHRM2 | 5 |
| Muscarinic receptor, M3 | CHRM3 | 5 |
| Muscarinic receptor, M4 | CHRM4 | 5 |
| Muscarinic receptor, M5 | CHRM5 | 5 |
| reductase | | |
| Neuropeptide Y | NPY | 5 |
| Neuropeptide Y receptor Y1 | NPY1R | 5 |
| Neuropeptide Y receptor Y2 | NPY2R | 5 |
| Nitric oxide synthase 1, NOS1 | NOS1 | 1 |
| Nitric oxide synthase 2, NOS2 | NOS2 | 1 |
| Nitric oxide synthase 3, NOS3 | NOS3 | 1 |
| Oncogene ELK1 | ELK1 | 6 |
| Oncogene ELK2 | ELK2 | 6 |
| Paired box homeotic gene 3 | PAX3 | 6 |
| Patched (Drosophila) homolog, PTCH | PTCH | 6 |
| Potassium inwardly-rectifying channel J1 | KCNJ1 | 5 |
| Potassium inwardly-rectifying channel J11 | KCNJ11 | 5 |
| Potassium voltage-gated channel A1 | KCNA1 | 5 |
| Potassium voltage-gated channel E1 | KCNE1 | 5 |
| Potassium voltage-gated channel Q1 | KCNQ1 | 5 |
| Potassium voltage-gated channel Q2 | KCNQ2 | 5 |
| Potassium voltage-gated channel Q3 | KCNQ3 | 5 |
| receptor) | | |
| Proopiomelanocortin | POMC | 5 |
| Prostasin, PRSS8 | PRSS8 | 1 |
| Ribosomal protein S4, X-linked | RPS4X | 1 |
| RIGUI | RIGUI | 6 |
| Serotonin receptor, 5HT1A | HTR1A | 5 |
| Serotonin receptor, 5HT1B | HTR1B | 5 |
| Serotonin receptor, 5HT1C | HTR1C | 5 |
| Serotonin receptor, 5HT1D | HTR1D | 5 |
| Serotonin receptor, 5HT1E | HTR1E | 5 |
| Serotonin receptor, 5HT1F | HTR1F | 5 |
| Serotonin receptor, 5HT2A | HTR2A | 5 |
| Serotonin receptor, 5HT2B | HTR2B | 5 |
| Serotonin receptor, 5HT2C | HTR2C | 5 |
| Serotonin receptor, 5HT3 | HTR3 | 5 |
| Serotonin receptor, 5HT4 | HTR4 | 5 |
| Serotonin receptor, 5HT5 | HTR5 | 5 |
| Serotonin receptor, 5HT6 | HTR6 | 5 |
| Serotonin receptor, 5HT7 | HTR7 | 5 |
| Sodium channel, non-voltage gated 1, alpha | SCNN1A | 5 |
| Sodium channel, non-voltage gated 1, beta | SCNN1B | 5 |
| Sodium channel, non-voltage gated 1, gamma | SCNN1G | 5 |
| Sodium channel, voltage gated, pe V, alpha | SCN5A | 5 |
| potypeptide | | |
| Sodium channel, voltage-gated, type 1, beta | SCN1B | 5 |
| polypeptide | | |
| Solute carrier family 6 (GAMMAAMINO- | SLC6A1 | 2 |
| BUTYRIC ACID transporter), member 1 | | |
| Solute carrier family 6 (neurotransmitter | SLC6A3 | 2 |
| transporter, dopamine), member 3 | | |
| Solute carrier family 6 (neurotransmifter | SLC6A2 | 2 |
| transporter, noradrenaline), member 2 | | |
| Sperm prolamine P1 | PRM1 | 6 |
| Sperm prolamine P2 | PRM2 | 6 |
| T-BOX 3 | TBX3 | 6 |
| Testis-specific protein Y | TSPY | 6 |
| Tyrosine hydroxylase | TH | 1 |
| UDP-glucuronosyltransferase 1 | ugt1d, UGT1 | 1 |
| UDP-glucuronosyltransferase 2 | UGT2 | 1 |
| Vasoactive intestinal polypeptide | VIP | 5 |
| Vasoactive intestinal polypeptide receptor | VIPR | 5 |
| Zona pellucida glycoprotein 1 | ZP1 | 6 |
| Zona pellucida glycoprotein 2 | ZP2 | 6 |
| Zona pellucida glycoprotein 3 | ZP3 | 6 |
| Zona pellucida receptor tyrosine kinase | ZRK | 6 |
| Zonadhosin | ZAN | 6, |

wherein the numbers listed in the column of protein function indicate:

| | |
|---|---|
| 1 | enzyme |
| 2 | transport and storage |
| 3 | structure |
| 4 | immunity |
| 5 | neuronal transmission |
| 6 | growth and differentiation |

4. The set of oligonucleotides according to claim 1, in which oligonucleotides containing up to 5% of the listed genes are not included..

5. The set of oligonucleotides according to claim 1, in which oligonucleotides containing at least 95% of the listed genes together with a limited number of additional unlisted oligonucleotides are included.

6. The set of oligonucleotides according to claim 1, in which up to 5% of the corresponding oligonucleotides of the listed genes are replaced by a complete set of 25% of other unlisted oligonucleotides.

7. The set of oligonucleotides for a target group of genes according to at least one of the preceding claims in which the chemically pretreated DNA sequence of the genes to be detected coincides at least up to 95% with the correspondingly pretreated DNA sequence of genes from the above list.

8. The set of oligonucleotides according to at least one of the preceding claims, wherein the chemical pretreatment is conducted by means of the solution of a bisulfite, hydrogen sulfite or disulfite.

9. The set of oligonucleotides according to at least one of the preceding claims, comprising a subgroup of the target groups of genes.

10. The set of oligonucleotides according to at least one of the preceding claims, in which the oligonucleotides are arranged on a support at known sites in a rectangular or hexagonal grid.

11. The set of oligonucleotides according to at least one of the preceding claims, wherein the oligonucleotides are arranged on a support which is comprised of silicon, glass, polystyrene, aluminum, steel, iron, copper, nickel, silver, or gold.

12. The set of oligonucleotides according to at least one of the preceding claims, wherein the oligonucleotide probes are labeled by means of their mass, electrostatics, charge or fluorescence, or are labeled with radionuclides.

13. Use of a set of oligonucleotides according to at least one of the preceding claims in a biological investigation for the detection of said gene variants with respect to DNA methylation.

14. A technical medical device, which contains a set of oligonucleotides according to one or more of claims 1 to 12, for use in an investigation for the detection of said gene variants, particularly as an indication of a higher risk of a patient or individual of developing symptoms and consequential signs of cancer or as an indication of a higher risk of the development of CNS malfunction,
damage or disorder, or for the patient or individual to experience symptoms and consequences of CNS malfunction, damage or disorder.

15. A technical medical device, which contains a set of oligonucleotides according to one or more of claims 1 to 12, for use in an investigation for the detection of variable gene expression and/or for the prognosis and for the management of patients, who suffer the risk of developing symptoms and consequential signs of cancer and/or for use in an investigation of whether a patient or individual may develop CNS malfunction, damage or disorder or whether it is probable that the patient or individual will experience symptoms and consequences of CNS malfunction, damage or disorder.

16. A method for the investigation of the DNA methylation profile of a patient or individual, which detects the presence or the lack of the relevant methylation variants from the target group of genes, by hybridizing a chemically pretreated nucleic acid sample of said patient or individual to a set of oligonucleotides according to one or more of claims 1-12 and relating the hybridization pattern to the variations.

17. Use of a set of oligonucleotides according to one or more of claims 1 to 12 or a device according to at least one of claims 14 or 15 to the prognosis and/or planned treatment for patients who suffer from a health problem or in whom the risks exists of the occurrence of a health problem.

18. The use according to claim 17, for the prognosis and/or planned treatment for patients who suffer from the consequences of undesired drug interactions or in whom the risk exists of the occurrence of undesired drug interactions, who suffer from the risk of developing symptoms and consequential signs of cancer, who are affected by an increased risk of CNS malfunction, damage or disorder, who suffer from the consequences of symptoms of aggression or behavioral disturbances or in whom the risk exists of the occurrence of symptoms of aggression or behavioral disturbances, who suffer from the consequences of clinical, psychological and social consequences of a brain lesion, or in whom the risk exists of the occurrence of consequences of clinical, psychological and social consequences of a brain lesion, who suffer from dementia and/or associated syndromes or in whom the risk exists of the occurrence of dementia and/or associated syndromes, who suffer from the symptoms and consequences of psychotic disturbances and personality disorders, or in whom the risk exists of the occurrence of psychotic disturbances and personality disorders, who suffer from the symptoms or consequences of cardiovascular disorder, malfunction or damage, or in whom the risk exists of the occurrence of symptoms or consequences of cardiovascular disorder, malfunction or damage, who suffer from the symptoms and consequences of malfunction, damage or disease of the gastrointestinal tract, or in whom the risk exists of the occurrence of symptoms and consequences of malfunction, damage or disease of the gastrointestinal tract, who experience clinical or social consequences which result from malfunction, damage or disease of the respiratory system or in whom the risk exists of clinical or social consequences which result from malfunction, damage or disease of the respiratory system, who suffer from symptoms and consequences of lesion, inflammation, infection, immunity and/or convalescence, or in whom the risk exists of the occurrence of symptoms and consequences of lesion, inflammation, infection, immunity and/or convalescence, who suffer from the consequences of malfunction, damage or disorder of the body as a consequence of an abnormality in the developmental process or in whom the risk exists of the occurrence of malfunction, damage or disorder of the body as a consequence of an abnormality in the developmental process, who suffer from the consequences of a malfunction, damage or disorder of the skin, the muscles, the connective tissue or the bones or in whom the risk exists of the occurrence of a malfunction, damage or disorder of the skin, the muscles, the connective tissue or the bones, who suffer from the consequences of endocrine and metabolic malfunction, damage or disorder or in whom the risk exists of the occurrence of endocrine and metabolic malfunction, damage or disorder, who suffer from the consequences of headaches or in whom the risk exists of the occurrence of headaches, and/or who suffer from the consequences of sexual malfunctions or in whom the risk exists of the occurrence of sexual malfunctions.

19. Use of a set of oligonucleotides according to one or more of claims 1 to 12 or a device according to at least one of claims 14 or 15 for predicting the probable therapeutic consequences of undesired drug interactions as a consequence of a therapeutic intervention and/or for predicting probable therapeutic consequences and adverse results as a consequence of a therapeutic intervention.

20. Use of a set of oligonucleotides according to one or more of claims 1 to 12 or a device according to at least one of claims 14 or 15 for predicting probable therapeutic risks or undesired drug interactions as a consequence of ingestion of specific medications.

21. Use of a set of oligonucleotides according to one or more of claims 1 to 12 or a device according to at least one of claims 14 or 15 for predicting probable symptoms in the case of occurrence of undesired drug interactions and the probability of the occurrence of consequential disorders or other symptoms and/or for the prediction of probable patterns of disorder symptoms and the probability of the occurrence of consequential disorders or other symptoms.

22. Use of a set of oligonucleotides according to one or more of claims 1 to 12 or a device according to at least one of claims 14 or 15 for the development of new strategies in therapeutic intervention and in clinical studies and/or for the prognosis or the management of patients who suffer from developing symptoms of aggression or behavioral disturbances or who belong to risk groups for said disturbances, and/or for modeling and evaluating the effects of disorders and precautionary health measures for individuals, ethnic groups, patient groups, and populations and/or for generating a model in order to estimate the risk for individuals, ethnic groups, patient groups, and populations, of developing symptoms and consequential signs of cancer, and/or to generate a model for the evaluation of the risk of developing symptoms and consequential signs of CNS malfunction, damage or disorder and/or for optimizing the therapeutic intervention.

23. A method for constructing a model for evaluating whether a health problem will occur or will probably occur in a patient, an individual or an ethnic group, which comprises the following steps:
a) A DNA sample is taken from a patient or individual in which a health problem has been diagnosed;
b) DNA samples taken from a control group of individuals, in which the presence of this health problem has been concluded diagnostically;
c) The samples obtained in a) and b) are analyzed to determine the DNA methylation variation within the target group of genes according to one or more of claims 1 to 12;
d) The frequency of alleles with different methylation patterns in samples from a) and b) is calculated;
e) The frequencies of the alleles in the samples from a) and b) are compared;
f) A statistical analysis is conducted of the results obtained under e) in order to obtain a model for evaluating the risk of the occurrence of health problems.

24. The method according to claim 23, wherein the health problem is selected from:
undesired drug interactions; cancer; symptoms and consequential signs of CNS malfunction, damage or disorder; developing symptoms of aggression or behavioral disturbances;
consequences of clinical, psychological and social consequences of a brain lesion; dementia and/or associated syndromes; psychotic disturbances and personality disorders; symptoms or consequences of cardiovascular disorder, malfunction or damage; symptoms and consequences of malfunction, damage or disease of the gastrointestinal tract; malfunction, damage or disease of the respiratory system;
symptoms and consequences of lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disorder; headaches; sexual malfunctions.

25. A method for evaluating whether a specific individual bears the risk of the occurrence of health problems, wherein the methylation profile is compared with the model constructed according to claim 23 or 24.

26. The method according to one or more of claims 16 or 23 to 25, wherein at least one of the steps is conducted by a computer.

27. A configured assay [kit] for utilizing the estimation of the risk of a patient or individual of experiencing adverse events and/or health problems, said kit containing:
a) Possibilities for testing for the presence or absence of key relevant polymorphic DNA variations of the core group of genes as they are defined in claims 1 to 9 in a sample of genomic DNA.
b) Reagents for use in the detection method.
c) A script, which describes the probability of a patient or individual of experiencing adverse events and/or health problems.

28. The configured assay [kit]for utilizing the estimation of the risk of a patient or individual of experiencing adverse events and/or health problems according to claim 27, further characterized in that the adverse event or the health problem is selected from:
undesired drug interactions; cancer; symptoms and consequential signs of CNS malfunction, damage or disorder; developing symptoms of aggression or behavioral disturbances;
consequences of clinical, psychological and social consequences of a brain lesion; dementia and/or associated syndromes; psychotic disturbances and personality disorders; symptoms or consequences of cardiovascular disorder, malfunction or damage; symptoms and consequences of malfunction, damage or disease of the gastrointestinal tract; malfunction, damage or disease of the respiratory system;
symptoms and consequences of lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disorder; headaches; sexual malfunctions.
